(19) United States
(12) Patent Application Publication
(10) Pub. No.: US 2018/0155344 A1
(43) Pub. Date: Jun. 7, 2018

(54) TRICYCLIC PIPERIDINE COMPOUNDS

(71) Applicant: ACTELION PHARMACEUTICALS LTD, Allschwil (CH)

(72) Inventors: Daniel BUR, Allschwil (CH); Corinna GRISOSTOMI, Allschwil (CH); Thierry KIMMERLIN, Allschwil (CH); Lubos REMEN, Allschwil (CH); Hervé SIENDT, Allschwil (CH); Magali VERCAUTEREN, Allschwil (CH); Richard WELFORD, Allschwil (CH)

(73) Assignee: ACTELION PHARMACEUTICALS LTD, Allschwil (CH)

(21) Appl. No.: 15/571,700

(22) PCT Filed: May 3, 2016
(86) PCT No.: PCT/EP2016/059818

§ 371 Date: Nov. 3, 2017

(30) Foreign Application Priority Data
May 4, 2015 (EP) PCT/EP2015/059687

(51) Int. Cl. C07D 471/14; A61P 11/00; A61P 9/00; A61P 9/12; A61P 11/06; A61P 19/10; A61P 1/00; A61P 37/06; A61P 1/10; A61P 1/04; A61P 7/02; A61P 25/20; A61P 29/00; A61P 3/10; A61P 37/00; A61P 1/16; A61P 35/00; A61P 7/04; A61P 25/06; A61P 25/00; A61P 1/08

(52) U.S. Cl. CPC C07D 471/14; A61P 1/08; A61P 9/00; A61P 9/12; A61P 11/06; A61P 19/10; A61P 1/00; A61P 37/06; A61P 1/10; A61P 1/04; A61P 7/02; A61P 25/20; A61P 29/00; A61P 3/10; A61P 37/00; A61P 1/16; A61P 35/00; A61P 7/04; A61P 25/06; A61P 25/00; A61P 11/00

(57) ABSTRACT

The present invention relates to compounds of the formula (I)

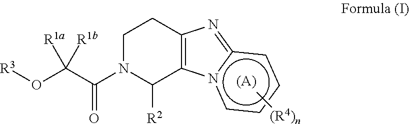

wherein R^{1a}, R^{1b}, R², R³, (R⁴)ₙ and ring (A) are as described in the description, to their preparation, to pharmaceutically acceptable salts thereof, and to their use as pharmaceuticals, to pharmaceutical compositions containing one or more compounds of formula (I), to methods for the preparation of such compounds of formula (I), and especially to their use as TPH modulators.

The present invention relates to novel tricyclic piperidine derivatives of Formula (I), and their use as pharmaceuticals. The invention also concerns related aspects including processes for the preparation of the compounds, pharmaceutical compositions containing one or more compounds of Formula (I), and especially their use as TPH inhibitors.

The biogenic amine serotonin (5HT) is a biochemical messenger and regulator that signals through 13 receptors which are distributed throughout the nervous system and peripheral organs. 5HT is synthesized in 2 steps from the dietary amino acid L-tryptophan (L-Tryp). The first and rate limiting step in the tryptophan-serotonin metabolism is the hydroxylation of L-Tryp by the non-heme pterin dependent oxygenase tryptophan hydroxylase (TPH).

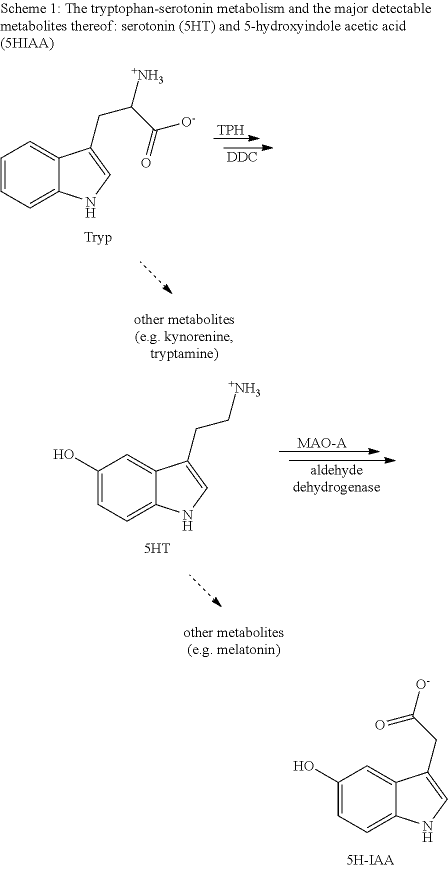

This is followed by rapid decarboxylation of 5-hydroxytryptophan by the enzyme aromatic amino acid decarboxylase (DDC). 5HT is further metabolized to 5-hydroxyindole acetic acid (5HIAA) by a combination of monoamine oxidase-A (MAO-A) and, subsequently, an aldehyde dehydrogenase. 5HIAA is excreted in the urine. An additional 5HT metabolic pathway in the pineal gland leads to production of melatonin which is involved in the circadian regulation of the sleep-wake cycle.

TPH comprises two isoforms: TPH2 is mainly expressed in neuronal cell types in the central nervous system (CNS), while TPH1 is mainly expressed in peripheral tissues, including the entrochromaffin cells (EC) in the gut, where it is responsible for synthesizing 5HT that is stored in circulating blood platelets. TPH1 and thus altered tryptophan-serotonin metabolism has been implicated as a potential drug target in a number of pathophysiologies such as lung diseases including e.g. chronic obstructive pulmonary disease (COPD), pulmonary embolism, interstitial lung disease such as lung fibrosis (Konigshoff, M. et al. (2010) “Increased expression of 5-hydroxytryptamine2A/B receptors in idiopathic pulmonary fibrosis: a rationale for therapeutic intervention.” Thorax 65(11): 949-955.), pulmonary hypertension (Ciuclan, L. et al. (2013) “Imatinib attenuates hypoxia-induced pulmonary arterial hypertension pathology via reduction in 5-hydroxytryptamine through inhibition of tryptophan hydroxylase 1 expression.” Am J Respir Crit Care Med 187(1): 78-89), radiation pneumonitis (including that giving rise to or contributing to pulmonary hypertension), asthma (Durk, T. et al. (2013). “Production of serotonin by tryptophan hydroxylase 1 and release via platelets contribute to allergic airway inflammation.” Am J Respir Crit Care Med 187(5): 476-485), adult respiratory distress syndrome (ARDS); osteoporosis (Yadav, V. K. et al. (2010) “Pharmacological inhibition of gut-derived serotonin synthesis is a potential bone anabolic treatment for osteoporosis.” Nat Med 16, 308-312); gastrointestinal disorders including inflammatory bowel disease, ulcerative colitis (Ghia, J. E. et al. (2009) “Serotonin has a key role in pathogenesis of experimental colitis.” Gastroenterology 137(5): 1649-1660), postinfectious irritable bowel syndrome, coeliac disease, idiopathic constipation, irritable bowel syndrome (Brown, P. M. et al. (2011) “The tryptophan hydroxylase inhibitor LX1031 shows clinical benefit in patients with nonconstipating irritable bowel syndrome”, Gastroenterology 141, 507-516), and carcinoid syndrome (Engelman, K., et al. (1967). “Inhibition of serotonin synthesis by para-chlorophenylalanine in patients with the carcinoid syndrome.” N Engl J Med 277(21): 1103-1108). Further examples are myxomatous valve disease (Lacerda, C. M. et al. (2012) “Local serotonin mediates cyclic strain-induced phenotype transformation, matrix degradation, and glycosaminoglycan synthesis in cultured sheep mitral valves.” Am J Physiol Heart Circ Physiol 302(10): H1983-1990); thrombosis; sleep disorders; pain; type1 and type 2 diabetes; liver disease including e.g. (viral-induced) hepatitis, fibrosis, transplantation, regeneration; acute and chronic hypertension; aortic and coronary artery disease; cancer, including e.g breast cancer (Pai V P et al. (2009) “Altered serotonin physiology in human breast cancers favors paradoxical growth and cell survival.” Breast Cancer Res. 11(6)), prostate cancer (Shinka T et al. (2011) “Serotonin synthesis and metabolism-related molecules in a human prostate cancer cell line.” Oncol Lett. March; 2(2):211-215) and neuroendocrine tumors (Hicks R J. (2010) “Use of molecular targeted agents for the diagnosis, staging and therapy of neuroendocrine malignancy.” Cancer Imaging. October 4; 10 Spec no A:S83-91); subarachnoid hemorrhage; abdominal migraine; CREST syndrome (calcinosis, Raynaud's phenomenon, esophageal dysfunction, sclerodactyly, telangiectasia); Gilbert's syndrome; nausea; serotonin syndrome; functional anorectal disorders; functional bloating; immune tolerance and inflammatory diseases including e.g. multiple sclerosis and systemic sclerosis (Nowak E C et al. (2012) “Tryptophan hydroxylase-1 regulates immune tolerance and inflammation.” J Exp Med. October 22; 209(11):2127-35; Dees C et al (2011) Platelet-derived serotonin links vascular disease and tissue fibrosis. J Exp Med. May 9; 208(5):961-72.).

TPH2 has been implicated as a potential drug target in a number of neurological health disorders including depression; anxiety including generalized anxiety disorder and social phobia; emetic disorders; migraine; substance abuse; attention deficit disorder (ADD); attention deficit hyperactivity disorder (ADHD); bipolar disorder; suicidal behavior; behavioral disorder; schizophrenia; Parkinson's disease; Huntigton's disease; autism; dyskinesia; eating disorders; type 2 diabetes; pain; Alzheimer's disease; sexual dysfunction; and brain tumors.

The role of 5HT in the brain as a neurotransmitter is well characterized. Brain 5HT is produced rapidly after uptake of circulating L-Tryp from the plasma (Hyyppa, M. T., et al. (1973) “Rapid accumulation of H3-serotonin in brains of rats receiving intraperitoneal H3-tryptophan: effects of 5,6-dihydroxytryptamine or female sex hormones”, J Neural Transm 34, 111-124). The production of brain 5HT was extensively probed in the 1990s and 2000s, with the most prominent tool being intra venous (i.v.) administration of ¹⁴C-1-methyl-tryptophan which is taken-up into the brain (Diksic, M. (2001) “Labelled alpha-methyl-L-tryptophan as a tracer for the study of the brain serotonergic system”, J Psychiatry Neurosci 26, 293-303; Diksic, M., and Young, S. N. (2001) “Study of the brain serotonergic system with labeled alpha-methyl-L-tryptophan”, J Neurochem 78, 1185-1200). A frequently noted advantage of this approach is that the produced ¹⁴C-1-methyl 5HT is not further metabolized and builds up in the brain. However, this and possible other disruptions of metabolism could equally lead to unwanted perturbations in the 5HT synthesis system caused simply by the additional methyl appendage.

In the periphery, 5HT is predominantly produced by TPH1 in a number of organs. The gut enterochromaffin cells are often cited to be the primary peripheral site of 5HT synthesis, where it plays roles amongst others in gut motor activity, visceral sensation and intestinal secretion (Bertrand, P. P., and Bertrand, R. L. (2010) “Serotonin release and uptake in the gastrointestinal tract”, Auton Neurosci 153, 47-57; Hasler, W. L. (2009) “Serotonin and the GI tract”, Curr Gastroenterol Rep 11, 383-391). Serotonin secreted from the EC eventually finds its way out of the tissue into the blood. There, 5HT is actively taken up by blood platelets, where it is stored. Activated platelets disgorge 5HT and it subsequently serves as a vasoconstrictor and helps to regulate hemostatis and blood clotting. Linder et al. (2009) recently characterized 5HT concentrations in a number of organs in the rat (Linder, A. E., et al. (2009) “Body distribution of infused serotonin in rats”, Clin Exp Pharmacol Physiol 36, 599-601). Notably the lung was found to have a similar 5HT concentration to the gut. Other researchers have measured TPH1 gene expression by qPCR and the results suggest that TPH1 is probably active in other organs including the thymus and the spleen (Walther, D. J. and M. Bader (2003). “A unique central tryptophan hydroxylase isoform.” Biochem Pharmacol 66(9): 1673-1680). Furthermore, significantly elevated 5HT concentrations are thought to be responsible for certain conditions associated with carcinoid tumors (known as carcinoid syndrome).

The earliest reported TPH inhibitor used in vivo was p-chlorophenylalanine (PCA). PCA was demonstrated to lower 5HT in both the gut (˜50% original) and the brain (˜20% original) after dosing of 200 mg/kg intra peritonial (i.p.) four times a day (qid) for 3 days (Weber, L. J. (1970) “p-Chlorophenylalanine depletion of gastrointestinal 5-hydroxytryptamine”, Biochem Pharmacol 19, 2169-2172). PCA has also shown utility in a xenograft model of cholangiocarcinoma, where a dramatic reduction in tumor volume was observed (Alpini, G., et al. (2008) “Serotonin metabolism is dysregulated in cholangiocarcinoma, which has implications for tumor growth”, Cancer Res 68, 9184-9193). Following the discovery of the peripheral TPH1 enzyme (Walther, D. J., et al. (2003) “Synthesis of serotonin by a second tryptophan hydroxylase isoform”, Science 299, 76), a number of studies indicating roles for peripheral 5HT in disease revealed the potential of TPH1 as a drug target. The company Lexicon Pharmaceuticals Ltd has synthesized and characterized a number of small molecule inhibitors of TPH1. LP533401 was demonstrated to lower gut 5HT in mice without effecting brain concentrations (Liu, Q., et al. (2008) “Discovery and characterization of novel tryptophan hydroxylase inhibitors that selectively inhibit serotonin synthesis in the gastrointestinal tract”, J Pharmacol Exp Ther 325, 47-55). LP533401 has been further characterized in both mouse and rat models of osteoporosis (Yadav, V. K., et al. (2010) “Pharmacological inhibition of gut-derived serotonin synthesis is a potential bone anabolic treatment for osteoporosis”, Nat Med 16, 308-312). LX1031 ((S)-2-Amino-3-(4-{2-amino-6-[(R)-2,2,2-trifluoro-1-(3′-methoxy-biphenyl-4-yl)-ethoxy]-pyrimidin-4-yl}-phenyl)-propionic acid, WO2007/089335) was the first TPH inhibitor from Lexicon Pharmaceuticals Ltd to enter clinical trials and similar to LP533401 lowers 5HT in the jejunum, with only a minor reduction observed in the colon and no effect on brain 5HT. In a phase IIA study LX1031 qid did not affect blood 5HT and had very modest effects on urinary 5HIAA (up to 30% reduction) (Brown, P. M., et al. (2011) “The tryptophan hydroxylase inhibitor LX1031 shows clinical benefit in patients with nonconstipating irritable bowel syndrome”, Gastroenterology 141, 507-516). A further small molecule inhibitor of TPH1 is LX1032 ((S)-2-Amino-3-[4-(2-amino-6-{(R)-1-[4-chloro-2-(3-methyl-pyrazol-1-yl)-phenyl]-2,2,2-trifluoro-ethoxy}-pyrimidin-4-yl)-phenyl]-propionic acid ethyl ester, WO2008/073933), which is disclosed to be in clinical studies for carcinoid syndrome.

The present invention, thus, provides novel tricyclic piperidine derivatives of formula (I) which are non-peptide inhibitors of human TPH potentially useful in the treatment of disorders relating to disease or disorder characterized by an altered rate of the tryptophan-serotonin metabolism, comprising especially lung fibrosis; pulmonary hypertension; asthma; osteoporosis; ulcerative colitis; irritable bowel syndrome; carcinoid syndrome; cancer including breast cancer, prostate cancer, and neuroendocrine tumors with elevated serotonin secretion (e.g carcinoid tumors); and inflammatory diseases including multiple sclerosis and systemic sclerosis.

1) In a first embodiment, the present invention relates to compounds of Formula (I)

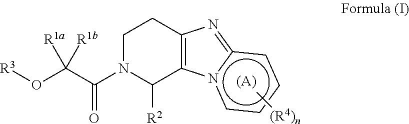

wherein

ring (A) represents a fused 6-membered aromatic ring containing the bridgehead nitrogen atom and optionally one additional ring nitrogen atom;

(R⁴)ₙ represents one or two optional substituents (i.e. n represents the integer 0, 1, or 2) independently selected from (C₁₋₄)alkyl (especially methyl, ethyl, tert.-butyl), (C₃₋₆)cycloalkyl (especially cyclopropyl), (C₁₋₃)trifluoroalkyl (especially trifluoromethyl), halogen (especially chloro), or phenyl;

R^{1a} and R^{1b} independently represent hydrogen, methyl, ethyl; or R^{1a} and R^{1b} together with the carbon atom to which they are attached to form a cyclopropyl ring;

R² represents aryl (especially phenyl), or heteroaryl (notably 5- or 6-membered heteroaryl, in particular pyridinyl, pyrimidinyl, pyrazolyl, thiazolyl, thiophenyl, oxazolyl, isoxazolyl, oxadiazolyl, thieno[2,3-b]pyridinyl, benzothiazolyl, imidazo[1,5-a]pyridinyl), wherein said aryl or heteroaryl independently is unsubstituted, or mono-, di-, or tri-substituted, wherein the substituents are independently selected from:
(C₁₋₄)alkyl (especially methyl, ethyl);
(C₁₋₄)alkoxy (especially methoxy, ethoxy);
(C₃₋₆)cycloalkyl, optionally containing one or two ring oxygen atoms (especially cyclopropyl);
(C₁₋₃)fluoroalkyl (especially trifluoromethyl);
(C₁₋₃)fluoroalkoxy (especially difluoromethoxy, trifluoromethoxy, 2,2,2-trifluoroethoxy);
halogen (especially fluoro, chloro);
cyano;
hydroxy;
—O(CH₂)₂—NR²¹R²², wherein
   R²¹ and R²² independently represent hydrogen or (C₁₋₃)alkyl (especially methyl); or
   R²¹ and R²² together with the nitrogen atom to which they are attached to form a 4- to 7-membered saturated ring, wherein said ring optionally contains one ring oxygen atom, and wherein said ring is optionally substituted with one or two fluorine substituents;
—(CH₂)ₚ—NR²³R²⁴, wherein p represents the integer 0 or 1; and
   R²³ and R²⁴ independently represent hydrogen or (C₁₋₃)alkyl (especially methyl); or
   R²³ and R²⁴ together with the nitrogen atom to which they are attached to form a 4- to 7-membered saturated ring, wherein said ring optionally contains one ring oxygen atom, and wherein said ring is optionally substituted with one or two fluorine substituents;
carboxy;
—CO—NR²⁵R²⁶, wherein R²⁵ and R²⁶ independently represent hydrogen or (C₁₋₄)alkyl (especially methyl);
—OCH₂—CO—(C₁₋₄)alkoxy (especially methoxycarbonyl-methoxy);
—CO—(C₁₋₄)alkoxy (especially methoxycarbonyl);
hydroxy-(C₁₋₄)alkyl (especially 2-hydroxypropan-2-yl);
(C₁₋₃)alkoxy-(C₁₋₄)alkyl (especially methoxymethyl, 2-methoxypropan-2-yl);
(C₂₋₄)alkoxy substituted with one or two hydroxy (especially 2-hydroxy-ethoxy, 3-hydroxy-propoxy, 2,3-dihydroxy-propoxy);
(C₁₋₃)alkoxy-(C₂₋₄)alkoxy (especially 2-methoxy-ethoxy, 3-methoxy-propoxy);
benzyloxy, wherein the phenyl group is optionally mono-substituted with methoxy; or
phenyl, optionally mono-substituted with halogen;
   or two of said substituents together form a bivalent group selected from —O—CH₂—O—, or —O—CH₂—CH₂—O— (it being understood that in such case no further substituent is present);

R³ represents aryl (especially naphthyl, phenyl), or 5- to 10-membered heteroaryl (notably 5- or 6-membered heteroaryl; especially pyrazolyl, isoquinolinyl, quinolinyl, imidazo[4,5-b]pyridinyl, pyridinyl or pyrimidinyl), wherein said aryl or heteroaryl independently is unsubstituted, or mono-, di-, or tri-substituted (especially mono- or di-substituted), wherein the substituents are independently selected from:
—NR³¹—SO₂—Y—R³², wherein
   R³¹ represents hydrogen or (C₁₋₃)alkyl; Y represents a direct bond; and R³² represents (C₁₋₄)alkyl (especially methyl), or (C₃₋₆)cycloalkyl (especially cyclopropyl); or
   R³¹ represents hydrogen or (C₁₋₃)alkyl; Y represents —NR^{Y}— wherein R^{Y} represents hydrogen or (C₁₋₃)alkyl; and R³² represents (C₁₋₄)alkyl (especially R⁴ represents hydrogen, Y represents —NH— or —N(CH₃)— and R³² represents (C₁₋₄)alkyl); or
   R³¹ and R³² together with the nitrogen and the —SO₂—Y-group to which they are attached to form a 5-, 6-, or 7-membered ring, wherein Y represents a direct bond or —NR^{Y}— wherein R^{Y} represents (C₁₋₃)alkyl (especially such ring is 1,1-dioxidoisothiazolidin-2-yl);
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen or methyl, and the other of R³³ and R³⁴ represents (C₁₋₄)alkyl, or (C₃₋₆)cycloalkyl];
—SO₂—R³⁵ wherein R³⁵ represents (C₁₋₅)alkyl;
(C₁₋₄)alkyl (especially methyl, ethyl);
(C₁₋₄)alkoxy;
(C₁₋₃)fluoroalkyl (especially trifluoromethyl);
(C₁₋₃)fluoroalkoxy (especially difluoromethoxy, trifluoromethoxy, 2,2,2-trifluoroethoxy);
(C₃₋₆)cycloalkyl, optionally containing one oxygen ring atom, and optionally mono-substituted with amino, —NH—(SO)—(C₁₋₄)alkyl, or morpholin-4-yl (especially cyclopropyl; or 3-amino-oxetan-3-yl, 3-(morpholin-4-yl)-oxetan-3-yl, or 3-((tert-butylsulfinyl)amino)-oxetan-3-yl);
halogen (especially fluoro, chloro);
cyano;
nitro;
hydroxy-(C₁₋₄)alkyl (especially hydroxymethyl);
—CO—(C₁₋₄)alkoxy (especially methoxy-carbonyl, ethoxy-carbonyl);
5-membered heteroaryl (especially oxazolyl, in particular oxazol-2-yl);
phenyl;
—(CH₂)ₘ—NR³⁶R³⁷; wherein m represents the integer 0 or 1; and
   R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
   R³⁶ and R³⁷ together with the nitrogen to which they are attached to form a saturated 3- to 7-membered monocyclic ring; wherein said ring optionally contains an oxygen ring atom or a group —NR¹¹— wherein R¹¹ represents (C₁₋₄)alkyl; and wherein said ring independently is optionally substituted with:
      one or two fluorine substituents; or
      one oxo substituent attached to a ring carbon atom in alpha position to a ring nitrogen atom (thus forming together with said nitrogen an amide group, or, in case a ring oxygen is additionaly adjacent, a carbamate group, or, in case second ring nitrogen is additionaly adjacent, an urea group)
   (notably such ring is aziridin-1-yl, azetidin-1-yl, pyrrolidin-1-yl, 2-oxo-pyrrolidin-1-yl, morpholin-4-yl, 3,3-difluoro-azetidin-1-yl, 4,4-difluoro-piperidin-1-yl, 2-oxo-piperazin-1-yl, or 1-methyl-piperazin-4-yl);
      or two of said substituents together form a bivalent group selected from —O—CH₂—O—; —O—CH₂—CH₂—O—; or —CH₂—CH₂—NR³⁸—CH₂—, wherein R³⁸ represents hydrogen, (C₁₋₄)alkyl, —CO—(C₁₋₄)alkoxy, or —CO—(C₁₋₄)alkyl wherein the (C₁₋₄)alkyl is optionally mono-substituted with hydroxy; and the remaining of said substituents, if present, is (C₁₋₄)alkyl;
      wherein in the particular case wherein R³ represents heteroaryl which is pyridinyl, such pyridinyl may additionally be present in form of the respective N-oxide.

The compounds of Formula (I) contain at least one and possibly more stereogenic or asymmetric centers, such as one or more asymmetric carbon atoms. The compounds of Formula (I) may thus be present as mixtures of stereoisomers or in stereoisomerically enriched form, preferably as essentially pure stereoisomers. Mixtures of stereoisomers may be separated in a manner known to a person skilled in the art.

In case a particular compound (or generic structure) is designated as (R)- or (S)-enantiomer, such designation is to be understood as referring to the respective compound (or generic structure) in enriched, especially essentially pure, enantiomeric form. Likewise, in case a specific asymmetric center in a compound is designated as being in (R)- or (S)-configuration or as being in a certain relative configuration, such designation is to be understood as referring to the compound that is in enriched, especially essentially pure, form with regard to the respective configuration of said asymmetric center.

The term “enriched”, for example when used in the context of enantiomers is understood in the context of the present invention to mean especially that the respective enantiomer is present in a ratio (mutatis mutandis: purity) of at least 70:30, and notably of at least 90:10 (mutatis mutandis: purity of 70%/90%) with respect to the respective other enantiomer. Preferably the term refers to the respective essentially pure enantiomer. The term “essentially”, for example when used in a term such as “essentially pure” is understood in the context of the present invention to mean especially that the respective stereoisomer/composition/compound etc. consists in an amount of at least 90, especially of at least 95, and notably of at least 99 percent by weight of the respective pure stereoisomer/composition/compound etc.

In some instances, the compounds of formula (I) may contain tautomeric forms. Such tautomeric forms are encompassed in the scope of the present invention.

Where the plural form is used for compounds, salts, pharmaceutical compositions, diseases or the like, this is intended to mean also a single compound, salt, disease or the like.

Any reference to a compound of Formula (I) is to be understood as referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example “Handbook of Phramaceutical Salts. Properties, Selection and Use.”, P. Heinrich Stahl, Camille G. Wermuth (Eds.), Wiley-VCH, 2008; and “Pharmaceutical Salts and Co-crystals”, Johan Wouters and Luc Quéré (Eds.), RSC Publishing, 2012.

The present invention also includes isotopically labelled, especially ²H (deuterium) labelled compounds of formula (I), which compounds are identical to the compounds of formula (I) except that one or more atoms have each been replaced by an atom having the same atomic number but an atomic mass different from the atomic mass usually found in nature. Isotopically labelled, especially ²H (deuterium) labelled compounds of formula (I) and salts thereof are within the scope of the present invention. Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment of the invention, the compounds of formula (I) are not isotopically labelled, or they are labelled only with one or more deuterium atoms. In a sub-embodiment, the compounds of formula (I) are not isotopically labelled at all. Isotopically labelled compounds of formula (I) may be prepared in analogy to the methods described hereinafter, but using the appropriate isotopic variation of suitable reagents or starting materials.

In case one or more substituent(s) are referred to as being optional, such substituent(s) may be absent (i.e. the parent group is unsubstituted and all positions of the parent group having a free valency are substituted with hydrogen), or the parent group is substituted with one or more of such substituent(s), wherein said substituent(s) is/are as explicitly defined.

In this patent application, a bond drawn as a dotted line shows the point of attachment of the radical drawn. For example, the radical drawn below

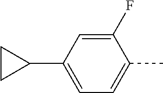

is the 2-fluoro-4-cyclopropyl-phenyl group.

Definitions provided herein are intended to apply uniformly to the compounds of formula (I) as defined in any one of embodiments 1) to 31), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

The term “halogen” means fluorine, chlorine, bromine or iodine, preferably fluorine or chlorine.

The term “alkyl”, used alone or in combination, refers to a straight or branched saturated hydrocarbon chain containing one to six carbon atoms. The term “(C_{x-y})alkyl” (x and y each being an integer), refers to an alkyl group as defined before containing x to y carbon atoms. For example a (C₁₋₄)alkyl group contains from one to four carbon atoms. Examples of (C₁₋₄)alkyl groups are methyl, ethyl, propyl, isopropyl, n-butyl, isobutyl, sec.-butyl and tert.-butyl. Preferred are methyl and ethyl. Most preferred is methyl.

The term “alkoxy”, used alone or in combination, refers to an alkyl-O— group wherein the alkyl refers to a straight or branched saturated hydrocarbon chain containing one to six carbon atoms. The term “(C_{x-y})alkoxy” (x and y each being an integer) refers to an alkoxy group as defined before containing x to y carbon atoms. For example a (C₁₋₄)alkoxy group means a group of the formula (C₁₋₄)alkyl-O— in which the term “(C₁₋₄)alkyl” has the previously given significance. Examples of (C₁₋₄)alkoxy groups are methoxy, ethoxy, n-propoxy, isopropoxy, n-butoxy, isobutoxy, sec.-butoxy and tert.-butoxy. Preferred is methoxy.

The term “(C₁₋₃)fluoroalkyl” refers to an alkyl group as defined before containing one to three carbon atoms in which one or more (and possibly all) hydrogen atoms have been replaced with fluorine. The term “(C_{x-y})fluoroalkyl” (x and y each being an integer) refers to a fluoroalkyl group as defined before containing x to y carbon atoms. For example a (C₁₋₃)fluoroalkyl group contains from one to three carbon atoms in which one to seven hydrogen atoms have been replaced with fluorine. Representative examples of (C₁₋₃)fluoroalkyl groups include trifluoromethyl, difluoromethyl, fluoromethyl, 2-fluoroethyl, 2,2-difluoroethyl, and 2,2,2-trifluoroethyl. Preferred are (C₁)fluoroalkyl groups such as especially trifluoromethyl or difluoromethyl.

The term “(C₁₋₃)fluoroalkoxy” refers to an alkoxy group as defined before containing one to three carbon atoms in which one or more (and possibly all) hydrogen atoms have been replaced with fluorine. The term “(C_{x-y})fluoroalkoxy” (x and y each being an integer) refers to a fluoroalkoxy group as defined before containing x to y carbon atoms. For example a (C₁₋₃)fluoroalkoxy group contains from one to three carbon atoms in which one to seven hydrogen atoms have been replaced with fluorine. Representative examples of (C₁₋₃)fluoroalkoxy groups include trifluoromethoxy, difluoromethoxy and 2,2,2-trifluoroethoxy. Preferred are (C₁)fluoroalkoxy groups such as trifluoromethoxy and difluoromethoxy.

The term “cycloalkyl”, used alone or in combination, refers to a saturated carbocyclic ring containing three to seven carbon atoms. The term “(C_{x-y})cycloalkyl” (x and y each being an integer), refers to a cycloalkyl group as defined before containing x to y carbon atoms. For example a (C₃₋₆)cycloalkyl group contains from three to six carbon atoms. Examples of cycloalkyl groups are cyclopropyl, cyclobutyl, cyclopentyl, and cyclohexyl. Preferred is cyclopropyl.

The term “cycloalkyl optionally containing one or two ring oxygen atoms”, used alone or in combination, e.g. for the substituents of groups “R²”, refers to a cycloalkyl group as defined before. In addition, one or two ring carbon atoms of said cycloalkyl may be replaced by a ring oxygen atom. Examples of such groups are especially cycloalkyl groups such as cyclopropyl, cyclobutyl, cyclopentyl, and cyclohexyl; as well as oxygen containing groups such as oxetanyl, tetrahydrofuranyl, tetrahydro-2H-pyranyl, 1,3-dioxolanyl, and 1,3-dioxan-2-yl. Preferred is cyclopropyl.

The term “cycloalkyl optionally containing one oxygen ring atom”, used alone or in combination, e.g. for the substituents of groups “R³”, refers to a cycloalkyl group as defined before. In addition, one ring carbon atom of said cycloalkyl may be replaced by a ring oxygen atom. Examples of such groups are cycloalkyl groups such as cyclopropyl, cyclobutyl, cyclopentyl, and cyclohexyl; as well as oxygen containing groups such as oxetanyl, tetrahydrofuranyl, and tetrahydro-2H-pyranyl. Preferred is oxetan-3-yl. Said groups are optionally mono-substituted (i.e. unsubstituted or mono-substituted) as explicitly defined. In case a oxetan-3-yl group is mono-substituted, such substituent is preferably attached in position 3 of the oxetan-3-yl group.

The term “aryl”, used alone or in combination, means phenyl or naphthyl, preferably phenyl. The above-mentioned aryl groups are unsubstituted or substituted as explicitly defined.

For the substituent “R²” representing aryl, the term especially means phenyl. The aryl group as used for the substituent “R²” is unsubstituted, or mono-, di-, or tri-substituted as explicitly defined; especially mono-, di-, or tri-substituted. Notably, the substituents of groups R² representing phenyl are independently selected from (C₁₋₄)alkyl; (C₁₋₄)alkoxy; (C₃₋₆)cycloalkyl; (C₁₋₃)fluoroalkyl; (C₁₋₃)fluoroalkoxy; or halogen; in particular from methyl, methoxy, cyclopropyl, trifluoromethyl, difluoromethoxy, trifluoromethoxy, fluoro, chloro, cyano, dimethylcarbamoyl, methoxycarbonyl, 2-hydroxypropan-2-yl, 2-methoxypropan-2-yl, 2-hydroxy-ethoxy, 3-hydroxy-propoxy, 2,3-dihydroxy-propoxy, 2-methoxy-ethoxy, and 3-methoxy-propoxy; especially from methyl, methoxy, cyclopropyl, trifluoromethyl, difluoromethoxy, fluoro, and chloro.

For the substituent “R³” representing aryl, the term means naphthyl or phenyl, especially phenyl. The aryl group as used for the substituent “R³” is unsubstituted, or mono-, di-, or tri-substituted as explicitly defined; notably, in case the substituent “R³” is a phenyl group, it is mono-, di-, or tri-substituted; especially di-substituted wherein one substituent is attached in para position with regard to the point of attachment to the rest of the molecule. In case the substituent “R³” is a naphthyl group, such group is especially unsubstituted, or mono-substituted with halogen or (C₁₋₄)alkyl.

The term “heteroaryl”, used alone or in combination, means a 5- to 10-membered monocyclic or bicyclic aromatic ring containing one to a maximum of four heteroatoms, each independently selected from oxygen, nitrogen and sulfur. Examples of such heteroaryl groups are 5-membered heteroaryl groups such as furanyl, oxazolyl, isoxazolyl, oxadiazolyl, thiophenyl, thiazolyl, isothiazolyl, thiadiazolyl, pyrrolyl, imidazolyl, pyrazolyl, triazolyl, tetrazolyl; 6-membered heteroaryl groups such as pyridinyl, pyrimidinyl, pyridazinyl, pyrazinyl; and 8- to 10-membered bicyclic heteroaryl groups such as indolyl, isoindolyl, benzofuranyl, isobenzofuranyl, benzothiophenyl, indazolyl, benzimidazolyl, benzoxazolyl, benzisoxazolyl, benzothiazolyl, benzoisothiazolyl, benzotriazolyl, benzoxadiazolyl, benzothiadiazolyl, thienopyridinyl, quinolinyl, isoquinolinyl, naphthyridinyl, cinnolinyl, quinazolinyl, quinoxalinyl, phthalazinyl, pyrrolopyridinyl, pyrazolopyridinyl, pyrazolopyrimidinyl, pyrrolopyrazinyl, imidazopyridinyl, imidazopyridazinyl, and imidazothiazolyl. The above-mentioned heteroaryl groups are unsubstituted or substituted as explicitly defined.

In case “R²” represents “heteroaryl”, the term means heteroaryl groups, notably 5- or 6-membered heteroaryl groups, as defined before. In one embodiment, the term especially refers to the 5- or 6-membered heteroaryl groups pyridinyl, pyrimidinyl, pyrazolyl, thiazolyl, thiophenyl, isoxazolyl, and oxadiazolyl; as well as to the bicyclic heteroaryl groups thieno[2,3-b]pyridinyl, benzothiazolyl, and imidazo[1,5-a]pyridinyl. The above-mentioned heteroaryl groups as used for the substitutent “R²” are unsubstituted or substituted as explicitly defined. Notably, the substituents of groups R² representing 5- or 6-membered heteroaryl are independently selected from (C₁₋₄)alkyl; (C₁₋₄)alkoxy; (C₃₋₆)cycloalkyl; (C₁₋₃)fluoroalkyl; (C₁₋₃)fluoroalkoxy; halogen; especially from methyl, methoxy, cyclopropyl, difluoromethyl, trifluoromethyl, difluoromethoxy, fluoro, and chloro. In case R² is a bicyclic heteroaryl group, such group is preferably unsubstituted.

In case “R³” represents “heteroaryl”, the term means heteroaryl groups, notably 5- or 6-membered heteroaryl groups (especially 6-membered heteroaryl groups containing one or two nitrogen atoms) as defined before. Examples are the 5- or 6-membered heteroaryl groups pyrazolyl, isoquinolinyl, pyridinyl and pyrimidinyl; as well as to the bicyclic heteroaryl groups quinolinyl, and imidazo[4,5-b]pyridinyl. In one embodiment, the term especially refers to pyridinyl or pyrimidinyl, in particular pyridinyl which is attached to the rest of the molecule in position 3 or pyrimidinyl which is attached to the rest of the molecule in position 5. The above-mentioned heteroaryl groups as used for the substitutent “R³” are unsubstituted or mono-, di-, or tri-substituted as explicitly defined. In case R² represents a 5- or 6-membered heteroaryl, such groups are especially mono- or di-substituted, wherein preferably, in case of a 6-membered heteroaryl, one substituent is attached in para position with regard to the point of attachment to the rest of the molecule. In case R³ is a bicyclic heteroaryl group, such group is preferably unsubstituted.

The term “cyano” refers to a group —CN.

An example of groups “—O(CH₂)₂—NR²¹R²²” as used for substituents of the group R² is 2-dimethylamino-ethoxy.

Examples of groups “—(CH₂)ₚ—NR²³R²⁴” as used for substituents of the group R² are amino, ethylamino, dimethylamino, and dimethylamino-methyl, as well as 3,3-difluoro-azetidin-1-yl and morpholin-4-yl; especially dimethylamino-methyl and morpholin-4-yl.

Examples of groups “—(CH₂)ₘ—NR³⁶R³⁷” as used for substituents of the group R³ are dimethylamino, (2-hydroxyethyl)-methylamino, (2-methoxyethyl)-methylamino, (2,2,2-trifluoroethyl)-methylamino, as well as aziridin-1-yl, morpholin-4-yl, morpholin-4-yl-methyl, and 1-methyl-piperazin-4-yl; especially dimethylamino and morpholin-4-yl.

Examples of groups “—CO—NR²⁵R²⁶” as used for substituents of the group R² are carbamoyl, methyl-carbamoyl, dimethyl-carbamoyl and diethyl-carbamoyl; especially dimethyl-carbamoyl.

Examples of groups “—CO—NR³³R³⁴” as used for substituents of the group R³ are carbamoyl, methyl-carbamoyl, dimethyl-carbamoyl, ethyl-(methyl)-carbamoyl, diethyl-carbamoyl, cyclopropyl-carbamoyl, cyclopropyl-(methyl)-carbamoyl, and isopropyl-(methyl)-carbamoyl; especially carbamoyl, methyl-carbamoyl, dimethyl-carbamoyl, and cyclopropyl-carbamoyl.

Examples of “hydroxy-(C₁₋₄)alkyl” groups are 2-hydroxypropan-2-yl for substituents of the group R², and hydroxymethyl for substituents of the group R³.

Examples of “(C₁₋₃)alkoxy-(C₁₋₄)alkyl” groups as used for substituents of the group R² are methoxymethyl, and 2-methoxypropan-2-yl.

Examples of “(C₂₋₄)alkoxy substituted with one or two hydroxy” groups as used for substituents of the group R² are 2-hydroxy-ethoxy, 3-hydroxy-propoxy, and 2,3-dihydroxy-propoxy.

An example of a “(C₁₋₃)alkoxy-(C₂₋₄)alkoxy group as used for substituents of the group R² is 2-methoxy-ethoxy.

Examples of a “—CO—(C₁₋₄)alkoxy” group as used for substituents of the group R² or R³ are methoxy-carbonyl and ethoxy-carbonyl.

It is understood that in groups “—NR³¹—SO₂—Y—R³², wherein R³¹ and R³² together with the nitrogen and the —SO₂—Y-group to which they are attached to form a 5-, 6-, or 7-membered ring” as used for substituents of the group R³, respectively, as used for the substituent R^{3a}, the ring fragment formed by R³¹ and R³² is carbocyclic and does not contain further heteroatoms (in addition to the —N—SO₂—Y— fragment which is part of the ring).

Examples of groups “—NR³¹—SO₂—Y—R³²” are methylsulfonamido, N-methyl-methylsulfonamido, cyclopropylsulfonamido, (N,N-dimethylsulfamoyl)-amino, and 1,1-dioxo-isothiazolidin-2-yl.

An example of a group “—SO₂—R³⁵” as used for substituents of the group R³, respectively, as used for the substituent R^{3a}, is methylsulfonyl.

Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range; or if a variable is defined as being an integer between 1 and 4, this means that the variable is the integer 1, 2, 3, or 4.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C. Besides, the term “room temperature” as used herein refers to a temperature of about 25° C.

Further embodiments of the invention are presented hereinafter.

2) A second aspect of the invention relates to compounds of Formula (I) according to embodiment 1), wherein the absolute configuration of the carbon atom carrying the substituent R² is as depicted in Formula (I_{E}):

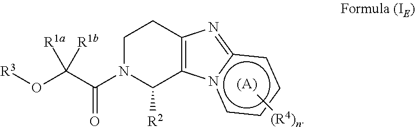

3) A further embodiment relates to compounds according to embodiments 1) or 2) wherein R^{1a} and R^{1b} both represent hydrogen.

4) A further embodiment relates to compounds according to any one of embodiments 1) to 3), wherein ring (A) represents

A) a fused 6-membered carbocyclic aromatic ring containing the bridgehead nitrogen atom; or

B) a fused 6-membered aromatic ring containing the bridgehead nitrogen atom and one additional ring nitrogen atom;

For avoidance of any doubt, it is understood that according to embodiment 4), the fragment

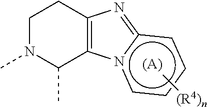

represents

A) the fragment

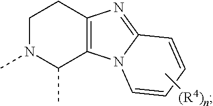

B) a fragment selected from the groups B1) to B4):

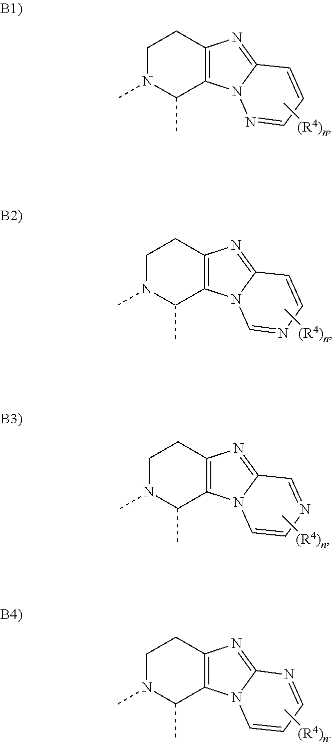

5) A further embodiment relates to compounds according to any one of embodiments 1) to 4), wherein (R⁴)ₙ represents one or two optional substituents (i.e. n represents the integer 0, 1, or 2) independently selected from (C₁₋₄)alkyl (especially methyl, ethyl, tert.-butyl), (C₁₋₃)trifluoroalkyl (especially trifluoromethyl), or halogen (especially chloro).

6) A further embodiment relates to compounds according to any one of embodiments 1) to 3), wherein the fragment

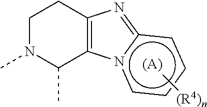

represents a fragment selected from:

A)

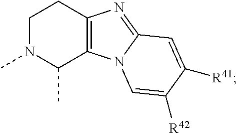

wherein R⁴¹ and R⁴² independently represent (C₁₋₄)alkyl (especially methyl, ethyl, tert.-butyl), (C₃₋₆)cycloalkyl (especially cyclopropyl), (C₁-3)trifluoroalkyl (especially trifluoromethyl), or halogen (especially chloro) (in a sub-embodiment, R⁴¹ represents hydrogen, (C₁₋₄)alkyl (especially methyl, ethyl, tert.-butyl), (C₃₋₆)cycloalkyl (especially cyclopropyl), (C₁₋₃)trifluoroalkyl (especially trifluoromethyl), or halogen (especially chloro); and R⁴² represents hydrogen or methyl); or

B)

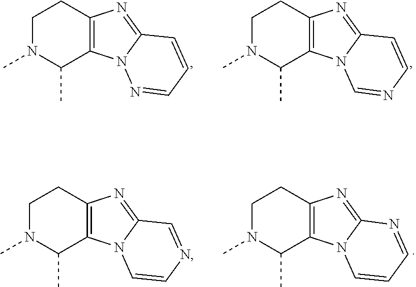

7) A further embodiment relates to compounds according to any one of embodiments 1) to 3), wherein the fragment

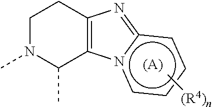

represents a fragment selected from:

A)

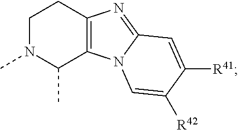

wherein R⁴¹ and R⁴² independently represent (C₁₋₄)alkyl (especially methyl, ethyl, tert.-butyl), (C₁₋₃)trifluoroalkyl (especially trifluoromethyl), or halogen (especially chloro) (in a sub-embodiment, R⁴¹ represents hydrogen, (C₁₋₄)alkyl (especially methyl, ethyl, tert.-butyl), (C₁₋₃)trifluoroalkyl (especially trifluoromethyl), or halogen (especially chloro); and R⁴² represents hydrogen or methyl); or

B)

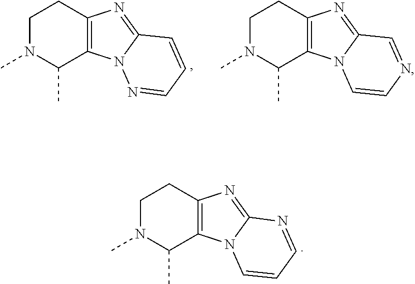

8) A further embodiment relates to compounds according to any one of embodiments 1) to 3), wherein the fragment

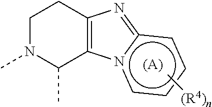

represents a fragment

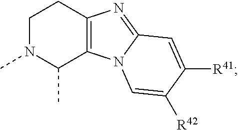

wherein R⁴¹ and R⁴² independently represent (C₁₋₄)alkyl (especially methyl, ethyl, tert.-butyl), (C₁₋₃)trifluoroalkyl (especially trifluoromethyl), or halogen (especially chloro) (in a sub-embodiment, R⁴¹ represents hydrogen, (C₁₋₄)alkyl (especially methyl, ethyl, tert.-butyl), (C₁₋₃)trifluoroalkyl (especially trifluoromethyl), or halogen (especially chloro); and R⁴² represents hydrogen or methyl).

9) A further embodiment relates to compounds according to any one of embodiments 1) to 8), wherein R² represents aryl (especially phenyl), or heteroaryl (notably 5- or 6-membered heteroaryl, in particular pyridinyl, pyrimidinyl, pyrazolyl, thiazolyl, thiophenyl, oxazolyl, isoxazolyl, oxadiazolyl, thieno[2,3-b]pyridinyl, benzothiazolyl, imidazo[1,5-a]pyridinyl), wherein said aryl or heteroaryl independently is unsubstituted, or mono-, di-, or tri-substituted, wherein the substituents are independently selected from:
(C₁₋₄)alkyl (especially methyl, ethyl);
(C₁₋₄)alkoxy (especially methoxy, ethoxy);
(C₃₋₆)cycloalkyl, optionally containing one or two ring oxygen atoms (especially cyclopropyl);
(C₁₋₃)fluoroalkyl (especially trifluoromethyl);
(C₁₋₃)fluoroalkoxy (especially difluoromethoxy, trifluoromethoxy, 2,2,2-trifluoroethoxy);
halogen;
cyano;
hydroxy;
—O(CH₂)₂—NR²¹R²², wherein
   R²¹ and R²² independently represent hydrogen or (C₁₋₃)alkyl (especially methyl);
—(CH₂)ₚ—NR²³R²⁴, wherein p represents the integer 0 or 1; and
   R²³ and R²⁴ independently represent hydrogen or (C₁₋₃)alkyl (especially methyl); or
   R²³ and R²⁴ together with the nitrogen atom to which they are attached to form a 4- to 7-membered saturated ring, wherein said ring optionally contains one ring oxygen atom;
—CO—NR²⁵R²⁶, wherein R²⁵ and R²⁶ independently represent hydrogen or (C₁₋₄)alkyl (especially methyl);
—OCH₂—CO—(C₁₋₄)alkoxy (especially methoxycarbonyl-methoxy);
—CO—(C₁₋₄)alkoxy (especially methoxycarbonyl);
hydroxy-(C₁₋₄)alkyl (especially 2-hydroxy-propan-2-yl);
(C₁₋₃)alkoxy-(C₁₋₄)alkyl (especially methoxymethyl, 2-methoxypropan-2-yl);
(C₂₋₄)alkoxy substituted with one or two hydroxy (especially 2-hydroxy-ethoxy, 3-hydroxy-propoxy, 2,3-dihydroxy-propoxy);
(C₁₋₃)alkoxy-(C₂₋₄)alkoxy (especially 2-methoxy-ethoxy, 3-methoxy-propoxy);
benzyloxy, wherein the phenyl group is optionally mono-substituted with methoxy; or
phenyl, optionally mono-substituted with halogen;
   or two of said substituents together form a bivalent group selected from —O—CH₂—O—, or —O—CH₂—CH₂—O— (it being understood that in such case no further substituent is present).

10) A further embodiment relates to compounds according to any one of embodiments 1) to 8), wherein R² represents aryl (especially phenyl), or heteroaryl (notably 5- or 6-membered heteroaryl, in particular pyridinyl, pyrimidinyl, pyrazolyl, thiazolyl, thiophenyl, oxazolyl, isoxazolyl, oxadiazolyl, thieno[2,3-b]pyridinyl, benzothiazolyl, imidazo[1,5-a]pyridinyl), wherein said aryl or heteroaryl independently is unsubstituted, or mono-, di-, or tri-substituted, wherein the substituents are independently selected from:
(C₁₋₄)alkyl (especially methyl, ethyl);
(C₁₋₄)alkoxy (especially methoxy, ethoxy);
(C₃₋₆)cycloalkyl, optionally containing one or two ring oxygen atoms (especially cyclopropyl);
(C₁₋₃)fluoroalkyl (especially trifluoromethyl);
(C₁₋₃)fluoroalkoxy (especially difluoromethoxy, trifluoromethoxy);
halogen;
cyano;
phenyl, optionally mono-substituted with halogen.

11) A further embodiment relates to compounds according to any one of embodiments 1) to 8), wherein R² represents phenyl, or heteroaryl selected from pyridinyl, pyrimidinyl, pyrazolyl, thiazolyl, thiophenyl, oxazolyl, isoxazolyl, oxadiazolyl, thieno[2,3-b]pyridinyl, benzothiazolyl, imidazo[1,5-a]pyridinyl; wherein said phenyl or heteroaryl independently is unsubstituted, or mono-, di-, or tri-substituted, wherein the substituents are independently selected from:
(C₁₋₄)alkyl (especially methyl, ethyl);
(C₁₋₄)alkoxy (especially methoxy, ethoxy);
(C₃₋₆)cycloalkyl, optionally containing one or two ring oxygen atoms (especially cyclopropyl);
(C₁₋₃)fluoroalkyl (especially trifluoromethyl);
(C₁₋₃)fluoroalkoxy (especially difluoromethoxy, trifluoromethoxy);
halogen;
cyano;
phenyl, optionally mono-substituted with halogen.

12) A further embodiment relates to compounds according to any one of embodiments 1) to 8), wherein R² represents phenyl, or heteroaryl selected from pyridinyl, pyrimidinyl, pyrazolyl, thiazolyl, thiophenyl, oxazolyl, isoxazolyl, oxadiazolyl, thieno[2,3-b]pyridinyl, benzothiazolyl, imidazo[1,5-a]pyridinyl; wherein said phenyl or heteroaryl independently is unsubstituted, or mono-, di-, or tri-substituted, wherein the substituents are independently selected from:
(C₁₋₄)alkyl (especially methyl);
(C₁₋₄)alkoxy (especially methoxy);
(C₃₋₆)cycloalkyl, optionally containing one or two ring oxygen atoms (especially cyclopropyl); or
halogen.

13) A further embodiment relates to compounds according to any one of embodiments 1) to 8), wherein

R² represents phenyl, wherein said phenyl is mono-, di-, or tri-substituted, wherein the substituents are independently selected from:
(C₁₋₄)alkyl (especially methyl, ethyl);
(C₁₋₄)alkoxy (especially methoxy, ethoxy);
(C₃₋₆)cycloalkyl, optionally containing one or two ring oxygen atoms (especially cyclopropyl);
(C₁₋₃)fluoroalkyl (especially trifluoromethyl);
(C₁₋₃)fluoroalkoxy (especially difluoromethoxy, trifluoromethoxy, 2,2,2-trifluoroethoxy);
halogen (especially fluoro, chloro);
cyano;
hydroxy;
—O(CH₂)₂—NR²¹R²², wherein R²¹ and R²² independently represent hydrogen or (C₁₋₃)alkyl (especially methyl);
—CO—NR²⁵R²⁶, wherein R²⁵ and R²⁶ independently represent hydrogen or (C₁₋₄)alkyl (especially methyl);
—CO—(C₁₋₄)alkoxy (especially methoxycarbonyl);
hydroxy-(C₁₋₄)alkyl (especially 2-hydroxy-propan-2-yl);
(C₁₋₃)alkoxy-(C₁₋₄)alkyl (especially methoxymethyl, 2-methoxypropan-2-yl);
(C₂₋₄)alkoxy substituted with one or two hydroxy (especially 2-hydroxy-ethoxy, 3-hydroxy-propoxy, 2,3-dihydroxy-propoxy);
(C₁₋₃)alkoxy-(C₂₋₄)alkoxy (especially 2-methoxy-ethoxy, 3-methoxy-propoxy);
benzyloxy, wherein the phenyl group is optionally mono-substituted with methoxy; or
   or two of said substituents together form a bivalent group selected from —O—CH₂—O—, or —O—CH₂—CH₂—O— (it being understood that in such case no further substituent is present);
   or R² represents 5-membered heteroaryl (notably pyrazolyl, thiazolyl, thiophenyl, oxazolyl, isoxazolyl, or oxadiazolyl; especially thiazolyl), wherein said heteroaryl is mono-, or di-substituted, wherein the substituents are independently selected from:
(C₁₋₄)alkyl (especially methyl, ethyl, isopropyl);
—(CH₂)ₚ—NR²³R²⁴, wherein p represents the integer 0 or 1; and
   R²³ and R²⁴ independently represent hydrogen or (C₁₋₃)alkyl (especially methyl); or
   R²³ and R²⁴ together with the nitrogen atom to which they are attached to form a 4- to 7-membered saturated ring, wherein said ring optionally contains one ring oxygen atom (especially morpholin-4-yl);
—CO—NR²⁵R²⁶, wherein R²⁵ and R²⁶ independently represent hydrogen or (C₁₋₃)alkyl (especially methyl);
phenyl, optionally mono-substituted with halogen;
   or R² represents 6-membered heteroaryl (notably pyridinyl, pyrimidinyl; especially pyridinyl), wherein said heteroaryl is unsubstituted, mono-, or di-substituted, wherein the substituents are independently selected from:
(C₁₋₄)alkyl (especially methyl, ethyl);
(C₁₋₄)alkoxy (especially methoxy, ethoxy);
(C₃₋₆)cycloalkyl, optionally containing one or two ring oxygen atoms (especially cyclopropyl);
(C₁₋₃)fluoroalkoxy (especially difluoromethoxy, trifluoromethoxy, 2,2,2-trifluoroethoxy);
halogen (especially fluoro, chloro);
   or R² represents unsubstituted 8- to 10-membered heteroaryl (notably thieno[2,3-b]pyridinyl, benzothiazolyl, imidazo[1,5-a]pyridinyl).

14) A further embodiment relates to compounds according to any one of embodiments 1) to 8), wherein

R² represents phenyl, wherein said phenyl is mono-, di-, or tri-substituted, wherein the substituents are independently selected from:
(C₁₋₄)alkyl (especially methyl);
(C₁₋₄)alkoxy (especially methoxy, ethoxy);
(C₃₋₆)cycloalkyl (especially cyclopropyl);
halogen (especially fluoro, chloro);
cyano;
—CO—NR²⁵R²⁶, wherein R²⁵ and R²⁶ represent (C₁₋₄)alkyl (especially methyl);
—CO—(C₁₋₄)alkoxy (especially methoxycarbonyl);
(C₂₋₄)alkoxy substituted with one or two hydroxy (especially 2-hydroxy-ethoxy, 3-hydroxy-propoxy, 2,3-dihydroxy-propoxy);
(C₁₋₃)alkoxy-(C₂₋₄)alkoxy (especially 2-methoxy-ethoxy, 3-methoxy-propoxy);
   or R² represents 5-membered heteroaryl (notably pyrazolyl, thiazolyl, thiophenyl, isoxazolyl, or oxadiazolyl; especially thiazolyl), wherein said heteroaryl is mono-, or di-substituted, wherein the substituents are independently selected from:
(C₁₋₄)alkyl (especially methyl, ethyl, isopropyl);
—CO—NR²⁵R²⁶, wherein R²⁵ and R²⁶ independently represent hydrogen or (C₁₋₃)alkyl (especially methyl);
phenyl, optionally mono-substituted with halogen;
   or R² represents 6-membered heteroaryl (notably pyridinyl, pyrimidinyl; especially pyridinyl), wherein said heteroaryl is unsubstituted, mono-, or di-substituted, wherein the substituents are independently selected from:
(C₁₋₄)alkyl (especially methyl, ethyl);
(C₁₋₄)alkoxy (especially methoxy, ethoxy);
(C₃₋₆)cycloalkyl (especially cyclopropyl);
halogen (especially fluoro, chloro);
   or R² represents unsubstituted thieno[2,3-b]pyridinyl, benzothiazolyl, or imidazo [1,5-a]pyridinyl.

15) A further embodiment relates to compounds according to any one of embodiments 1) to 8), wherein

R² represents phenyl, wherein said phenyl is mono-, di-, or tri-substituted, wherein the substituents are independently selected from:
(C₁₋₄)alkyl (especially methyl);
(C₁₋₄)alkoxy (especially methoxy);
(C₃₋₆)cycloalkyl (especially cyclopropyl);
halogen (especially fluoro, chloro);
(C₂₋₄)alkoxy substituted with one or two hydroxy (especially 2-hydroxy-ethoxy, 3-hydroxy-propoxy, 2,3-dihydroxy-propoxy);
   or R² represents 5-membered heteroaryl (notably pyrazolyl, thiazolyl, thiophenyl, isoxazolyl, or oxadiazolyl; especially thiazolyl), wherein said 5-membered heteroaryl is mono-, or di-substituted, wherein the substituents are independently selected from:
(C₁₋₄)alkyl (especially methyl); or
phenyl, optionally mono-substituted with halogen;
   or R² represents 6-membered heteroaryl (notably pyridinyl, pyrimidinyl; especially pyridinyl), wherein said 6-membered heteroaryl is unsubstituted, mono-, or di-substituted, wherein the substituents are independently selected from:
(C₁₋₄)alkyl (especially methyl);
(C₁₋₄)alkoxy (especially methoxy);
(C₃₋₆)cycloalkyl (especially cyclopropyl);
halogen (especially fluoro, chloro).

16) A further embodiment relates to compounds according to any one of embodiments 1) to 15), wherein R³ represents aryl (especially phenyl), or 5- to 10-membered heteroaryl (especially pyrazolyl, isoquinolinyl, quinolinyl, imidazo[4,5-b]pyridinyl, or pyridinyl), wherein said aryl or heteroaryl independently is unsubstituted, or mono-, di-, or tri-substituted (especially mono- or di-substituted), wherein the substituents are independently selected from:
—NR³¹—SO₂—Y—R³², wherein R³¹ represents hydrogen; Y represents a direct bond; and R³² represents (C₁₋₄)alkyl (especially methyl);
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen or methyl, and the other of R³³ and R³⁴ represents (C₁₋₄)alkyl, or (C₃₋₆)cycloalkyl];
—SO₂—R³⁵ wherein R³⁵ represents (C₁₋₅)alkyl;
(C₁₋₄)alkyl (especially methyl, ethyl);
(C₁₋₃)fluoroalkyl (especially trifluoromethyl);
(C₁₋₃)fluoroalkoxy (especially trifluoromethoxy);
(C₃₋₆)cycloalkyl, optionally containing one oxygen ring atom, and optionally mono-substituted with amino, —NH—(SO)—(C₁₋₄)alkyl, or morpholin-4-yl (especially cyclopropyl; or 3-amino-oxetan-3-yl, 3-(morpholin-4-yl)-oxetan-3-yl, or 3-((tert-butylsulfinyl)amino)-oxetan-3-yl);
halogen;
cyano;
nitro;
hydroxy-(C₁₋₄)alkyl (especially hydroxymethyl);
—CO—(C₁₋₄)alkoxy (especially ethoxy-carbonyl);
oxazolyl (in particular oxazol-2-yl);
phenyl;
—(CH₂)ₘ—NR³⁶R³⁷; wherein m represents the integer 0 or 1; and
   R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
   R³⁶ and R³⁷ together with the nitrogen to which they are attached to form a saturated 3- to 7-membered monocyclic ring; wherein said ring optionally contains an oxygen ring atom or a group —NR¹¹— wherein R¹¹ represents (C₁₋₄)alkyl; (notably such ring is aziridin-1-yl, morpholin-4-yl, or 1-methyl-piperazin-4-yl);
      or two of said substituents together form a bivalent group selected from —O—CH₂—O—; or —CH₂—CH₂—NR³⁸—CH₂—, wherein R³⁸ represents hydrogen, (C₁₋₄)alkyl, —CO—(C₁₋₄)alkoxy, or —CO—(C₁₋₄)alkyl wherein the (C₁₋₄)alkyl is optionally mono-substituted with hydroxy; and the remaining of said substituents, if present, is (C₁₋₄)alkyl.

17) A further embodiment relates to compounds according to any one of embodiments 1) to 15), wherein R³ represents aryl (especially phenyl), or 5- to 10-membered heteroaryl (pyrazolyl, isoquinolinyl, quinolinyl, or pyridinyl), wherein said aryl or heteroaryl independently is unsubstituted, or mono-, or di-substituted (especially mono- or di-substituted), wherein the substituents are independently selected from:
—NR³¹—SO₂—Y—R³², wherein
   R³¹ represents hydrogen or (C₁₋₃)alkyl; Y represents a direct bond; and R³² represents (C₁₋₄)alkyl (especially methyl), or (C₃₋₆)cycloalkyl (especially cyclopropyl); or
   R³¹ represents hydrogen or (C₁₋₃)alkyl; Y represents —NR^{Y}— wherein R^{Y} represents hydrogen or (C₁₋₃)alkyl; and R³² represents (C₁₋₄)alkyl (especially R⁴ represents hydrogen, Y represents —NH— or —N(CH₃)— and R³² represents (C₁₋₄)alkyl); or
   R³¹ and R³² together with the nitrogen and the —SO₂—Y-group to which they are attached to form a 5-, 6-, or 7-membered ring, wherein Y represents a direct bond or —NR^{Y}— wherein R^{Y} represents (C₁₋₃)alkyl (especially such ring is 1,1-dioxidoisothiazolidin-2-yl);
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen or methyl, and the other of R³³ and R³⁴ represents (C₁₋₄)alkyl, or (C₃₋₆)cycloalkyl];
—SO₂—R³⁵ wherein R³⁵ represents (C₁₋₅)alkyl;
(C₁₋₄)alkyl (especially methyl, ethyl);
(C₁₋₃)fluoroalkyl (especially trifluoromethyl);
(C₃₋₆)cycloalkyl, optionally containing one oxygen ring atom, and optionally mono-substituted with amino, —NH—(SO)—(C₁₋₄)alkyl, or morpholin-4-yl (especially cyclopropyl; or 3-amino-oxetan-3-yl, 3-(morpholin-4-yl)-oxetan-3-yl, or 3-((tert-butylsulfinyl)amino)oxetan-3-yl);
halogen;
cyano;
—(CH₂)ₘ—NR³⁶R³⁷; wherein m represents the integer 0 or 1; and
   R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
   R³⁶ and R³⁷ together with the nitrogen to which they are attached to form a saturated 3- to 7-membered monocyclic ring; wherein said ring optionally contains an oxygen ring atom or a group —NR¹¹— wherein R¹¹ represents (C₁₋₄)alkyl; (notably such ring is aziridin-1-yl, azetidin-1-yl, pyrrolidin-1-yl, morpholin-4-yl, or 1-methyl-piperazin-4-yl);
      or two of said substituents together form a bivalent group selected from —O—CH₂—O—; —O—CH₂—CH₂—O—; or —CH₂—CH₂—NR³⁸—CH₂—, wherein R³⁸ represents hydrogen, (C₁₋₄)alkyl, —CO—(C₁₋₄)alkoxy, or —CO—(C₁₋₄)alkyl wherein the (C₁₋₄)alkyl is optionally mono-substituted with hydroxy; and the remaining of said substituents, if present, is (C₁₋₄)alkyl.

18) A further embodiment relates to compounds according to any one of embodiments 1) to 15), wherein R³ represents aryl (especially phenyl), or 5- to 10-membered heteroaryl selected from pyrazolyl, isoquinolinyl, quinolinyl, or pyridinyl, wherein said aryl or heteroaryl independently is unsubstituted, or mono-, or di-substituted (especially mono- or di-substituted), wherein the substituents are independently selected from:
—NR³¹—SO₂—Y—R³², wherein
   R³¹ represents hydrogen or (C₁₋₃)alkyl; Y represents a direct bond; and R³² represents (C₁₋₄)alkyl (especially methyl), or (C₃₋₆)cycloalkyl (especially cyclopropyl); or
   R³¹ and R³² together with the nitrogen and the —SO₂—Y-group to which they are attached to form a 5-, 6-, or 7-membered ring, wherein Y represents a direct bond or —NR^{Y}— wherein R^{Y} represents (C₁₋₃)alkyl (especially such ring is 1,1-dioxidoisothiazolidin-2-yl);
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen or methyl, and the other of R³³ and R³⁴ represents (C₁₋₄)alkyl, or (C₃₋₆)cycloalkyl];
—SO₂—R³⁵ wherein R³⁵ represents (C₁₋₅)alkyl;
(C₁₋₄)alkyl (especially methyl, ethyl);
(C₃₋₆)cycloalkyl, optionally containing one oxygen ring atom, and optionally mono-substituted with amino, or morpholin-4-yl (especially cyclopropyl; or 3-amino-oxetan-3-yl, or 3-(morpholin-4-yl)-oxetan-3-yl,);
halogen;
—(CH₂)ₘ—NR³⁶R³⁷; wherein m represents the integer 0 or 1; and
   R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
   R³⁶ and R³⁷ together with the nitrogen to which they are attached to form a saturated 3- to 7-membered monocyclic ring; wherein said ring optionally contains an oxygen ring atom or a group —NR¹¹— wherein R¹¹ represents (C₁₋₄)alkyl;
   (notably such ring is aziridin-1-yl, azetidin-1-yl, pyrrolidin-1-yl, morpholin-4-yl, or 1-methyl-piperazin-4-yl);
      or two of said substituents together form a bivalent group selected from —O—CH₂—O—; —O—CH₂—CH₂—O—; or —CH₂—CH₂—NR³⁸—CH₂—, wherein R³⁸ represents hydrogen, (C₁₋₄)alkyl, —CO—(C₁₋₄)alkoxy, or —CO—(C₁₋₄)alkyl wherein the (C₁₋₄)alkyl is optionally mono-substituted with hydroxy; and the remaining of said substituents, if present, is (C₁₋₄)alkyl.

19) A further embodiment relates to compounds according to any one of embodiments 1) to 15), wherein R³ represents naphthyl or phenyl, or 5- to 10-membered heteroaryl selected from pyrazolyl, isoquinolinyl, quinolinyl, or pyridinyl, wherein said aryl or heteroaryl independently is unsubstituted, or mono-, or di-substituted (especially mono- or di-substituted), wherein the substituents are independently selected from:
—NR³¹—SO₂—Y—R³², wherein
   R³¹ represents hydrogen or (C₁₋₃)alkyl; Y represents a direct bond; and R³² represents (C₁₋₄)alkyl (especially methyl), or (C₃₋₆)cycloalkyl (especially cyclopropyl); or
   R³¹ and R³² together with the nitrogen and the —SO₂—Y-group to which they are attached to form a 1,1-dioxidoisothiazolidin-2-yl group;
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen or methyl, and the other of R³³ and R³⁴ represents (C₁₋₄)alkyl, or (C₃₋₆)cycloalkyl];
—SO₂—R³⁵ wherein R³⁵ represents (C₁₋₅)alkyl;
(C₁₋₄)alkyl (especially methyl, ethyl);
(C₃₋₆)cycloalkyl, optionally containing one oxygen ring atom, and optionally mono-substituted with amino, or morpholin-4-yl (especially cyclopropyl; or 3-amino-oxetan-3-yl, or 3-(morpholin-4-yl)-oxetan-3-yl,);
halogen;
—(CH₂)ₘ—NR³⁶R³⁷; wherein m represents the integer 0 or 1; and
   R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
   R³⁶ and R³⁷ together with the nitrogen to which they are attached to form a saturated 3- to 7-membered monocyclic ring; wherein said ring optionally contains an oxygen ring atom or a group —NR¹¹— wherein R¹¹ represents (C₁₋₄)alkyl;
   (notably such ring is aziridin-1-yl, azetidin-1-yl, pyrrolidin-1-yl, morpholin-4-yl, or 1-methyl-piperazin-4-yl);
      or two of said substituents together form a bivalent group selected from —O—CH₂—O—; —O—CH₂—CH₂—O—; or —CH₂—CH₂—NR³⁸—CH₂—, wherein R³⁸ represents hydrogen, (C₁₋₄)alkyl, —CO—(C₁₋₄)alkoxy, or —CO—(C₁₋₄)alkyl wherein the (C₁₋₄)alkyl is optionally mono-substituted with hydroxy; and the remaining of said substituents, if present, is (C₁₋₄)alkyl.

20) A further embodiment relates to compounds according to any one of embodiments 1) to 15), wherein R³ represents phenyl or pyridinyl, wherein said aryl or heteroaryl independently is mono-, or di-substituted, wherein the substituents are independently selected from:
—NR³¹—SO₂—Y—R³², wherein
   R³¹ represents hydrogen; Y represents a direct bond; and R³² represents (C₁₋₄)alkyl (especially methyl), or (C₃₋₆)cycloalkyl (especially cyclopropyl);
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen or methyl, and the other of R³³ and R³⁴ represents (C₁₋₄)alkyl, or (C₃₋₆)cycloalkyl];
(C₁₋₄)alkyl (especially methyl, ethyl);
(C₃₋₆)cycloalkyl (especially cyclopropy);
halogen; or
—(CH₂)ₘ—NR³⁶R³⁷; wherein m represents the integer 0 or 1; and
   R³⁶ and R³⁷ together with the nitrogen to which they are attached to form a morpholin-4-yl.

21) A further embodiment relates to compounds according to any one of embodiments 1) to 15), wherein R³ represents a fragment

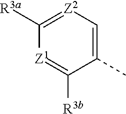

wherein

Z¹ and Z² independently represent CH or N;

R^{3a} represents:
—NR³¹—SO₂—Y—R³², wherein
   R³¹ represents hydrogen or (C₁₋₃)alkyl; Y represents a direct bond; and R³² represents (C₁₋₄)alkyl (especially methyl), or (C₃₋₆)cycloalkyl (especially cyclopropyl); or
   R³¹ represents hydrogen; Y represents —NR¹— wherein R^{Y1} represents hydrogen or (C₁₋₃)alkyl; and R³² represents (C₁₋₄)alkyl (especially R³¹ represents hydrogen, Y represents —NH— or —N(CH₃)— and R³² represents (C₁₋₄)alkyl); or
   R³¹ and R³² together with the nitrogen and the —SO₂—Y-group to which they are attached to form 1,1-dioxidoisothiazolidin-2-yl group;
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen, methyl or ethyl, and the other of R³³ and R³⁴ represents (C₁₋₄)alkyl (especially methyl or ethyl), or (C₃₋₆)cycloalkyl (especially cyclopropyl)];
—SO₂—R³⁵ wherein R³⁵ represents (C₁₋₅)alkyl;
—(CH₂)ₘ—NR³⁶R³⁷; wherein m represents the integer 0 or 1 (especially m represents 0); and
   R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
   R³⁶ and R³⁷ together with the nitrogen to which they are attached to form a saturated 3- to 7-membered monocyclic ring (especially a 4- to 6-membered monocyclic ring); wherein said ring optionally contains an oxygen ring atom or a group —NR¹¹— wherein R¹¹ represents (C₁₋₄)alkyl; (notably such ring is aziridin-1-yl, azetidin-1-yl, pyrrolidin-1-yl, morpholin-4-yl, or 1-methyl-piperazin-4-yl); and

R^{3b} represents (C₁₋₄)alkyl (especially methyl or ethyl); halogen (especially fluoro or chloro); or (C₃₋₆)cycloalkyl (especially cyclopropyl) [notably R^{3b} represents (C₁₋₄)alkyl (especially methyl or ethyl); or halogen (especially fluoro or chloro)].

22) A further embodiment relates to compounds according to embodiment 21), wherein Z¹ and Z² both represent CH; or Z¹ and Z² both represent N; or Z¹ represents N and Z² represents CH.

23) A further embodiment relates to compounds according to any one of embodiments 7) to 17), wherein Z¹ and Z² both represent CH.

24) A further embodiment relates to compounds according to embodiment 21), wherein Z¹ and Z² both represent N; or Z¹ represents N and Z² represents CH.

25) A further embodiment relates to compounds according to embodiment 21), wherein Z¹ represents N and Z² represents CH.

26) A further embodiment relates to compounds according to embodiment 21), wherein Z¹ and Z² both represent N.

27) A further embodiment relates to compounds according to any one of embodiments 21) to 26), wherein R^{3a} represents:
—NR³¹—SO₂—Y—R³², wherein
   R³¹ represents hydrogen or (C₁₋₃)alkyl; Y represents a direct bond; and R³² represents (C₁₋₄)alkyl (especially methyl), or (C₃₋₆)cycloalkyl (especially cyclopropyl); or
   R³¹ and R³² together with the nitrogen and the —SO₂—Y-group to which they are attached to form a 1,1-dioxidoisothiazolidin-2-yl group;
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen, methyl or ethyl, and the other of R³³ and R³⁴ represents (C₁₋₄)alkyl (especially methyl or ethyl), or (C₃₋₆)cycloalkyl (especially cyclopropyl)];
—SO₂—R³⁵ wherein R³⁵ represents (C₁₋₅)alkyl;
—(CH₂)ₘ—NR³⁶R³⁷; wherein m represents the integer 0 or 1 (especially m represents 0); and
   R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
   R³⁶ and R³⁷ together with the nitrogen to which they are attached to form a saturated 3- to 6-membered monocyclic ring; wherein said ring optionally contains an oxygen ring atom or a group —NR¹¹— wherein R¹¹ represents (C₁₋₄)alkyl; (notably such ring is aziridin-1-yl, morpholin-4-yl, or 1-methyl-piperazin-4-yl); and

R^{3b} represents (C₁₋₄)alkyl (especially methyl or ethyl); halogen (especially fluoro or chloro); or (C₃₋₆)cycloalkyl (especially cyclopropyl) [notably R^{3b} represents (C₁₋₄)alkyl (especially methyl or ethyl); or halogen (especially fluoro or chloro)].

28) A further embodiment relates to compounds according to any one of embodiments 21) to 26), wherein R^{3a} represents:
—NR³¹—SO₂—Y—R³², wherein
   R³¹ represents hydrogen or (C₁₋₃)alkyl; Y represents a direct bond; and R³² represents (C₁₋₄)alkyl (especially methyl), or (C₃₋₆)cycloalkyl (especially cyclopropyl); or
   R³¹ and R³² together with the nitrogen and the —SO₂—Y-group to which they are attached to form a 1,1-dioxidoisothiazolidin-2-yl group;
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen, methyl or ethyl, and the other of R³³ and R³⁴ represents (C₁₋₄)alkyl (especially methyl or ethyl), or (C₃₋₆)cycloalkyl (especially cyclopropyl)];
—SO₂—R³⁵ wherein R³⁵ represents (C₁₋₅)alkyl;

and

R^{3b} represents (C₁₋₄)alkyl (especially methyl or ethyl); halogen (especially fluoro or chloro); or (C₃₋₆)cycloalkyl (especially cyclopropyl) [notably R^{3b} represents (C₁₋₄)alkyl (especially methyl or ethyl); or halogen (especially fluoro or chloro)].

29) A further embodiment relates to compounds according to any one of embodiments 21) to 26), wherein R^{3a} represents:
—NR³¹—SO₂—Y—R³², wherein
   R³¹ represents hydrogen; Y represents a direct bond; and R³² represents (C₁₋₄)alkyl (especially methyl); or
—CO—NR³³R³⁴, wherein R³³ and R³⁴ independently represent hydrogen, (C₁₋₄)alkyl, or (C₃₋₆)cycloalkyl [especially one of R³³ and R³⁴ represents hydrogen, methyl or ethyl, and the other of R³³ and R³⁴ represents (C₁₋₄)alkyl (especially methyl or ethyl), or (C₃₋₆)cycloalkyl (especially cyclopropyl)];

and

R^{3b} represents (C₁₋₄)alkyl (especially methyl or ethyl); halogen (especially fluoro or chloro); or (C₃₋₆)cycloalkyl (especially cyclopropyl) [notably R^{3b} represents (C₁₋₄)alkyl (especially methyl or ethyl); or halogen (especially fluoro or chloro)].

30) The invention, thus, relates to compounds of the formula (I) as defined in embodiment 1), or to such compounds further limited by the characteristics of any one of embodiments 2) to 29), under consideration of their respective dependencies; to pharmaceutically acceptable salts thereof; and to the use of such compounds as medicaments especially in the treatment of diseases or disorders characterized by an altered rate of the tryptophan-serotonin metabolism. Especially the following embodiments relating to the compounds of formula (I) are thus possible and intended and herewith specifically disclosed in individualized form:

1, 2+1, 3+1, 3+2+1, 6+1, 6+2+1, 6+3+1, 6+3+2+1, 8+1, 8+2+1, 8+3+1, 8+3+2+1, 13+1, 13+2+1, 13+3+1, 13+3+2+1, 13+6+1, 13+6+2+1, 13+6+3+1, 13+6+3+2+1, 13+8+1, 13+8+2+1, 13+8+3+1, 13+8+3+2+1, 14+1, 14+2+1, 14+3+1, 14+3+2+1, 14+6+1, 14+6+2+1, 14+6+3+1, 14+6+3+2+1, 14+8+1, 14+8+2+1, 14+8+3+1, 14+8+3+2+1, 15+1, 15+2+1, 15+3+1, 15+3+2+1, 15+6+1, 15+6+2+1, 15+6+3+1, 15+6+3+2+1, 15+8+1, 15+8+2+1, 15+8+3+1, 15+8+3+2+1, 16+1, 16+2+1, 16+3+1, 16+3+2+1, 16+6+1, 16+6+2+1, 16+6+3+1, 16+6+3+2+1, 16+8+1, 16+8+2+1, 16+8+3+1, 16+8+3+2+1, 16+13+1, 16+13+2+1, 16+13+3+1, 16+13+3+2+1, 16+13+6+1, 16+13+6+2+1, 16+13+6+3+1, 16+13+6+3+2+1, 16+13+8+1, 16+13+8+2+1, 16+13+8+3+1, 16+13+8+3+2+1, 19+1, 19+2+1, 19+3+1, 19+3+2+1, 19+6+1, 19+6+2+1, 19+6+3+1, 19+6+3+2+1, 19+8+1, 19+8+2+1, 19+8+3+1, 19+8+3+2+1, 19+14+1, 19+14+2+1, 19+14+3+1, 19+14+3+2+1, 19+14+6+1, 19+14+6+2+1, 19+14+6+3+1, 19+14+6+3+2+1, 19+14+8+1, 19+14+8+2+1, 19+14+8+3+1, 19+14+8+3+2+1, 20+1, 20+2+1, 20+3+1, 20+3+2+1, 20+6+1, 20+6+2+1, 20+6+3+1, 20+6+3+2+1, 20+8+1, 20+8+2+1, 20+8+3+1, 20+8+3+2+1, 20+15+1, 20+15+2+1, 20+15+3+1, 20+15+3+2+1, 20+15+6+1, 20+15+6+2+1, 20+15+6+3+1, 20+15+6+3+2+1, 20+15+8+1, 20+15+8+2+1, 20+15+8+3+1, 20+15+8+3+2+1, 21+1, 21+2+1, 21+3+1, 21+3+2+1, 21+6+1, 21+6+2+1, 21+6+3+1, 21+6+3+2+1, 21+8+1, 21+8+2+1, 21+8+3+1, 21+8+3+2+1, 21+14+1, 21+14+2+1, 21+14+3+1, 21+14+3+2+1, 21+14+6+1, 21+14+6+2+1, 21+14+6+3+1, 21+14+6+3+2+1, 21+14+8+1, 21+14+8+2+1, 21+14+8+3+1, 21+14+8+3+2+1, 21+15+1, 21+15+2+1, 21+15+3+1, 21+15+3+2+1, 21+15+6+1, 21+15+6+2+1, 21+15+6+3+1, 21+15+6+3+2+1, 21+15+8+1, 21+15+8+2+1, 21+15+8+3+1, 21+15+8+3+2+1, 28+21+1, 28+21+2+1, 28+21+3+1, 28+21+3+2+1, 28+21+6+1, 28+21+6+2+1, 28+21+6+3+1, 28+21+6+3+2+1, 28+21+8+1, 28+21+8+2+1, 28+21+8+3+1, 28+21+8+3+2+1, 28+21+14+1, 28+21+14+2+1, 28+21+14+3+1, 28+21+14+3+2+1, 28+21+14+6+1, 28+21+14+6+2+1, 28+21+14+6+3+1, 28+21+14+6+3+2+1, 28+21+14+8+1, 28+21+14+8+2+1, 28+21+14+8+3+1, 28+21+14+8+3+2+1, 28+21+15+1, 28+21+15+2+1, 28+21+15+3+1, 28+21+15+3+2+1, 28+21+15+6+1, 28+21+15+6+2+1, 28+21+15+6+3+1, 28+21+15+6+3+2+1, 28+21+15+8+1, 28+21+15+8+2+1, 28+21+15+8+3+1, 28+21+15+8+3+2+1, 29+21+1, 29+21+2+1, 29+21+3+1, 29+21+3+2+1, 29+21+6+1, 29+21+6+2+1, 29+21+6+3+1, 29+21+6+3+2+1, 29+21+8+1, 29+21+8+2+1, 29+21+8+3+1, 29+21+8+3+2+1, 29+21+14+1, 29+21+14+2+1, 29+21+14+3+1, 29+21+14+3+2+1, 29+21+14+6+1, 29+21+14+6+2+1, 29+21+14+6+3+1, 29+21+14+6+3+2+1, 29+21+14+8+1, 29+21+14+8+2+1, 29+21+14+8+3+1, 29+21+14+8+3+2+1, 29+21+15+1, 29+21+15+2+1, 29+21+15+3+1, 29+21+15+3+2+1, 29+21+15+6+1, 29+21+15+6+2+1, 29+21+15+6+3+1, 29+21+15+6+3+2+1, 29+21+15+8+1, 29+21+15+8+2+1, 29+21+15+8+3+1, 29+21+15+8+3+2+1.

In the list above, the numbers refer to the embodiments according to their numbering provided hereinabove whereas “+” indicates the dependency from another embodiment. The different individualized embodiments are separated by commas. In other words, “21+14+1” for example refers to embodiment 21) depending on embodiment 14), depending on embodiment 1), i.e. embodiment “21+14+1” corresponds to the compounds of embodiment 1) further limited by the features of the embodiments 14) and 21).

31) A further embodiment relates to compounds of Formula (I) selected from:
ethyl 5-(2-(1-(3,4-dimethoxyphenyl)-3,4-dihydroimidazo[1,2-a:5,4-c′]dipyridin-2(1H)-yl)-2-oxoethoxy)-4-ethyl-2-methylbenzoate;
2-(2,4-dichlorophenoxy)-1-(1-(3,4-dimethoxyphenyl)-3,4-dihydroimidazo[1,2-a:5,4-c′]dipyridin-2(1H)-yl)ethan-1-one;
1-(1-(3,4-dimethoxyphenyl)-3,4-dihydroimidazo[1,2-a:5,4-c′]dipyridin-2(1H)-yl)-2-(naphthalen-2-yloxy)ethan-1-one;
1-(1-(3,4-dimethoxyphenyl)-3,4-dihydroimidazo[1,2-a:5,4-c′]dipyridin-2(1H)-yl)-2-((1-methylnaphthalen-2-yl)oxy)ethan-1-one;
1-(1-(3,4-dimethoxyphenyl)-3,4-dihydroimidazo[1,2-a:5,4-c′]dipyridin-2(1H)-yl)-2-((1-ethylnaphthalen-2-yl)oxy)ethan-1-one;
2-((1-bromonaphthalen-2-yl)oxy)-1-(1-(3,4-dimethoxyphenyl)-3,4-dihydroimidazo[1,2-a:5,4-c′]dipyridin-2(1H)-yl)ethan-1-one;
2-((1-chloronaphthalen-2-yl)oxy)-1-(1-(3,4-dimethoxyphenyl)-3,4-dihydroimidazo[1,2-a:5,4-c′]dipyridin-2(1H)-yl)ethan-1-one;
1-(1-(6-chloro-2-fluoropyridin-3-yl)-3,4-dihydroimidazo[1,2-a:5,4-c′]dipyridin-2(1H)-yl)-2-((2-chloro-6-morpholinopyridin-3-yl)oxy)ethan-1-one;
2-((2-chloro-6-morpholinopyridin-3-yl)oxy)-1-(1-(5-cyclopropyl-3-fluoropyridin-2-yl)-3,4-dihydroimidazo[1,2-a:5,4-c′]dipyridin-2(1H)-yl)ethan-1-one;
2-(2-chloro-4-morpholinophenoxy)-1-(1-(5-cyclopropyl-3-fluoropyridin-2-yl)-3,4-dihydroimidazo[1,2-a:5,4-c′]dipyridin-2(1H)-yl)ethan-1-one;
2-((2-chloro-6-morpholinopyridin-3-yl)oxy)-1-(1-(6-cyclopropyl-2-fluoropyridin-3-yl)-3,4-dihydroimidazo[1,2-a:5,4-c′]dipyridin-2(1H)-yl)ethan-1-one;
1-(1-(5-chloro-3-fluoropyridin-2-yl)-3,4-dihydroimidazo[1,2-a:5,4-c′]dipyridin-2(1H)-yl)-2-((2-chloro-6-morpholinopyridin-3-yl)oxy)ethan-1-one;
methyl-4-(2-(2-((2-chloro-6-(methylsulfonamido)pyridin-3-yl)oxy)acetyl)-1,2,3,4-tetrahydroimidazo[1,2-a:5,4-c′]dipyridin-1-yl)-3-fluorobenzoate;
methyl-4-(2-(2-((2-chloro-6-(cyclopropylcarbamoyl)pyridin-3-yl)oxy)acetyl)-1,2,3,4-tetrahydroimidazo[1,2-a:5,4-c′]dipyridin-1-yl)-3-fluorobenzoate;
6-chloro-N-cyclopropyl-5-(2-(1-(2-fluoro-4-(methoxymethyl)phenyl)-3,4-dihydroimidazo[1,2-a:5,4-c′]dipyridin-2(1H)-yl)-2-oxoethoxy)picolinamide;
N-(6-chloro-5-(2-(1-(2-fluoro-4-(methoxymethyl)phenyl)-3,4-dihydroimidazo[1,2-a:5,4-c′]dipyridin-2(1H)-yl)-2-oxoethoxy)pyridin-2-yl)methanesulfonamide;
6-chloro-N-cyclopropyl-5-(2-(1-(2-fluoro-4-(trifluoromethoxy)phenyl)-3,4-dihydroimidazo[1,2-a:5,4-c′]dipyridin-2(1H)-yl)-2-oxoethoxy)picolinamide;
N-(6-chloro-5-(2-(1-(4-cyano-2-fluorophenyl)-3,4-dihydroimidazo[1,2-a:5,4-c′]dipyridin-2(1H)-yl)-2-oxoethoxy)pyridin-2-yl)methanesulfonamide;
6-chloro-5-(2-(1-(4-cyano-2-fluorophenyl)-3,4-dihydroimidazo[1,2-a:5,4-c′]dipyridin-2(1H)-yl)-2-oxoethoxy)-N-cyclopropylpicolinamide;
6-chloro-N-cyclopropyl-5-(2-(1-(2-fluoro-4-(2-methoxyethoxy)phenyl)-3,4-dihydroimidazo[1,2-a:5,4-c′]dipyridin-2(1H)-yl)-2-oxoethoxy)picolinamide;
N-(6-chloro-5-(2-(1-(2-fluoro-4-(2-methoxyethoxy)phenyl)-3,4-dihydroimidazo[1,2-a:5,4-c′]dipyridin-2(1H)-yl)-2-oxoethoxy)pyridin-2-yl)methanesulfonamide;
6-chloro-N-cyclopropyl-5-(2-(1-(2-fluoro-4-(2-hydroxyethoxy)phenyl)-3,4-dihydroimidazo[1,2-a:5,4-c′]dipyridin-2(1H)-yl)-2-oxoethoxy)picolinamide;
N-(6-Chloro-5-(2-(1-(2-fluoro-4-(2-hydroxyethoxy)phenyl)-3,4-dihydroimidazo[1,2-a:5,4-c′]dipyridin-2(1H)-yl)-2-oxoethoxy)pyridin-2-yl)methanesulfonamide;
2-((2-chloro-6-(morpholinomethyl)pyridin-3-yl)oxy)-1-(1-(2-fluoro-4-methoxyphenyl)-3,4-dihydroimidazo[1,2-a:5,4-c′]dipyridin-2(1H)-yl)ethan-1-one;
N-(6-chloro-5-(2-(1-(2-fluoro-4-methoxyphenyl)-3,4-dihydroimidazo[1,2-a:5,4-c′]dipyridin-2(1H)-yl)-2-oxoethoxy)pyridin-2-yl)methanesulfonamide;
6-chloro-N-cyclopropyl-5-(2-(1-(2-fluoro-4-methoxyphenyl)-3,4-dihydroimidazo[1,2-a:5,4-c′]dipyridin-2(1H)-yl)-2-oxoethoxy)picolinamide;
1-(1-(4-chloro-2-fluorophenyl)-3,4-dihydroimidazo[1,2-a:5,4-c′]dipyridin-2(1H)-yl)-2-(4-chloro-2-methylphenoxy)ethan-1-one;
1-(1-(4-chloro-2-fluorophenyl)-3,4-dihydroimidazo[1,2-a:5,4-c′]dipyridin-2(1H)-yl)-2-((2-chloro-6-((2-hydroxyethyl)(methyl)amino)pyridin-3-yl)oxy)ethan-1-one;
1-(1-(4-chloro-2-fluorophenyl)-3,4-dihydroimidazo[1,2-a:5,4-c′]dipyridin-2(1H)-yl)-2-((2-chloro-6-(methyl (2,2,2-trifluoroethyl)amino)pyridin-3-yl)oxy)ethan-1-one;
1-(1-(4-chloro-2-fluorophenyl)-3,4-dihydroimidazo[1,2-a:5,4-c′]dipyridin-2(1H)-yl)-2-((2-chloro-6-(dimethylamino)pyridin-3-yl)oxy)ethan-1-one;
5-(2-(1-(4-chloro-2-fluorophenyl)-3,4-dihydroimidazo[1,2-a:5,4-c′]dipyridin-2(1H)-yl)-2-oxoethoxy)-N,6-dicyclopropylpicolinamide;
1-(1-(4-chloro-2-fluorophenyl)-3,4-dihydroimidazo[1,2-a:5,4-c′]dipyridin-2(1H)-yl)-2-((2-ethylpyridin-3-yl)oxy)ethan-1-one;
1-(1-(4-chloro-2-fluorophenyl)-3,4-dihydroimidazo[1,2-a:5,4-c′]dipyridin-2(1H)-yl)-2-((2-chloro-6-(morpholinomethyl)pyridin-3-yl)oxy)ethan-1-one;
1-(1-(4-chloro-2-fluorophenyl)-3,4-dihydroimidazo[1,2-a:5,4-c′]dipyridin-2(1H)-yl)-2-((2-chloro-6-morpholinopyridin-3-yl)oxy)ethan-1-one;
1-(1-(4-chloro-2-fluorophenyl)-3,4-dihydroimidazo[1,2-a:5,4-c′]dipyridin-2(1H)-yl)-2-((2-ethyl-6-methyl pyridin-3-yl)oxy)ethan-1-one;
N-(6-chloro-5-(2-(1-(4-chloro-2-fluorophenyl)-3,4-dihydroimidazo[1,2-a:5,4-c′]dipyridin-2(1H)-yl)-2-oxoethoxy)pyridin-2-yl)methanesulfonamide;
1-(1-(4-chloro-2-fluorophenyl)-3,4-dihydroimidazo[1,2-a:5,4-c′]dipyridin-2(1H)-yl)-2-((2-chloro-6-((2-methoxyethyl)(methyl)amino)pyridin-3-yl)oxy)ethan-1-one;
2-(2-chloro-4-morpholinophenoxy)-1-(1-(4-cyclopropyl-2-fluorophenyl)-3,4-dihydroimidazo[1,2-a:5,4-c′]dipyridin-2(1H)-yl)ethan-1-one;
2-((2-chloro-6-morpholinopyridin-3-yl)oxy)-1-(1-(4-cyclopropyl-2-fluorophenyl)-3,4-dihydroimidazo[1,2-a:5,4-c′]dipyridin-2(1H)-yl)ethan-1-one;
1-(1-(4-cyclopropyl-2-fluorophenyl)-3,4-dihydroimidazo[1,2-a:5,4-c′]dipyridin-2(1H)-yl)-2-((6-(dimethylamino)-2-methylpyridin-3-yl)oxy)ethan-1-one;
2-((2-chloropyridin-3-yl)oxy)-1-(1-(4-cyclopropyl-2-fluorophenyl)-3,4-dihydroimidazo[1,2-a:5,4-c′]dipyridin-2(1H)-yl)ethan-1-one;
1-(1-(4-cyclopropyl-2-fluorophenyl)-3,4-dihydroimidazo[1,2-a:5,4-c′]dipyridin-2(1H)-yl)-2-((2-ethyl-6-methyl pyridin-3-yl)oxy)ethan-1-one;
1-(1-(4-cyclopropyl-2-fluorophenyl)-3,4-dihydroimidazo[1,2-a:5,4-c′]dipyridin-2(1H)-yl)-2-((2-(trifluoromethyl)pyridin-3-yl)oxy)ethan-1-one;
2-((2-chloro-6-morpholinopyridin-3-yl)oxy)-1-(1-(3-phenyl-1,2,4-oxadiazol-5-yl)-3,4-dihydroimidazo[1,2-a:5,4-c′]dipyridin-2(1H)-yl)ethan-1-one;
N-(6-chloro-5-(2-(1-(4-chloro-2-fluorophenyl)-8-fluoro-3,4-dihydroimidazo[1,2-a:5,4-c′]dipyridin-2(1H)-yl)-2-oxoethoxy)pyridin-2-yl)methanesulfonamide;
N-(5-(2-(1-(4-chloro-2-fluorophenyl)-8-fluoro-3,4-dihydroimidazo[1,2-a:5,4-c′]dipyridin-2(1H)-yl)-2-oxoethoxy)-6-ethylpyridin-2-yl)methanesulfonamide;
N-(5-(2-(1-(2,4-dimethylthiazol-5-yl)-8-fluoro-3,4-dihydroimidazo[1,2-a:5,4-c′]dipyridin-2(1H)-yl)-2-oxoethoxy)-6-ethylpyridin-2-yl)methanesulfonamide;
1-(7-chloro-1-(3,4-dimethoxyphenyl)-8-methyl-3,4-dihydroimidazo[1,2-a:5,4-c′]dipyridin-2(1H)-yl)-2-(naphthalen-2-yloxy)ethan-1-one;
N-(6-chloro-5-(2-(1-(4-chloro-2-fluorophenyl)-7-fluoro-3,4-dihydroimidazo[1,2-a:5,4-c′]dipyridin-2(1H)-yl)-2-oxoethoxy)pyridin-2-yl)methanesulfonamide;
2-(2-chloro-4-morpholinophenoxy)-1-(9-(4-cyclopropyl-2-fluorophenyl)-6,9-dihydropyrido[4′,3′:4,5]imidazo[1,2-b]pyridazin-8(7H)-yl)ethan-1-one;
2-((2-chloro-6-morpholinopyridin-3-yl)oxy)-1-(9-(4-cyclopropyl-2-fluorophenyl)-6,9-dihydropyrido[4′,3′:4,5]imidazo[1,2-b]pyridazin-8(7H)-yl)ethan-1-one;
2-((2-chloro-6-morpholinopyridin-3-yl)oxy)-1-(9-(5-cyclopropyl-3-fluoropyridin-2-yl)-6,9-dihydropyrido[4′,3′:4,5]imidazo[1,2-b]pyridazin-8(7H)-yl)ethan-1-one;
5-(8-(2-((2-Chloro-6-morpholinopyridin-3-yl)oxy)acetyl)-6,7,8,9-tetrahydropyrido[4′,3′:4,5]imidazo[1,2-b]pyridazin-9-yl)-N,N-dimethylthiophene-3-carboxamide;
N-(5-(2-(9-(2,4-dimethylthiazol-5-yl)-6,9-dihydropyrido[4′,3′:4,5]imidazo[1,2-b]pyridazin-8(7H)-yl)-2-oxoethoxy)-6-ethylpyridin-2-yl)methanesulfonamide;
2-((2-chloro-6-morpholinopyridin-3-yl)oxy)-1-(1-(4-cyclopropyl-2-fluorophenyl)-3,4-dihydropyrido[4′,3′:4,5]imidazo[1,2-a]pyrazin-2(1H)-yl)ethan-1-one;
2-((2-chloro-6-morpholinopyridin-3-yl)oxy)-1-(6-(4-cyclopropyl-2-fluorophenyl)-8,9-dihydropyrido[4′,3′:4,5]imidazo[1,2-a]pyrimidin-7(6H)-yl)ethan-1-one;
1-(7-chloro-1-(3,4-dimethoxyphenyl)-3,4-dihydroimidazo[1,2-a:5,4-c′]dipyridin-2(1H)-yl)-2-(naphthalen-2-yloxy)ethan-1-one hydrochloride;
1-(7-chloro-1-(3,4-dimethoxyphenyl)-3,4-dihydroimidazo[1,2-a:5,4-c′]dipyridin-2(1H)-yl)-2-(2-chloro-4-(morpholinomethyl)phenoxy)ethan-1-one;
1-(7-chloro-1-(3,4-dimethoxyphenyl)-3,4-dihydroimidazo[1,2-a:5,4-c′]dipyridin-2(1H)-yl)-2-(2,4-dichlorophenoxy)ethan-1-one;
1-(7-chloro-1-(3,4-dimethoxyphenyl)-3,4-dihydroimidazo[1,2-a:5,4-c′]dipyridin-2(1H)-yl)-2-(2-chloro-4-morpholinophenoxy)ethan-1-one;
1-(7-chloro-1-(3-((R)-2,3-dihydroxypropoxy)-4-methoxyphenyl)-3,4-dihydroimidazo[1,2-a:5,4-c′]dipyridin-2(1H)-yl)-2-(naphthalen-2-yloxy)ethan-1-one;
1-(7-chloro-1-(3-((R)-2,3-dihydroxypropoxy)-4-methoxyphenyl)-3,4-dihydroimidazo[1,2-a:5,4-c′]dipyridin-2(1H)-yl)-2-(2-chloro-4-(morpholinomethyl)phenoxy)ethan-1-one;
1-(7-chloro-1-(4-methoxy-3-(3-methoxypropoxy)phenyl)-3,4-dihydroimidazo[1,2-a:5,4-c′]dipyridin-2(1H)-yl)-2-(naphthalen-2-yloxy)ethan-1-one;
1-(7-chloro-1-(3-(3-hydroxypropoxy)-4-methoxyphenyl)-3,4-dihydroimidazo[1,2-a:5,4-c′]dipyridin-2(1H)-yl)-2-(naphthalen-2-yloxy)ethan-1-one;
1-(7-chloro-1-(3-(2-hydroxyethoxy)-4-methoxyphenyl)-3,4-dihydroimidazo[1,2-a:5,4-c′]dipyridin-2(1H)-yl)-2-(naphthalen-2-yloxy)ethan-1-one;
1-(7-chloro-1-(3-(2-hydroxyethoxy)-4-methoxyphenyl)-3,4-dihydroimidazo[1,2-a:5,4-c′]dipyridin-2(1H)-yl)-2-(2-chloro-4-(morpholinomethyl)phenoxy)ethan-1-one;
Methyl 2-(5-(7-chloro-2-(2-(2-chloro-4-(morpholinomethyl) phenoxy)acetyl)-1,2,3,4-tetrahydroimidazo[1,2-a:5,4-c′]dipyridin-1-yl)-2-methoxyphenoxy)acetate;
N-(6-chloro-5-(2-(7-chloro-1-(2-fluoro-4-methylphenyl)-3,4-dihydroimidazo[1,2-a:5,4-c′]dipyridin-2(1H)-yl)-2-oxoethoxy)pyridin-2-yl)methanesulfonamide;
1-(7-chloro-1-(2-fluoro-4-methoxyphenyl)-3,4-dihydroimidazo[1,2-a:5,4-c′]dipyridin-2(1H)-yl)-2-(2-chloro-4-(morpholinomethyl)phenoxy)ethan-1-one;
1-(7-chloro-1-(2-fluoro-4-methoxyphenyl)-3,4-dihydroimidazo[1,2-a:5,4-c′]dipyridin-2(1H)-yl)-2-((2-chloro-6-(morpholinomethyl)pyridin-3-yl)oxy)ethan-1-one;
1-((1R)-7-chloro-1-(2-fluoro-4-methoxyphenyl)-3,1a-dihydroimidazo[1,2-a:5,4-c′]dipyridin-2(1H)-yl)-2-(2-chloro-4-morpholinophenoxy)ethan-1-one;
N-(6-chloro-5-(2-(7-chloro-1-(4-chloro-2-fluorophenyl)-3,4-dihydroimidazo[1,2-a:5,4-c′]dipyridin-2(1H)-yl)-2-oxoethoxy)pyridin-2-yl)methanesulfonamide;
1-((1R)-7-chloro-1-(6-methoxypyridin-3-yl)-3,1a-dihydroimidazo[1,2-a:5,4-c′]dipyridin-2(1H)-yl)-2-(2-chloro-4-morpholinophenoxy)ethan-1-one;
1-(7-chloro-1-(4-methoxy-3-((4-methoxybenzyl)oxy)phenyl)-3,4-dihydroimidazo[1,2-a:5,4-c′]dipyridin-2(1H)-yl)-2-(naphthalen-2-yloxy)ethan-1-one;
1-(7-chloro-1-(3-((S)-2,3-dihydroxypropoxy)-4-methoxyphenyl)-3,4-dihydroimidazo[1,2-a:5,4-c′]dipyridin-2(1H)-yl)-2-(naphthalen-2-yloxy)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-5-methylphenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(2-chloro-3-(trifluoromethyl)phenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-((2-chloropyridin-3-yl)oxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-((2-bromopyrid in-3-yl)oxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(2,4-dichlorophenoxy)-1-(1-(4,5-dimethoxyphenyl)-7-(trifluoromethyl)-3,4-dihydroimidazo[1,2-a:5,4-c′]dipyridin-2(1H)-yl)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(isoquinolin-7-yloxy)ethan-1-one;
2-((4-chloro-1-methyl-5-(trifluoromethyl)-1H-pyrazol-3-yl)oxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-((2-chloro-6-(morpholinomethyl)pyridin-3-yl)oxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(4-chloro-2-ethylphenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(2-chloro-4-(trifluoromethyl)phenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
1-(1-(3,4-dimethoxyphenyl)-7-(trifluoromethyl)-3,4-dihydroimidazo[1,2-a:5,4-c′]dipyridin-2(1H)-yl)-2-((2-ethylpyridin-3-yl)oxy)ethan-1-one;
2-(4-bromo-2-ethylphenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(2-ethyl-4-(4-methylpiperazin-1-yl)phenoxy)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(2-ethyl-4-morpholinophenoxy)ethan-1-one;
2-(4-(aziridin-1-yl)-2-ethylphenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(p-tolyloxy)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(2-(trifluoromethyl)phenoxy)ethan-1-one;
1-(1-(3,4-dimethylphenyl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(3,4-dimethylphenyl)-7-(trifluoromethyl)-3,4-dihydroimidazo[1,2-a:5,4-c′]dipyridin-2(1H)-yl)ethan-1-one;
1-(1-(3-(difluoromethoxy)-4-methoxyphenyl)-7-(trifluoromethyl)-3,4-dihydroimidazo[1,2-a:5,4-c′]dipyridin-2(1H)-yl)-2-(naphthalen-2-yloxy)ethan-1-one;
1-(1-(4-methoxy-3-(trifluoromethoxy)phenyl)-7-(trifluoromethyl)-3,4-dihydroimidazo[1,2-a:5,4-c′]dipyridin-2(1H)-yl)-2-(naphthalen-2-yloxy)ethan-1-one;
1-(1-(4-(2-(dimethylamino)ethoxy)-3-methoxyphenyl)-7-(trifluoromethyl)-3,4-dihydroimidazo[1,2-a:5,4-c′]dipyridin-2(1H)-yl)-2-(naphthalen-2-yloxy)ethan-1-one;
1-(1-(2-fluoro-4,5-dimethoxyphenyl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(3-morpholinooxetan-3-yl)phenoxy)-1-(1-(2-fluoro-4,5-dimethoxyphenyl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(2-fluoro-4,5-dimethoxyphenyl)-7-(trifluoromethyl)-3,4-dihydroimidazo[1,2-a:5,4-c′]dipyridin-2(1H)-yl)ethan-1-one;
tert-butyl 7-(2-(1-(2-fluoro-4,5-dimethoxyphenyl)-7-(trifluoromethyl)-3,4-dihydroimidazo[1,2-a:5,4-c′]dipyridin-2(1H)-yl)-2-oxoethoxy)-3,4-dihydroisoquinoline-2(1H)-carboxylate;
1-(1-(2-fluoro-4,5-dimethoxyphenyl)-7-(trifluoromethyl)-3,4-dihydroimidazo[1,2-a:5,4-c′]dipyridin-2(1H)-yl)-2-((1,2,3,4-tetrahydroisoquinolin-7-yl)oxy)ethan-1-one;
1-(1-(2-fluoro-4,5-dimethoxyphenyl)-7-(trifluoromethyl)-3,4-dihydroimidazo[1,2-a:5,4-c′]dipyridin-2(1H)-yl)-2-((2-methyl-1,2,3,4-tetrahydroisoquinolin-7-yl)oxy)ethan-1-one;
2-((2-acetyl-1,2,3,4-tetrahydroisoquinolin-7-yl)oxy)-1-(1-(2-fluoro-4,5-dimethoxyphenyl)-7-(trifluoromethyl)-3,4-dihydroimidazo[1,2-a:5,4-c′]dipyridin-2(1H)-yl)ethan-1-one;
1-(1-(2-fluoro-4,5-dimethoxyphenyl)-7-(trifluoromethyl)-3,4-dihydroimidazo[1,2-a:5,4-c′]dipyridin-2(1H)-yl)-2-((2-(2-hydroxyacetyl)-1,2,3,4-tetrahydroisoquinolin-7-yl)oxy)ethan-1-one;
N-(3-(3-chloro-4-(2-(1-(2-fluoro-4,5-dimethoxyphenyl)-7-(trifluoromethyl)-3,4-dihydroimidazo[1,2-a:5,4-c′]dipyridin-2(1H)-yl)-2-oxoethoxy)phenyl)oxetan-3-yl)-2-methylpropane-2-sulfinamide;
2-(4-(3-aminooxetan-3-yl)-2-chlorophenoxy)-1-(1-(2-fluoro-4,5-dimethoxyphenyl)-7-(trifluoromethyl)-3,4-dihydroimidazo[1,2-a:5,4-c′]dipyridin-2(1H)-yl)ethan-1-one;
1-(1-(5,6-dimethoxypyridin-3-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(2-fluoropyridin-3-yl)-7-(trifluoromethyl)-3,4-dihydroimidazo[1,2-a:5,4-c′]dipyridin-2(1H)-yl)ethan-1-one;
1-(1-(2-fluoropyridin-3-yl)-7-(trifluoromethyl)-3,4-dihydroimidazo[1,2-a:5,4-c′]dipyridin-2(1H)-yl)-2-(naphthalen-2-yloxy)ethan-1-one;
1-(1-(5,6-dimethoxypyridin-2-yl)-7-(trifluoromethyl)-3,4-dihydroimidazo[1,2-a:5,4-c′]dipyridin-2(1H)-yl)-2-(naphthalen-2-yloxy)ethan-1-one;
1-(1-(3-fluoropyridin-2-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-morpholinophenoxy)-1-(1-(3-fluoropyridin-2-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(2-fluoro-4-methylphenyl)-7-(trifluoromethyl)-3,4-dihydroimidazo[1,2-a:5,4-c′]dipyridin-2(1H)-yl)ethan-1-one;
2-((2-chloropyridin-3-yl)oxy)-1-(1-(2-fluoro-4-methylphenyl)-7-(trifluoromethyl)-3,4-dihydroimidazo[1,2-a:5,4-c′]dipyridin-2(1H)-yl)ethan-1-one;
2-((4-chloro-1-methyl-5-(trifluoromethyl)-1H-pyrazol-3-yl)oxy)-1-(1-(2-fluoro-4-methylphenyl)-7-(trifluoromethyl)-3,4-dihydroimidazo[1,2-a:5,4-c′]dipyridin-2(1H)-yl)ethan-1-one;
2-((2-ethyl-6-methylpyridin-3-yl)oxy)-1-(1-(2-fluoro-4-methylphenyl)-7-(trifluoromethyl)-3,4-dihydroimidazo[1,2-a:5,4-c′]dipyridin-2(1H)-yl)ethan-1-one;
N-(6-chloro-5-(2-(1-(2-fluoro-4-methylphenyl)-7-(trifluoromethyl)-3,4-dihydroimidazo[1,2-a:5,4-c′]dipyridin-2(1H)-yl)-2-oxoethoxy)pyridin-2-yl)methanesulfonamide;
2-(naphthalen-2-yloxy)-1-(1-(p-tolyl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(p-tolyl)-7-(trifluoromethyl)-3,4-dihydroimidazo[1,2-a:5,4-c′]dipyridin-2(1H)-yl)ethan-1-one;
1-(1-(4-methoxyphenyl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(4-ethoxyphenyl)-7-(trifluoromethyl)-3,4-dihydroimidazo[1,2-a:5,4-c′]dipyridin-2(1H)-yl)ethan-1-one;
2-(2,3-dimethyl-4-(morpholinomethyl)phenoxy)-1-(1-(4-ethoxyphenyl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(4-(difluoromethoxy)phenyl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(7-(trifluoromethyl)-1-(4-(trifluoromethyl)phenyl)-3,4-dihydroimidazo[1,2-a:5,4-c′]dipyridin-2(1H)-yl)ethan-1-one;
1-(1-(2-fluoro-4-methoxyphenyl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(2-fluoro-4-methoxyphenyl)-7-(trifluoromethyl)-3,4-dihydroimidazo[1,2-a:5,4-c′]dipyridin-2(1H)-yl)ethan-1-one;
2-(2,3-dimethyl-4-(morpholinomethyl)phenoxy)-1-(1-(2-fluoro-4-methoxyphenyl)-7-(trifluoromethyl)-3,4-dihydroimidazo[1,2-a:5,4-c′]dipyridin-2(1H)-yl)ethan-1-one;
2-((2-acetyl-5-methyl-1,2,3,4-tetrahydroisoquinolin-6-yl)oxy)-1-(1-(2-fluoro-4-methoxyphenyl)-7-(trifluoromethyl)-3,4-dihydroimidazo[1,2-a:5,4-c′]dipyridin-2(1H)-yl)ethan-1-one;
1-(1-(4-chlorophenyl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(4-chlorophenyl)-7-(trifluoromethyl)-3,4-dihydroimidazo[1,2-a:5,4-c′]dipyridin-2(1H)-yl)ethan-1-one;
1-(1-(4-(aminomethyl)phenyl)-7-(trifluoromethyl)-3,4-dihydroimidazo[1,2-a:5,4-c′]dipyridin-2(1H)-yl)-2-(naphthalen-2-yloxy)ethan-1-one;
1-(1-(2-fluorophenyl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(2-fluorophenyl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(6-chloropyridin-3-yl)-7-(trifluoromethyl)-3,4-dihydroimidazo[1,2-a:5,4-c′]dipyridin-2(1H)-yl)ethan-1-one;
1-(1-(6-chloropyridin-3-yl)-7-(trifluoromethyl)-3,4-dihydroimidazo[1,2-a:5,4-c′]dipyridin-2(1H)-yl)-2-(naphthalen-2-yloxy)ethan-1-one;
1-(1-(6-methylpyridin-3-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(6-methyl pyridin-3-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(6-methoxy-4-methyl pyridin-3-yl)-7-(trifluoromethyl)-3,4-dihydroimidazo[1,2-a:5,4-c′]dipyridin-2(1H)-yl)ethan-1-one;
1-(1-(4-chloro-2-fluorophenyl)-7-(trifluoromethyl)-3,4-dihydroimidazo[1,2-a:5,4-c′]dipyridin-2(1H)-yl)-2-((2-chloropyridin-3-yl)oxy)ethan-1-one;
1-(1-(4-chloro-2-fluorophenyl)-7-(trifluoromethyl)-3,4-dihydroimidazo[1,2-a:5,4-c′]dipyridin-2(1H)-yl)-2-(2-chloro-4-(morpholinomethyl)phenoxy)ethan-1-one;
2-((4-chloro-1-methyl-5-(trifluoromethyl)-1H-pyrazol-3-yl)oxy)-1-(1-(4-chloro-2-fluorophenyl)-7-(trifluoromethyl)-3,4-dihydroimidazo[1,2-a:5,4-c′]dipyridin-2(1H)-yl)ethan-1-one;
1-(1-(4-chloro-2-fluorophenyl)-7-(trifluoromethyl)-3,4-dihydroimidazo[1,2-a:5,4-c′]dipyridin-2(1H)-yl)-2-((2-ethyl-6-methyl pyridin-3-yl)oxy)ethan-1-one;
((1R)-1-(4-chloro-2-fluorophenyl)-7-(trifluoromethyl)-3,1a-dihydroimidazo[1,2-a:5,4-c′]dipyridin-2(1H)-yl)-2-(2-chloro-4-morpholinophenoxy)ethan-1-one;
1-(1-(4-chloro-2-fluorophenyl)-7-(trifluoromethyl)-3,4-dihydroimidazo[1,2-a:5,4-c′]dipyridin-2(1H)-yl)-2-((2-chloro-6-morpholinopyridin-3-yl)oxy)ethan-1-one;
N-(6-chloro-5-(2-(1-(4-chloro-2-fluorophenyl)-7-(trifluoromethyl)-3,4-dihydroimidazo[1,2-a:5,4-c′]dipyridin-2(1H)-yl)-2-oxoethoxy)pyridin-2-yl)methanesulfonamide;
2-(2-chloro-4-(morpholinomethyl)phenoxy)-1-(1-(1-phenyl-1H-pyrazol-4-yl)-7-(trifluoromethyl)-3,4-dihydroimidazo[1,2-a:5,4-c′]dipyridin-2(1H)-yl)ethan-1-one;
2-(2,3-dimethyl-4-(morpholinomethyl)phenoxy)-1-(1-(1-phenyl-1H-pyrazol-4-yl)-7-(trifluoromethyl)-3,4-dihydroimidazo[1,2-a:5,4-c′]dipyridin-2(1H)-yl)ethan-1-one;
1-(1-(benzo[d]thiazol-2-yl)-7-(trifluoromethyl)-3,4-dihydroimidazo[1,2-a:5,4-c′]dipyridin-2(1H)-yl)-2-(naphthalen-2-yloxy)ethan-1-one;
1-(1-(benzo[d]thiazol-2-yl)-7-(trifluoromethyl)-3,4-dihydroimidazo[1,2-a:5,4-c′]dipyridin-2(1H)-yl)-2-(2-chloro-4-(morpholinomethyl)phenoxy)ethan-1-one;
2-(2-chloro-4-(morpholinomethyl)phenoxy)-1-(1-(5-phenylisoxazol-3-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(5-(4-fluorophenyl)isoxazol-3-yl)-7-(trifluoromethyl)-3,4-dihydroimidazo[1,2-a:5,4-c′]dipyridin-2(1H)-yl)ethan-1-one;
2-(naphthalen-2-yloxy)-1-(1-(thieno[2,3-b]pyridin-2-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(thieno[2,3-b]pyridin-2-yl)-7-(trifluoromethyl)-3,4-dihydroimidazo[1,2-a:5,4-c′]dipyridin-2(1H)-yl)ethan-1-one;
1-(1-(4-cyclopropyl-2-fluorophenyl)-7-(trifluoromethyl)-3,4-dihydroimidazo[1,2-a:5,4-c′]dipyridin-2(1H)-yl)-2-((2-ethyl-6-methyl pyridin-3-yl)oxy)ethan-1-one;
2-(2-chloro-4-morpholinophenoxy)-1-(1-(4-cyclopropyl-2-fluorophenyl)-7-(trifluoromethyl)-3,4-dihydroimidazo[1,2-a:5,4-c′]dipyridin-2(1H)-yl)ethan-1-one;
1-(1-(4-cyclopropyl-2-fluorophenyl)-7-(trifluoromethyl)-3,4-dihydroimidazo[1,2-a:5,4-c′]dipyridin-2(1H)-yl)-2-((2-ethyl-6-methyl-1-(l1-oxidanyl)-1l4-pyridin-3-yl)oxy)ethan-1-one;
1-(1-(5-methylpyridin-2-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(5-methyl pyridin-2-yl)-7-(trifluoromethyl)-3,4-dihydroimidazo[1,2-a:5,4-c′]dipyridin-2(1H)-yl)ethan-1-one;
1-(1-(3,5-dimethyl isoxazol-4-yl)-7-(trifluoromethyl)-3,4-dihydroimidazo[1,2-a:5,4-c′]dipyridin-2(1H)-yl)-2-(naphthalen-2-yloxy)ethan-1-one;
1-(1-(5-methoxypyridin-2-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(5-methoxypyridin-2-yl)-7-(trifluoromethyl)-3,4-dihydroimidazo[1,2-a:5,4-c′]dipyridin-2(1H)-yl)ethan-1-one;
1-(1-(2-methoxypyrimidin-5-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(2-methyl pyridin-4-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(5-chloropyridin-2-yl)-7-(trifluoromethyl)-3,4-dihydroimidazo[1,2-a:5,4-c′]dipyridin-2(1H)-yl)ethan-1-one;
2-((2-chloro-6-(oxazol-2-yl)pyridin-3-yl)oxy)-1-(1-(2,4-dimethylthiazol-5-yl)-7-(trifluoromethyl)-3,4-dihydroimidazo[1,2-a:5,4-c′]dipyridin-2(1H)-yl)ethan-1-one;
6-chloro-N-cyclopropyl-5-(2-(1-(2,4-dimethylthiazol-5-yl)-7-(trifluoromethyl)-3,4-dihydroimidazo[1,2-a:5,4-c′]dipyridin-2(1H)-yl)-2-oxoethoxy)-N-methylpicolinamide;
1-(1-(6-methoxypyridin-3-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(6-methoxypyridin-3-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(6-ethoxypyridin-3-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(6-(2,2,2-trifluoroethoxy)pyridin-3-yl)-7-(trifluoromethyl)-3,4-dihydroimidazo[1,2-a:5,4-c′]dipyridin-2(1H)-yl)ethan-1-one;
1-(1-(3-hydroxy-4-methoxyphenyl)-7-(trifluoromethyl)-3,4-dihydroimidazo[1,2-a:5,4-c′]dipyridin-2(1H)-yl)-2-(naphthalen-2-yloxy)ethan-1-one;
2-(2,4-dichlorophenoxy)-1-(1-(3,4-dimethoxyphenyl)-7-methyl-3,4-dihydroimidazo[1,2-a:5,4-c′]dipyridin-2(1H)-yl)ethan-1-one;
1-(1-(3,4-dimethoxyphenyl)-7-methyl-3,4-dihydroimidazo[1,2-a:5,4-c′]dipyridin-2(1H)-yl)-2-(naphthalen-2-yloxy)ethan-1-one;
1-(7-(tert-butyl)-1-(3,4-dimethoxyphenyl)-3,4-dihydroimidazo[1,2-a:5,4-c′]dipyridin-2(1H)-yl)-2-(2,4-dichlorophenoxy)ethan-1-one;
2-(2,4-dichlorophenoxy)-1-(1-(3,4-dimethoxyphenyl)-7-ethyl-8-methyl-3,4-dihydroimidazo[1,2-a:5,4-c′]dipyridin-2(1H)-yl)ethan-1-one;
1-(1-(3,4-dimethoxyphenyl)-7-ethyl-8-methyl-3,4-dihydroimidazo[1,2-a:5,4-c′]dipyridin-2(1H)-yl)-2-(naphthalen-2-yloxy)ethan-1-one;
1-(1-(3,4-dimethoxyphenyl)-7-ethyl-8-methyl-3,4-dihydroimidazo[1,2-a:5,4-c′]dipyridin-2(1H)-yl)-2-((1-ethylnaphthalen-2-yl)oxy)ethan-1-one;
1-(1-(3,4-dimethoxyphenyl)-7-ethyl-8-methyl-3,4-dihydroimidazo[1,2-a:5,4-c′]dipyridin-2(1H)-yl)-2-((1-methylnaphthalen-2-yl)oxy)ethan-1-one hydrochloride;
2-(2,4-dichlorophenoxy)-1-(1-(3,4-dimethoxyphenyl)-8-methyl-3,4-dihydroimidazo[1,2-a:5,4-c′]dipyridin-2(1H)-yl)ethan-1-one;
1-(1-(3,4-dimethoxyphenyl)-8-methyl-3,4-dihydroimidazo[1,2-a:5,4-c′]dipyridin-2(1H)-yl)-2-(naphthalen-2-yloxy)ethan-1-one; and
1-(1-(3,4-dimethoxyphenyl)-8-phenyl-3,4-dihydroimidazo[1,2-a:5,4-c′]dipyridin-2(1H)-yl)-2-(naphthalen-2-yloxy)ethan-1-one.

The compounds of compounds of formula (I) and (II) as defined in any one of embodiments 1) to 31) and their pharmaceutically acceptable salts can be used as medicaments, e.g. in the form of pharmaceutical compositions for enteral (such especially oral, e.g. in form of a tablet or capsule) or parenteral administration (including intravenous, intraperitoneal, subcutaneous, or topical application, or inhalation).

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The present invention also relates to a method for the prevention or treatment of a disease or disorder mentioned herein comprising administering to a subject in need thereof a pharmaceutically active amount of a compound of formula (I) as defined in any one of embodiments 1) to 31). The invention thus also relates to a method of reducing the level of peripheral serotonin in a subject in need thereof, comprising administering to said subject a pharmaceutically active amount of a compound of formula (I) as defined in any one of embodiments 1) to 31).

In a preferred embodiment of the invention, the administered amount of such a compound of formula (I) as defined in any one of embodiments 1) to 31) is comprised between 1 mg and 1000 mg per day, particularly between 5 mg and 500 mg per day, more particularly between 10 mg and 400 mg per day.

For avoidance of any doubt, if compounds are described as being useful for the prevention or treatment of certain diseases, such compounds are likewise suitable for use in the preparation of a medicament for the prevention or treatment of said diseases.

Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range; or if a variable is defined as being an integer between 1 and 4, this means that the variable is the integer 1, 2, 3, or 4.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C. Besides, the term “room temperature” as used herein refers to a temperature of 25° C.

The compounds according to formula (I) are useful for the prevention or treatment of diseases or disorders characterized by an altered rate of the tryptophan-serotonin metabolism.

The term “disease or disorder characterized by an altered rate of the tryptophan-serotonin metabolism” refers to a neurological or peripheral disease or disorder characterized by an altered rate of the tryptophan-serotonin metabolism, wherein the rate limiting step of said tryptophan-serotonin metabolism is the hydroxylation of L-Tryp catalyzed by TPH and where an inhibitor of a TPH enzyme is required.

Examples of such diseases or disorders characterized by an altered rate of the tryptophan-serotonin metabolism are preferably peripheral diseases or disorders where the rate limiting step of said tryptophan-serotonin metabolism is the hydroxylation of L-Tryp catalyzed by TPH1 and where an inhibitor of a TPH1 is required. Particular examples are lung disease including interstitial lung disease (such as lung fibrosis), chronic obstructive pulmonary disease (COPD), pulmonary embolism, pulmonary hypertension including pulmonary arterial hypertension, radiation pneumonitis (including that giving rise to or contributing to pulmonary hypertension), asthma, and adult respiratory distress syndrome (ARDS); osteoporosis; gastrointestinal disorders including inflammatory bowel disease, postinfectious irritable bowel syndrome, coeliac disease, idiopathic constipation, and irritable bowel syndrome; ulcerative colitis; carcinoid syndrome; myxomatous valve disease; thrombosis; sleep disorders; pain; type1 and type 2 diabetes; immune disorders; liver disease (including (viral-induced) hepatitis fibrosis, transplantation, regeneration); acute and chronic hypertension; cancer including breast cancer, prostate cancer, and neuroendocrine tumors with elevated serotonin secretion (e.g carcinoid tumors); subarachnoid hemorrhage; abdominal migraine; CREST syndrome (calcinosis, Raynaud's phenomenon, esophageal dysfunction, sclerodactyly, telangiectasia); Gilbert's syndrome; nausea; serotonin syndrome; functional anorectal disorders; functional bloating; and inflammatory diseases including multiple sclerosis and systemic sclerosis. Notably examples are lung fibrosis; pulmonary hypertension including pulmonary arterial hypertension; asthma; osteoporosis; ulcerative colitis; irritable bowel syndrome; carcinoid syndrome; cancer including breast cancer, prostate cancer, and neuroendocrine tumors with elevated serotonin secretion (e.g carcinoid tumors); and inflammatory diseases including multiple sclerosis and systemic sclerosis.

Further examples of such diseases or disorders characterized by an altered rate of the tryptophan-serotonin metabolism are neurological health disorders where the rate limiting step of said tryptophan-serotonin metabolism is the hydroxylation of L-Tryp catalyzed by TPH2 and where an inhibitor of a TPH2 is required. Particular examples are depression; anxiety including generalized anxiety disorder and social phobia; emetic disorders; migraine; substance abuse; attention deficit disorder (ADD); attention deficit hyperactivity disorder (ADHD); bipolar disorder; suicidal behavior; behavioral disorder; schizophrenia; Parkinson's disease; Huntigton's disease; autism; dyskinesia; eating disorders; type 2 diabetes; pain; Alzheimer's disease; sexual dysfunction; and brain tumors.

### Preparation of Compounds of Formula (I)

General Preparation Routes:

The present compounds can be prepared by well known literature methods, by the methods given below, by the methods given in the experimental part or by analogous methods. Optimum reaction conditions may vary with the particular reactants or solvents used, but such conditions can be determined by a person skilled in the art by routine optimisation procedures. In some cases the final product may be further modified, for example, by manipulation of substituents to give a new final product. These manipulations may include, but are not limited to, reduction, oxidation, alkylation, acylation, and hydrolysis reactions which are commonly known to those skilled in the art. In some cases the order of carrying out the following reaction schemes, and/or reaction steps, may be varied to facilitate the reaction or to avoid unwanted reaction products. In the general sequence of reactions outlined below, the generic groups X, R, R^{1a}, R^{1b}, R² and R³ are as defined for formula (I). In some instances the generic groups X, R² and R³ may be incompatible with the assembly illustrated in the schemes below and so will require the use of protecting groups (PG). The use of protecting groups is well known in the art (see for example “Protective Groups in Organic Synthesis”, T. W. Greene, P. G. M. Wuts, Wiley-lnterscience, 1999). For the purposes of this discussion, it will be assumed that such protecting groups as necessary are in place. The compounds obtained may also be converted into pharmaceutically acceptable salts thereof in a manner known per se.

The compounds of the formula (I) may be prepared by the coupling of the amine of the structure 1 with the acid of the structure 2. Intermediate compounds of structure 2, 3 and 4 or their precursors are either commercially available or are prepared according to procedures known to a person skilled in the art or in analogy to the methods described in the experimental section below.

Compounds of structure 1 can be acylated with acid derivatives of structure 2 as depicted in scheme 2; for example using the corresponding acid chlorides or active esters in presence of a base like TEA or DIPEA in DCM, or using an the in situ activation method such as a well known amide-coupling reagent such as COMU, TBTU, HATU, EDC, DCC or PyBOP and a base like DIPEA or TEA in a solvent such as DCM, MeCN or DMF to deliver the compounds of Formula (I).

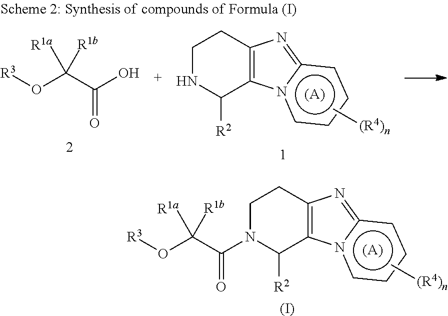

Alternatively, the desired residues R² and/or R³ may also be introduced in later steps that follow the amide coupling of the appropriate precursor amine of structure 1 with the appropriate acid derivatives of structure 2.

### Preparation of Compounds of Structure 1

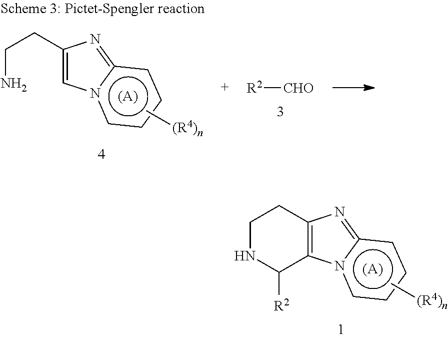

Compounds of the Structure 1 can be prepared by a reaction of amines of the Structure 4 with an aldehyde of the Structure 3 under acidic or basic conditions (Pictet-Spengler reaction, scheme 3) in a solvent such THF, toluene or the like.

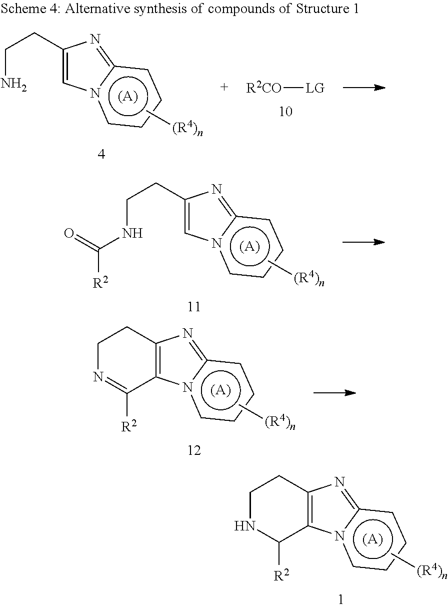

Alternatively, compounds of Structure 1 can by prepared using the three-step procedure depicted in scheme 4. In a typical reaction procedure, a compound of Structure 4 is dissolved in a solvent such as DCM, THF or water is reacted with an activated acid derivative of structure 10 (LG represents a leaving group) and a base such as NaOH, K₂CO₃, TEA or DIPEA at 0° C. to room temperature, according to procedures well known in the art. Subsequently, the amide of Structure 11 is cyclized with POCl₃, COCl₂, ZnCl₂ or the like in DCM, toluene or the like to deliver the imine of Structure 12, which may be reduced using a reducing agent such as NaBH₄, NaBH(OAc)₃, NaBH₃CN or hydrogen in presence of a suitable catalyst. Conditions such as hydrogenation or transfer hydrogenation in presence of a chiral catalyst may allow for an enantiospecific reduction of the compounds of structure 12 to the appropriate enantiomerically enriched compunds of structure 1.

### Preparation of Compounds of Structure 2

Acids of structure 2 may be prepared via alkylation reaction of the corresponding alcohol with halogen-acetic acid ester derivatives and subsequent hydrolysis of the ester to the acid. Under acidic or basic conditions. Alterantively, compounds of the Structure 2 may be prepared by alkylation of the corresponding alcohol under Mitsunobu reaction condition using hydroxyacetic acid derivatives in the presence of diethyl azodicarboxylate and the like in a solvent like toluene, DCM, THF and the like and subsequent hydrolysis of the ester to the acid under acidic or basic conditions.

### Preparation of Compounds of Structure 3

Aldehydes of structure 3 may be prepared by an oxidation of the corresponding alcohol derivatives, or by a reduction of the corresponding carbocylic acids or their derivatives thereof like esters, nitriles and the like. Aldehydes of structure 3 may also be prepared from corresponding halogen-precursors via halogen-metal exchange like nBuli and the like and subsequent formylation with DMF and the like.

### Preparation of Compounds of Structure 4

Amines of structure 4 or their precursors are either commercially available or can prepared according to procedures known to a person skilled in the art or in analogy to the methods described in the experimental part below.

Whenever the compounds of formula (I) are obtained in the form of mixtures of enantiomers, the enantiomers can be separated using methods known to one skilled in the art: e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in presence or absence of an amine such as triethylamine, diethylamine) and eluent B (hexane), at a flow rate of 0.8 to 150 mL/min.

Experimental Section:

### Abbreviations (as Used Herein and in the Description Above)

aq. aqueous
Bu butyl (such as in nBuLi=n-butyl lithium)
CC column chromatography on silica gel
conc. Concentrated
DCC 1,3-dicyclohexylcarbodiimide
DCM dichloromethane
DIPEA N-ethyldiisopropylamine
DME 1,2-dimethoxyethane
DMF dimethylformamide
DMP Dess-Martin periodinane
DMSO dimethylsulfoxide
DTT dithiothreitol
EA ethyl acetate
E. coli. Escherichia coli
Eq equivalent
Et ethyl
EtOH ethanol
FC flash chromatography
h hour(s)
HATU 2-(1H-7-Azabenzotriazol-1-yl)-1,1,3,3-tetramethyl uronium hexafluorophos-phate methanaminium
HOBt 1-hydroxybenzotriazole, hydrate
HPLC high performance liquid chromatography
LC liquid chromatography
M molarity [mol L⁻¹]
Me methyl
MeCN acetonitrile
MeOH methanol
MS mass spectroscopy
min. minute(s)
N normality
NFSI N-fluorobenzenesulfonimide
NMP N-methyl-2-pyrrolidon
NaOtBu sodium tert. (tertiary) butoxide
org. organic
Pd/C palladium on carbon
Ph phenyl
PTSA p-Toluenesulfonic acid
rt room temperature
Sat. Saturated
TBAF tetrabutylammonium fluoride
TBDMSCI tert-butyldimethylsilyl chloride
TBME tert-butylmethylether
TBTU O-benzotriazol-1-yl-N,N,N′,N′-tetramethyluronium tetrafluoroborate
tBu tert-butyl=tertiary butyl
TEA triethylamine
TFA trifluoroacetic acid
THF tetrahydrofuran
TMSCI trimethylsilyl chloride
TMSI iodotrimethylsilane (trimethylsilyl iodide)
TPP triphenylphosphine
Tris tris(hydroxymethyl)aminomethane
t_{R} retention time

I. Chemistry

The following examples illustrate the preparation of biologically active compounds of the invention but do not at all limit the scope thereof.

General:

All temperatures are stated in degrees Celsius (° C.). Unless otherwise indicated, the reactions take place at RT under an nitrogen atmosphere and are run in a flame dried round-bottomed flask equipped with a magnetic stir bar.

If not explicitly indicated otherwise, example compounds have been synthesized in racemic form.

Characterization Methods Used:

The LC-MS and GC-MS retention times have been obtained using the following elution conditions:

A) LC-MS (A):

Zorbax SB-Aq, 3.5 μm, 4.6×50 mm column thermostated at 40° C. The two elution solvents were as follows: solvent A=water+0.04% TFA; solvent B=acetonitrile. The eluent flow rate was 4.5 ml/min and the characteristics of the eluting mixture proportion in function of the time t from start of the elution are summarized in the table below (a linear gradient being used between two consecutive time points):

| | t (min) | | | | | |
|---|---|---|---|---|---|---|
| | 0 | 0.08 | 1.07 | 1.57 | 1.67 | 1.70 |
| Solvent A (%) | 95 | 95 | 5 | 5 | 95 | 95 |
| Solvent B (%) | 5 | 5 | 95 | 95 | 5 | 5 |

B) LC-MS (B):

Waters Atlantis T3, 5 μm, 4.6×30 mm column thermostated at 40° C. The two elution solvents were as follows: solvent A=water+0.04% TFA; solvent B=acetonitrile. The eluent flow rate was 4.5 ml/min and the characteristics of the eluting mixture proportion in function of the time t from start of the elution are summarized in the table below (a linear gradient being used between two consecutive time points):

| | t (min) | | | | | |
|---|---|---|---|---|---|---|
| | 0 | 0.08 | 1.07 | 1.57 | 1.67 | 1.70 |
| Solvent A (%) | 95 | 95 | 5 | 5 | 95 | 95 |
| Solvent B (%) | 5 | 5 | 95 | 95 | 5 | 5 |

C) LC-MS (C):

Agilent Zorbax Extend C18, 5 μm, 4.6×50 mm column thermostated at 40° C. The two elution solvents were as follows: solvent A=water+[NH₃]=13 mmol/l; solvent B=acetonitrile. The eluent flow rate was 4.5 ml/min and the characteristics of the eluting mixture proportion in function of the time t from start of the elution are summarized in the table below (a linear gradient being used between two consecutive time points):

| | t (min) | | | | |
|---|---|---|---|---|---|
| | 0 | 0.75 | 1.45 | 1.55 | 1.6 |
| Solvent A (%) | 95 | 5 | 5 | 95 | 95 |
| Solvent B (%) | 5 | 95 | 95 | 5 | 5 |

D) LC-MS (D):

Dionex Ultimate, column thermostated at 50° C. The two elution solvents were as follows: solvent A=water+0.05% NH₄OH; solvent B=acetonitrile. The eluent flow rate was 4.5 ml/min and the characteristics of the eluting mixture proportion in function of the time t from start of the elution are summarized in the table below (a linear gradient being used between two consecutive time points):

| | t (min) | | | | | |
|---|---|---|---|---|---|---|
| | 0 | 0.01 | 2.00 | 2.30 | 2.35 | 2.60 |
| Solvent A (%) | 95 | 95 | 5 | 5 | 95 | 95 |
| Solvent B (%) | 5 | 5 | 95 | 95 | 5 | 5 |

E) LC-MS (E):

Waters XBridge C18, 2.5 μm, 4.6×30 mm column thermostated at 40° C. The two elution solvents were as follows: solvent A=water+0.04% TFA; solvent B=acetonitrile. The eluent flow rate was 4.5 ml/min and the characteristics of the eluting mixture proportion in function of the time t from start of the elution are summarized in the table below (a linear gradient being used between two consecutive time points):

| | t (min) | | | | | |
|---|---|---|---|---|---|---|
| | 0 | 0.08 | 1.07 | 1.57 | 1.67 | 1.70 |
| Solvent A (%) | 95 | 95 | 5 | 5 | 95 | 95 |
| Solvent B (%) | 5 | 5 | 95 | 95 | 5 | 5 |

F) GC-MS (A)

Zebron ZB-5 MS, 15 m x 0.25 mm ID, 0.25 um film, 2.0 ml/min. The carrier gas is Helium and the chemical ionization occurs with CH₄ as reagent gas. Temp. gradient: 60-300° C. from 0 to 4.0 min and 300° C. isotherm from 4.0 to 5.0 min.

Non-Chiral Preparative Methods Used:

The purifications by preparative LC-MS have been performed using the conditions described hereafter.

E) Preparative LC-MS (I):

A X-Bridge column (Waters C18, 10 μm OBD, 30×75 mm) was used. The two elution solvents were as follows: solvent A=water+0.5% NH₄OH; solvent B=acetonitrile. The eluent flow rate was 75 mL/min and the characteristics of the eluting mixture proportion in function of the time t from start of the elution are summarized in the tables below (a linear gradient being used between two consecutive time points):

t (min)

| | 0 | 0.01 | 4.0 | 6.0 | 6.2 | 6.6 |
|---|---|---|---|---|---|---|
| Solvent A (%) | 90 | 90 | 5 | 5 | 90 | 90 |
| Solvent B (%) | 10 | 10 | 95 | 95 | 10 | 10 |

### Preparation of the Compounds of Structure 1

Method A

All intermediates of the structure 1 have been prepared in analogy to the following procedure:

A solution of (2-imidazo[1,2-a]pyridin-2-ylethyl)amine (75 mg), 2-fluoro-3-pyridinecarboxaldehyde (59 mg) and TFA (8 μl) in toluene (3 ml) was stirred at 80° C. overnight. The mixture was diluted with 1N aq. NaOH and EA, the layers were separated and the aq. phase was extracted with EA. The combined org. layers were washed with sat. aq. NaCl, dried over MgSO₄, filtrated off and evaporated in vacuo. The crude was purified by CC (Büchi Sepacore, 2 g cartridge, solvent A: DCM, solvent B: 7N NH₃ in MeOH, gradient in % B: 0 to 1, flow rate: 5 ml/min) to afford 87 mg of a yellow solid. LC-MS (A) t_{R}=0.25 min; [M+H]+: 269.05.

### Preparation of the Compounds of Formula (I)

### Method B

### Example 3.10.1: 1-(1-(3,4-dimethylphenyl)-7-(trifluoromethyl)-3,4-dihydroimidazo[1,2-a:5,4-c′]dipyridin-2(1H)-yl)-2-(naphthalen-2-yloxy)ethan-1-one

To a solution of 2-(naphthalen-2-yloxy)acetic acid (18 mg) in DCM (2 ml) was added DMAP (2.2 mg), HOBT (12 mg), EDCI (35 mg) and DIPEA (37 μl). The reaction mixture was stirred at rt for 30 min. The 1-(3,4-dimethylphenyl)-7-(trifluoromethyl)-1,2,3,4-tetrahydroimidazo[1,2-a:5,4-c′]dipyridine (25 mg) was added and the mixture was stirred at rt overnight. The mixture was diluted with DCM and washed with aq. HCl (1N) and sat. aq. NaHCO₃. The org. phase was dried over MgSO₄, filtrated off and evaporated in vacuo. The crude was purified by preparative LC-MS (I) to afford 29 mg od a colourless solid. LC-MS (A) t_{R}=0.95 min; [M+H]+: 530.17.

### Method C

### Example 1.2.1: 2-(2-chloro-4-(morpholinomethyl)phenoxy)-1-(1-(2-fluoropyridin-3-yl)-3,4-dihydroimidazo[1,2-a:5,4-c′]dipyridin-2(1H)-yl)ethan-1-one

To a solution of 2-(2-chloro-4-(morpholinomethyl)phenoxy)acetic acid (TFA salt) (40 mg) in DMF (1 ml) was added TBTU (34 mg). The mixture was stirred at rt for 30 min. The 1-(2-fluoropyridin-3-yl)-1,2,3,4-tetrahydroimidazo[1,2-a:5,4-c′]dipyridine (27 mg) and DIPEA (68.5 μl) were added and the mixture was stirred at rt for 2 h. The crude was purified by preparative LC-MS (I) to afford 29 mg of a colourless solid. LC-MS (A): t_{R}=0.50 min; [M+H]⁺: 536.02.

Following examples were synthesized starting from the appropriate acid derivative and amine following the method B or C. LC-MS data are listed in table 1 below. The LC-MS conditions used were LC-MS (A).

TABLE 1

| | | | | IC₅₀ |
|---|---|---|---|---|
| Example | Name | t_{R} | [M + H]⁺ | [nM] |
| 1.1.1 | 1-(1-(3,4-dimethoxyphenyl)-3,4-dihydroimidazo[1,2-a:5,4- | 0.82 | 472.47 | 418 |
| | c′]dipyridin-2(1H)-yl)-2-(2,5-dimethylphenoxy)ethan-1-one | | | |
| 1.1.2 | 1-(1-(3,4-dimethoxyphenyl)-3,4-dihydroimidazo[1,2-a:5,4- | 0.82 | 472.15 | 180 |
| | c′]dipyridin-2(1H)-yl)-2-(2,4-dimethylphenoxy)ethan-1-one | | | |
| 1.1.3 | 1-(1-(3,4-dimethoxyphenyl)-3,4-dihydroimidazo[1,2-a:5,4- | 0.83 | 494.15 | 134 |
| | c′]dipyridin-2(1H)-yl)-2-(naphthalen-1-yloxy)ethan-1-one | | | |
| 1.1.4 | 2-(2,4-dichlorophenoxy)-1-(1-(3,4-dimethoxyphenyl)-3,4- | 0.84 | 512.08 | 69 |
| | dihydroimidazo[1,2-a:5,4-c′]dipyridin-2(1H)-yl)ethan-1-one | | | |
| 1.1.5 | 1-(1-(3,4-dimethoxyphenyl)-3,4-dihydroimidazo[1,2-a:5,4- | 0.67 | 494.33 | 24 |
| | c′]dipyridin-2(1H)-yl)-2-(naphthalen-2-yloxy)ethan-1-one | | | |
| 1.1.6 | 1-(1-(3,4-dimethoxyphenyl)-3,4-dihydroimidazo[1,2-a:5,4- | 0.70 | 508.34 | 12 |
| | c′]dipyridin-2(1H)-yl)-2-((1-methylnaphthalen-2-yl)oxy)ethan-1-one | | | |
| 1.1.7 | 1-(1-(3,4-dimethoxyphenyl)-3,4-dihydroimidazo[1,2-a:5,4- | 0.73 | 522.32 | 4 |
| | c′]dipyridin-2(1H)-yl)-2-((1-ethylnaphthalen-2-yl)oxy)ethan-1-one | | | |
| 1.1.8 | 2-((1-bromonaphthalen-2-yl)oxy)-1-(1-(3,4-dimethoxyphenyl)-3,4- | 0.69 | 571.99 | 9 |
| | dihydroimidazo[1,2-a:5,4-c′]dipyridin-2(1H)-yl)ethan-1-one | | | |
| 1.1.9 | 2-((1-chloronaphthalen-2-yl)oxy)-1-(1-(3,4-dimethoxyphenyl)-3,4- | 0.71 | 528.11 | 8 |
| | dihydroimidazo[1,2-a:5,4-c′]dipyridin-2(1H)-yl)ethan-1-one | | | |
| 1.1.10 | ethyl 5-(2-(1-(3,4-dimethoxyphenyl)-3,4-dihydroimidazo[1,2-a:5,4- | 0.74 | 557.86 | 92 |
| | c′]dipyridin-2(1H)-yl)-2-oxoethoxy)4-ethyl-2-methylbenzoate | | | |
| 1.2.1 | 2-(2-chloro-4-(morpholinomethyl)phenoxy)-1-(1-(2-fluoropyridin-3- | 0.50 | 536.02 | 209 |
| | yl)-3,4-dihydroimidazo[1,2-a:5,4-c′]dipyridin-2(1H)-yl)ethan-1-one | | | |
| 1.3.1 | 2-((2-chloro-6-(morpholinomethyl)pyridin-3-yl)oxy)-1-(1-(2-fluoro-4- | 0.54 | 566.05 | 84 |
| | methoxyphenyl)-3,4-dihydroimidazo[1,2-a:5,4-c′]dipyridin-2(1H)- | | | |
| | yl)ethan-1-one | | | |
| 1.3.2 | N-(6-chloro-5-(2-(1-(2-fluoro-4-methoxyphenyl)-3,4- | 0.65 | 560.06 | 40 |
| | dihydroimidazo[1,2-a:5,4-c′]dipyridin-2(1H)-yl)-2-oxoethoxy)pyridin- | | | |
| | 2-yl)methanesulfonamide | | | |
| 1.3.3 | 6-chloro-N-cyclopropyl-5-(2-(1-(2-fluoro-4-methoxyphenyl)-3,4- | 0.69 | 550.11 | 19 |
| | dihydroimidazo[1,2-a:5,4-c′]dipyridin-2(1H)-yl)-2- | | | |
| | oxoethoxy)picolinamide | | | |
| 1.4.1 | 1-(1-(4-chloro-2-fluorophenyl)-3,4-dihydroimidazo[1,2-a:5,4- | 0.80 | 484.07 | 18 |
| | c′]dipyridin-2(1H)-yl)-2-(4-chloro-2-methylphenoxy)ethan-1-one | | | |
| 1.4.2 | 1-(1-(4-chloro-2-fluorophenyl)-3,4-dihydroimidazo[1,2-a:5,4- | 0.54 | 464.98 | 55 |
| | c′]dipyridin-2(1H)-yl)-2-((2-ethylpyridin-3-yl)oxy)ethan-1-one | | | |
| 1.4.3 | 1-(1-(4-chloro-2-fluorophenyl)-3,4-dihydroimidazo[1,2-a:5,4- | 0.69 | 471.05 | 271 |
| | c′]dipyridin-2(1H)-yl)-2-((2-chloropyridin-3-yl)oxy)ethan-1-one | | | |
| 1.4.4 | 1-(1-(4-chloro-2-fluorophenyl)-3,4-dihydroimidazo[1,2-a:5,4- | 0.56 | 569.99 | 90 |
| | c′]dipyridin-2(1H)-yl)-2-((2-chloro-6-(morpholinomethyl)pyridin-3- | | | |
| | yl)oxy)ethan-1-one | | | |
| 1.4.5 | 1-(1-(4-chloro-2-fluorophenyl)-3,4-dihydroimidazo[1,2-a:5,4- | 0.75 | 556.39 | 5 |
| | c′]dipyridin-2(1H)-yl)-2-((2-chloro-6-morpholinopyridin-3- | | | |
| | yl)oxy)ethan-1-one | | | |
| 1.4.6 | 1-(1-(4-chloro-2-fluorophenyl)-3,4-dihydroimidazo[1,2-a:5,4- | 0.56 | 479.37 | 78 |
| | c′]dipyridin-2(1H)-yl)-2-((2-ethyl-6-methylpyridin-3-yl)oxy)ethan-1- | | | |
| | one | | | |
| 1.4.7 | N-(6-chloro-5-(2-(1-(4-chloro-2-fluorophenyl)-3,4- | 0.67 | 563.83 | 10 |
| | dihydroimidazo[1,2-a:5,4-c′]dipyridin-2(1H)-yl)-2-oxoethoxy)pyridin- | | | |
| | 2-yl)methanesulfonamide | | | |
| 1.4.8 | 1-(1-(4-chloro-2-fluorophenyl)-3,4-dihydroimidazo[1,2-a:5,4- | 0.75 | 558.17 | 39 |
| | c′]dipyridin-2(1H)-yl)-2-((2-chloro-6-((2- | | | |
| | methoxyethyl)(methyl)amino)pyridin-3-yl)oxy)ethan-1-one | | | |
| 1.4.9 | N-(5-(2-(1-(4-chloro-2-fluorophenyl)-3,4-dihydroimidazo[1,2-a:5,4- | 0.70 | 570.34 | 364 |
| | c′]dipyridin-2(1H)-yl)-2-oxoethoxy)-6-cyclopropylpyridin-2- | | | |
| | yl)methanesulfonamide | | | |
| 1.4.10 | 1-(1-(4-chloro-2-fluorophenyl)-3,4-dihydroimidazo[1,2-a:5,4- | 0.69 | 544.07 | 87 |
| | c′]dipyridin-2(1H)-yl)-2-((2-chloro-6-((2- | | | |
| | hydroxyethyl)(methyl)amino)pyridin-3-yl)oxy)ethan-1-one | | | |
| 1.4.11 | 1-(1-(4-chloro-2-fluorophenyl)-3,4-dihydroimidazo[1,2-a:5,4- | 0.81 | 581.85 | 14 |
| | c′]dipyridin-2(1H)-yl)-2-((2-chloro-6-(methyl(2,2,2- | | | |
| | trifluoroethyl)amino)pyridin-3-yl)oxy)ethan-1-one | | | |
| 1.4.12 | 1-(1-(4-chloro-2-fluorophenyl)-3,4-dihydroimidazo[1,2-a:5,4- | 0.75 | 514.06 | 58 |
| | c′]dipyridin-2(1H)-yl)-2-((2-chloro-6-(dimethylamino)pyridin-3- | | | |
| | yl)oxy)ethan-1-one | | | |
| 1.4.13 | 5-(2-(1-(4-chloro-2-fluorophenyl)-3,4-dihydroimidazo[1,2-a:5,4- | 0.75 | 56010 | 36 |
| | c′]dipyridin-2(1H)-yl)-2-oxoethoxy)-N,6-dicyclopropylpicolinamide | | | |
| 1.4.14 | 1-(1-(4-chloro-2-fluorophenyl)-3,4-dihydroimidazo[1,2-a:5,4- | 0.69 | 549.00 | 287 |
| | c′]dipyridin-2(1H)-yl)-2-((2-chloro-6-(methylsulfonyl)pyridin-3- | | | |
| | yl)oxy)ethan-1-one | | | |
| 1.4.15 | 6-chloro-5-(1-(1-(4-chloro-2-fluorophenyl)-1,2,3,4- | 0.70 | 568.02 | 323 |
| | tetrahydroimidazo[1,2-a:5,4-c′]dipyridine-2-carbonyl)cyclopropoxy)- | | | |
| | N,N-dimethylpicolinamide | | | |
| 1.4.16 | N-(6-chloro-5-(1-(1-(4-chloro-2-fluorophenyl)-1,2,3,4- | 0.70 | 589.94 | 488 |
| | tetrahydroimidazo[1,2-a:5,4-c′]dipyridine-2- | | | |
| | carbonyl)cyclopropoxy)pyridin-2-yl)methanesulfonamide | | | |
| 1.4.17 | 6-chloro-5-((1-(1-(4-chloro-2-fluorophenyl)-3,4-dihydroimidazo[1,2- | 0.71 | 570.06 | 949 |
| | a:5,4-c′]dipyridin-2(1H)-yl)-2-methyl-1-oxopropan-2-yl)oxy)-N,N- | | | |
| | dimethylpicolinamide | | | |
| 1.5.1 | 2-(2-chloro-4-morpholinophenoxy)-1-(1-(4-cyclopropyl-2- | 0.77 | 560.94 | 9 |
| | fluorophenyl)-3,4-dihydroimidazo[1,2-a:5,4-c′]dipyridin-2(1H)- | | | |
| | yl)ethan-1-one | | | |
| 1.5.2 | 2-((2-chloro-6-morpholinopyridin-3-yl)oxy)-1-(1-(4-cyclopropyl-2- | 0.76 | 561.78 | 6 |
| | fluorophenyl)-3,4-dihydroimidazo[1,2-a:5,4-c′]dipyridin-2(1H)- | | | |
| | yl)ethan-1-one | | | |
| 1.5.3 | 1-(1-(4-cyclopropyl-2-fluorophenyl)-3,4-dihydroimidazo[1,2-a:5,4- | 0.59 | 500.01 | 42 |
| | c′]dipyridin-2(1H)-yl)-2-((6-(dimethylamino)-2-methylpyridin-3- | | | |
| | yl)oxy)ethan-1-one | | | |
| 1.5.4 | 2-((2-chloropyridin-3-yl)oxy)-1-(1-(4-cyclopropyl-2-fluorophenyl)-3,4- | 0.72 | 476.89 | 34 |
| | dihydroimidazo[1,2-a:5,4-c′]dipyridin-2(1H)-yl)ethan-1-one | | | |
| 1.5.5 | 1-(1-(4-cyclopropyl-2-fluorophenyl)-3,4-dihydroimidazo[1,2-a:5,4- | 0.59 | 484.98 | 29 |
| | c′]dipyridin-2(1H)-yl)-2-((2-ethyl-6-methylpyridin-3-yl)oxy)ethan-1- | | | |
| | one | | | |
| 1.5.6 | 1-(1-(4-cyclopropyl-2-fluorophenyl)-3,4-dihydroimidazo[1,2-a:5,4- | 0.76 | 510.93 | 13 |
| | c′]dipyridin-2(1H)-yl)-2-((2-(trifluoromethyl)pyridin-3-yl)oxy)ethan-1- | | | |
| | one | | | |
| 1.6.1 | 2-((2-chloro-6-morpholinopyridin-3-yl)oxy)-1-(1-(2-morpholinothiazol- | 0.65 | 596.53 | 387 |
| | 5-yl)-3,4-dihydroimidazo[1,2-a:5,4-c′]dipyridin-2(1H)-yl)ethan-1-one | | | |
| 1.7.1 | 2-((2-chloro-6-morpholinopyridin-3-yl)oxy)-1-(1-(4- | 0.55 | 567.46 | 101 |
| | ((dimethylamino)methyl)thiophen-2-yl)-3,4-dihydroimidazo[1,2-a:5,4- | | | |
| | c′]dipyridin-2(1H)-yl)ethan-1-one | | | |
| 1.8.1 | 2-((2-chloro-6-morpholinopyridin-3-yl)oxy)-1-(1-(3-phenyl-1,2,4- | 0.77 | 572.46 | 16 |
| | oxadiazol-5-yl)-3,4-dihydroimidazo[1,2-a:5,4-c′]dipyridin-2(1H)- | | | |
| | yl)ethan-1-one | | | |
| 1.9.1 | 1-(1-(3-hydroxy-4-methoxyphenyl)-3,4-dihydroimidazo[1,2-a:5,4- | 0.72 | 480.02 | 230 |
| | c′]dipyridin-2(1H)-yl)-2-(naphthalen-2-yloxy)ethan-1-one | | | |
| 1.10.1 | 2-(2,4-dichlorophenoxy)-1-(1-(3,4-dimethylphenyl)-3,4- | 0.82 | 480.00 | 451 |
| | dihydroimidazo[1,2-a:5,4-c′]dipyridin-2(1H)-yl)ethan-1-one | | | |
| 1.11.1 | 1-(1-(6-chloro-2-fluoropyridin-3-yl)-3,4-dihydroimidazo[1,2-a:5,4- | 0.71 | 557.36 | 7 |
| | c′]dipyridin-2(1H)-yl)-2-((2-chloro-6-morpholinopyridin-3- | | | |
| | yl)oxy)ethan-1-one | | | |
| 1.11.2 | 1-(1-(6-chloro-2-fluoropyridin-3-yl)-3,4-dihydroimidazo[1,2-a:5,4- | 0.51 | 466.25 | 123 |
| | c′]dipyridin-2(1H)-yl)-2-((2-ethylpyridin-3-yl)oxy)ethan-1-one | | | |
| 1.11.3 | 1-(1-(6-chloro-2-fluoropyridin-3-yl)-3,4-dihydroimidazo[1,2-a:5,4- | 0.52 | 480.40 | 241 |
| | c′]dipyridin-2(1H)-yl)-2-((2-ethyl-6-methylpyridin-3-yl)oxy)ethan-1- | | | |
| | one | | | |
| 1.12.1 | 2-((2-chloro-6-morpholinopyridin-3-yl)oxy)-1-(1-(5-cyclopropyl-3- | 0.72 | 563.48 | 8 |
| | fluoropyridin-2-yl)-3,4-dihydroimidazo[1,2-a:5,4-c′]dipyridin-2(1H)- | | | |
| | yl)ethan-1-one | | | |
| 1.12.2 | 2-(2-chloro-4-morpholinophenoxy)-1-(1-(5-cyclopropyl-3- | 0.72 | 562.42 | 23 |
| | fluoropyridin-2-yl)-3,4-dihydroimidazo[1,2-a:5,4-c′]dipyridin-2(1H)- | | | |
| | yl)ethan-1-one | | | |
| 1.12.3 | 1-(1-(5-cyclopropyl-3-fluoropyridin-2-yl)-3,4-dihydroimidazo[1,2- | 0.53 | 486.48 | 146 |
| | a:5,4-c′]dipyridin-2(1H)-yl)-2-((2-ethyl-6-methylpyridin-3- | | | |
| | yl)oxy)ethan-1-one | | | |
| 1.13.1 | 2-((2-chloro-6-morpholinopyridin-3-yl)oxy)-1-(1-(6-cyclopropyl-2- | 0.64 | 563.47 | 8 |
| | fluoropyridin-3-yl)-3,4-dihydroimidazo[1,2-a:5,4-c′]dipyridin-2(1H)- | | | |
| | yl)ethan-1-one | | | |
| 1.14.1 | 1-(1-(5-chloro-3-fluoropyridin-2-yl)-3,4-dihydroimidazo[1,2-a:5,4- | 0.71 | 557.40 | 4 |
| | c′]dipyridin-2(1H)-yl)-2-((2-chloro-6-morpholinopyridin-3- | | | |
| | yl)oxy)ethan-1-one | | | |
| 1.14.2 | methyl (R)-6-chloro-5-(2-(1-(5-chloro-3-fluoropyridin-2-yl)-3,4- | 0.66 | 530.26 | 222 |
| | dihydroimidazo[1,2-a:5,4-c′]dipyridin-2(1H)-yl)-2- | | | |
| | oxoethoxy)picolinate | | | |
| 1.15.1 | methyl-4-(2-(2-((2-chloro-6-(methylsulfonamido)pyridin-3- | 0.65 | 587.99 | 71 |
| | yl)oxy)acetyl)-1,2,3,4-tetrahydroimidazo[1,2-a:5,4-c′]dipyridin-1-yl)-3- | | | |
| | fluorobenzoate | | | |
| 1.15.2 | methyl-4-(2-(2-((2-chloro-6-(cyclopropylcarbamoyl)pyridin-3- | 0.6 | 577.95 | 49 |
| | yl)oxy)acetyl)-1,2,3,4-tetrahydroimidazo[1,2-a:5,4-c′]dipyridin-1-yl)-3- | | | |
| | fluorobenzoate | | | |
| 1.16.1 | 6-chloro-N-cyclopropyl-5-(2-(1-(2-fluoro-4-(2-hydroxypropan-2- | 0.64 | 578.17 | 244 |
| | yl)phenyl)-3,4-dihydroimidazo[1,2-a:5,4-c′]dipyridin-2(1H)-yl)-2- | | | |
| | oxoethoxy)picolinamide | | | |
| 1.16.2 | N-(6-chloro-5-(2-(1-(2-fluoro-4-(2-hydroxypropan-2-yl)phenyl)-3,4- | 0.60 | 588.11 | 546 |
| | dihydroimidazo[1,2-a:5,4-c′]dipyridin-2(1H)-yl)-2-oxoethoxy)pyridin- | | | |
| | 2-yl)methanesulfonamide | | | |
| 1.17.1 | 6-chloro-N-cyclopropyl-5-(2-(1-(2-fluoro-4-(2-methoxypropan-2- | 0.7 | 592.20 | 140 |
| | yl)phenyl)-3,4-dihydroimidazo[1,2-a:5,4-c′]dipyridin-2(1H)-yl)-2- | | | |
| | oxoethoxy)picolinamide | | | |
| 1.17.2 | N-(6-chloro-5-(2-(1-(2-fluoro-4-(2-methoxypropan-2-yl)phenyl)-3,4- | 0.67 | 602.16 | 591 |
| | dihydroimidazo[1,2-a:5,4-c′]pyridin-2(1H)-yl)-2-oxoethoxy)pyridin- | | | |
| | 2-yl)methanesulfonamide | | | |
| 1.18.1 | 6-chloro-N-cyclopropyl-5-(2-(1-(2-fluoro-4-(methoxymethyl)phenyl)- | 0.68 | 564.14 | 38 |
| | 3,4-dihydroimidazo[1,2-a:5,4-c′]dipyridin-2(1H)-yl)-2- | | | |
| | oxoethoxy)picolinamide | | | |
| 1.18.2 | N-(6-chloro-5-(2-(1-(2-fluoro-4-(methoxymethyl)phenyl)-3,4- | 0.64 | 574.10 | 40 |
| | dihydroimidazo[1,2-a:5,4-c′]dipyridin-2(1H)-yl)-2-oxoethoxy)pyridin- | | | |
| | 2-yl)methanesulfonamide | | | |
| 1.19.1 | N-(6-chloro-5-(2-(1-(2-fluoro-4-(trifluoromethoxy)phenyl)-3,4- | 0.71 | 614.08 | 113 |
| | dihydroimidazo[1,2-a:5,4-c′]dipyridin-2(1H)-yl)-2-oxoethoxy)pyridin- | | | |
| | 2-yl)methanesulfonamide0.71 | | | |
| 1.19.2 | 6-chloro-N-cyclopropyl-5-(2-(1-(2-fluoro-4-(trifluoromethoxy)phenyl)- | 0.75 | 604.22 | 18 |
| | 3,4-dihydroimidazo[1,2-a:5,4-c′]dipyridin-2(1H)-yl)-2- | | | |
| | oxoethoxy)picolinamide | | | |
| 1.20.1 | N-(6-chloro-5-(2-(1-(4-cyano-2-fluorophenyl)-3,4-dihydroimidazo[1,2- | 0.63 | 555.05 | 98 |
| | a:5,4-c′]dipyridin-2(1H)-yl)-2-oxoethoxy)pyridin-2- | | | |
| | yl)methanesulfonamide | | | |
| 1.20.2 | 6-chloro-5-(2-(1-(4-cyano-2-fluorophenyl)-3,4-dihydroimidazo[1,2- | 0.67 | 545.12 | 31 |
| | a:5,4-c′]dipyridin-2(1H)-yl)-2-oxoethoxy)-N-cyclopropylpicolinamide | | | |
| 1.21.1 | 6-chloro-N-cyclopropyl-5-(2-(1-(2-fluoro-4-(2- | 0.69 | 594.16 | 54 |
| | methoxyethoxy)phenyl)-3,4-dihydroimidazo[1,2-a:5,4-c′]dipyridin- | | | |
| | 2(1H)-yl)-2-oxoethoxy)picolinamide | | | |
| 1.21.2 | N-(6-chloro-5-(2-(1-(2-fluoro-4-(2-methoxyethoxy)phenyl)-3,4- | 0.65 | 604.25 | 79 |
| | dihydroimidazo[1,2-a:5,4-c′]dipyridin-2(1H)-yl)-2-oxoethoxy)pyridin- | | | |
| | 2-yl)methanesulfonamide | | | |
| 2.1.1 | 1-(7-chloro-1-(3,4-dimethoxyphenyl)-3,4-dihydroimidazo[1,2-a:5,4- | 0.70 | 527.99 | 39 |
| | c′]dipyridin-2(1H)-yl)-2-(naphthalen-2-yloxy)ethan-1-one | | | |
| | hydrochloride | | | |
| 2.1.2 | 1-(7-chloro-1-(3,4-dimethoxyphenyl)-3,4-dihydroimidazo[1,2-a:5,4- | 0.59 | 611.07 | 66 |
| | c′]dipyridin-2(1H)-yl)-2-(2-chloro-4- | | | |
| | (morpholinomethyl)phenoxy)ethan-1-one | | | |
| 2.1.4 | 1-(7-chloro-1-(3,4-dimethoxyphenyl)-3,4-dihydroimidazo[1,2-a:5,4- | 0.67 | 557.12 | 390 |
| | c′]dipyridin-2(1H)-yl)-2-(2-chloro-4-nitrophenoxy)ethan-1-one | | | |
| 2.1.5 | 1-(7-chloro-1-(3,4-dimethoxyphenyl)-3,4-dihydroimidazo[1,2-a:5,4- | 0.57 | 612.05 | 316 |
| | c′]dipyridin-2(1H)-yl)-2-((2-chloro-6-(morpholinomethyl)pyridin-3- | | | |
| | yl)oxy)ethan-1-one | | | |
| 2.1.6 | 1-(7-chloro-1-(3,4-dimethoxyphenyl)-3,4-dihydroimidazo[1,2-a:5,4- | 0.80 | 545.64 | 53 |
| | c′]dipyridin-2(1H)-yl)-2-(2,4-dichlorophenoxy)ethan-1-one | | | |
| 2.1.7 | 1-(7-chloro-1-(3,4-dimethoxyphenyl)-3,4-dihydroimidazo[1,2-a:5,4- | 0.62 | 597.11 | 34 |
| | c′]dipyridin-2(1H)-yl)-2-(2-chloro-4-morpholinophenoxy)ethan-1-one | | | |
| 2.2.1 | 1-(7-chloro-1-(2-fluoropyridin-3-yl)-3,4-dihydroimidazo[1,2-a:5,4- | 0.56 | 569.98 | 269 |
| | c′]dipyridin-2(1H)-yl)-2-(2-chloro-4- | | | |
| | (morpholinomethyl)phenoxy)ethan-1-one | | | |
| 2.2.2 | 1-((1R)-7-chloro-1-(2-fluoropyridin-3-yl)-3,10a-dihydroimidazo[1,2- | 0.71 | 555.96 | 124 |
| | a:5,4-c′]dipyridin-2(1H)-yl)-2-(2-chloro-4-morpholinophenoxy)ethan- | | | |
| | 1-one | | | |
| 2.3.1 | 1-(7-chloro-1-(2-fluoro-4-methoxyphenyl)-3,4-dihydroimidazo[1,2- | 0.62 | 599.02 | 8 |
| | a:5,4-c′]dipyridin-2(1H)-yl)-2-(2-chloro-4- | | | |
| | (morpholinomethyl)phenoxy)ethan-1-one | | | |
| 2.3.2 | 1-(7-chloro-1-(2-fluoro-4-methoxyphenyl)-3,4-dihydroimidazo[1,2- | 0.60 | 600.04 | 59 |
| | a:5,4-c′]dipyridin-2(1H)-yl)-2-((2-chloro-6-(morpholinomethyl)pyridin- | | | |
| | 3-yl)oxy)ethan-1-one | | | |
| 2.3.3 | 1-(7-chloro-1-(2-fluoro-4-methoxyphenyl)-3,4-dihydroimidazo[1,2- | 0.83 | 571.94 | 208 |
| | a:5,4-c′]dipyridin-2(1H)-yl)-2-((4-chloro-1-methyl-5-(trifluoromethyl)- | | | |
| | 1H-pyrazol-3-yl)oxy)ethan-1-one | | | |
| 2.3.4 | 1-((1R)-7-chloro-1-(2-fluoro-4-methoxyphenyl)-3,10a- | 0.78 | 584.98 | 12 |
| | dihydroimidazo[1,2-a:5,4-c′]dipyridin-2(1H)-yl)-2-(2-chloro-4- | | | |
| | morpholinophenoxy)ethan-1-one | | | |
| 2.4.1 | N-(6-chloro-5-(2-(7-chloro-1-(4-chloro-2-fluorophenyl)-3,4- | 0.73 | 597.85 | 8 |
| | dihydroimidazo[1,2-a:5,4-c′]dipyridin-2(1H)-yl)-2-oxoethoxy)pyridin- | | | |
| | 2-yl)methanesulfonamide | | | |
| 2.5.1 | 1-((1R)-7-chloro-1-(6-methoxypyridin-3-yl)-3,10a- | 0.73 | 568.02 | 89 |
| | dihydroimidazo[1,2-a:5,4-c′]dipyridin-2(1H)-yl)-2-(2-chloro-4- | | | |
| | morpholinophenoxy)ethan-1-one | | | |
| 2.7.1 | 1-(7-chloro-1-(4-methoxy-3-((4-methoxybenzyl)oxy)phenyl)-3,4- | 0.79 | 634.24 | 60 |
| | dihydroimidazo[1,2-a:5,4-c′]dipyridin-2(1H)-yl)-2-(naphthalen-2- | | | |
| | yloxy)ethan-1-one | | | |
| 2.15.1 | N-(6-chloro-5-(2-(7-chloro-1-(2-fluoro-4-methylphenyl)-3,4- | 0.72 | 577.83 | 51 |
| | dihydroimidazo[1,2-a:5,4-c′]dipyridin-2(1H)-yl)-2-oxoethoxy)pyridin- | | | |
| | 2-yl)methanesulfonamide | | | |
| 3.1.1 | 1-(1-(3,4-dimethoxyphenyl)-7-(trifluoromethyl)-3,4- | 0.82 | 562.25 | 15 |
| | dihydroimidazo[1,2-a:5,4-c′]dipyridin-2(1H)-yl)-2-(naphthalen-2- | | | |
| | yloxy)ethan-1-one | | | |
| 3.1.31 | 2-(4-bromo-2-ethylphenoxy)-1-(1-(3,4-dimethoxyphenyl)-7- | 0.93 | 620.13 | 43 |
| | (trifluoromethyl)-3,4-dihydroimidazo[1,2-a:5,4-c′]dipyridin-2(1H)- | | | |
| | yl)ethan-1-one | | | |
| 3.2.1 | 2-(2-chloro-4-(morpholinomethyl)phenoxy)-1-(1-(2-fluoropyridin-3- | 0.66 | 604.17 | 43 |
| | yl)-7-(trifluoromethyl)-3,4-dihydroimidazo[1,2-a:5,4-c′]dipyridin- | | | |
| | 2(1H)-yl)ethan-1-one | | | |
| 3.2.2 | 1-(1-(2-fluoropyridin-3-yl)-7-(trifluoromethyl)-3,4-dihydroimidazo[1,2- | 0.88 | 521.14 | 82 |
| | a:5,4-c′]dipyridin-2(1H)-yl)-2-(naphthalen-2-yloxy)ethan-1-one | | | |
| 3.2.3 | 1-(1-(2-fluoropyridin-3-yl)-7-(trifluoromethyl)-3,4-dihydroimidazo[1,2- | 0.85 | 485.12 | 251 |
| | a:5,4-c′]dipyridin-2(1H)-yl)-2-(p-tolyloxy)ethan-1-one | | | |
| 3.3.1 | 1-(1-(2-fluoro-4-methoxyphenyl)-7-(trifluoromethyl)-3,4- | 0.92 | 549.85 | 5 |
| | dihydroimidazo[1,2-a:5,4-c′]dipyridin-2(1H)-yl)-2-(naphthalen-2- | | | |
| | yloxy)ethan-1-one | | | |
| 3.3.2 | 2-(2-chloro-4-(morpholinomethyl)phenoxy)-1-(1-(2-fluoro-4- | 0.72 | 633.07 | 7 |
| | methoxyphenyl)-7-(trifluoromethyl)-3,4-dihydroimidazo[1,2-a:5,4- | | | |
| | c′]dipyridin-2(1H)-yl)ethan-1-one | | | |
| 3.3.3 | 2-(2,3-dimethyl-4-(morpholinomethyl)phenoxy)-1-(1-(2-fluoro-4- | 0.73 | 627.25 | 8 |
| | methoxyphenyl)-7-(trifluoromethyl)-3,4-dihydroimidazo[1,2-a:5,4- | | | |
| | c′]dipyridin-2(1H)-yl)ethan-1-one | | | |
| 3.3.4 | tert-butyl 6-(2-(1-(2-fluoro-4-methoxyphenyl)-7-(trifluoromethyl)-3,4- | 0.98 | 669.18 | 178 |
| | dihydroimidazo[1,2-a:5,4-c′]dipyridin-2(1H)-yl)-2-oxoethoxy)-5- | | | |
| | methyl-3,4-dihydroisoquinoline-2(1H)-carboxylate | | | |
| 3.4.1 | 1-(1-(4-chloro-2-fluorophenyl)-7-(trifluoromethyl)-3,4- | 0.88 | 538.89 | 44 |
| | dihydroimidazo[1,2-a:5,4-c′]dipyridin-2(1H)-yl)-2-((2-chloropyridin-3- | | | |
| | yl)oxy)ethan-1-one | | | |
| 3.4.2 | 1-(1-(4-chloro-2-fluorophenyl)-7-(trifluoromethyl)-3,4- | 0.75 | 637.02 | 7 |
| | dihydroimidazo[1,2-a:5,4-c′]dipyridin-2(1H)-yl)-2-(2-chloro-4- | | | |
| | (morpholinomethyl)phenoxy)ethan-1-one | | | |
| 3.4.3 | 2-((4-chloro-1-methyl-5-(trifluoromethyl)-1H-pyrazol-3-yl)oxy)-1-(1- | 0.97 | 609.71 | 64 |
| | (4-chloro-2-fluorophenyl)-7-(trifluoromethyl)-3,4-dihydroimidazo[1,2- | | | |
| | a:5,4-c′]dipyridin-2(1H)-yl)ethan-1-one | | | |
| 3.4.4 | 1-(1-(4-chloro-2-fluorophenyl)-7-(trifluoromethyl)-3,4- | 0.73 | 547.08 | 21 |
| | dihydroimidazo[1,2-a:5,4-c′]dipyridin-2(1H)-yl)-2-((2-ethyl-6- | | | |
| | methylpyridin-3-yl)oxy)ethan-1-one | | | |
| 3.4.5 | ((1R)-1-(4-chloro-2-fluorophenyl)-7-(trifluoromethyl)-3,10a- | 0.93 | 623.05 | 6 |
| | dihydroimidazo[1,2-a:5,4-c′]dipyridin-2(1H)-yl)-2-(2-chloro-4- | | | |
| | morpholinophenoxy)ethan-1-one | | | |
| 3.4.6 | 1-(1-(4-chloro-2-fluorophenyl)-7-(trifluoromethyl)-3,4- | 0.92 | 625.71 | 5 |
| | dihydroimidazo[1,2-a:5,4-c′]dipyridin-2(1H)-yl)-2-((2-chloro-6- | | | |
| | morpholinopyridin-3-yl)oxy)ethan-1-one | | | |
| 3.4.7 | N-(6-chloro-5-(2-(1-(4-chloro-2-fluorophenyl)-7-(trifluoromethyl)-3,4- | 0.85 | 631.76 | 5 |
| | dihydroimidazo[1,2-a:5,4-c′]dipyridin-2(1H)-yl)-2-oxoethoxy)pyridin- | | | |
| | 2-yl)methanesulfonamide | | | |
| 3.5.1 | 1-(1-(4-cyclopropyl-2-fluorophenyl)-7-(trifluoromethyl)-3,4- | 0.75 | 553.19 | 11 |
| | dihydroimidazo[1,2-a:5,4-c′]dipyridin-2(1H)-yl)-2-((2-ethyl-6- | | | |
| | methylpyridin-3-yl)oxy)ethan-1-one | | | |
| 3.5.2 | 2-(2-chloro-4-morpholinophenoxy)-1-(1-(4-cyclopropyl-2- | 0.94 | 629.23 | 6 |
| | fluorophenyl)-7-(trifluoromethyl)-3,4-dihydroimidazo[1,2-a:5,4- | | | |
| | c′]dipyridin-2(1H)-yl)ethan-1-one | | | |
| 3.5.3 | 1-(1-(4-cyclopropyl-2-fluorophenyl)-7-(trifluoromethyl)-3,4- | 0.86 | 569.49 | 85 |
| | dihydroimidazo[1,2-a:5,4-c′]dipyridin-2(1H)-yl)-2-((2-ethyl-6-methyl- | | | |
| | 1-(I1-oxidanyl)-1I4-pyridin-3-yl)oxy)ethan-1-one | | | |
| 3.5.4 | 2-((2-chloro-1-(I1-oxidanyl)-1I4-pyridin-3-yl)oxy)-1-(1-(4-cyclopropyl- | 0.79 | 561.37 | 210 |
| | 2-fluorophenyl)-7-(trifluoromethyl)-3,4-dihydroimidazo[1,2-a:5,4- | | | |
| | c′]dipyridin-2(1H)-yl)ethan-1-one | | | |
| 3.6.1 | 1-(1-(6-methoxypyridin-3-yl)-7-(trifluoromethyl)-3,4- | 0.88 | 533.04 | 52 |
| | dihydroimidazo[1,2-a:5,4-c′]dipyridin-2(1H)-yl)-2-(naphthalen-2- | | | |
| | yloxy)ethan-1-one | | | |
| 3.6.2 | 2-(2-chloro-4-(morpholinomethyl)phenoxy)-1-(1-(6-methoxypyridin-3- | 0.67 | 616.16 | 50 |
| | yl)-7-(trifluoromethyl)-3,4-dihydroimidazo[1,2-a:5,4-c′]dipyridin- | | | |
| | 2(1H)-yl)ethan-1-one | | | |
| 3.7.1 | 2-(2-chloro-4-(morpholinomethyl)phenoxy)-1-(1-(6-ethoxypyridin-3- | 0.70 | 630.21 | 18 |
| | yl)-7-(trifluoromethyl)-3,4-dihydroimidazo[1,2-a:5,4-c′]dipyridin- | | | |
| | 2(1H)-yl)ethan-1-one | | | |
| 3.8.1 | 2-(2-chloro-4-(morpholinomethyl)phenoxy)-1-(1-(6-(2,2,2- | 0.76 | 684.16 | 86 |
| | trifluoroethoxy)pyridin-3-yl)-7-(trifluoromethyl)-3,4- | | | |
| | dihydroimidazo[1,2-a:5,4-c′]dipyridin-2(1H)-yl)ethan-1-one | | | |
| 3.9.1 | 1-(1-(3-hydroxy-4-methoxyphenyl)-7-(trifluoromethyl)-3,4- | 0.85 | 548.00 | 45 |
| | dihydroimidazo[1,2-a:5,4-c′]dipyridin-2(1H)-yl)-2-(naphthalen-2- | | | |
| | yloxy)ethan-1-one | | | |
| 3.10.1 | 1-(1-(3,4-dimethylphenyl)-7-(trifluoromethyl)-3,4-dihydroimidazo[1,2- | 0.95 | 530.17 | 33 |
| | a:5,4-c′]dipyridin-2(1H)-yl)-2-(naphthalen-2-yloxy)ethan-1-one | | | |
| 3.10.2 | 2-(2-chloro-4-(morpholinomethyl)phenoxy)-1-(1-(3,4- | 0.75 | 613.01 | 23 |
| | dimethylphenyl)-7-(trifluoromethyl)-3,4-dihydroimidazo[1,2-a:5,4- | | | |
| | c′]dipyridin-2(1H)-yl)ethan-1-one | | | |
| 3.10.3 | 2-((2-chloropyridin-3-yl)oxy)-1-(1-(3,4-dimethylphenyl)-7- | 0.86 | 515.14 | 239 |
| | (trifluoromethyl)-3,4-dihydroimidazo[1,2-a:5,4-c′]dipyridin-2(1H)- | | | |
| | yl)ethan-1-one | | | |
| 3.11.1 | 1-(1-(4-hydroxy-3-methoxyphenyl)-7-(trifluoromethyl)-3,4- | 0.84 | 548.04 | 318 |
| | dihydroimidazo[1,2-a:5,4-c′]dipyridin-2(1H)-yl)-2-(naphthalen-2- | | | |
| | yloxy)ethan-1-one | | | |
| 3.12.1 | 1-(1-(3-(difluoromethoxy)-4-methoxyphenyl)-7-(trifluoromethyl)-3,4- | 0.94 | 597.82 | 41 |
| | dihydroimidazo[1,2-a:5,4-c′]dipyridin-2(1H)-yl)-2-(naphthalen-2- | | | |
| | yloxy)ethan-1-one | | | |
| 3.13.1 | 1-(1-(4-methoxy-3-(trifluoromethoxy)phenyl)-7-(trifluoromethyl)-3,4- | 0.97 | 616.06 | 75 |
| | dihydroimidazo[1,2-a:5,4-c′]dipyridin-2(1H)-yl)-2-(naphthalen-2- | | | |
| | yloxy)ethan-1-one | | | |
| 3.14.1 | 1-(1-(4-(difluoromethoxy)-3-methoxyphenyl)-7-(trifluoromethyl)-3,4- | 0.94 | 597.83 | 106 |
| | dihydroimidazo[1,2-a:5,4-c′]dipyridin-2(1H)-yl)-2-(naphthalen-2- | | | |
| | yloxy)ethan-1-one | | | |
| 3.15.1 | 1-(1-(4-(2-(dimethylamino)ethoxy)-3-methoxyphenyl)-7- | 0.73 | 619.13 | 70 |
| | (trifluoromethyl)-3,4-dihydroimidazo[1,2-a:5,4-c′]dipyridin-2(1H)-yl)- | | | |
| | 2-(naphthalen-2-yloxy)ethan-1-one | | | |
| 3.16.1 | 1-(1-(benzo[d][1,3]dioxol-5-yl)-7-(trifluoromethyl)-3,4- | 0.91 | 546.18 | 150 |
| | dihydroimidazo[1,2-a:5,4-c′]dipyridin-2(1H)-yl)-2-(naphthalen-2- | | | |
| | yloxy)ethan-1-one | | | |
| 3.17.1 | 1-(1-(2,3-dihydrobenzo[b][1,4]dioxin-6-yl)-7-(trifluoromethyl)-3,4- | 0.91 | 560.11 | 138 |
| | dihydroimidazo[1,2-a:5,4-c′]dipyridin-2(1H)-yl)-2-(naphthalen-2- | | | |
| | yloxy)ethan-1-one | | | |
| 3.18.1 | 1-(1-(2-fluoro-4,5-dimethoxyphenyl)-7-(trifluoromethyl)-3,4- | 0.90 | 580.12 | 5 |
| | dihydroimidazo[1,2-a:5,4-c′]dipyridin-2(1H)-yl)-2-(naphthalen-2- | | | |
| | yloxy)ethan-1-one | | | |
| 3.18.2 | 2-(2-chloro-4-(morpholinomethyl)phenoxy)-1-(1-(2-fluoro-4,5- | 0.70 | 663.07 | 5 |
| | dimethoxyphenyl)-7-(trifluoromethyl)-3,4-dihydroimidazo[1,2-a:5,4- | | | |
| | c′]dipyridin-2(1H)-yl)ethan-1-one | | | |
| 3.18.3 | tert-butyl 7-(2-(1-(2-fluoro-4,5-dimethoxyphenyl)-7-(trifluoromethyl)- | 0.93 | 685.26 | 9 |
| | 3,4-dihydroimidazo[1,2-a:5,4-c′]dipyridin-2(1H)-yl)-2-oxoethoxy)-3,4- | | | |
| | dihydroisoquinoline-2(1H)-carboxylate | | | |
| 3.18.8 | N-(3-(3-chloro-4-(2-(1-(2-fluoro-4,5-dimethoxyphenyl)-7- | 0.84 | 738.91 | 48 |
| | (trifluoromethyl)-3,4-dihydroimidazo[1,2-a:5,4-c′]dipyridin-2(1H)-yl)- | | | |
| | 2-oxoethoxy)phenyl)oxetan-3-yl)-2-methylpropane-2-sulfinamide | | | |
| 3.19.1 | 1-(1-(5,6-dimethoxypyridin-3-yl)-7-(trifluoromethyl)-3,4- | 0.88 | 563.11 | 30 |
| | dihydroimidazo[1,2-a:5,4-c′]dipyridin-2(1H)-yl)-2-(naphthalen-2- | | | |
| | yloxy)ethan-1-one | | | |
| 3.20.1 | 1-(1-(5,6-dimethoxypyridin-2-yl)-7-(trifluoromethyl)-3,4- | 0.89 | 563.03 | 53 |
| | dihydroimidazo[1,2-a:5,4-c′]dipyridin-2(1H)-yl)-2-(naphthalen-2- | | | |
| | yloxy)ethan-1-one | | | |
| 3.21.1 | 1-(1-(3-fluoropyridin-2-yl)-7-(trifluoromethyl)-3,4-dihydroimidazo[1,2- | 0.86 | 521.12 | 69 |
| | a:5,4-c′]dipyridin-2(1H)-yl)-2-(naphthalen-2-yloxy)ethan-1-one | | | |
| 3.21.2 | 2-(2-chloro-4-(morpholinomethyl)phenoxy)-1-(1-(3-fluoropyridin-2- | 0.64 | 604.11 | 345 |
| | yl)-7-(trifluoromethyl)-3,4-dihydroimidazo[1,2-a:5,4-c′]dipyridin- | | | |
| | 2(1H)-yl)ethan-1-one | | | |
| 3.21.3 | 2-(2-chloro-4-morpholinophenoxy)-1-(1-(3-fluoropyridin-2-yl)-7- | 0.80 | 590.47 | 88 |
| | (trifluoromethyl)-3,4-dihydroimidazo[1,2-a:5,4-c′]dipyridin-2(1H)- | | | |
| | yl)ethan-1-one | | | |
| 3.21.4 | 2-(4-chloro-2-methylphenoxy)-1-(1-(3-fluoropyridin-2-yl)-7- | 0.87 | 519.28 | 464 |
| | (trifluoromethyl)-3,4-dihydroimidazo[1,2-a:5,4-c′]dipyridin-2(1H)- | | | |
| | yl)ethan-1-one | | | |
| 3.22.1 | 2-(2-chloro-4-(morpholinomethyl)phenoxy)-1-(1-(2-fluoro-4- | 0.74 | 617.13 | 7 |
| | methylphenyl)-7-(trifluoromethyl)-3,4-dihydroimidazo[1,2-a:5,4- | | | |
| | c′]dipyridin-2(1H)-yl)ethan-1-one | | | |
| 3.22.2 | 2-((2-chloropyridin-3-yl)oxy)-1-(1-(2-fluoro-4-methylphenyl)-7- | 0.86 | 519.11 | 44 |
| | (trifluoromethyl)-3,4-dihydroimidazo[1,2-a:5,4-c′]dipyridin-2(1H)- | | | |
| | yl)ethan-1-one | | | |
| 3.22.3 | 2-((4-chloro-1-methyl-5-(trifluoromethyl)-1H-pyrazol-3-yl)oxy)-1-(1- | 0.96 | 589.99 | 33 |
| | (2-fluoro-4-methylphenyl)-7-(trifluoromethyl)-3,4-dihydroimidazo[1,2- | | | |
| | a:5,4-c′]dipyridin-2(1H)-yl)ethan-1-one | | | |
| 3.22.4 | 2-((2-ethyl-6-methylpyridin-3-yl)oxy)-1-(1-(2-fluoro-4-methylphenyl)- | 0.72 | 527.17 | 17 |
| | 7-(trifluoromethyl)-3,4-dihydroimidazo[1,2-a:5,4-c′]dipyridin-2(1H)- | | | |
| | yl)ethan-1-one | | | |
| 3.22.5 | N-(6-chloro-5-(2-(1-(2-fluoro-4-methylphenyl)-7-(trifluoromethyl)-3,4- | 0.83 | 611.95 | 15 |
| | dihydroimidazo[1,2-a:5,4-c′]dipyridin-2(1H)-yl)-2-oxoethoxy)pyridin- | | | |
| | 2-yl)methanesulfonamide | | | |
| 3.23.1 | 2-(naphthalen-2-yloxy)-1-(1-(p-tolyl)-7-(trifluoromethyl)-3,4- | 0.94 | 516.03 | 67 |
| | dihydroimidazo[1,2-a:5,4-c′]dipyridin-2(1H)-yl)ethan-1-one | | | |
| 3.23.2 | 2-(2-chloro-4-(morpholinomethyl)phenoxy)-1-(1-(p-tolyl)-7- | 0.73 | 599.07 | 32 |
| | (trifluoromethyl)-3,4-dihydroimidazo[1,2-a:5,4-c′]dipyridin-2(1H)- | | | |
| | yl)ethan-1-one | | | |
| 3.24.1 | 1-(1-(4-methoxyphenyl)-7-(trifluoromethyl)-3,4-dihydroimidazo[1,2- | 0.91 | 532.02 | 44 |
| | a:5,4-c′]dipyridin-2(1H)-yl)-2-(naphthalen-2-yloxy)ethan-1-one | | | |
| 3.24.2 | 2-((2-chloropyridin-3-yl)oxy)-1-(1-(4-methoxyphenyl)-7- | 0.81 | 517.12 | 429 |
| | (trifluoromethyl)-3,4-dihydroimidazo[1,2-a:5,4-c′]dipyridin-2(1H)- | | | |
| | yl)ethan-1-one | | | |
| 3.25.1 | 1-(1-(4-ethoxyphenyl)-7-(trifluoromethyl)-3,4-dihydroimidazo[1,2- | 0.93 | 546.12 | 104 |
| | a:5,4-c′]dipyridin-2(1H)-yl)-2-(naphthalen-2-yloxy)ethan-1-one | | | |
| 3.25.2 | 2-(2-chloro-4-(morpholinomethyl)phenoxy)-1-(1-(4-ethoxyphenyl)-7- | 0.73 | 629.10 | 20 |
| | (trifluoromethyl)-3,4-dihydroimidazo[1,2-a:5,4-c′]dipyridin-2(1H)- | | | |
| | yl)ethan-1-one | | | |
| 3.25.3 | 2-(2,3-dimethyl-4-(morpholinomethyl)phenoxy)-1-(1-(4- | 0.74 | 623.26 | 51 |
| | ethoxyphenyl)-7-(trifluoromethyl)-3,4-dihydroimidazo[1,2-a:5,4- | | | |
| | c′]dipyridin-2(1H)-yl)ethan-1-one | | | |
| 3.26.1 | 1-(1-(4-(difluoromethoxy)phenyl)-7-(trifluoromethyl)-3,4- | 0.94 | 568.01 | 120 |
| | dihydroimidazo[1,2-a:5,4-c′]dipyridin-2(1H)-yl)-2-(naphthalen-2- | | | |
| | yloxy)ethan-1-one | | | |
| 3.26.2 | 2-(2-chloro-4-(morpholinomethyl)phenoxy)-1-(1-(4- | 0.74 | 651.08 | 54 |
| | (difluoromethoxy)phenyl)-7-(trifluoromethyl)-3,4-dihydroimidazo[1,2- | | | |
| | a:5,4-c′]dipyridin-2(1H)-yl)ethan-1-one | | | |
| 3.27.1 | 2-(2-chloro-4-(morpholinomethyl)phenoxy)-1-(1-(4- | 0.78 | 669.09 | 135 |
| | (trifluoromethoxy)phenyl)-7-(trifluoromethyl)-3,4-dihydroimidazo[1,2- | | | |
| | a:5,4-c′]dipyridin-2(1H)-yl)ethan-1-one | | | |
| 3.28.1 | 2-(naphthalen-2-yloxy)-1-(7-(trifluoromethyl)-1-(4- | 0.97 | 570.01 | 154 |
| | (trifluoromethyl)phenyl)-3,4-dihydroimidazo[1,2-a:5,4-c′]dipyridin- | | | |
| | 2(1H)-yl)ethan-1-one | | | |
| 3.28.2 | 2-(2-chloro-4-(morpholinomethyl)phenoxy)-1-(7-(trifluoromethyl)-1- | 0.77 | 653.19 | 77 |
| | (4-(trifluoromethyl)phenyl)-3,4-dihydroimidazo[1,2-a:5,4-c′]dipyridin- | | | |
| | 2(1H)-yl)ethan-1-one | | | |
| 3.29.1 | 1-(1-(4-fluorophenyl)-7-(trifluoromethyl)-3,4-dihydroimidazo[1,2- | 0.92 | 519.96 | 312 |
| | a:5,4-c′]dipyridin-2(1H)-yl)-2-(naphthalen-2-yloxy)ethan-1-one | | | |
| 3.29.2 | 2-(2-chloro-4-(morpholinomethyl)phenoxy)-1-(1-(4-fluorophenyl)-7- | 0.71 | 603.14 | 202 |
| | (trifluoromethyl)-3,4-dihydroimidazo[1,2-a:5,4-c′]dipyridin-2(1H)- | | | |
| | yl)ethan-1-one | | | |
| 3.30.1 | 1-(1-(4-chlorophenyl)-7-(trifluoromethyl)-3,4-dihydroimidazo[1,2- | 0.95 | 535.96 | 59 |
| | a:5,4-c′]dipyridin-2(1H)-yl)-2-(naphthalen-2-yloxy)ethan-1-one | | | |
| 3.30.2 | 2-(2-chloro-4-(morpholinomethyl)phenoxy)-1-(1-(4-chlorophenyl)-7- | 0.74 | 619.13 | 20 |
| | (trifluoromethyl)-3,4-dihydroimidazo[1,2-a:5,4-c′]dipyridin-2(1H)- | | | |
| | yl)ethan-1-one | | | |
| 3.31.1 | 1-(1-(4-(aminomethyl)phenyl)-7-(trifluoromethyl)-3,4- | 0.92 | 527.00 | 73 |
| | dihydroimidazo[1,2-a:5,4-c′]dipyridin-2(1H)-yl)-2-(naphthalen-2- | | | |
| | yloxy)ethan-1-one | | | |
| 3.31.2 | 1-(1-(4-(aminomethyl)phenyl)-7-(trifluoromethyl)-3,4- | 0.70 | 609.69 | 145 |
| | dihydroimidazo[1,2-a:5,4-c′]dipyridin-2(1H)-yl)-2-(2-chloro-4- | | | |
| | (morpholinomethyl)phenoxy)ethan-1-one | | | |
| 3.32.1 | 1-(1-(3-methoxyphenyl)-7-(trifluoromethyl)-3,4-dihydroimidazo[1,2- | 0.92 | 532.02 | 228 |
| | a:5,4-c′]dipyridin-2(1H)-yl)-2-(naphthalen-2-yloxy)ethan-1-one | | | |
| 3.33.1 | 1-(1-(4-fluoro-3-methylphenyl)-7-(trifluoromethyl)-3,4- | 0.94 | 533.91 | 127 |
| | dihydroimidazo[1,2-a:5,4-c′]dipyridin-2(1H)-yl)-2-(naphthalen-2- | | | |
| | yloxy)ethan-1-one | | | |
| 3.33.2 | 2-(2-chloro-4-(morpholinomethyl)phenoxy)-1-(1-(4-fluoro-3- | 0.74 | 617.08 | 110 |
| | methylphenyl)-7-(trifluoromethyl)-3,4-dihydroimidazo[1,2-a:5,4- | | | |
| | c′]dipyridin-2(1H)-yl)ethan-1-one | | | |
| 3.34.1 | 1-(1-(3,4-difluorophenyl)-7-(trifluoromethyl)-3,4-dihydroimidazo[1,2- | 0.94 | 538.11 | 486 |
| | a:5,4-c′]dipyridin-2(1H)-yl)-2-(naphthalen-2-yloxy)ethan-1-one | | | |
| 3.34.2 | 2-(2-chloro-4-(morpholinomethyl)phenoxy)-1-(1-(3,4-difluorophenyl)- | 0.74 | 621.03 | 242 |
| | 7-(trifluoromethyl)-3,4-dihydroimidazo[1,2-a:5,4-c′]dipyridin-2(1H)- | | | |
| | yl)ethan-1-one | | | |
| 3.35.1 | 1-(1-(2-fluorophenyl)-7-(trifluoromethyl)-3,4-dihydroimidazo[1,2- | 0.92 | 520.12 | 17 |
| | a:5,4-c′]dipyridin-2(1H)-yl)-2-(naphthalen-2-yloxy)ethan-1-one | | | |
| 3.35.2 | 2-(2-chloro-4-(morpholinomethyl)phenoxy)-1-(1-(2-fluorophenyl)-7- | 0.71 | 603.19 | 16 |
| | (trifluoromethyl)-3,4-dihydroimidazo[1,2-a:5,4-c′]dipyridin-2(1H)- | | | |
| | yl)ethan-1-one | | | |
| 3.35.3 | 2-((4-chloro-1-methyl-5-(trifluoromethyl)-1H-pyrazol-3-yl)oxy)-1-(1- | 0.93 | 576.08 | 338 |
| | (2-fluorophenyl)-7-(trifluoromethyl)-3,4-dihydroimidazo[1,2-a:5,4- | | | |
| | c′]dipyridin-2(1H)-yl)ethan-1-one | | | |
| 3.36.1 | 2-(naphthalen-2-yloxy)-1-(1-(pyridin-3-yl)-7-(trifluoromethyl)-3,4- | 0.78 | 503.18 | 208 |
| | dihydroimidazo[1,2-a:5,4-c′]dipyridin-2(1H)-yl)ethan-1-one | | | |
| 3.36.2 | 2-(2-chloro-4-(morpholinomethyl)phenoxy)-1-(1-(pyridin-3-yl)-7- | 0.59 | 586.14 | 488 |
| | (trifluoromethyl)-3,4-dihydroimidazo[1,2-a:5,4-c′]dipyridin-2(1H)- | | | |
| | yl)ethan-1-one | | | |
| 3.37.1 | 2-(2-chloro-4-(morpholinomethyl)phenoxy)-1-(1-(6-chloropyridin-3- | 0.69 | 620.13 | 50 |
| | yl)-7-(trifluoromethyl)-3,4-dihydroimidazo[1,2-a:5,4-c′]dipyridin- | | | |
| | 2(1H)-yl)ethan-1-one | | | |
| 3.37.2 | 1-(1-(6-chloropyridin-3-yl)-7-(trifluoromethyl)-3,4-dihydroimidazo[1,2- | 0.90 | 537.11 | 57 |
| | a:5,4-c′]dipyridin-2(1H)-yl)-2-(naphthalen-2-yloxy)ethan-1-one | | | |
| 3.38.1 | 1-(1-(6-methylpyridin-3-yl)-7-(trifluoromethyl)-3,4- | 0.75 | 517.06 | 26 |
| | dihydroimidazo[1,2-a:5,4-c′]dipyridin-2(1H)-yl)-2-(naphthalen-2- | | | |
| | yloxy)ethan-1-one | | | |
| 3.38.2 | 2-(2-chloro-4-(morpholinomethyl)phenoxy)-1-(1-(6-methylpyridin-3- | 0.57 | 600.15 | 92 |
| | yl)-7-(trifluoromethyl)-3,4-dihydroimidazo[1,2-a:5,4-c′]dipyridin- | | | |
| | 2(1H)-yl)ethan-1-one | | | |
| 3.39.1 | 2-(2-chloro-4-(morpholinomethyl)phenoxy)-1-(1-(6-methoxy-4- | 0.68 | 630.20 | 46 |
| | methylpyridin-3-yl)-7-(trifluoromethyl)-3,4-dihydroimidazo[1,2-a:5,4- | | | |
| | c′]dipyridin-2(1H)-yl)ethan-1-one | | | |
| 3.40.1 | 1-(1-(1,3-dimethyl-1H-pyrazol-5-yl)-7-(trifluoromethyl)-3,4- | 0.82 | 520.19 | 432 |
| | dihydroimidazo[1,2-a:5,4-c′]dipyridin-2(1H)-yl)-2-(naphthalen-2- | | | |
| | yloxy)ethan-1-one | | | |
| 3.41.1 | 2-(naphthalen-2-yloxy)-1-(1-(1-phenyl-1H-pyrazol-4-yl)-7- | 0.92 | 568.05 | 328 |
| | (trifluoromethyl)-3,4-dihydroimidazo[1,2-a:5,4-c′]dipyridin-2(1H)- | | | |
| | yl)ethan-1-one | | | |
| 3.41.2 | 2-(2-chloro-4-(morpholinomethyl)phenoxy)-1-(1-(1-phenyl-1H- | 0.72 | 651.10 | 24 |
| | pyrazol-4-yl)-7-(trifluoromethyl)-3,4-dihydroimidazo[1,2-a:5,4- | | | |
| | c′]dipyridin-2(1H)-yl)ethan-1-one | | | |
| 3.41.3 | 2-(2,3-dimethyl-4-(morpholinomethyl)phenoxy)-1-(1-(1-phenyl-1H- | 0.72 | 645.26 | 68 |
| | pyrazol-4-yl)-7-(trifluoromethyl)-3,4-dihydroimidazo[1,2-a:5,4- | | | |
| | c′]dipyridin-2(1H)-yl)ethan-1-one | | | |
| 3.42.1 | 1-(1-(4-methylthiazol-2-yl)-7-(trifluoromethyl)-3,4- | 0.90 | 522.95 | 260 |
| | dihydroimidazo[1,2-a:5,4-c′]dipyridin-2(1H)-yl)-2-(naphthalen-2- | | | |
| | yloxy)ethan-1-one | | | |
| 3.42.2 | 2-(2-chloro-4-(morpholinomethyl)phenoxy)-1-(1-(4-methylthiazol-2- | 0.68 | 606.09 | 427 |
| | yl)-7-(trifluoromethyl)-3,4-dihydroimidazo[1,2-a:5,4-c′]dipyridin- | | | |
| | 2(1H)-yl)ethan-1-one | | | |
| 3.43.1 | 2-(2-chloro-4-(morpholinomethyl)phenoxy)-1-(1-(imidazo[1,5- | 0.64 | 625.05 | 130 |
| | a]pyridin-3-yl)-7-(trifluoromethyl)-3,4-dihydroimidazo[1,2-a:5,4- | | | |
| | c′]dipyridin-2(1H)-yl)ethan-1-one | | | |
| 3.43.2 | 1-(1-(imidazo[1,5-a]pyridin-3-yl)-7-(trifluoromethyl)-3,4- | 0.84 | 542.14 | 209 |
| | dihydroimidazo[1,2-a:5,4-c′]dipyridin-2(1H)-yl)-2-(naphthalen-2- | | | |
| | yloxy)ethan-1-one | | | |
| 3.44.1 | 1-(1-(benzo[d]thiazol-2-yl)-7-(trifluoromethyl)-3,4- | 0.89 | 559.06 | 76 |
| | dihydroimidazo[1,2-a:5,4-c′]dipyridin-2(1H)-yl)-2-(naphthalen-2- | | | |
| | yloxy)ethan-1-one | | | |
| 3.44.2 | 1-(1-(benzo[d]thiazol-2-yl)-7-(trifluoromethyl)-3,4- | 0.67 | 641.91 | 23 |
| | dihydroimidazo[1,2-a:5,4-c′]dipyridin-2(1H)-yl)-2-(2-chloro-4- | | | |
| | (morpholinomethyl)phenoxy)ethan-1-one | | | |
| 3.45.1 | 1-(1-(5-methylisoxazol-3-yl)-7-(trifluoromethyl)-3,4- | 0.80 | 507.17 | 349 |
| | dihydroimidazo[1,2-a:5,4-c′]dipyridin-2(1H)-yl)-2-(naphthalen-2- | | | |
| | yloxy)ethan-1-one | | | |
| 3.46.1 | 2-(2-chloro-4-(morpholinomethyl)phenoxy)-1-(1-(5-phenylisoxazol-3- | 0.69 | 652.19 | 22 |
| | yl)-7-(trifluoromethyl)-3,4-dihydroimidazo[1,2-a:5,4-c′]dipyridin- | | | |
| | 2(1H)-yl)ethan-1-one | | | |
| 3.46.2 | 2-(naphthalen-2-yloxy)-1-(1-(5-phenylisoxazol-3-yl)-7- | 0.91 | 569.13 | 206 |
| | (trifluoromethyl)-3,4-dihydroimidazo[1,2-a:5,4-c′]dipyridin-2(1H)- | | | |
| | yl)ethan-1-one | | | |
| 3.47.1 | 1-(1-(5-(4-fluorophenyl)isoxazol-3-yl)-7-(trifluoromethyl)-3,4- | 0.91 | 587.21 | 368 |
| | dihydroimidazo[1,2-a:5,4-c′]dipyridin-2(1H)-yl)-2-(naphthalen-2- | | | |
| | yloxy)ethan-1-one | | | |
| 3.47.2 | 2-(2-chloro-4-(morpholinomethyl)phenoxy)-1-(1-(5-(4- | 0.71 | 670.19 | 41 |
| | fluorophenyl)isoxazol-3-yl)-7-(trifluoromethyl)-3,4- | | | |
| | dihydroimidazo[1,2-a:5,4-c′]dipyridin-2(1H)-yl)ethan-1-one | | | |
| 3.48.1 | 2-(2-chloro-4-(morpholinomethyl)phenoxy)-1-(1-(imidazo[1,5- | 0.64 | 625.02 | 214 |
| | a]pyridin-1-yl)-7-(trifluoromethyl)-3,4-dihydroimidazo[1,2-a:5,4- | | | |
| | c′]dipyridin-2(1H)-yl)ethan-1-one | | | |
| 3.49.1 | 2-(naphthalen-2-yloxy)-1-(1-(thieno[2,3-b]pyridin-2-yl)-7- | 0.93 | 558.82 | 47 |
| | (trifluoromethyl)-3,4-dihydroimidazo[1,2-a:5,4-c′]dipyridin-2(1H)- | | | |
| | yl)ethan-1-one | | | |
| 3.49.2 | 2-(2-chloro-4-(morpholinomethyl)phenoxy)-1-(1-(thieno[2,3-b]pyridin- | 0.88 | 641.84 | 13 |
| | 2-yl)-7-(trifluoromethyl)-3,4-dihydroimidazo[1,2-a:5,4-c′]dipyridin- | | | |
| | 2(1H)-yl)ethan-1-one | | | |
| 3.50.1 | 1-(1-(5-methylpyridin-2-yl)-7-(trifluoromethyl)-3,4- | 0.87 | 517.05 | 58 |
| | dihydroimidazo[1,2-a:5,4-c′]dipyridin-2(1H)-yl)-2-(naphthalen-2- | | | |
| | yloxy)ethan-1-one | | | |
| 3.50.2 | 2-(2-chloro-4-(morpholinomethyl)phenoxy)-1-(1-(5-methylpyridin-2- | 0.66 | 600.16 | 100 |
| | yl)-7-(trifluoromethyl)-3,4-dihydroimidazo[1,2-a:5,4-c′]dipyridin- | | | |
| | 2(1H)-yl)ethan-1-one | | | |
| 3.50.3 | 2-((1-methyl-3-phenyl-1H-pyrazol-5-yl)oxy)-1-(1-(5-methylpyridin-2- | 0.71 | 547.13 | 468 |
| | yl)-7-(trifluoromethyl)-3,4-dihydroimidazo[1,2-a:5,4-c′]dipyridin- | | | |
| | 2(1H)-yl)ethan-1-one | | | |
| 3.51.1 | 1-(1-(3,5-dimethylisoxazol-4-yl)-7-(trifluoromethyl)-3,4- | 0.92 | 520.77 | 87 |
| | dihydroimidazo[1,2-a:5,4-c′]dipyridin-2(1H)-yl)-2-(naphthalen-2- | | | |
| | yloxy)ethan-1-one | | | |
| 3.51.2 | 2-(2-chloro-4-(morpholinomethyl)phenoxy)-1-(1-(3,5- | 0.86 | 603.90 | 217 |
| | dimethylisoxazol-4-yl)-7-(trifluoromethyl)-3,4-dihydroimidazo[1,2- | | | |
| | a:5,4-c′]dipyridin-2(1H)-yl)ethan-1-one | | | |
| 3.52.1 | 1-(1-(5-methoxypyridin-2-yl)-7-(trifluoromethyl)-3,4- | 0.89 | 533.10 | 90 |
| | dihydroimidazo[1,2-a:5,4-c′]dipyridin-2(1H)-yl)-2-(naphthalen-2- | | | |
| | yloxy)ethan-1-one | | | |
| 3.52.2 | 2-(2-chloro-4-(morpholinomethyl)phenoxy)-1-(1-(5-methoxypyridin-2- | 0.68 | 616.16 | 74 |
| | yl)-7-(trifluoromethyl)-3,4-dihydroimidazo[1,2-a:5,4-c′]dipyridin- | | | |
| | 2(1H)-yl)ethan-1-one | | | |
| 3.53.1 | 1-(1-(2-methylpyrimidin-5-yl)-7-(trifluoromethyl)-3,4- | 0.72 | 518.04 | 161 |
| | dihydroimidazo[1,2-a:5,4-c′]dipyridin-2(1H)-yl)-2-(naphthalen-2- | | | |
| | yloxy)ethan-1-one | | | |
| 3.54.1 | 1-(1-(2-ethylpyrimidin-5-yl)-7-(trifluoromethyl)-3,4- | 0.77 | 531.99 | 203 |
| | dihydroimidazo[1,2-a:5,4-c′]dipyridin-2(1H)-yl)-2-(naphthalen-2- | | | |
| | yloxy)ethan-1-one | | | |
| 3.54.2 | 2-(2-chloro-4-(morpholinomethyl)phenoxy)-1-(1-(2-ethylpyrimidin-5- | 0.55 | 615.07 | 415 |
| | yl)-7-(trifluoromethyl)-3,4-dihydroimidazo[1,2-a:5,4-c′]dipyridin- | | | |
| | 2(1H)-yl)ethan-1-one | | | |
| 3.55.1 | 1-(1-(2-methoxypyrimidin-5-yl)-7-(trifluoromethyl)-3,4- | 0.75 | 534.08 | 67 |
| | dihydroimidazo[1,2-a:5,4-c′]dipyridin-2(1H)-yl)-2-(naphthalen-2- | | | |
| | yloxy)ethan-1-one | | | |
| 3.55.2 | 2-(2-chloro-4-(morpholinomethyl)phenoxy)-1-(1-(2- | 0.54 | 617.16 | 266 |
| | methoxypyrimidin-5-yl)-7-(trifluoromethyl)-3,4-dihydroimidazo[1,2- | | | |
| | a:5,4-c′]dipyridin-2(1H)-yl)ethan-1-one | | | |
| 3.56.1 | 1-(1-(2-chloropyridin-4-yl)-7-(trifluoromethyl)-3,4-dihydroimidazo[1,2- | 0.92 | 536.97 | 195 |
| | a:5,4-c′]dipyridin-2(1H)-yl)-2-(naphthalen-2-yloxy)ethan-1-one | | | |
| 3.56.2 | 2-(2-chloro-4-(morpholinomethyl)phenoxy)-1-(1-(2-chloropyridin-4- | 0.70 | 620.03 | 295 |
| | yl)-7-(trifluoromethyl)-3,4-dihydroimidazo[1,2-a:5,4-c′]dipyridin- | | | |
| | 2(1H)-yl)ethan-1-one | | | |
| 3.57.1 | 1-(1-(2-methylpyridin-4-yl)-7-(trifluoromethyl)-3,4- | 0.75 | 517.00 | 155 |
| | dihydroimidazo[1,2-a:5,4-c′]dipyridin-2(1H)-yl)-2-(naphthalen-2- | | | |
| | yloxy)ethan-1-one | | | |
| 3.57.2 | 2-(2-chloro-4-(morpholinomethyl)phenoxy)-1-(1-(2-methylpyridin-4- | 0.57 | 600.07 | 35 |
| | yl)-7-(trifluoromethyl)-3,4-dihydroimidazo[1,2-a:5,4-c′]dipyridin- | | | |
| | 2(1H)-yl)ethan-1-one | | | |
| 3.58.1 | 2-(2-chloro-4-(morpholinomethyl)phenoxy)-1-(1-(5-chloropyridin-2- | 0.70 | 620.10 | 30 |
| | yl)-7-(trifluoromethyl)-3,4-dihydroimidazo[1,2-a:5,4-c′]dipyridin- | | | |
| | 2(1H)-yl)ethan-1-one | | | |
| 3.59.1 | 2-((2-chloro-6-(oxazol-2-yl)pyridin-3-yl)oxy)-1-(1-(2,4- | 0.84 | 589.14 | 22 |
| | dimethylthiazol-5-yl)-7-(trifluoromethyl)-3,4-dihydroimidazo[1,2- | | | |
| | a:5,4-c′]dipyridin-2(1H)-yl)ethan-1-one | | | |
| 3.59.2 | 6-chloro-N-cyclopropyl-5-(2-(1-(2,4-dimethylthiazol-5-yl)-7- | 0.82 | 619.18 | 42 |
| | (trifluoromethyl)-3,4-dihydroimidazo[1,2-a:5,4-c′]dipyridin-2(1H)-yl)- | | | |
| | 2-oxoethoxy)-N-methylpicolinamide | | | |
| 4.1.1 | 2-(2,4-dichlorophenoxy)-1-(1-(3,4-dimethoxyphenyl)-7-methyl-3,4- | 0.89 | 526.03 | 32 |
| | dihydroimidazo[1,2-a:5,4-c′]dipyridin-2(1H)-yl)ethan-1-one | | | |
| 4.1.2 | 1-(1-(3,4-dimethoxyphenyl)-7-methyl-3,4-dihydroimidazo[1,2-a:5,4- | 0.69 | 508.04 | 10 |
| | c′]dipyridin-2(1H)-yl)-2-(naphthalen-2-yloxy)ethan-1-one | | | |
| 5.1.1 | 1-(7-(tert-butyl)-1-(3,4-dimethoxyphenyl)-3,4-dihydroimidazo[1,2- | 0.78 | 568.11 | 10 |
| | a:5,4-c′]dipyridin-2(1H)-yl)-2-(2,4-dichlorophenoxy)ethan-1-one | | | |
| 6.1.1 | 2-(2,4-dichlorophenoxy)-1-(1-(3,4-dimethoxyphenyl)-7-ethyl-8- | 0.76 | 554.18 | 16 |
| | methyl-3,4-dihydroimidazo[1,2-a:5,4-c′]dipyridin-2(1H)-yl)ethan-1- | | | |
| | one | | | |
| 6.1.2 | 1-(1-(3,4-dimethoxyphenyl)-7-ethyl-8-methyl-3,4-dihydroimidazo[1,2- | 0.74 | 536.32 | 9 |
| | a:5,4-c′]dipyridin-2(1H)-yl)-2-(naphthalen-2-yloxy)ethan-1-one | | | |
| 6.1.3 | 1-(1-(3,4-dimethoxyphenyl)-7-ethyl-8-methyl-3,4-dihydroimidazo[1,2- | 0.79 | 564.45 | 9 |
| | a:5,4-c′]dipyridin-2(1H)-yl)-2-((1-ethylnaphthalen-2-yl)oxy)ethan-1- | | | |
| | one | | | |
| 6.1.4 | 1-(1-(3,4-dimethoxyphenyl)-7-ethyl-8-methyl-3,4-dihydroimidazo[1,2- | 0.76 | 550.42 | 8 |
| | a:5,4-c′]dipyridin-2(1H)-yl)-2-((1-methylnaphthalen-2-yl)oxy)ethan-1- | | | |
| | one hydrochloride | | | |
| 7.1.1 | 2-(2,4-dichlorophenoxy)-1-(1-(3,4-dimethoxyphenyl)-8-methyl-3,4- | 0.70 | 526.23 | 89 |
| | dihydroimidazo[1,2-a:5,4-c′]dipyridin-2(1H)-yl)ethan-1-one | | | |
| 7.1.2 | 1-(1-(3,4-dimethoxyphenyl)-8-methyl-3,4-dihydroimidazo[1,2-a:5,4- | 0.68 | 508.32 | 38 |
| | c′]dipyridin-2(1H)-yl)-2-(naphthalen-2-yloxy)ethan-1-one | | | |
| 8.1.1 | 1-(1-(3,4-dimethoxyphenyl)-8-phenyl-3,4-dihydroimidazo[1,2-a:5,4- | 0.76 | 570.40 | 38 |
| | c′]dipyridin-2(1H)-yl)-2-(naphthalen-2-yloxy)ethan-1-one | | | |
| 9.1.1 | 1-(8-chloro-1-(3,4-dimethoxyphenyl)-3,4-dihydroimidazo[1,2-a:5,4- | 0.72 | 528.24 | 145 |
| | c′]dipyridin-2(1H)-yl)-2-(naphthalen-2-yloxy)ethan-1-one | | | |
| 10.1.1 | 1-(8-bromo-1-(3,4-dimethoxyphenyl)-3,4-dihydroimidazo[1,2-a:5,4- | 0.73 | 572.04 | 124 |
| | c′]dipyridin-2(1H)-yl)-2-(naphthalen-2-yloxy)ethan-1-one | | | |
| 11.1.1 | 1-(1-(3,4-dimethoxyphenyl)-8-fluoro-3,4-dihydroimidazo[1,2-a:5,4- | 0.70 | 512.21 | 122 |
| | c′]dipyridin-2(1H)-yl)-2-(naphthalen-2-yloxy)ethan-1-one | | | |
| 11.2.1 | N-(6-chloro-5-(2-(1-(4-chloro-2-fluorophenyl)-8-fluoro-3,4- | 0.71 | 582.03 | 72 |
| | dihydroimidazo[1,2-a:5,4-c′]dipyridin-2(1H)-yl)-2-oxoethoxy)pyridin- | | | |
| | 2-yl)methanesulfonamide | | | |
| 11.2.2 | N-(5-(2-(1-(4-chloro-2-fluorophenyl)-8-fluoro-3,4-dihydroimidazo[1,2- | 0.72 | 575.85 | 34 |
| | a:5,4-c′]dipyridin-2(1H)-yl)-2-oxoethoxy)-6-ethylpyridin-2- | | | |
| | yl)methanesulfonamide | | | |
| 11.3.1 | N-(5-(2-(1-(2,4-dimethylthiazol-5-yl)-8-fluoro-3,4-dihydroimidazo[1,2- | 0.64 | 558.76 | 48 |
| | a:5,4-c′]dipyridin-2(1H)-yl)-2-oxoethoxy)-6-ethylpyridin-2- | | | |
| | yl)methanesulfonamide | | | |
| 12.1.1 | 1-(1-(3,4-dimethoxyphenyl)-8-(trifluoromethyl)-3,4- | 0.81 | 561.79 | 183 |
| | dihydroimidazo[1,2-a:5,4-c′]dipyridin-2(1H)-yl)-2-(naphthalen-2- | | | |
| | yloxy)ethan-1-one | | | |
| 13.1.1 | 1-(7-chloro-1-(3,4-dimethoxyphenyl)-8-methyl-3,4- | 0.72 | 542.34 | 40 |
| | dihydroimidazo[1,2-a:5,4-c′]dipyridin-2(1H)-yl)-2-(naphthalen-2- | | | |
| | yloxy)ethan-1-one | | | |
| 14.1.1 | N-(6-chloro-5-(2-(1-(4-chloro-2-fluorophenyl)-7-fluoro-3,4- | 0.69 | 582.02 | 74 |
| | dihydroimidazo[1,2-a:5,4-c′]dipyridin-2(1H)-yl)-2-oxoethoxy)pyridin- | | | |
| | 2-yl)methanesulfonamide | | | |
| 15.1.1 | N-(6-chloro-5-(2-(1-(4-chloro-2-fluorophenyl)-6-fluoro-3,4- | 0.77 | 582.02 | 2650 |
| | dihydroimidazo[1,2-a:5,4-c′]dipyridin-2(1H)-yl)-2-oxoethoxy)pyridin- | | | |
| | 2-yl)methanesulfonamide | | | |
| 16.1.1 | 2-(2-chloro-4-morpholinophenoxy)-1-(9-(4-cyclopropyl-2- | 0.87 | 562.46 | 19 |
| | fluorophenyl)-6,9-dihydropyrido[4′,3′:4,5]imidazo[1,2-b]pyridazin- | | | |
| | 8(7H)-yl)ethan-1-one | | | |
| 16.1.2 | 2-((2-chloro-6-morpholinopyridin-3-yl)oxy)-1-(9-(4-cyclopropyl-2- | 0.89 | 562.98 | 8 |
| | fluorophenyl)-6,9-dihydropyrido[4′,3′:4,5]imidazo[1,2-b]pyridazin- | | | |
| | 8(7H)-yl)ethan-1-one | | | |
| 16.2.1 | 2-((2-chloro-6-morpholinopyridin-3-yl)oxy)-1-(9-(5-cyclopropyl-3- | 0.81 | 564.45 | 14 |
| | fluoropyridin-2-yl)-6,9-dihydropyrido[4′,3′:4,5]imidazo[1,2- | | | |
| | b]pyridazin-8(7H)-yl)ethan-1-one | | | |
| 16.2.2 | 1-(9-(5-cyclopropyl-3-fluoropyridin-2-yl)-6,9- | 0.62 | 486.94 | 128 |
| | dihydropyrido[4′,3′:4,5]imidazo[1,2-b]pyridazin-8(7H)-yl)-2-((2-ethyl- | | | |
| | 6-methylpyridin-3-yl)oxy)ethan-1-one | | | |
| 16.2.3 | 1-(9-(5-cyclopropyl-3-fluoropyridin-2-yl)-6,9- | 0.79 | 513.31 | 144 |
| | dihydropyrido[4′,3′:4,5]imidazo[1,2-b]pyridazin-8(7H)-yl)-2-((2- | | | |
| | (trifluoromethyl)pyridin-3-yl)oxy)ethan-1-one | | | |
| 16.2.4 | 2-((6-(aminomethyl)-2-chloropyridin-3-yl)oxy)-1-(9-(5-cyclopropyl-3- | 0.81 | 504.45 | 401 |
| | fluoropyridin-2-yl)-6,9-dihydropyrido[4′,3′:4,5]imidazo[1,2- | | | |
| | b]pyridazin-8(7H)-yl)ethan-1-one | | | |
| 16.2.5 | 2-(2-chloro-4-(trifluoromethyl)phenoxy)-1-(9-(5-cyclopropyl-3- | 0.91 | 546.33 | 238 |
| | fluoropyridin-2-yl)-6,9-dihydropyrido[4′,3′:4,5]imidazo[1,2- | | | |
| | b]pyridazin-8(7H)-yl)ethan-1-one | | | |
| 16.2.6 | 2-((2-chloro-6-(trifluoromethyl)pyridin-3-yl)oxy)-1-(9-(5-cyclopropyl-3- | 0.87 | 547.06 | 290 |
| | fluoropyridin-2-yl)-6,9-dihydropyrido[4′,3′:4,5]imidazo[1,2- | | | |
| | b]pyridazin-8(7H)-yl)ethan-1-one | | | |
| 16.4.1 | N-(6-chloro-5-(2-(9-(4-chloro-2-fluorophenyl)-6,9- | 0.77 | 564.82 | 167 |
| | dihydropyrido[4′,3′:4,5]imidazo[1,2-b]pyridazin-8(7H)-yl)-2- | | | |
| | oxoethoxy)pyridin-2-yl)methanesulfonamide | | | |
| 16.5.1 | N-(5-(2-(9-(2,4-dimethylthiazol-5-yl)-6,9- | 0.69 | 541.98 | 31 |
| | dihydropyrido[4′,3′:4,5]imidazo[1,2-b]pyridazin-8(7H)-yl)-2- | | | |
| | oxoethoxy)-6-ethylpyridin-2-yl)methanesulfonamide | | | |
| 17.1.1 | 2-((2-chloro-6-morpholinopyridin-3-yl)oxy)-1-(1-(4-cyclopropyl-2- | 1.06 | 562.97 | 29 |
| | fluorophenyl)-3,4-dihydropyrido[4′,3′:4,5]imidazo[1,2-a]pyrazin- | | | |
| | 2(1H)-yl)ethan-1-one | | | |
| 17.2.1 | N-(6-chloro-5-(2-(1-(4-chloro-2-fluorophenyl)-3,4- | 0.87 | 564.97 | 214 |
| | dihydropyrido[4′,3′:4,5]imidazo[1,2-a]pyrazin-2(1H)-yl)-2- | | | |
| | oxoethoxy)pyridin-2-yl)methanesulfonamide | | | |
| 17.3.1 | N-(5-(2-(1-(2,4-dimethylthiazol-5-yl)-3,4- | 0.79 | 541.93 | 461 |
| | dihydropyrido[4′,3′:4,5]imidazo[1,2-a]pyrazin-2(1H)-yl)-2-oxoethoxy)- | | | |
| | 6-ethylpyridin-2-yl)methanesulfonamide | | | |
| 18.1.1 | 2-((2-chloro-6-morpholinopyridin-3-yl)oxy)-1-(6-(4-cyclopropyl-2- | 0.83 | 563.45 | 56 |
| | fluorophenyl)-8,9-dihydropyrido[4′,3′:4,5]imidazo[1,2-a]pyrimidin- | | | |
| | 7(6H)-yl)ethan-1-one | | | |

### Synthesis of Amines of the Structure 4

Amines of structure 4 or their precursors are either commercially available or can prepared according to procedures known to a person skilled in the art or in analogy to the methods described in the experimental part below.

### Synthesis of Aldehydes of Structure 3

### Aldehyde 1: 4-Chloro-2-fluorobenzaldehyde

### (4-Chloro-2-fluorophenyl)methanol

To a solution of 4-chloro-2-fluorobenzoic acid (300 mg) in THF (15 ml) was added at 0° C. LiAIH₄ (130 mg). The suspension was stirred at 0° C. for 16 h. The reaction mixture was diluted with EA and aq. solution of potassium sodium tartrat and stirred for 1 h at rt. The layers were separated and the org. phase was further washed with water. The combined org. layers were dried over MgSO₄, filtrated off and evaporated in vacuo. The crude was purified by CC (Büchi Sepacore, 5 g cartridge, solvent A: DCM, solvent B: 3N ammonia in MeOH, gradient in % B: 0 to 5, flow rate: 6.0 ml/min) to afford 224 mg of colourless oil. LC-MS (A) t_{R}=0.68 min; [M+H]⁺: not visible.

### 4-Chloro-2-fluorobenzaldehyde

To a solution of (4-chloro-2-fluorophenyl)methanol (222 mg) in MeCN (20 ml) was added MnO₂ (480 mg). The mixture was stirred for 24 h. The mixture was filtered over celite, the org. layer was dried over MgSO₄ and evaporated in vacuo. The crude aldehyde was used without purification in the next step. LC-MS (A): t_{R}=0.76 min; [M+H]⁺: not visible.

### Aldehyde 2: 4-Cyclopropyl-2-fluorobenzaldehyde

### Methyl 4-bromo-2-fluorobenzoate

A solution of 4-bromo-2-fluorobenzoyl chloride (15 ml) in MeOH (200 ml) was stirred at rt for 18 h. The reaction mixture was evaporated in vacuo. The residue was diluted with DCM and sat. aq. NaHCO₃. The layers were separated, the aq. layer was extracted with DCM, the combined org. layers were dried over MgSO₄, filtrated off and evaporated in vacuo. The crude (25 g of a white solid) was used without purification in the next step. LC-MS (A) t_{R}=0.84 min; [M+H]⁺: not visible.

### Methyl 4-cyclopropyl-2-fluorobenzoate

To a solution of methyl 4-bromo-2-fluorobenzoate (25 g) in THF (500 ml) were added potassium cyclopropyltrifluoroborate (15.9 g), cesium carbonate (105 g) and water (50 ml). The solution was degassed under argon and (1,1′-bis(diphenylphosphino)ferrocene) dichloropalladium (II) dichloromethane adduct (8.8 g) was finally added. The reaction mixture was stirred at 70° C. overnight. The mixture was diluted with water and TBME, the layers were separated. The aq. layer was extracted with TBME and the combined org. layers were washed with sat. aq. NaCl, dried over MgSO₄, filtrated off and evaporated in vacuo. The crude was purified by CC (Büchi Sepacore, 350 g cartridge, solvent A: Heptane, solvent B: EA, gradient in % B: 1 to 20, flow rate: 100 ml/min) to afford 19.2 g of yellow oil. LC-MS (A) t_{R}=0.87 min; [M+H]⁺: 195.45.

### 4-Cyclopropyl-2-fluorobenzaldehyde

This aldehyde has been prepared from methyl 4-cyclopropyl-2-fluorobenzoate according to the reductionoxidation procedure described for aldehyde 1. LC-MS (A): t_{R}=0.83 min; [M+H]⁺: not visible.

### Aldehyde 3: 6-Chloro-2-fluoronicotinaldehyde

To a solution of diisopropylamine (5.26 ml) in THF (70 ml) was added at −78° C. nBuLi 1.6M in hexanes (21.6 ml). The mixture was stirred at 0° C. for 45 min. 2-Chloro-6-fluoropyridine (3.5 g) in THF (36 ml) was added dropwise at −78° C. over 1 h under nitrogen to the previous mixture and the reaction mixture was stirred at −78° C. for 1.5 h. DMF (4.12 ml) was added dropwise over 1 h and the reaction mixture was stirred an additional 1.5 h. HCl 2M in diethylether (45 ml) was added slowly at −78° C., water (30 ml) was added and the layers were separated. The aq. phase was extracted with EA and the combined org. layers were washed with sat. aq. NaCl, dried over Na₂SO₄, filtrated off and evaporated in vacuo. The crude (4.4 g of an orange solid) was used without purification in the next step. GC-MS (A): t_{R}=1.55 min; [M+H]⁺: 159.80.

### Aldehyde 4: 5-Cyclopropyl-3-fluoropicolinaldehyde

This aldehyde has been prepared from (5-cyclopropyl-3-fluoropyridin-2-yl)methanol according to the procedure described for aldehyde 1 (2.step). LC-MS (A): t_{R}=0.68 min; [M+H]⁺: 166.25.

### Aldehyde 5: 5-Chloro-3-fluoropicolinaldehyde

### Methyl 5-chloro-3-fluoropicolinate

To a solution of 5-chloro-3-fluoropyridine-2-carboxylic acid (6 g) in MeOH (120 ml) was added (trimethylsilyl)diazomethane 2M in diethyl ether (48.6 ml). The reaction mixture was stirred at rt for 1 h. The mixture was evaporated in vacuo. The crude compound (5.65 g of a brown solid) was used without purification in the next step. LC-MS (A) t_{R}=0.64 min; [M+H]⁺: 190.19.

### (5-Chloro-3-fluoropyridin-2-yl)methanol

To a solution of methyl 5-chloro-3-fluoropicolinate (1.05 g) in THF (25 ml) was added at 0° C. lithium borohydride 2M in THF (5.6 ml). The reaction mixture was stirred at 0° C. for 1 h. The mixture was diluted with sat. aq. NaHCO₃ and EA, the layers were separated and the aq. phase was washed with EA. The combined org. layers were washed with sat. aq. NaCl, dried over MgSO₄, filtrated off and evaporated in vacuo. The crude was purified by CC (Büchi Sepacore, 50 g cartridge, solvent A: DCM, solvent B: MeOH, gradient in % B: 0 to 5, flow rate: 30 ml/min) to afford 2.70 g of a yellow solid. LC-MS (A) t_{R}=0.50 min; [M+H]⁺: 161.95.

### 5-Chloro-3-fluoropicolinaldehyde

This aldehyde has been prepared from (5-chloro-3-fluoropyridin-2-yl)methanol according to the procedure described for aldehyde 1 (2.step). LC-MS (A): t_{R}=0.59 min; [M+H]⁺: not visible.

### Aldehyde 6: 6-Cyclopropyl-2-fluoronicotinaldehyde

To a solution of 6-chloro-2-fluoronicotinaldehyde (1.21 g) in THF (40 ml) were added potassium cyclopropyltrifluoroborate (1.13 g), cesium carbonate (7.43 g), (1,1′-bis(diphenylphosphino)ferrocene)dichloropalladium (II) dichloromethane adduct (621 mg) and water (4 ml). The reaction mixture was stirred at 73° C. for 2 h and at 63° C. overnight. The mixture was diluted with water and TBME at rt and the layers were separated. The aq. layer was extracted with TBME and the combined org. layers were washed with 1N aq. HCl (15 ml) and sat. aq. NaCl, dried over Na₂SO₄, filtrated off and evaporated in vacuo. The crude was purified by CC (Flash Master, solvent A: heptane, solvent B: EA, gradient in % B: 20, flow rate: 15 ml/min) to afford 501 mg of a yellow oil. GC-MS (A) t_{R}=2.05 min; [M+H]⁺: 165.90.

### Aldehyde 7: 4-Methoxy-3-((4-methoxybenzyl)oxy)benzaldehyde

A solution of 3-hydroxy-4-methoxybenzaldehyde (100 g), 4-methoxybenzylchloride (101 g) and potassium carbonate (180 g) in DMF (750 ml) was stirred at rt over 3 days. The reaction mixture was filtrated off and the solid was washed with EA (200 ml). The filtrate was diluted with EA and extracted with water. The aq. layer was extracted three times with EA, the combined org. layers were washed with sat. aq. NaCl, dried over MgSO₄, filtrated off and evaporated in vacuo. The crude was recrystallized from heptane to afford 153.47 g of a white solid. LC-MS (A) t_{R}=0.87 min; [M+H]⁺: 273. [M+AcCN]: 314.30.

### Aldehyde 8: 6-Ethoxynicotinaldehyde

This aldehyde has been prepared from 6-ethoxynicotinic acid according to the procedures described for aldehyde 1 (2.step). LC-MS (A): t_{R}=0.68 min; [M+H]⁺: 152.28.

### Aldehyde 9: 6-(2,2,2-Trifluoroethoxy)nicotinaldehyde

This aldehyde has been prepared from 6-(2,2,2-trifluoroethoxy)nicotinic acid according to the reductionoxidation procedure described for aldehyde 1. LC-MS (A): t_{R}=0.80 min; [M+H]⁺: not visible.

### Aldehyde 10: 4-(2-(Dimethylamino)ethoxy)-3-methoxybenzaldehyde

To a solution of 4-hydroxy-3-methoxybenzaldehyde (10 g) in DMF (500 ml) were added potassium carbonate (18.20 g) and 2-chloro-N,N-dimethylethylamine hydrochloride (14.08 g). The reaction mixture was stirred at 80° C. for 1 h30 min. The suspension was filtrated off and the filtrate was diluted with Et₂O and sat. aq. NaCl. The aq. layer was extracted with Et₂O, the combined org. layers were dried over MgSO₄, filtrated off and evaporated in vacuo. The crude was purified by CC (Büchi Sepacore, 70 g cartridge, solvent A: DCM, solvent B: MeOH, gradient in % B: 2 to 4, flow rate: 20 ml/min) to afford 7.3 g of a yellow oil. LC-MS (A): t_{R}=0.60 min; [M+H]⁺: 224.48.

### Aldehyde 11: 5,6-Dimethoxypicolinaldehyde

This aldehyde has been prepared from (5,6-dimethoxypyridin-2-yl)methanol according to the procedure described for aldehyde 1 (2.step). LC-MS (A): t_{R}=0.61 min; [M+H]⁺: 168.00.

### Aldehyde 12: 2-fluoro-4-(2-hydroxypropan-2-yl)benzaldehyde

### Methyl 4-(dimethoxymethyl)-3-fluorobenzoate

A solution of methyl 3-fluoro-4-formylbenzoate (1 g), trimethylorthoformate (4 ml) and PTSA monohydrate (9 mg) was stirred at 70° c. for 3 h. The reaction mixture was evaporated in vacuo. The mixture was diluted with sat. aq. NaCl and DCM. The layers were separated and the org. phase was washed with sat. aq. NaCl. The combined org. layers were dried over MgSO₄, filtrated off and evaporated in vacuo. The crude product (1.26 g of a yellowish oil) was used in the next step without purification. LC-MS (A): t_{R}=0.82 min; [M+H]⁺: not visible.

### 2-(4-(Dimethoxymethyl)-3-fluorophenyl)propan-2-ol

To a solution methyl 4-(dimethoxymethyl)-3-fluorobenzoate (400 mg) in THF (715 ml) was added at −78° C. MeMgBr 3M in Et₂O (2.1 ml). The reaction mixture was stirred at rt for 4 h. The mixture was diluted with aq. Rochelle salt, EA and water and the layers were separated. The aq. phase was extracted with EA and the combined org. layers were washed with sat. aq. NaCl, dried over Na₂SO₄, filtrated off and evaporated in vacuo. The crude was purified by CC (Büchi Sepacore, 5 g cartridge, solvent A: EA, solvent B: heptane, gradient in % B: 5 to 15, flow rate: 10 ml/min) to afford 330 mg of a yellow oil. LC-MS (A) t_{R}=0.71 min; [M+H]⁺: not visible.

### 2-fluoro-4-(2-hydroxypropan-2-yl)benzaldehyde

To a solution of 2-(4-(dimethoxymethyl)-3-fluorophenyl)propan-2-ol (330 mg) in THF (15 ml) was added at 0° C. aq. 2M HCl (2.2 ml) and the mixture was stirred at rt for 1 h. The reaction mixture was evaporated in vacuo. The mixture was diluted with sat. aq. NaCl and DCM. The layers were separated and the org. phase was washed with sat. aq. NaCl. The combined org. layers were dried over MgSO₄, filtrated off and evaporated in vacuo. The crude product (1.26 g of a yellowish oil) was used in the next step without purification. LC-MS (A): t_{R}=0.66 min; [M+H]⁺: not visible.

### Aldehyde 13: 2-Fluoro-4-(2-methoxypropan-2-yl)benzaldehyde

### 1-(Dimethoxymethyl)-2-fluoro-4-(2-methoxypropan-2-yl)benzene

To a solution of 2-(4-(dimethoxymethyl)-3-fluorophenyl)propan-2-ol (330 mg) in THF (10 ml) was added at 0° C. NaH (60% suspension, 76 mg) and the mixture was stirred at 0° C. for 30 min. Mel (0.185 ml) was added and the mixture was stirred at rt for 18 h. The mixture was diluted with sat. aq. NH₄Cl and EA. The layers were separated and the org. phase was washed with sat. aq. NaCl. The combined org. layers were dried over MgSO₄, filtrated off and evaporated in vacuo. The crude was purified by CC (Büchi Sepacore, 10 g cartridge, solvent A: EA, solvent B: heptane, gradient in % B: 1 to 12, flow rate: 15 ml/min) to afford 254 mg of a colourless oil. LC-MS (A) t_{R}=0.85 min; [M+H]⁺: not visible.

### 2-Fluoro-4-(2-methoxypropan-2-yl)benzaldehyde

This aldehyde has been prepared from 1-(dimethoxymethyl)-2-fluoro-4-(2-methoxypropan-2-yl)benzene according to the procedure described for aldehyde 12 (3.step). LC-MS (A): t_{R}=0.80 min; [M+H]⁺: not visible.

### Aldehyde 14: 2-Fluoro-4-(methoxymethyl)benzaldehyde

### (4-(Dimethoxymethyl)-3-fluorophenyl)methanol

This compound has been prepared from methyl 4-(dimethoxymethyl)-3-fluorobenzoate according to the procedure described for aldehyde 1 (1.step). LC-MS (A): t_{R}=0.62 min; [M+H]⁺: not visible.

### 2-Fluoro-4-(methoxymethyl)benzaldehyde

This aldehyde has been prepared from (4-(dimethoxymethyl)-3-fluorophenyl)methanol according to the procedures described for aldehyde 13. LC-MS (A): t_{R}=0.71 min; [M+H]⁺: not visible.

### Aldehyde 15: 2-Fluoro-4-(2-methoxyethoxy)benzaldehyde

A solution of 2-fluoro-4-hydroxybenzaldehyde (200 mg), bromo(methoxy)methane (0.201 ml) and K₂CO₃ (592 mg) in DMF (5 ml) was stirred at 60° C. for 2 h. The mixture was diluted with DCM and water. The layers were separated and the org. phase was washed with sat. aq. NaCl. The combined org. layers were dried over MgSO₄, filtrated off and evaporated in vacuo. The crude product (250 mg of a yellow oil) was used in the next step without purification. LC-MS (A): t_{R}=0.72 min; [M+H]⁺: 199.15.

### Aldehyde 16: 4-(2-(Benzyloxy)ethoxy)-2-fluorobenzaldehyde

This aldehyde has been prepared from 2-fluoro-4-hydroxybenzaldehyde and ((2-bromoethoxy)methyl)benzene according to the procedures described for aldehyde 15. LC-MS (A): t_{R}=0.91 min; [M+H]⁺: 275.13.

### Aldehyde 17: Methyl 5-formylthiophene-3-carboxylate

### Methyl 5-(1,3-dioxolan-2-yl)thiophene-3-carboxylate

To a solution of 2-(4-bromothien-2-yl)-1,3-dioxolane (5 g) in diethyl ether (200 ml) was added at −78° C. nBuLi 1.6M in hexanes (16 ml) under nitrogen. The mixture was stirred at −78° C. for 15 min. Methyl chloroformate (16.6 ml) was added dropwise and the reaction mixture was stirred at −78° C. for 1 h under nitrogen. The mixture was diluted with sat. aq. NH₄Cl and EA, the layers were separated and the aq. phase was extracted with EA. The combined org. layers were dried over MgSO₄, filtrated off and evaporated in vacuo. The crude was purified by CC (Büchi Sepacore, 50 g cartridge, solvent A: Heptane, solvent B: EA, gradient in % B: 0 to 5, flow rate: 30 ml/min) to afford 2.89 g of a colourless oil. LC-MS (A) t_{R}=0.72 min; [M+H]⁺: 214.85.

### Methyl 5-formylthiophene-3-carboxylate

This aldehyde has been prepared from methyl 5-(1,3-dioxolan-2-yl)thiophene-3-carboxylate according to the procedure described for aldehyde 12 (3. step). LC-MS (A): t_{R}=0.66 min; [M+H]⁺: not visible.

### Synthesis of acids of Structure 2

### Acid 1: 2-((2-Ethyl-6-methylpyridin-3-yl)oxy)acetic acid

### Tert-butyl 2-((2-ethyl-6-methylpyridin-3-yl)oxy)acetate

To a solution of 2-ethyl-3-hydroxy-6-methylpyridine (2 g) in THF (40 ml) was added NaH (763 mg) portionwise at 0° C. After 30 min tert-butyl bromoacetate (2.15 ml) was added and the mixture was stirred overnight at rt. The reaction mixture was diluted with EA and sat. aq. NH₄Cl. The layers were separated and the org. phase was washed with sat. aq. NaCl. The combined org. layers were dried over MgSO₄, filtrated off and evaporated in vacuo. The crude was purified by CC (Büchi Sepacore, 50 g cartridge, solvent A: Heptane, solvent B: EA, gradient in % B: 1 to 5, flow rate: 30 ml/min) to afford 3.90 g of a colourless oil. LC-MS (A): t_{R}=0.61 min; [M+H]⁺: 252.10.

### 2-((2-Ethyl-6-methylpyridin-3-yl)oxy)acetic acid

To a solution of tert-butyl 2-((2-ethyl-6-methylpyridin-3-yl)oxy)acetate (3.90 g) in DCM (50 ml) was added TFA (14 ml) at 0° C. and the reaction was stirred for 2.5 h at rt. The mixture was evaporated in vacuo. The crude product was washed with Et₂O. The crude product (3.9 g of a colourless solid) was used in the next step without purification. LC-MS (A): t_{R}=0.37 min; [M+H]⁺: 196.13.

### Acid 2: 2-(2-Chloro-4-morpholinophenoxy)acetic acid

### 4-Bromo-2-chloro-1-(methoxymethoxy)benzene

To a solution of 4-bromo-chlorophenol (1.1 g) in DCM (55 ml) was added at 0° C. DIPEA (1.36 ml) and chloromethyl methyl ether (0.44 ml). The mixture was stirred at 0° C. for 1 h and overnight at rt. The reaction mixture was diluted with EA and 1N aq. KHSO₄. The layers were separated and the org. phase was washed with water and sat. aq. NaCl. The combined org. layers were dried over MgSO₄, filtrated off and evaporated in vacuo. The crude (1.43 g of a colourless oil) was used in the next step without purification. LC-MS (A): t_{R}=0.90 min; [M+H]⁺: not visible.

### 4-(3-Chloro-4-(methoxymethoxy)phenyl)morpholine

A solution of 4-bromo-2-chloro-1-(methoxymethoxy)benzene (1.43 g), morpholine (0.65 ml), sodium tert-butoxide (765 mg), 2-biphenyl di-tert-butylphosphine (679 mg) and tris(dibenzy lidenaceton)dipalladium (52 mg) in toluene (50 ml) was stirred under nitrogen at 80° C. for 3 h. The mixture was filtered through celite and evaporated in vacuo. The crude was purified by CC (Flash Master, 20 g cartridge, solvent A: Heptane, solvent B: EA, gradient in % B:0 to 4, flow rate: 15 ml/min) to afford 1.18 g of an yellow oil. LC-MS (A): t_{R}=0.78 min; [M+H]⁺: 257.97.

### 2-Chloro-4-morpholinophenol hydrochloride

To a solution of 4-(3-chloro-4-(methoxymethoxy)phenyl)morpholine (900 mg) in EA (7 ml) and MeOH (1.8 ml) was added a solution of HCl 4M in dioxane (1.7 ml) and the mixture was stirred at rt overnight. The mixture was evaporated in vacuo. The resulting oil was suspended in diethyl ether and sonicated. The solid was filtrated off and dried in vacuo to afford 852 mg of a beige solid. LC-MS (A): t_{R}=0.54 min; [M+H]⁺: 214.01.

### Tert-butyl 2-(2-chloro-4-morpholinophenoxy)acetate

This ester has been prepared from 2-chloro-4-morpholinophenol hydrochloride according to the procedure described for acid 1 (1.step). LC-MS (A): t_{R}=0.91 min; [M+H]⁺: 328.13.

### 2-(2-Chloro-4-morpholinophenoxy)acetic acid

This acid has been prepared from tert-butyl 2-(2-chloro-4-morpholinophenoxy)acetate according to the procedure described for acid 1 (2.step). LC-MS (A): t_{R}=0.63 min; [M+H]⁺: 272.02.

### Acid 3: 2-((2-Ethylpyridin-3-yl)oxy)acetic acid

### 2-Bromopyridin-3-yl acetate

A solution of 2-bromo-3-pyridinol (3 g) in acetic anhydride (90 ml) was stirred at 140° C. for 5 min. The mixture was evaporated in vacuo. The residue was diluted with DCM and sat. aq. NaHCO₃. The layers were separated, the aq. phase was washed with DCM and the combined org. layers were washed with sat. aq. NaCl, dried over MgSO₄, filtrated off and evaporated in vacuo. The crude was purified by CC (Büchi Sepacore, 50 g cartridge, solvent A: DCM, solvent B: MeOH, gradient in % B: 1 to 3, flow rate: 30 ml/min) to afford 3.35 g of an orange oil. LC-MS (A): t_{R}=0.67 min; [M+H]⁺: 216.95.

### 2-(2-(Trimethylsilyl)ethyl)pyridin-3-yl acetate

To a solution of 2-bromopyridin-3-yl acetate (3.32 g) in THF (90 ml) were added triethylamine (11.8 ml), trimethylsilylacetylene (6.9 ml), copper iodid (150 mg) and bis(triphenyl-phosphin)palladium(II)-dichlorid (1.62 g). The reaction mixture was stirred at rt for 35 min. The mixture was diluted with EA and water. The layers were separated, the org. phase was washed with sat. aq. NH₄Cl. and sat. aq. NaCl, dried over MgSO₄, filtrated off and evaporated in vacuo. The crude was purified by CC (Büchi Sepacore, 70 g cartridge, solvent A: Heptane, solvent B: EA, gradient in % B: 6 to 40, flow rate: 35 ml/min) to afford 2.78 g of an brown oil. LC-MS (A): t_{R}=0.88 min; [M+H]⁺: 234.04.

### 2-Ethynylpyridin-3-ol

To a solution of 2-(2-(trimethylsilyl)ethyl)pyridin-3-yl acetate (2.78 g) in THF (40 ml) was added at 0° C. TBAF 1M in THF (18 ml). The reaction mixture was stirred at 0° C. for 50 min. The mixture was diluted with EA and water. The layers were separated, the org. phase was washed with sat. aq. NH₄Cl. and sat. aq. NaCl, dried over MgSO₄, filtrated off and evaporated in vacuo. The crude was purified by CC (Büchi Sepacore, 50 g cartridge, solvent A: DCM, solvent B: MeOH, gradient in % B: 1 to 4, flow rate: 30 ml/min) to afford 0.77 g of a yellow solid. LC-MS (A): t_{R}=0.31 min; [M+H]⁺: 120.33.

### 2-Ethylpyridin-3-ol

To a solution of 2-ethynylpyridin-3-ol (0.77 g) in EtOH (10 ml) was added platinoxid (IV) (110 mg). The reaction mixture was stirred under hydrogen at rt for 1 h40. The mixture was filtered through celite, washed with EtOH and evaporated in vacuo. The crude was purified by CC (Büchi Sepacore, 50 g cartridge, solvent A: DCM, solvent B: MeOH, gradient in % B: 0 to 7, flow rate: 30 ml/min) to afford 0.995 g of a yellow solid. LC-MS (A): t_{R}=0.31 min; [M+H]⁺: 124.05.

### Tert-butyl 2-((2-ethylpyridin-3-yl)oxy)acetate

This ester has been prepared from 2-ethylpyridin-3-ol according to the procedure described for acid 1 (1.step). LC-MS (A): t_{R}=0.59 min; [M+H]⁺: 238.19.

### 2-((2-Ethylpyridin-3-yl)oxy)acetic acid

This acid has been prepared from tert-butyl 2-((2-ethylpyridin-3-yl)oxy)acetate. according to the procedure described for acid 1 (2.step). LC-MS (A): t_{R}=0.29 min; [M+H]⁺: 182.16.

### Acid 4: 2-((2-Chloro-6-morpholinopyridin-3-yl)oxy)acetic acid

### 2-Chloro-6-iodo-3-(methoxymethoxy)pyridine

To a solution of 2-chloro-6-iodo-3-pyridinol (5 g) in DCM (100 ml) were added at 0° C. DIPEA (5 ml) and chloromethyl methyl ether (1.7 ml). The reaction mixture was stirred at 0° C. for 1 h. The mixture was washed with 1M aq. KHSO₄. The layers were separated, the aq. phase was washed with DCM and the combined org. layers were washed with sat. aq. NaCl., dried over MgSO₄, filtrated off and evaporated in vacuo. The crude was purified by CC (Büchi Sepacore, 50 g cartridge, solvent A: Heptane, solvent B: EA, gradient in % B: 0 to 2, flow rate: 15 ml/min) to afford 5.52 g of a colourless oil. LC-MS (A): t_{R}=0.83 min; [M+H]⁺: 299.99.

### 4-(6-Chloro-5-(methoxymethoxy)pyridin-2-yl)morpholine

To a solution of 2-chloro-6-iodo-3-(methoxymethoxy)pyridine (5.95 g) in DMSO (100 ml) were added morpholine (8.57 ml), copper iodide (3.71 g), L-proline (4.04 g) and potassium carbonate (6.19 g). The mixture was stirred at 80° C. for 1 h. The reaction mixture was diluted with sat. aq. NaCl and EA. The layers were separated, the aq. phase was washed with EA and the combined org. layers were dried over MgSO₄, filtrated off and evaporated in vacuo. The crude was purified by CC (Büchi Sepacore, 50 g cartridge, solvent A: Heptane, solvent B: EA, gradient in % B: 2 to 5, flow rate: 30 ml/min) to afford 4.14 g of a colourless oil. LC-MS (A): t_{R}=0.80 min; [M+H]⁺: 258.90.

### 2-Chloro-6-morpholinopyridin-3-ol hydrochloride

This alcohol has been prepared from 4-(6-chloro-5-(methoxymethoxy)pyridin-2-yl)morpholine according to the procedure described for acid 2 (3.step). LC-MS (A): t_{R}=0.62 min; [M+H]⁺: 215.14.

### Tert-butyl 2-((2-chloro-6-morpholinopyridin-3-yl)oxy)acetate

This ester has been prepared from 2-chloro-6-morpholinopyridin-3-ol dihydrochloride according to the procedure described for acid 1 using DMF instead of THF. LC-MS (A): t_{R}=0.91 min; [M+H]⁺: 328.98.

### 2-((2-Chloro-6-morpholinopyridin-3-yl)oxy)acetic acid

This acid has been prepared from tert-butyl 2-((2-chloro-6-morpholinopyridin-3-yl)oxy)acetate according to the procedure described for acid 1 (2.step). LC-MS (A): t_{R}=0.66 min; [M+H]⁺: 273.04.

### Acid 5: 2-((2-Chloro-6-(methylsulfonamido)pyridin-3-yl)oxy)acetic acid

### Tert-butyl 2-((2-chloro-6-iodopyridin-3-yl)oxy)acetate

This ester has been prepared from 2-chloro-6-iodo-3-pyridinol according to the procedure described for acid 4 (4.step). LC-MS (A): t_{R}=0.94 min; [M+H]⁺: 369.66.

### Tert-butyl 2-((2-chloro-6-(methylsulfonamido)pyridin-3-yl)oxy)acetate

To a solution of tert-butyl 2-((2-chloro-6-iodopyridin-3-yl)oxy)acetate (7.07 g) in DMF (150 ml) were added methansulfonamide (1.80 g), copper iodide (550 mg), (trans)-N,N′-dimethyl-1,2-cyclohexanediamine (0.90 ml) and potassium carbonate (5.3 g). The reaction mixture was stirred at 100° C. for 1 h45. The reaction mixture was diluted with EA and sat. aq. NH₄Cl. The layers were separated, the aq. phase was washed with EA and the combined org. layers were washed with sat. aq. NaCl, dried over Na₂SO₄, filtrated off and evaporated in vacuo. The crude was purified by CC (Büchi Sepacore, 100 g cartridge, solvent A: Heptane, solvent B: EA, gradient in % B: 2 to 25, flow rate: 40 ml/min) to afford 3.01 g of a white solid. LC-MS (A): t_{R}=0.81 min; [M+H]⁺: 337.04.

### 2-((2-Chloro-6-(methylsulfonamido)pyridin-3-yl)oxy)acetic acid

This acid has been prepared from tert-butyl 2-((2-chloro-6-(methylsulfonamido)pyridin-3-yl)oxy)acetate according to the procedure described for acid 1 (2.step). LC-MS (A): t_{R}=0.53 min; [M+H]⁺: 281.06.

### Acid 6: 2-(2-Chloro-4-(morpholinomethyl)phenoxy)acetic acid

### Tert-butyl 2-(2-chloro-4-formylphenoxy)acetate

To a solution of 3-chlor-4-hydroxybenzaldehyde (12.84 g) in MeCN was added NaI (1.23 g) and K₂CO₃ (12.47 g). The mixture was stirred at 80° C. for 45 min. Tert-butyl bromoacetate (8 g) was added dropwise and the mixture was stirred at 80° C. for 15 h. After cooling to the RT was the reaction mixture diluted with water and DCM. The layers were separated, the aq. phase was washed with EA and the combined org. layers were dried over Na₂SO₄, filtrated off and evaporated in vacuo. The crude was used without further purification in the next step. LC-MS (A): t_{R}=0.91 min; [M+H]⁺: not visible.

### Tert-butyl 2-(2-chloro-4-(morpholinomethyl)phenoxy)acetate

To a solution of tert-butyl 2-(2-chloro-4-formylphenoxy)acetate (4.5 mg) and moprholine (2.5 ml) in MeCN (45 ml) was added natriumtriacetoxyborhydrid (7.4 g). The mixture was stirred at rt overnight. The reaction mixture was diluted with sat. aq. NaHCO₃ and EA. The layers were separated, the aq. phase was washed with EA and the combined org. layers were dried over MgSO₄, filtrated off and evaporated in vacuo. The crude was purified by CC (EA:heptane=1:1) to afford 5.13 g of a colourless oil. LC-MS (A): t_{R}=0.66 min; [M+H]⁺: 342.16.

### 2-(2-Chloro-4-(morpholinomethyl)phenoxy)acetic acid

This compound was prepared from tert-butyl 2-(2-chloro-4-(morpholinomethyl)pheno xy)acetate according to the procedure described for acid 1 (2.step). LC-MS (A): t_{R}=0.44 min; [M+H]⁺: 286.15.

### Acid 7: 2-((4-Chloro-1-methyl-5-(trifluoromethyl)-1H-pyrazol-3-yl)oxy)acetic acid

### Methyl 2-((4-chloro-1-methyl-5-(trifluoromethyl)-1H-pyrazol-3-yl)oxy)acetate

This compound has been prepared from 4-chloro-1-methyl-5-(trifluoromethyl)-1H-pyrazol-3-ol and methyl bromoacetate according to procedure described for acid 1 (1.step). LC-MS (A): t_{R}=0.87 min; [M+H]⁺: 272.97.

### 2-((4-Chloro-1-methyl-5-(trifluoromethyl)-1H-pyrazol-3-yl)oxy) acetic acid

A solution of methyl 2-((4-chloro-1-methyl-5-(trifluoromethyl)-1H-pyrazol-3-yl)oxy)acetate (900 mg) in MeOH (15 ml) and 2.5M aq. NaOH (15 ml) was stirred at rt for 90 min. The MeOH was evaporated in vacuo and the mixture was diluted with DCM and 3M aq. HCl. The layers were separated and the org. phase was evaporated in vacuo to afford 797 mg of a colourless solid, which was used in the next step without purification. LC-MS (A): t_{R}=0.75 min; [M+H]⁺: 258.89.

### Acid 8: 3-(Carboxymethoxy)-2-ethyl-6-methylpyridine 1-oxide

### Methyl 2-((2-ethyl-6-methylpyridin-3-yl)oxy)acetate

This compound has been prepared from 2-ethyl-6-methylpyridin-3-ol according to procedure described for acid 7 (1.step). LC-MS (A): t_{R}=0.47 min; [M+H]⁺:210.07.

### 2-Ethyl-3-(2-methoxy-2-oxoethoxy)-6-methylpyridine 1-oxide

A solution methyl 2-((2-ethyl-6-methylpyridin-3-yl)oxy)acetate (335 mg) and MCPBA (470 mg) in DCM (6 ml) was stirred at rt for 15 min. The mixture was evaporated in vacuo. The crude was purified by CC (Büchi Sepacore, 10 g cartridge, solvent A: EA, solvent B: heptane, gradient in % B: 2 to 15, flow rate: 9 ml/min) to afford 314 mg of a colourless solid. LC-MS (A): t_{R}=0.59 min; [M+H]⁺: 226.30.

### 3-(Carboxymethoxy)-2-ethyl-6-methylpyridine 1-oxide

This compound has been prepared from 2-ethyl-3-(2-methoxy-2-oxoethoxy)-6-methylpyridine 1-oxide according to procedure described for acid 7 (2.step). LC-MS (A): t_{R}=0.47 min; [M+H]⁺: 212.34.

### Acid 9: 2-((2-(Trifluoromethyl)pyridin-3-yl)oxy)acetic acid

### 2-(Trifluoromethyl)pyridin-3-ol

To a solution of 1.6M nBuLi in hexane (0.94 ml) in THF (2.7 ml) was added at −78° C. 2,2,6,6-tetramethylpiperidin (0.28 ml) followed by 2-trifluoromethylpyridine (0.14 ml). The reaction was stirred at −78° C. for 17 h. Trimethylborate (0.32 ml) was added and the reaction was stirred at −78° c. for 2 h. Peracetic acid was added (0.39 ml, 39% solution in AcOH) and the reaction mixture was allowed to warm to 0° C. under stirring for 3 h. The reaction mixture was diluted with sat. aq. Na₂SO₃ and DCM. The layers were separated, the aq. phase was washed with EA and the combined org. layers were dried over MgSO₄, filtrated off and evaporated in vacuo. The crude was purified by FC (solvent A: DCM, solvent B: MeOH, gradient in % B: 2) to afford 114 mg of an orange oil. LC-MS (A): t_{R}=0.46 min; [M+H]⁺: 164.20.

### 2-((2-(Trifluoromethyl)pyridin-3-yl)oxy)acetic acid

This compound was prepared from 2-(trifluoromethyl)pyridin-3-ol according to the procedures described for acid 1 (1-2.step). LC-MS (A): t_{R}=0.49 min; [M+H]⁺: 221.98.

### Acid 10: 2-((2-Chloropyridin-3-yl)oxy)acetic acid

### Methyl 2-((2-chloropyridin-3-yl)oxy)acetate

This compound was prepared from 2-chloropyridin-3-ol according to the procedures described for acid 7 (1-2.steps). LC-MS (A): t_{R}=0.65 min; [M+H]⁺: 202.04.

### 2-((2-chloropyridin-3-yl)oxy)acetic acid

This compound was prepared from methyl 2-((2-chloropyridin-3-yl)oxy)acetate according to the procedures described for acid 4 (4.step). LC-MS (A): t_{R}=0.50 min; [M+H]⁺: 188.18.

### Acid 11: 2-((6-(Dimethylamino)-2-methylpyridin-3-yl)oxy)acetic acid

### 4,6-Dibromo-2-methylpyridin-3-ol

To a suspension of 2-methylpyridin-3-ol (500 mg) in MeCN (30 ml) was added at 0° C. NBS (1.7 g). The reaction was stirred at 0° C. for 2 h. The solvent was evaporated in vacuo. The crude was purified by CC (Büchi Sepacore, 20 g cartridge, solvent A: DCM, solvent B: 7N NH₃ in MeOH, gradient in % B: 1 to 3, flow rate: 10 ml/min) to afford 1.0 g of a yellow solid. LC-MS (A): t_{R}=0.71 min; [M+H]⁺: 267.83.

### 6-Bromo-2-methylpyridin-3-ol

To a solution of 4,6-dibromo-2-methylpyridin-3-ol (1.0 g) in THF (20 ml) was added at −78° C. 1.6M nBuLi in hexane (4.7 ml). The reaction was stirred for 2 h. Water was added (9 ml) and the reaction mixture was allowed to warm up to rt. The reaction mixture was diluted with sat. aq. NH₄Cl and EA. The layers were separated, the aq. phase was washed with EA and the combined org. layers were dried over MgSO₄, filtrated off and evaporated in vacuo. The crude was purified by CC (Büchi Sepacore, 20 g cartridge, solvent A: DCM, solvent B: 7N NH₃ in MeOH, gradient in % B: 1 to 3, flow rate: 10 ml/min) to afford 550 mg of a colourless solid. LC-MS (A): t_{R}=0.58 min; [M+H]⁺: 188.03.

### 2-((6-(Dimethylamino)-2-methylpyridin-3-yl)oxy)acetic acid

This compound was prepared from 6-bromo-2-methylpyridin-3-ol according to the procedures described for acid 4 (1-5.step), using dimethylamine instead of morpholine in the Buchwald coupling. LC-MS (A): t_{R}=0.40 min; [M+H]⁺: 211.22.

### Acid 12: 2-((2-Chloro-6-(morpholinomethyl)pyridin-3-yl)oxy)acetic acid

### 2-Chloro-6-(hydroxymethyl)pyridin-3-ol

To a solution of 2-chloropyridin-3-ol (25 g) and NaHCO₃ (2.92 g) in water (22.5 ml) was added at 90° C. aq. 37%-solution of formaldehyde portionwise (4×1.2 ml during 6 h) and the mixture was stirred for 26 h. Water was added (20 ml) at rt followed by addition of 1N aq. sol. of HCl (100 ml) to maintain the pH=1. The solid precipitate was filtrated off. The aq. phase was washed with EA and the combined org. layers were dried over MgSO₄, filtrated off and evaporated in vacuo. The crude material (3.0 g) of was used in the next step without purification LC-MS (A): t_{R}=0.48 min; [M+H]⁺: 160.20.

### 6-Chloro-5-hydroxypicolinaldehyde

This compound was prepared from 2-chloro-6-(hydroxymethyl)pyridin-3-ol according to the procedures described for aldehyde 1 (2.step). LC-MS (A): t_{R}=0.70 min; [M+H]⁺: not visible.

### 2-Chloro-6-(morpholinomethyl)pyridin-3-ol

This compound has been prepared from 6-chloro-5-hydroxypicolinaldehyde according to the procedure described for acid 6 (2.step). LC-MS (A): t_{R}=0.36 min; [M+H]⁺: 229.14.

### 2-((2-Chloro-6-(morpholinomethyl)pyridin-3-yl)oxy)acetic acid

This compound was prepared from 2-chloro-6-(morpholinomethyl)pyridin-3-ol in 2 steps according to the procedures described for acid 1. LC-MS (A): t_{R}=0.37 min; [M+H]⁺: 287.12.

### Acid 13: 2-((2-Chloro-6-(methoxycarbonyl)pyridin-3-yl)oxy) acetic acid

### 2-chloro-6-iodo-3-(methoxymethoxy)pyridine

This compound has been prepared from 2-chloro-6-iodopyridin-3-ol according to the procedure described for acid 2 (1.step). LC-MS (A): t_{R}=0.84 min; [M+H]⁺: 299.82.

### 6-Chloro-5-(methoxymethoxy)picolinic acid

To a solution of 2-chloro-6-iodo-3-(methoxymethoxy)pyridine (5.85 g) in toluene (80 ml) under nitrogen was added at −78° C. nBuLi 1.6M in hexanes (16 ml) under nitrogen. The mixture was stirred at −78° C. for 30 min. The reaction mixture was poured into CO_{2 (s)}. After the addition, 1N aq. NaOH (30 ml) was added and the aq. layer was extracted with diethyl ether. The layers were separated, the aq. phase was acidified at 0° C. with 2N aq. HCl until pH 1 and washed with DCM. The org. layer was dried over MgSO₄, filtrated off and evaporated in vacuo. The crude (3.73 g of a beige solid) was used in the next step without purification. LC-MS (A): t_{R}=0.58 min; [M+H]⁺: 217.98.

### Methyl 6-chloro-5-(methoxymethoxy)picolinate

To a solution of 6-chloro-5-(methoxymethoxy)picolinic acid (1.63 g) in MeOH (60 ml) was added dropwise at RT a 2.0 M solution of trimethylsilyldiazomethane in hexane (18.8 ml). The mixture was stirred at RT. Two other portions of trimethylsilyldiazomethane solution were added: (1.9 ml after 2 h and 1.9 ml after additional 3 h). 3 h after the last addition was the solvent evaporated in vacuo. The crude was purified by CC (Büchi Sepacore, 50 g cartridge, solvent A: heptane, solvent B: EA, gradient in % B: 1 to 9, flow rate: 30 ml/min) to afford 1.3 g of a yellow oil. LC-MS (A): t_{R}=0.71 min; [M+H]⁺: 232.09.

### 2-((2-Chloro-6-(methoxycarbonyl)pyridin-3-yl)oxy) acetic acid

This compound has been prepared from methyl 6-chloro-5-(methoxymethoxy)picolinate according to the procedures described for acid 4 (3-5.step). LC-MS (A): t_{R}=0.55 min; [M+H]⁺: 246.15.

### Acid 14: 3-(Carboxymethoxy)-2-chloropyridine 1-oxide

### 2-chloro-3-(2-methoxy-2-oxoethoxy)pyridine 1-oxide

This compound has been prepared from methyl 2-((2-chloropyridin-3-yl)oxy)acetate according to the procedures described for acid 8 (2-3.step). LC-MS (A): t_{R}=0.32 min; [M+H]⁺: 204.03.

### Acid 15: 2-((2-(tert-butoxycarbonyl)-5-methyl-1,2,3,4-tetrahydroisoquinolin-6-yl)oxy) acetic acid

### 3-Methoxy-2-methylbenzaldehyde

To a solution of N,N,N′-trimethylethylenediamine (7.1 ml) in toluene (140 ml) was added dropwise at 0° C. nBuLi 1.6M in hexanes (33 ml) under nitrogen. The mixture was stirred at rt for 1 h. 3-Methoxybenzaldehyde (6.27 ml) was added at 0° C. and the reaction mixture was stirred at rt for 1 h. Phenyllithium 1.8M in di-N-butylether (86 ml) was added at 0° C. and the reaction mixture was stirred at rt overnight. The mixture was cooled down to −75° C. and iodomethane (19.2 ml) was slowly added. The solution was stirred at rt for 4 h. The mixture was diluted in cold 10% aq. HCl and the aq. layer was washed three times with EA. The combined org. layers were washed with sat. aq. NaCl, dried over MgSO₄, filtrated off and evaporated in vacuo. The crude was purified by CC (Büchi Sepacore, 100 g cartridge, solvent A: Heptane, solvent B: EA, gradient in % B: 1 to 7, flow rate: 40 ml/min) to afford 5.75 g of a yellow oil. LC-MS (A): t_{R}=0.78 min; [M+H]⁺: not visible.

### (E)-1-Methoxy-2-methyl-3-(2-nitrovinyl)benzene

To a solution of 3-methoxy-2-methylbenzaldehyde (1 g) in nitromethane (20 ml) was added ammonium acetate (310 mg). The reaction mixture was stirred at 100° C. for 1 h. The solution was evaporated in vacuo and the residue was diluted with EA (50 ml). The org. layer was washed twice with sat. aq. NaCl, the combined org. layers were dried over MgSO₄, filtrated off and evaporated in vacuo. The crude was purified by CC (Büchi Sepacore, 50 g cartridge, solvent A: heptane, solvent B: EA, gradient in % B: 1, flow rate: 30 ml/min) to afford 975 mg of a yellow oil. LC-MS (A): t_{R}=0.89 min; [M+H]⁺: not visible.

### 2-(3-Methoxy-2-methylphenyl)ethanamine

To a solution of lithium borohydride 2M in THF (10 ml) was added TMSCI (5.05 ml). After stirring for 2 min, a solution of (E)-1-methoxy-2-methyl-3-(2-nitrovinyl)benzene (970 mg) in THF (20 ml) was added. The reaction mixture was stirred at rt overnight. The mixture was cooled down to 0° C. and MeOH (10 ml) was added. The solution was evaporated in vacuo and the residue was diluted with DCM (30 ml). The org. layer was washed with 25% aq. NaOH and sat. aq. NaCl, dried over MgSO₄, filtrated off and evaporated in vacuo. The crude was purified by CC (Büchi Sepacore, 20 g cartridge, solvent A: DCM, solvent B: NH₃ 7N in MeOH, gradient in % B: 0 to 5, flow rate: 20 ml/min) to afford 466 mg of a colourless oil. LC-MS (A): t_{R}=0.49 min; [M+H]⁺: 166.06.

### N-(3-Methoxy-2-methylphenethyl)formamide

A solution of 2-(3-methoxy-2-methylphenyl)ethanamine (463 mg) in ethyl formate (4 ml) was stirred at 60° C. overnight. The solution was evaporated in vacuo and the residue was purified by CC (Büchi Sepacore, 10 g cartridge, solvent A: heptane, solvent B: EA, gradient in % B: 2 to 15, flow rate: 15 ml/min) to afford 608 mg of a colourless oil. LC-MS (A): t_{R}=0.69 min; [M+H]⁺: 194.15.

### 6-Methoxy-5-methyl-3,4-dihydroisoquinoline-2(1H)-carbaldehyde

To a solution of N-(3-methoxy-2-methylphenethyl)formamide (600 mg) in formic acid (6.2 ml) was added paraformaldehyde (400 mg). The reaction mixture was stirred at 100° C. for 20 min. The solution was evaporated in vacuo and the residue was purified by CC (Büchi Sepacore, 10 g cartridge, solvent A: DCM, solvent B: NH₃ 7N in MeOH, gradient in % B: 0 to 5, flow rate: 15 ml/min) to afford 535 mg of a white solid. LC-MS (A): t_{R}=0.75 min; [M+H]⁺: 206.10.

### 5-Methyl-1,2,3,4-tetrahydroisoquinolin-6-ol

To a solution of 6-methoxy-5-methyl-3,4-dihydroisoquinoline-2(1H)-carbaldehyde (530 mg) in DCM (15 ml) cooled down to 0° C. was added borontribromide 1M in DCM (12.2 ml). The reaction mixture was stirred at 0° C. for 1 h25 min. MeOH (10 ml) was slowly added and the mixture was stirred at rt over 9 days. The solution was evaporated in vacuo and the residue was purified by CC (Büchi Sepacore, 20 g cartridge, solvent A: DCM, solvent B: NH₃ 7N in MeOH, gradient in % B: 1 to 10, flow rate: 20 ml/min) to afford 370 mg of a white solid. LC-MS (A): t_{R}=0.36 min; [M+H]⁺: 164.07.

### Tert-butyl 6-hydroxy-5-methyl-3,4-dihydroisoquinoline-2(1H)-carboxylate

To a solution of 5-methyl-1,2,3,4-tetrahydroisoquinolin-6-ol (355 mg) in 1N aq. NaOH (3.6 ml) was added di-tert-butyldicarbonate (850 mg) in dioxane (20 ml). The reaction mixture was stirred at rt for 5 min. The mixture was diluted with DCM (20 ml) and 1N aq. HCl (5 ml). The aq. layer was extracted with DCM and the combined org. layers were washed with sat. aq. NaCl., dried over MgSO₄, filtrated off and evaporated in vacuo. The crude was purified by CC (Büchi Sepacore, 10 g cartridge, solvent A: DCM, solvent B: MeOH, gradient in % B: 0 to 5, flow rate: 6 ml/min) to afford 295 mg of a yellow oil. LC-MS (A): t_{R}=0.84 min; [M+H]⁺: 264.12.

### 2-((2-(Tert-butoxycarbonyl)-5-methyl-1,2,3,4-tetrahydroisoquinolin-6-yl)oxy)acetic acid

This compound has been prepared tert-butyl 6-hydroxy-5-methyl-3,4-dihydroisoquinoline-2(1H)-carboxylate according to procedures described for acid 7 (1-2.step). LC-MS (A): t_{R}=0.84 min; [M+H]⁺: not visible.

### Acid 16: 2-(2-chloro-4-(3-(1,1-dimethylethylsulfinamido)oxetan-3-yl)phenoxy)acetic acid

### 2-Methyl-N-(oxetan-3-ylidene)propane-2-sulfinamide

To a solution of oxetan-3-one (759 mg) in THF (30 ml) were added 2-methyl-2-propane-sulfinamide (1.25 g) and titanium (IV) ethoxide (4.4 ml). The reaction mixture was stirred at 50° C. for 4 h. The mixture was diluted with sat. aq. NaCl (200 ml) and the suspension was filtrated through a pad of celite and washed with EA. Both layers of the filtrate were separated and the aq. layer was extracted with EA. The combined org. layers were dried over MgSO₄, filtrated off and evaporated in vacuo. The crude was purified by CC (Flash Master, solvent A: heptane, solvent B: EA, gradient in % B: 20, flow rate: 20 ml/min) to afford 1.18 g of a yellow oil. LC-MS (A): t_{R}=0.55 min; [M+H]⁺: 176.26.

### (4-Bromo-2-chlorophenoxy) (tert-butyl)dimethylsilane

To a solution of 4-bromo-2-chlorophenol (500 mg) in DMF (10 ml) cooled down to 0° C. were added DIPEA (0.83 ml) and TBDMSCI (545 mg). The reaction mixture was stirred at rt for 3 h. The solution was diluted with sat. aq. NH₄Cl and EA. The layers were separated and the aq. layer was extracted twice with EA. The combined org. layers were washed with sat. aq. NaCl, dried over MgSO₄, filtrated off and evaporated in vacuo. The crude was purified by CC (Büchi Sepacore, 10 g cartridge, solvent A: heptane, solvent B: EA, gradient in % B: 0, flow rate: 15 ml/min) to afford 708 mg of a colourless oil. LC-MS (A): t_{R}=1.12 min; [M+H]⁺: not visible.

### N-(3-(4-((tert-butyldimethylsilyl)oxy)-3-chlorophenyl)oxetan-3-yl)-2-methylpropane-2-sulfinamide

To a solution of (4-bromo-2-chlorophenoxy)(tert-butyl)dimethylsilane (606 mg) in THF (10 ml) cooled down to −78° C. was added nBuLi 1.6M in hexanes (1.1 ml). The resulting mixture was stirred at −78° C. for 45 min. A solution 2-methyl-N-(oxetan-3-ylidene)propane-2-sulfinamide (220 mg) in THF (2 ml) cooled down at −78° C. was added to the previous solution. The reaction mixture was stirred at −78° C. for 15 min and then at rt overnight. The solution was diluted with sat. aq. NH₄Cl, water and EA. The layers were separated and the aq. layer was extracted with EA. The combined org. layers were dried over MgSO₄, filtrated off and evaporated in vacuo. The crude was purified by CC (Büchi Sepacore, 10 g cartridge, solvent A: heptane, solvent B: EA, gradient in % B: 1 to 30, flow rate: 15 ml/min) to afford 447 mg of an orange oil. LC-MS (A): t_{R}=1.01 min; [M+H]⁺: 417.74.

### N-(3-(3-chloro-4-hydroxyphenyl)oxetan-3-yl)-2-methylpropane-2-sulfinamide

To a solution of N-(3-(4-((tert-butyldimethylsilyl)oxy)-3-chlorophenyl)oxetan-3-yl)-2-methylpropane-2-sulfinamide (440 mg) in THF (9 ml) cooled down to 0° C. was added TBAF (330 mg). The reaction mixture was stirred at rt overnight. The next morning, TBAF (137 mg) was added again and the mixture was stirred at rt for 5 h30 min. The suspension was filtrated off and the filtrate was evaporated in vacuo. The crude was purified by CC (Büchi Sepacore, 10 g cartridge, solvent A: DCM, solvent B: NH₃ 7N in MeOH, gradient in % B: 1 to 2, flow rate: 15 ml/min) to afford 270 mg of a yellow solid. LC-MS (A): t_{R}=0.64 min; [M+H]⁺: 304.12.

### Methyl 2-(2-chloro-4-(3-(1,1-dimethylethylsulfinamido)oxetan-3-yl)phenoxy)acetate

To a solution of N-(3-(3-chloro-4-hydroxyphenyl)oxetan-3-yl)-2-methylpropane-2-sulfinamide (270 mg) in MeOH (5 ml) cooled down to 0° C. was added potassium hydroxide (55 mg). The mixture was stirred at 0° C. for 30 min. Methylbromoacetate (0.09 ml) was added and the reaction mixture was stirred at rt for 4 h. 8*0.04 ml of methylbromoacetate were added until the reaction was completed. The solution was diluted with sat. aq. NH₄Cl and EA. The layers were separated and the aq. layer was extracted twice with EA. The combined org. layers were washed with sat. aq. NaCl, dried over MgSO₄, filtrated off and evaporated in vacuo. The crude was purified by CC (Büchi Sepacore, 10 g cartridge, solvent A: DCM, solvent B: NH₃ 7N in MeOH, gradient in % B: 1 to 2, flow rate: 10 ml/min) to afford 220 mg of a beige foam. LC-MS (A): t_{R}=0.75 min; [M+H]⁺: 375.91.

### 2-(2-Chloro-4-(3-(1,1-dimethylethylsulfinamido)oxetan-3-yl)phenoxy)acetic acid

This compound has been prepared from methyl 2-(2-chloro-4-(3-(1,1-dimethylethylsulfinamido)oxetan-3-yl)phenoxy)acetate according to procedures described for acid 7 (2.step). LC-MS (A): t_{R}=0.65 min; [M+H]⁺: 362.02.

### Acid 17: 2-((1-ethylnaphthalen-2-yl)oxy)acetic acid

### 1-Ethyl-2-methoxynaphthalene

To a solution of 2M aq. Na₂CO₃ (4.12 ml) were added 1-bromo-2-methoxynaphtalene, tetrakis(triphenylphosphine)palladium (0) (73 mg) and triethylborane 1M in THF (12.7 ml). The reaction mixture was stirred at 90° C. for 20 h. The solution was diluted with water and EA. The org. layer was washed with sat. aq. NaCl, dried over MgSO₄, filtrated off and evaporated in vacuo. The crude was purified by CC (Flash Master, 40 g cartridge, solvent A: heptane, solvent B: EA, gradient in % B: 10 to 20, flow rate: 25 ml/min) to afford 245 mg of a yellow oil.

### 1-Ethylnaphthalen-2-ol

To a solution of 1-ethyl-2-methoxynaphthalene (245 mg) in DCM (10 ml) was added borontribromide (0.35 ml). The reaction mixture was stirred under reflux for 2 h, then cooled down to rt and hydrolized with 5% aq. HCl. The layers were separated and the aq. layer was extracted with DCM. The combined org. layers were washed with 1N aq. NaOH, dried over MgSO₄, filtrated off and evaporated in vacuo. The crude was purified by CC (Flash Master, 12 g cartridge, solvent A: heptane, solvent B: EA, gradient in % B: 0 to 20, flow rate: 15 ml/min) to afford 142 mg of a yellow solid. LC-MS (B): t_{R}=0.86 min; [M+H]⁺: not visible.

### 2-((1-Ethylnaphthalen-2-yl)oxy)acetic acid

This compound has been prepared from 1-ethylnaphthalen-2-ol according to procedures described for acid 4 (4-5.step). LC-MS (B): t_{R}=0.83 min; [M+H]⁺: not visible.

### Acid 18: 2-(2,3-dimethyl-4-(morpholinomethyl)phenoxy)acetic acid

### 4-Hydroxy-2,3-dimethylbenzaldehyde

This compound has been prepared from 4-methoxy-2,3-dimethylbenzaldehyde according to procedures described for acid 17 (2.step). LC-MS (A): t_{R}=0.68 min; [M+AcCN]⁺: 192.27.

### 2,3-Dimethyl-4-(morpholinomethyl)phenol

This compound has been prepared 4-hydroxy-2,3-dimethylbenzaldehyde according to procedures described for acid 6 (2.step). LC-MS (A): t_{R}=0.46 min; [M+H]⁺: 222.25.

### 2-(2,3-dimethyl-4-(morpholinomethyl)phenoxy)acetic acid

This compound has been prepared from 2,3-dimethyl-4-(morpholinomethyl)phenol according to the procedures described for acid 1 (1-2.step). LC-MS (A): t_{R}=0.50 min; [M+H]⁺: 280.09.

### Acid 19: 2-((1-methylnaphthalen-2-yl)oxy)acetic acid

### 2-Methoxy-1-methylnaphthalene

To a solution of 1-bromo-2-methoxynaphtalene (500 mg) in DMF (10 ml) were added potassium carbonate (736 mg), dimethyl zinc (2.6 ml) and (1,1′-bis(diphenylphosphino)ferrocene)dichloropalladium (II) dichloromethane adduct (35 mg). The reaction mixture was stirred at 90° C. for 24 h. The mixture was diluted with water and EA, the layers were separated. The aq. layer was extracted with EA and the combined org. layers were washed with sat. aq. NaCl, dried over MgSO₄, filtrated off and evaporated in vacuo. The crude was purified by CC (Flash Master, 25 g cartridge, solvent A: Heptane, solvent B: EA, gradient in % B: 0 to 10, flow rate: 20 ml/min) to afford 532 mg of a yellow oil. LC-MS (B) t_{R}=0.99 min; [M+H]⁺: not visible.

### 2-((1-Methylnaphthalen-2-yl)oxy)acetic acid

This acid has been prepared from 2-methoxy-1-methylnaphthalene according to the procedures described for acid 17 (2-4.step). LC-MS (B): t_{R}=0.79 min; [M+H]⁺: not visible.

### Acid 20: 2-((1-chloronaphthalen-2-yl)oxy)acetic acid

This acid has been prepared from 1-chloronaphthalen-2-ol according to the procedure described for 4 (4-5.step). LC-MS (B): t_{R}=0.79 min; [M+H]⁺: not visible.

### Acid 21: 2-((2-(tert-butoxycarbonyl)-1,2,3,4-tetrahydroisoquinolin-7-yl)oxy)acetic acid

This acid has been prepared from 1,2,3,4-tetrahydroisoquinolin-7-ol hydrobromid according to the procedures described for 15 (7-9.step). LC-MS (A): t_{R}=0.80 min; [M+H]⁺: not visible.

### Acid 22: 2-(5-(ethoxycarbonyl)-2-ethyl-4-methylphenoxy)acetic acid

### 5-Bromo-2-ethyl-4-methylphenol

To a solution of 1-(4-bromo-2-hydroxy-5-methylphenyl)ethan-1-one (3 g) and triethylamine (2.19 ml) in THF (13 ml) cooled down to 0° C. was added ethyl chloroformate (1.5 ml) dropwise. The suspension was stirred at 0° C. for 30 min, then filtrated and washed with THF. The liquid layer was slowly added to a solution of NaBH₄ (2 g) in water (21 ml) at 5-15° C. The reaction mixture was stirred at rt overnight. The solution was diluted with water and acidified with 1N aq. HCl. The aq. layer was extracted with EA and the org. layer was washed with 10% aq. NaOH, dried over MgSO₄, filtrated off and evaporated in vacuo. The crude (2.85 g of a colourless oil) was used in the next step without purification. LC-MS (B): t_{R}=0.91 min; [M+H]⁺: not visible.

### 1-(Benzyloxy)-5-bromo-2-ethyl-4-methylbenzene

To a solution of 5-bromo-2-ethyl-4-methylphenol (2.85 g) in THF (65 ml) were added cesium carbonate (4.56 g) and benzylbromide (1.69 ml). The reaction mixture was stirred at rt overnight. The mixture was evaporated in vacuo, the residue was diluted with water and EA. The org. layer was washed with sat. aq. NaHCO₃ and sat. aq. NaCl, dried over MgSO₄, filtrated off and evaporated in vacuo. The crude was purified by CC (Flash Master, 40 g cartridge, solvent A: heptane, solvent B: EA, gradient in % B: 0 to 5, flow rate: 25 ml/min) to afford 2.73 g of a colourless oil. LC-MS (B): t_{R}=1.20 min; [M+H]⁺: not visible.

### Ethyl 5-(benzyloxy)-4-ethyl-2-methylbenzoate

To a solution of 1-(benzyloxy)-5-bromo-2-ethyl-4-methylbenzene (1.27 g) in THF (28 ml) cooled down to −78° C. was added nBuLi 2.5M in hexanes (3 ml). The mixture was stirred at −78° C. for 1 h and ethyl chloroformate (0.8 ml) in THF (8 ml) was added. The reaction mixture was stirred at rt for 4 h. The solution was diluted with EA and 1N aq. HCl. The layers were separated and the org. layer was washed with sat. aq. NaCl, dried over MgSO₄, filtrated off and evaporated in vacuo. The crude was purified by CC (Flash Master, 40 g cartridge, solvent A: heptane, solvent B: EA, gradient in % B: 0 to 10, flow rate: 25 ml/min) to afford 783 mg of a yellow oil. LC-MS (B): t_{R}=1.16 min; [M+H]⁺: 299.01.

### Ethyl 4-ethyl-5-hydroxy-2-methylbenzoate

To a solution of ethyl 5-(benzyloxy)-4-ethyl-2-methylbenzoate (783 mg) in MeOH (10 ml) was added under argon Pd/C (56 mg). The reaction mixture was hydrogenated at rt overnight. The mixture was filtrated through a pad of celite and the liquid phase was evaporated in vacuo. The crude (457 mg of a yellow solid) was used in the next step without purification. LC-MS (B): t_{R}=0.87 min; [M+H]⁺: not visible.

### Ethyl 5-(2-(benzyloxy)-2-oxoethoxy)-4-ethyl-2-methylbenzoate

To a solution of ethyl 4-ethyl-5-hydroxy-2-methylbenzoate (222 mg) in THF (4 ml) were added cesium carbonate (380 mg) and benzyl bromoacetate (0.18 ml). The reaction mixture was stirred at rt for 1 h30 min. The suspension was diluted with EA, cooled down to 0° C. and sat. aq. NH₄Cl was added. The layers were separated, the org. layer was washed with sat. aq. NaCl, dried over MgSO₄, filtrated off and evaporated in vacuo. The crude was purified by CC (Flash Master, 24 g cartridge, solvent A: heptane, solvent B: EA, gradient in % B: 0 to 10, flow rate: 20 ml/min) to afford 187.5 mg of a colourless oil. LC-MS (E): t_{R}=1.04 min; [M+H]⁺: 356.90.

### 2-(5-(Ethoxycarbonyl)-2-ethyl-4-methylphenoxy)acetic acid

To a solution of ethyl 5-(2-(benzyloxy)-2-oxoethoxy)-4-ethyl-2-methylbenzoate (187.5 mg) in MeOH (2 ml) was added 10% palladium hydroxide on charcoal (15 mg). The reaction mixture was hydrogenated at rt for 2 h30 min. The suspension was filtrated through a pad of celite and the liquid phase was evaporated in vacuo to afford 120.5 mg of a white solid. LC-MS (E): t_{R}=0.79 min; [M+H]⁺: not visible.

### Acid 23: 2-((1-methyl-3-phenyl-1H-pyrazol-5-yl)oxy)acetic acid

### Methyl 2-((1-methyl-3-phenyl-1H-pyrazol-5-yl)oxy)acetate

This ester has been prepared from 1-methyl-3-phenyl-1H-pyrazol-5-ol according to the procedures described for acid 7 (1-2.steps). LC-MS (A): t_{R}=0.65 min; [M+H]⁺: 233.04.

### Acid 24: 2-((2-Chloro-6-(cyclopropylcarbamoyl)pyridin-3-yl)oxy)acetic acid

### 6-Chloro-N-cyclopropyl-5-(methoxymethoxy)picolinamide

This amide has been prepared from 6-chloro-5-(methoxymethoxy)picolinic acid and cyclopropylamine according to the method C. LC-MS (A): t_{R}=0.75 min; [M+H]⁺: 257.16.

### 2-((2-Chloro-6-(cyclopropylcarbamoyl)pyridin-3-yl)oxy)acetic acid

This acid has been prepared from 6-chloro-N-cyclopropyl-5-(methoxymethoxy)picolinamide according to the procedures described for acid 2 (3-5.steps). LC-MS (A): t_{R}=0.65 min; [M+H]⁺: 233.04.

### Acid 25: 2-((2-Chloro-6-(methyl(2,2,2-trifluoroethyl)amino)pyridin-3-yl)oxy)acetic acid

### 6-Chloro-5-(methoxymethoxy)-N-methylpyridin-2-amine

This compound has been prepared 2-chloro-6-iodopyridin-3-ol and methylamine according to the procedures described for acid 4 (1-2.steps). LC-MS (A): t_{R}=0.69 min; [M+H]⁺: 203.20.

### N-(6-Chloro-5-(methoxymethoxy)pyridin-2-yl)-2,2,2-trifluoro-N-methylacetamide

A solution of 6-chloro-5-(methoxymethoxy)-N-methylpyridin-2-amine (61 mg), trifluoroacetic acid anhydride (63 μl) and DIPEA (103 μl) in DCM was stirred at 0° C. for 90 min. The solution was diluted with DCM and 1N aq. HCl. The layers were separated and the org. layer was washed with sat. aq. NaCl, dried over MgSO₄, filtrated off and evaporated in vacuo. The crude was purified by CC (Büchi Sepacore, 2 g cartridge, solvent A: heptane, solvent B: EA, gradient in % B: 1 to 5, flow rate: 6 ml/min) to afford 76 mg of a yellow oil. LC-MS (A): t_{R}=0.84 min; [M+H]⁺: 299.03.

### 2-Chloro-6-(methyl(2,2,2-trifluoroethyl)amino)pyridin-3-ol

A solution of N-(6-chloro-5-(methoxymethoxy)pyridin-2-yl)-2,2,2-trifluoro-N-methylacetamide (105 mg) and borane-methylsulfide complex (2M sol. in THF, 1.75 ml) in THF (7 ml) was stirred at 50° C. for ca. 40 h. The solution was diluted with EA and 1N aq. NaOH. The layers were separated and the org. layer was washed with sat. aq. NaCl, dried over MgSO₄, filtrated off and evaporated in vacuo. The crude (80 mg of a yellowish oil) was used in the next step without purification. LC-MS (A): t_{R}=0.80 min; [M+H]⁺: 241.06.

### 2-((2-Chloro-6-(methyl(2,2,2-trifluoroethyl)amino)pyridin-3-yl)oxy)acetic acid

This acid has been prepared from 2-chloro-6-(methyl(2,2,2-trifluoroethyl)amino)pyridin-3-ol according to the procedures described for acid 1. LC-MS (A): t_{R}=0.80 min; [M+H]⁺: 299.08.

### Acid 26: 2-((2-Chloro-6-(dimethylamino)pyridin-3-yl)oxy)acetic acid

### 6-chloro-5-(methoxymethoxy)-N,N-dimethylpyridin-2-amine

This compound has been prepared from 2-chloro-6-iodopyridin-3-ol and dimethylamine according to the procedures described for acid 25 (1-2. steps). LC-MS (A): t_{R}=0.82 min; [M+H]⁺: 217.34.

### 2-((2-Chloro-6-(dimethylamino)pyridin-3-yl)oxy)acetic acid

This acid has been prepared from 6-chloro-5-(methoxymethoxy)-N,N-dimethylpyridin-2-amine according to the procedures described for acid 2 (3-5. steps). LC-MS (A): t_{R}=0.67 min; [M+H]⁺: 231.25.

### Acid 27: 2-((2-Cyclopropyl-6-(cyclopropylcarbamoyl)pyridin-3-yl)oxy)acetic acid

### 3-(Benzyloxy)-2-chloro-6-iodopyridine

A solution of 2-chloro-6-iodopyridin-3-ol (40.4 g), K₂CO₃ (33 g) and BnBr (20 ml) in DMF was stirred at 60° C. for 2 h. The solution was diluted at rt with EA and aq. NH₄Cl. The layers were separated and the org. layer was washed with water and with sat. aq. NaCl, dried over MgSO₄, filtrated off and evaporated in vacuo. The crude was triturated with heptane/EA mixture to afford 43.2 g of a colourless solid. LC-MS (A): t_{R}=0.97 min; [M+H]⁺: 345.79.

### 5-(Benzyloxy)-6-chloropicolinic acid

This compound has been prepared from 3-(benzyloxy)-2-chloro-6-iodopyridine according to the procedures described for acid 13 (2. step). LC-MS (A): t_{R}=0.77 min; [M+H]⁺: 263.99.

### 5-(Benzyloxy)-6-chloro-N-cyclopropylpicolinamide

This compound has been prepared from 5-(benzyloxy)-6-chloropicolinic acid and cyclopropylamine according to the method C. LC-MS (A): t_{R}=0.90 min; [M+H]⁺: 302.99.

### 6-Chloro-N-cyclopropyl-5-hydroxypicolinamide

A solution of 5-(benzyloxy)-6-chloro-N-cyclopropylpicolinamide (13.7 g) and Pd/C (1.4 g) in MeOH (600 ml) was stirred under hydrogen at 1 bar at rt for 20 min. The mixture was filtrated off and evaporated in vacuo. The crude was triturated with DCM to afford 7.5 g of a colourless solid. LC-MS (A): t_{R}=0.62 min; [M+H]⁺: 213.07.

### N,6-Dicyclopropyl-5-hydroxypicolinamide

A solution of 6-chloro-N-cyclopropyl-5-hydroxypicolinamide (100 mg), cyclopropylboronic acid (25 mg), Pd(PPh₃)₄(34 mg) and K₂CO₃ (62 mg) in dioxane (4 ml) was stirred at 120° C. for 3 days. During this time cyclopropylboronic acid (2×75 mg), Pd(PPh₃)₄(2×34 mg) were added. The mixture was filtrated off and evaporated in vacuo. The crude was purified by preparative LC-MS [preparative LC-MS (1)] to afford 50 mg of a yellowish solid. LC-MS (A): t_{R}=0.70 min; [M+H]⁺: 219.13.

### 2-((2-Cyclopropyl-6-(cyclopropylcarbamoyl)pyridin-3-yl)oxy)acetic acid

This acid has been prepared from N,6-dicyclopropyl-5-hydroxypicolinamide according to the procedures described for acid 1 (1-2. steps). LC-MS (A): t_{R}=0.70 min; [M+H]⁺: 277.13.

### Acid 28: 2-((2-chloro-6-((2-methoxyethyl)(methyl)amino)pyridin-3-yl)oxy)acetic acid

### Tert-butyl 2-((2-chloro-6-((2-methoxyethyl)(methyl)amino)pyridin-3-yl)oxy)acetate

This compound has been prepared from tert-butyl 2-((2-chloro-6-iodopyridin-3-yl)oxy)acetate and N-(2-methoxyethyl)methylamine according to the procedure described for acid 4 (2.step). LC-MS (A): t_{R}=0.94 min; [M+H]⁺: 331.18.

### 2-((2-Chloro-6-((2-methoxyethyl)(methyl)amino)pyridin-3-yl)oxy)acetic acid

This acid has been prepared from tert-butyl 2-((2-chloro-6-((2-methoxyethyl)(methyl)amino)pyridin-3-yl)oxy)acetate according to the procedures described for acid 1 (2. steps). LC-MS (A): t_{R}=0.70 min; [M+H]⁺: 275.09.

### Acid 29: 2-((2-Cyclopropyl-6-(methylsulfonamido)pyridin-3-yl)oxy)acetic acid

### Tert-butyl 2-((2-cyclopropyl-6-(methylsulfonamido)pyridin-3-yl)oxy)acetate

This compound has been prepared from tert-butyl 2-((2-chloro-6-(methylsulfonamido)pyridin-3-yl)oxy)acetate according to the procedure described for acid 27 (5. step).

### 2-((2-Cyclopropyl-6-(methylsulfonamido)pyridin-3-yl)oxy)acetic acid

This acid has been prepared from tert-butyl 2-((2-cyclopropyl-6-(methylsulfonamido)pyridin-3-yl)oxy)acetate according to the procedure described for acid 1 (2. steps). LC-MS (A): t_{R}=0.62 min; [M+H]⁺: 287.10.

### Acid 30: 2-((2-Ethyl-6-(methylsulfonamido)pyridin-3-yl)oxy)acetic acid

### Tert-butyl 2-((2-ethyl-6-(methylsulfonamido)pyridin-3-yl)oxy)acetate

A solution of tert-butyl 2-((2-chloro-6-(methylsulfonamido)pyridin-3-yl)oxy)acetate (192 mg), diethylzinc (1M in hexanes, 1.0 ml) and (1,1′-bis(diphenylphosphino) ferrocene) dichloropalladium (II) dichloromethane (15 mg) in dioxane (8 ml) was stirred at 85° C. for 90 min. The solution was diluted at rt with EA and water. The layers were separated and the org. layer was washed with water and with sat. aq. NaCl, dried over MgSO₄, filtrated off and evaporated in vacuo. The crude was purified by preparative LC-MS [preparative LC-MS (I)] to afford 180 mg of a brown solid. LC-MS (A): t_{R}=0.83 min; [M+H]⁺: 331.26.

### 2-((2-Ethyl-6-(methylsulfonamido)pyridin-3-yl)oxy)acetic acid

This acid has been prepared from tert-butyl 2-((2-ethyl-6-(methylsulfonamido)pyridin-3-yl)oxy)acetate according to the procedure described for acid 1 (2. steps). LC-MS (A): t_{R}=0.55 min; [M+H]⁺: 275.01.

### Acid 31: 2-(2-Chloro-4-(trifluoromethyl)phenoxy)acetic acid

This acid has been prepared from 2-chloro-4-(trifluoromethyl)phenol according to the procedures described for acid 7 (1-2. steps). LC-MS (A): t_{R}=1.15 min; [M+H]⁺: not visible.

### Acid 32: 2-((2-Chloro-6-(trifluoromethyl)pyridin-3-yl)oxy)acetic acid

This acid has been prepared from 2-chloro-6-(trifluoromethyl)pyridin-3-ol according to the procedures described for acid 1 (1-2. steps). LC-MS (A): t_{R}=0.72 min; [M+H]⁺: 256.01.

### Acid 33: 2-((2-Chloro-6-cyanopyridin-3-yl)oxy)acetic acid

### 6-Chloro-5-(methoxymethoxy)picolinamide

To a solution of 6-chloro-5-(methoxymethoxy)picolinic acid (3.6 g) in THF (80 ml) were added at 0° C. triethylamine (6 ml) and methylchloroformate (3 ml). The mixture was stirred at 0° C. for 30 min. Ammonium hydroxide 25% in water (20 ml) was added and the reaction mixture was stirred at rt for 10 min. The reaction mixture was diluted with EA and water. The layers were separated, the org. phase was washed with sat. aq. NaCl, dried over MgSO₄, filtrated off and evaporated in vacuo. The crude was purified by CC (Büchi Sepacore, 50 g cartridge, solvent A: DCM, solvent B: MeOH, gradient in % B: 1 to 3, flow rate: 35 ml/min) to afford 2.4 g of a white solid. LC-MS (A): t_{R}=0.63 min; [M+H]⁺: 217.03.

### 6-Chloro-5-(methoxymethoxy)picolinonitrile

To a solution of 6-chloro-5-(methoxymethoxy)picolinamide (2.4 g) in DCM (100 ml) was added Burgess reagent (6 g). The reaction mixture was stirred at rt for 3 h. The reaction mixture was diluted with DCM and sat. aq. NaHCO₃. The layers were separated, the org. phase was washed with water and sat. aq. NaCl, dried over MgSO₄, filtrated off and evaporated in vacuo. The crude was purified by CC (Büchi Sepacore, 50 g cartridge, solvent A: Heptane, solvent B: EA, gradient in % B: 1 to 20, flow rate: 30 ml/min) to afford 1.72 g of a colourless oil. LC-MS (A): t_{R}=0.76 min; [M+H]⁺: not visible.

### 2-((2-Chloro-6-cyanopyridin-3-yl)oxy)acetic acid

This acid has been prepared from 6-chloro-5-(methoxymethoxy)picolinonitrile according to the procedure described for acid 2 (1-3.step). LC-MS (A): t_{R}=0.59 min; [M+H]⁺: not visible.

### Acid 34: 2-((2-Chloro-6-iodopyridin-3-yl)oxy)acetic acid

This acid has been prepared from tert-butyl 2-((2-chloro-6-iodopyridin-3-yl)oxy)acetate according to the procedure described for acid 1 (2.step). LC-MS (A): t_{R}=0.69 min; [M+H]⁺: 313.82.

### Acid 35: 2-((2-Chloro-6-(methylsulfonyl)pyridin-3-yl)oxy)acetic acid

### Tert-butyl 2-((2-chloro-6-(methylsulfonyl)pyridin-3-yl)oxy)acetate

A mixture of tert-butyl 2-((2-chloro-6-iodopyridin-3-yl)oxy)acetate (see synthesis of acid 5, 1.step) (100 mg), sodium methanesulfinate (33 mg) and CuI (155 mg) in DMSO (5 ml) was stirred at 100° C. for 30 min. The mixture was diluted at rt with EA and aq. sat. NH₄Cl. The layers were separated and the aq. phase was washed twice with EA. The combined org. layers were washed with aq. sat. NaCl, dried over MgSO₄, filtrated off and evaporated in vacuo. The crude was with column chromatography (solvent A: Heptane, solvent B: EA, gradient in % B: 5 to 100) to afford 70 mg of a colourless solid. LC-MS (A): t_{R}=0.82 min; [M+H]⁺: 322.05.

### 2-((2-Chloro-6-(methylsulfonyl)pyridin-3-yl)oxy)acetic acid

This compound has been prepared from tert-butyl 2-((2-chloro-6-(methylsulfonyl)pyridin-3-yl)oxy)acetate according to the procedure described for acid 1 (2.step). LC-MS (A): t_{R}=0.51 min; [M+H]⁺: 265.39.

### Acid 36: 1-((2-Chloro-6-(((methylthio)peroxy)amino)pyridin-3-yl)oxy)cyclopropane-1-carboxylic acid

### Tert-butyl 4-bromo-2-((2-chloro-6-iodopyridin-3-yl)oxy)butanoate

To a solution of 2-chloro-6-iodopyridin-3-ol (500 mg) in DMF (10 ml) was added at 0° C. NaH (115 mg, 60% dispersion in mineral oil) and the mixture was stirred at this temperature for 30 min. Methyl 2,4-dibromobutanoate (0.400 ml) was added and the mixture was stirred at rt for 6 h. The mixture was diluted with heptane and aq. sat. NaHCO₃. The layers were separated and the aq. phase was washed twice with heptane. The combined org. layers were washed with aq. sat. NaCl, dried over MgSO₄, filtrated off and evaporated in vacuo. The crude was purified by CC (Büchi Sepacore, 20 g cartridge, solvent A: Heptane, solvent B: EA, gradient in % B: 1 to 5, flow rate: 20 ml/min) to afford 417 mg of a colourless oil. LC-MS (A): t_{R}=1.02 min; [M+H]⁺: 475.82.

### Tert-butyl 1-((2-chloro-6-iodopyridin-3-yl)oxy)cyclopropane-1-carboxylate

To a solution of tert-butyl 1-((2-chloro-6-iodopyridin-3-yl)oxy)cyclopropane-1-carboxylate (415 mg) in THF (10 mL) was added at −20° C. potassium tert-butoxide (106 mg) and the mixture was stirred for 15 min. The mixture was diluted with EA and water. The layers were separated and the aq. phase was washed twice with EA. The combined org. layers were washed with aq. sat. NaCl, dried over MgSO₄, filtrated off and evaporated in vacuo. The crude (307 mg of a yellowish oil) was used in the next step without purification LC-MS (A): t_{R}=0.98 min; [M+H]⁺: 396.02.

### Tert-butyl 1-((2-chloro-6-(((methylthio)peroxy)amino)pyridin-3-yl)oxy)cyclopropane-1-carboxylate

This compound has been prepared from tert-butyl 1-((2-chloro-6-iodopyridin-3-yl)oxy)cyclopropane-1-carboxylate according to the procedure described for acid 5 (2.step). LC-MS (A): t_{R}=0.86 min; [M+H]⁺: 363.11.

### 1-((2-Chloro-6-(((methylthio)peroxy)amino)pyridin-3-yl)oxy)cyclopropane-1-carboxylic acid

This compound has been prepared from tert-butyl 1-((2-chloro-6-(((methylthio)peroxy)amino)pyridin-3-yl)oxy)cyclopropane-1-carboxylate according to the procedure described for acid 1 (2.step). LC-MS (A): t_{R}=0.63 min; [M+H]⁺: 306.89.

### Acid 37: 1-((2-Chloro-6-(dimethylcarbamoyl)pyridin-3-yl)oxy)cyclopropane-1-carboxylic acid

### 5-(1-Tert-butoxycarbonyl)cyclopropoxy)-6-chloropicolinic acid

This compound has been prepared from tert-butyl 1-((2-chloro-6-iodopyridin-3-yl)oxy)cyclopropane-1-carboxylate (for the synthesis see acid 36, 2.step) according to the procedure described for acid 13 (2.step). LC-MS (A): t_{R}=0.80 min; [M+H]⁺: 314.02.

### Tert-butyl 1-((2-chloro-6-(dimethylcarbamoyl)pyridin-3-yl)oxy)cyclopropane-1-carboxylate

This amide has been prepared from 5-(1-tert-butoxycarbonyl)cyclopropoxy)-6-chloropicolinic acid and dimethylamine according to the method C. LC-MS (A): t_{R}=0.85 min; [M+H]⁺: 341.10.

### 1-((2-Chloro-6-(dimethylcarbamoyl)pyridin-3-yl)oxy)cyclopropane-1-carboxylic acid

This compound has been prepared from tert-butyl 1-((2-chloro-6-(dimethylcarbamoyl)pyridin-3-yl)oxy)cyclopropane-1-carboxylate according to the procedure described for acid 1 (2.step). LC-MS (A): t_{R}=0.61 min; [M+H]⁺: 285.07.

### Acid 38: 2-((2-Chloro-6-(dimethylcarbamoyl)pyridin-3-yl)oxy)-2-methylpropanoic acid

### Tert-butyl 2-((2-chloro-6-iodopyridin-3-yl)oxy)-2-methylpropanoate

This compound has been prepared from 2-chloro-6-iodopyridin-3-ol and tert-butyl 2-bromo-2-methylpropanoate according to the procedure described for acid 4 (4.step). LC-MS (A): t_{R}=1.01 min; [M+H]⁺: 398.02.

### 2-((2-Chloro-6-(dimethylcarbamoyl)pyridin-3-yl)oxy)-2-methylpropanoic acid

This compound has been prepared from tert-butyl 2-((2-chloro-6-iodopyridin-3-yl)oxy)-2-methylpropanoate according to the procedures described for acid 37 (step 3-5). LC-MS (A): t_{R}=0.63 min; [M+H]⁺: 287.08.

### Acid 39: 2-((2-Chloro-6-(oxazol-2-yl)pyridin-3-yl)oxy)acetic acid

### 2-(6-Chloro-5-(methoxymethoxy)pyridin-2-yl)oxazole

To a solution of 2-chloro-6-iodo-3-(methoxymethoxy)pyridine (1 g) in DMF (10 mL) were added 2-(tri-n-butylstannyl)oxazole (2.4 g) and tetrakis(triphenylphosphine)palladium (20 mg). The mixture was stirred at 120° C. for 1 h. The solvent was evaporated in vacuo and the remaining crude was purified by CC (Büchi Sepacore, 20 g cartridge, solvent A: Heptane, solvent B: EA, gradient in % B: 1 to 20, flow rate: 20 mL/min) to afford 420 mg of a white solid. LC-MS (A): t_{R}=0.74 min; [M+H]⁺: 240.96.

### 2-((2-Chloro-6-(oxazol-2-yl)pyridin-3-yl)oxy)acetic acid

This acid has been prepared from 2-(6-chloro-5-(methoxymethoxy)pyridin-2-yl)oxazole according to the procedures described for acid 2 (steps 3-5). LC-MS (A): t_{R}=0.59 min; [M+H]⁺: 255.14.

### Acid 40: 2-((2-chloro-6-(cyclopropyl(methyl)carbamoyl)pyridin-3-yl)oxy)acetic acid

### 6-Chloro-N-cyclopropyl-5-(methoxymethoxy)-N-methylpicolinamide

This compound has been prepared from 6-chloro-5-(methoxymethoxy)picolinic acid according to procedures described for acid 15 (steps 1-4) using N-methylcyclopropanamine instead of cyclopropylamine in the amide coupling. LC-MS (A): t_{R}=0.59 min; [M+H]⁺: 285.15.

Following compounds were prepared by modified synthetic routes. The LC-MS conditions used were LC-MS (A).

### Example 1.1.11: 5-(2-(1-(3,4-dimethoxyphenyl)-3,4-dihydroimidazo[1,2-a:5,4-c′]dipyridin-2(1H)-yl)-2-oxoethoxy)-4-ethyl-2-methylbenzamide

### 5-(2-(1-(3,4-dimethoxyphenyl)-3,4-dihydroimidazo[1,2-a:5,4-c′]dipyridin-2(1H)-yl)-2-oxoethoxy)-4-ethyl-2-methylbenzoic acid

This compound has been prepared from example 1.1.10 according to the procedure described for acid 7 (2.step). LC-MS (E): t_{R}=0.63 min; [M+H]⁺: 529.93.

### 5-(2-(1-(3,4-dimethoxyphenyl)-3,4-dihydroimidazo[1,2-a:5,4-c′]dipyridin-2(1H)-yl)-2-oxoethoxy)-4-ethyl-2-methylbenzamide

This compound has been prepared from 5-(2-(1-(3,4-dimethoxyphenyl)-3,4-dihydroimidazo[1,2-a:5,4-c′]dipyridin-2(1H)-yl)-2-oxoethoxy)-4-ethyl-2-methylbenzoic acid and ammonia according to the method C. LC-MS (E): t_{R}=0.57 min; [M+H]⁺: 529.07.

### Example 1.1.12: 5-(2-(1-(3,4-dimethoxyphenyl)-3,4-dihydroimidazo[1,2-a:5,4-c′]dipyridin-2(1H)-yl)-2-oxoethoxy)-4-ethyl-N,2-dimethylbenzamide

This compound has been prepared from 5-(2-(1-(3,4-dimethoxyphenyl)-3,4-dihydroimidazo[1,2-a:5,4-c′]dipyridin-2(1H)-yl)-2-oxoethoxy)-4-ethyl-2-methylbenzoic acid and methylamine according to the method C. LC-MS (E): t_{R}=0.58 min; [M+H]⁺: 543.06.

### Example 1.4.10:1-(1-(4-Chloro-2-fluorophenyl)-3,4-dihydroimidazo[1,2-a:5,4-c′]dipyridin-2(1H)-yl)-2-((2-chloro-6-((2-hydroxyethyl)(methyl)amino)pyridin-3-yl)oxy)ethan-1-one

### 1-(1-(4-Chloro-2-fluorophenyl)-3,4-dihydroimidazo[1,2-a:5,4-c′]dipyridin-2(1H)-yl)-2-((2-chloro-6-iodopyridin-3-yl)oxy)ethan-1-one

This compound has been prepared from 1-(4-chloro-2-fluorophenyl)-1,2,3,4-tetrahydroimidazo[1,2-a:5,4-c′]dipyridine and acid 34 according to the Method C. LC-MS (A): t_{R}=0.75 min; [M+H]⁺: 597.04.

### 1-(1-(4-Chloro-2-fluorophenyl)-3,4-dihydroimidazo[1,2-a:5,4-c′]dipyridin-2(1H)-yl)-2-((2-chloro-6-((2-hydroxyethyl)(methyl)amino)pyridin-3-yl)oxy)ethan-1-one

This compound has been prepared from 1-(1-(4-chloro-2-fluorophenyl)-3,4-dihydroimidazo[1,2-a:5,4-c′]dipyridin-2(1H)-yl)-2-((2-chloro-6-iodopyridin-3-yl)oxy)ethan-1-one and 2-(methylamino)ethan-1-ol according to the procedure described for acid 4 (2.step). LC-MS (A): t_{R}=0.69 min; [M+H]⁺: 544.07.

### Example 1.22.1: 6-chloro-N-cyclopropyl-5-(2-(1-(2-fluoro-4-(2-hydroxyethoxy)phenyl)-3,4-dihydroimidazo[1,2-a:5,4-c′]dipyridin-2(1H)-yl)-2-oxoethoxy)picolinamide

### 1-(4-(2-(Benzyloxy)ethoxy)-2-fluorophenyl)-1,2,3,4-tetrahydroimidazo[1,2-a:5,4-c]dipyridine

This compound has been prepared from aldehyde 16 and 2-(imidazo[1,2-a]pyridin-2-yl)ethan-1-amine according to the Method A. LC-MS (A): t_{R}=0.56 min; [M+H]⁺: 418.15.

### 5-(2-(1-(4-(2-(Benzyloxy)ethoxy)-2-fluorophenyl)-3,4-dihydroimidazo[1,2-a:5,4-c]dipyridin-2(1H)-yl)-2-oxoethoxy)-6-chloro-N-cyclopropylpicolinamide

This compound has been prepared from 1-(4-(2-(benzyloxy)ethoxy)-2-fluorophenyl)-1,2,3,4-tetrahydroimidazo[1,2-a:5,4-c′]dipyridine and acid 24 according to the Method C. LC-MS (A): t_{R}=0.79 min; [M+H]⁺: 670.29.

### 6-chloro-N-cyclopropyl-5-(2-(1-(2-fluoro-4-(2-hydroxyethoxy)phenyl)-3,4-dihydroimidazo[1,2-a:5,4-c′]dipyridin-2(1H)-yl)-2-oxoethoxy)picolinamide

A solution of 5-(2-(1-(4-(2-(Benzyloxy)ethoxy)-2-fluorophenyl)-3,4-dihydroimidazo[1,2-a:5,4-c′]dipyridin-2(1H)-yl)-2-oxoethoxy)-6-chloro-N-cyclopropylpicolinamide (33 mg) and TMSI (17 μl) in DCM (Iml) was stirred at rt for 24 h. MeOH was added and the mixture was evaporated in vacuo. The residue was diluted with EA (50 ml), aq. NaHSO₃ and 1N aq. NaOH. The org. layer was washed twice with sat. aq. NaCl, the combined org. layers were dried over MgSO₄, filtrated off and evaporated in vacuo. The crude was purified by preparative LC-MS [Preparative LC-MS (1)] to afford 5 mg of a colourless solid. LC-MS (A): t_{R}=0.63 min; [M+H]⁺: 579.95.

### Example 1.22.2: N-(6-Chloro-5-(2-(1-(2-fluoro-4-(2-hydroxyethoxy)phenyl)-3,4-dihydroimidazo[1,2-a:5,4-c′]dipyridin-2(1H)-yl)-2-oxoethoxy)pyridin-2-yl)methanesulfonamide

### N-(5-(2-(1-(4-(2-(Benzyloxy)ethoxy)-2-fluorophenyl)-3,4-dihydroimidazo[1,2-a:5,4-c′]dipyridin-2(1H)-yl)-2-oxoethoxy)-6-chloropyridin-2-yl) methanesulfonamide

This compound has been prepared from 1-(4-(2-(benzyloxy)ethoxy)-2-fluorophenyl)-1,2,3,4-tetrahydroimidazo[1,2-a:5,4-c′]dipyridine and acid 5 according to the Method C. LC-MS (A): t_{R}=0.75 min; [M+H]⁺: 680.30.

### N-(6-Chloro-5-(2-(1-(2-fluoro-4-(2-hydroxyethoxy)phenyl)-3,4-dihydroimidazo[1,2-a:5,4-c′]dipyridin-2(1H)-yl)-2-oxoethoxy)pyridin-2-yl)methanesulfonamide

This compound has been prepared from N-(5-(2-(1-(4-(2-(benzyloxy)ethoxy)-2-fluorophenyl)-3,4-dihydroimidazo[1,2-a:5,4-c′]dipyridin-2(1H)-yl)-2-oxoethoxy)-6-chloropyridin-2-yl)methanesulfonamide according to the procedure described for example 1.22.1 (3.step). LC-MS (A): t_{R}=0.58 min; [M+H]⁺: 590.22.

### Example 2.1.3: 1-(7-chloro-1-(3,4-dimethoxyphenyl)-3,4-dihydroimidazo[1,2-a:5,4-c′]dipyridin-2(1H)-yl)-2-(2-chloro-4-(hydroxymethyl)phenoxy)ethan-1-one

### 3-chloro-4-(2-(7-chloro-1-(3,4-dimethoxyphenyl)-3,4-dihydroimidazo[1,2-a:5,4-c′]dipyridin-2(1H)-yl)-2-oxoethoxy)benzaldehyde

This compound has been prepared from 7-chloro-1-(3,4-dimethoxyphenyl)-1,2,3,4-tetrahydroimidazo[1,2-a:5,4-c′]dipyridine and 2-chloro-4-formyl-phenoxy)acetic acid according to the method C. LC-MS (E): t_{R}=0.63 min; [M+H]⁺: 539.83.

### 1-(7-chloro-1-(3,4-dimethoxyphenyl)-3,4-dihydroimidazo[1,2-a:5,4-c′]dipyridin-2(1H)-yl)-2-(2-chloro-4-(hydroxymethyl)phenoxy)ethan-1-one

To a solution of 3-chloro-4-(2-(7-chloro-1-(3,4-dimethoxyphenyl)-3,4-dihydroimidazo[1,2-a:5,4-c′]dipyridin-2(1H)-yl)-2-oxoethoxy)benzaldehyde (80 mg) in MeOH (1 ml) was added at 0° C. sodium borohydride (7 mg). The reaction mixture was stirred at rt for 4 h. Sodium borohydrid (2 mg) was added again and the mixture was stirred at rt for 1 h30 min. The solution was diluted with water and evaporated in vacuo. The remaining aq. solution was extracted with EA and DCM, the combined org. layers were dried over MgSO₄, filtrated off and evaporated in vacuo. The residue was diluted with a little bit of DCM, the product precipitated and was dried to afford 23.8 mg of a white solid. LC-MS (E): t_{R}=0.55 min; [M+H]⁺: 542.09.

### Example 2.6.1:1-(7-chloro-1-(3-isopropyl-1,2,4-oxadiazol-5-yl)-3,4-dihydroimidazo[1,2-a:5,4-c′]dipyridin-2(1H)-yl)-2-((2-chloro-6-morpholinopyridin-3-yl)oxy)ethan-1-one

### Ethyl 7-chloro-1,2,3,4-tetrahydroimidazo[1,2-a:5,4-c′]dipyridine-1-carboxylate

This compound has been prepared from 2-(7-chloroimidazo[1,2-a]pyridin-2-yl)ethan-1-amine and ethyl 2-oxoacetate according to the method A. LC-MS (A): t_{R}=0.46 min; [M+H]⁺: 280.06.

### 2-(Tert-butyl) 1-ethyl 7-chloro-3,4-dihydroimidazo[1,2-a:5,4-c]dipyridine-1,2(1H)-dicarboxylate

To a solution of ethyl 7-chloro-1,2,3,4-tetrahydroimidazo[1,2-a:5,4-c′]dipyridine-1-carboxylate (1.43 g) in DCM (30 ml) were added at 0° C. di-tert-butyldicarbonate (1.6 g) and DIPEA (2.6 ml). The reaction mixture was stirred at rt for 16 h. The mixture was diluted with sat. aq. NH₄Cl. The aq. layer was extracted with DCM and the combined org. layers were washed with sat. aq. NaCl., dried over MgSO₄, filtrated off and evaporated in vacuo. The crude was purified by CC (Büchi Sepacore, 20 g cartridge, solvent A: heptane, solvent B: EA, gradient in % B: 1 to 10, flow rate: 20 ml/min) to afford 941 mg of a yellow oil. LC-MS (A): t_{R}=0.72 min; [M+H]⁺: 380.30.

### 2-(tert-butoxycarbonyl)-7-chloro-1,2,3,4-tetrahydroimidazo[1,2-a:5,4-c′]dipyridine-1-carboxylic acid

This compound has been prepared from 2-(tert-butyl) 1-ethyl 7-chloro-3,4-dihydroimidazo[1,2-a:5,4-c′]dipyridine-1,2(1H)-dicarboxylate according to the procedure described for acid 7. LC-MS (A): t_{R}=0.59 min; [M+H]⁺: 352.31.

### Tert-butyl 7-chloro-1-(3-isopropyl-1,2,4-oxadiazol-5-yl)-3,4-dihydroimidazo[1,2-a:5,4-c′]dipyridine-2(1H)-carboxylate

This compound has been prepared from 2-(tert-butoxycarbonyl)-7-chloro-1,2,3,4-tetrahydroimidazo[1,2-a:5,4-c′]dipyridine-1-carboxylic acid and N-hydroxyisobutanamidine according to method B. LC-MS (A): t_{R}=0.81 min; [M+H]⁺: 418.27.

### 5-(7-Chloro-1,2, 3,4-tetrahydroimidazo[1,2-a:5,4-c′]dipyridin-1-yl)-3-isopropyl-1,2, 4-oxadiazole

This amine has been prepared from tert-butyl 7-chloro-1-(3-isopropyl-1,2,4-oxadiazol-5-yl)-3,4-dihydroimidazo[1,2-a:5,4-c′]dipyridine-2(1H)-carboxylate according to the procedure described for acid 1 (2.step). LC-MS (A): t_{R}=0.56 min; [M+H]⁺: 318.37.

### 1-(7-chloro-1-(3-isopropyl-1,2,4-oxadiazol-5-yl)-3,4-dihydroimidazo[1,2-a:5,4-c′]dipyridin-2(1H)-yl)-2-((2-chloro-6-morpholinopyridin-3-yl)oxy)ethan-1-one

This compound has been prepared from 5-(7-chloro-1,2,3,4-tetrahydroimidazo[1,2-a:5,4-c′]dipyridin-1-yl)-3-isopropyl-1,2,4-oxadiazole according to the method C. LC-MS (A): t_{R}=0.82 min; [M+H]⁺: 572.46.

### Example 2.8.1: 1-(7-chloro-1-(3-hydroxy-4-methoxyphenyl)-3,4-dihydroimidazo[1,2-a:5,4-c′]dipyridin-2(1H)-yl)-2-(naphthalen-2-yloxy)ethan-1-one

This compound has been prepared from example 2.7.1 according to the procedure described for acid 1 (2.step). LC-MS (A): t_{R}=0.66 min; [M+H]⁺: 514.12.

### Example 2.8.2: 1-(7-chloro-1-(3-hydroxy-4-methoxyphenyl)-3,4-dihydroimidazo[1,2-a:5,4-c′]dipyridin-2(1H)-yl)-2-(2-chloro-4-(morpholinomethyl)phenoxy)ethan-1-one

### 3-chloro-4-(2-(7-chloro-1-(4-methoxy-3-((4-methoxybenzyl)oxy)phenyl)-3,4-dihydroimidazo[1,2-a:5,4-c′]dipyridin-2(1H)-yl)-2-oxoethoxy)benzaldehyde

This compound has been prepared from 7-chloro-1-(4-methoxy-3-((4-methoxy-benzyl)oxy)phenyl)-1,2,3,4-tetrahydroimidazo[1,2-a:5,4-c′]dipyridine and 2-(2-chloro-4-formylphenoxy)acetic acid according to the method B. LC-MS (A): t_{R}=0.83 min; [M+H]⁺: 645.83.

### 1-(7-chloro-1-(4-methoxy-3-((4-methoxybenzyl)oxy)phenyl)-3,4-dihydroimidazo[1,2-a:5,4-c′]dipyridin-2(1H)-yl)-2-(2-chloro-4-(morpholinomethyl)phenoxy)ethan-1-one

This compound has been prepared from 3-chloro-4-(2-(7-chloro-1-(4-methoxy-3-((4-methoxybenzyl)oxy)phenyl)-3,4-dihydroimidazo[1,2-a:5,4-c′]dipyridin-2(1H)-yl)-2-oxoethoxy)benzaldehyde according to the procedure described for acid 6. LC-MS (A): t_{R}=0.56 min; [M+H]⁺: 597.02.

### 1-(7-chloro-1-(3-hydroxy-4-methoxyphenyl)-3,4-dihydroimidazo[1,2-a:5,4-c′]dipyridin-2(1H)-yl)-2-(2-chloro-4-(morpholinomethyl)phenoxy)ethan-1-one

This compound has been prepared from 1-(7-chloro-1-(4-methoxy-3-((4-methoxybenzyl)oxy)phenyl)-3,4-dihydroimidazo[1,2-a:5,4-c′]dipyridin-2(1H)-yl)-2-(2-chloro-4-(morpholinomethyl)phenoxy)ethan-1-one according to the procedure described for acid 1 (2.step). LC-MS (A): t_{R}=0.56 min; [M+H]⁺: 597.02.

### Example 2.9.1: 1-(7-chloro-1-(3-((S)-2,3-dihydroxypropoxy)-4-methoxyphenyl)-3,4-dihydroimidazo[1,2-a:5,4-c′]dipyridin-2(1H)-yl)-2-(naphthalen-2-yloxy)ethan-1-one (mixture of 1-(R)- and 1-(S)-epimers)

To a solution of 1-(7-chloro-1-(3-hydroxy-4-methoxyphenyl)-3,4-dihydroimidazo[1,2-a:5,4-c′]dipyridin-2(1H)-yl)-2-(naphthalen-2-yloxy)ethan-1-one (example 2.8.1) (40 mg) in dioxane (0.2 ml) were added (S)-(+)glycidol (0.005 ml) and potassium carbonate (21.5 mg). The reaction mixture was stirred in a sealed tube under argon at 90° C. overnight. The mixture was evaporated in vacuo and the residue was diluted with DCM and water. The aq. layer was extracted with DCM and the combined org. layers were dried over MgSO₄, filtrated off and evaporated in vacuo. The crude was triturated with a mixture of heptane and EA and centrifugated. The resulting solid was diluted with EA (2 ml) and 1N HCl in EA (0.05 ml), the mixture was stirred for 5 min and evaporated in vacuo to afford 20 mg of a white solid (hydrochloric salt). LC-MS (A): t_{R}=0.60 min; [M+H]⁺: 588.14.

### Example 2.10.1: 1-(7-chloro-1-(3-((R)-2,3-dihydroxypropoxy)-4-methoxyphenyl)-3,4-dihydroimidazo[1,2-a:5,4-c′]dipyridin-2(1H)-yl)-2-(naphthalen-2-yloxy)ethan-1-one (mixture of 1-(R)- and 1-(S)-epimers)

This compound has been prepared from example 2.8.1 and (R)-(+)glycidol according to the procedures described for example 2.9.1. LC-MS (A): t_{R}=0.60 min; [M+H]⁺: 588.05.

### Example 2.10.2: 1-(7-chloro-1-(3-((R)-2,3-dihydroxypropoxy)-4-methoxyphenyl)-3,4-dihydroimidazo[1,2-a:5,4-c′]dipyridin-2(1H)-yl)-2-(2-chloro-4-(morpholinomethyl)phenoxy)ethan-1-one (mixture of 1-(R)- and 1-(S)-epimers)

This compound has been prepared from example 2.8.2 and (R)-(+)glycidol according to the procedures described for example 2.9.1. LC-MS (A): t_{R}=0.54 min; [M+H]⁺: 356.98.

### Example 2.11.1: 1-(7-chloro-1-(4-methoxy-3-(3-methoxypropoxy)phenyl)-3,4-dihydroimidazo[1,2-a:5,4-c′]dipyridin-2(1H)-yi)-2-(naphthalen-2-yloxy)ethan-1-one

To a solution of 1-(7-chloro-1-(3-hydroxy-4-methoxyphenyl)-3,4-dihydroimidazo[1,2-a:5,4-c′]dipyridin-2(1H)-yl)-2-(naphthalen-2-yloxy)ethan-1-one (example 2.8.1) (15 mg) in NMP (0.3 ml) was added NaH (1.2 mg). The mixture was stirred at rt for 20 min. 1-Bromo-3-methoxypropane (4.5 mg) was added and the reaction mixture was stirred at 90° C. for 1 h30 min and then at rt overnight. The mixture was evaporated in vacuo and the residue was diluted with EA and sat. aq. NaHCO₃. The aq. layer was extracted with EA, the combined org. layers were dried over MgSO₄, filtrated off and evaporated in vacuo. The resulting solid was diluted with EA and 1N HCl in EA (0.026 ml), the suspension was cooled down to 0° C., stirred for 30 min, centrifugated and filtrated off. The solid was then suspended in a solution of ether and hexane (1:1), sonicated and filtrated off. 2 mg of a brown solid were recovered. LC-MS (A): t_{R}=0.73 min; [M+H]⁺: 585.85.

### Example 2.12.1: 1-(7-chloro-1-(3-(3-hydroxypropoxy)-4-methoxyphenyl)-3,4-dihydroimidazo[1,2-a:5,4-c′]dipyridin-2(1H)-yl)-2-(naphthalen-2-yloxy)ethan-1-one

This compound has been prepared from example 2.8.1 and 3-bromo-1-propanol according to the procedure described for example 2.11.1. LC-MS (A): t_{R}=0.64 min; [M+H]⁺: 572.14.

### Example 2.13.1: 1-(7-chloro-1-(3-(2-hydroxyethoxy)-4-methoxyphenyl)-3,4-dihydroimidazo[1,2-a:5,4-c′]dipyridin-2(1H)-yl)-2-(naphthalen-2-yloxy)ethan-1-one

This compound has been prepared from example 2.8.1 and 2-bromoethanol according to the procedure described for example 2.11.1. LC-MS (E):): t_{R}=0.62 min; [M+H]⁺: 558.14.

### Example 2.13.2:1-(7-chloro-1-(3-(2-hydroxyethoxy)-4-methoxyphenyl)-3,4-dihydroimidazo[1,2-a:5,4-c′]dipyridin-2(1H)-yl)-2-(2-chloro-4-(morpholinomethyl)phenoxy)ethan-1-one

### 1-(1-(3-(2-((tert-butyldimethylsilyl)oxy)ethoxy)-4-methoxyphenyl)-7-chloro-3,4-dihydroimidazo[1,2-a:5,4-c′]dipyridin-2(1H)-yl)-2-(2-chloro-4-(morpholinomethyl)phenoxy)ethan-1-one

This compound has been prepared from example 2.8.2 and (2-bromoethoxy)(tert-butyl)dimethylsilane according to the procedure described for example 2.11.1 using DMF as a solvent instead of NMP. LC-MS (A): t_{R}=0.78 min; [M+H]⁺: 755.30.

### 1-(7-chloro-1-(3-(2-hydroxyethoxy)-4-methoxypheny)-3,4-dihydroimidazo[1,2-a:5,4-c′]dipyridin-2(1H)-yl)-2-(2-chloro-4-(morpholinomethyl)phenoxy)ethan-1-one

To a solution of 1-(1-(3-(2-((tert-butyldimethylsilyl)oxy)ethoxy)-4-methoxyphenyl)-7-chloro-3,4-dihydroimidazo[1,2-a:5,4-c′]dipyridin-2(1H)-yl)-2-(2-chloro-4-(morpholinomethyl)phenol-xy)ethan-1-one (0.063 mg) in THF (3 ml) was added TBAF on silica gel at 0° C. and the mixture was stirred for 24 h. The mixture was filtrated off and the crude was purified by CC (Büchi Sepacore, 2 g cartridge, solvent A: DCM, solvent B: 7N NH₃ inMeOH, gradient in % B: 1 to 5, flow rate: 7 ml/min) to afford 38 mg of a colourless foam. LC-MS (A): t_{R}=0.56 min; [M+H]⁺: 641.15.

### Example 2.14.1: Methyl 2-(5-(7-chloro-2-(2-(2-chloro-4-(morpholinomethyl) phenoxy)acetyl)-1,2,3,4-tetrahydroimidazo[1,2-a:5,4-c′]dipyridin-1-yl)-2-methoxyphenoxy)acetate

This compound has been prepared example 2.8.2 according to the procedure described for acid 7. LC-MS (A): t_{R}=0.60 min; [M+H]⁺: 669.11.

### Example 3.1.2: 2-(2,4-Dichlorophenoxy)-1-(1-(3,4-dimethoxyphenyl)-7-(trifluoromethyl)-3,4-dihydroimidazo[1,2-a:5,4-c′]dipyridin-2(1H)-yl)ethan-1-one

### 2-(1-(3,4-Dimethoxyphenyl)-7-(trifluoromethyl)-3,4-dihydroimidazo[1,2-a:5,4-c′]dipyridin-2(1H)-yl)-2-oxoethyl acetate

To a solution of acetoxyacetic acid (42 mg) in DCM (1.6 ml) was added DMAP (11 mg), HOBT (58 mg), EDC.HCl (171 mg) and DIPEA (0.18 ml). The reaction mixture was stirred at rt for 30 min. The 1-(3,4-dimethoxyphenyl)-7-(trifluoromethyl)-1,2,3,4-tetrahydroimidazo[1,2-a:5,4-c′]dipyridine (160 mg) was added and the mixture was stirred at rt overnight. The mixture was diluted with DCM and extracted with aq. 1N HCl. The layers were separated, the org. layer was washed with sat. aq. NaHCO₃ and sat. aq. NaCl, dried over MgSO₄, filtrated off and evaporated in vacuo. The crude was purified by CC (Büchi Sepacore, 5 g cartridge, solvent A: DCM, solvent B: MeOH, gradient in % B: 1 to 3, flow rate: 8 ml/min) to afford 143 mg of a beige foam. LC-MS (A): t_{R}=0.72 min; [M+H]⁺: 477.97.

### 1-(1-(3,4-dimethoxyphenyl)-7-(trifluoromethyl)-3,4-dihydroimidazo[1,2-a:5,4-c]dipyridin-2(1H)-yl)-2-hydroxyethan-1-one

A solution 2-(1-(3,4-dimethoxyphenyl)-7-(trifluoromethyl)-3,4-dihydroimidazo[1,2-a:5,4-c′]dipyridin-2(1H)-yl)-2-oxoethyl acetate (140 mg) and K₂CO₃ (82 mg) in MeOH (3 ml) was stirred at 0° C. for 4 h. The mixture was diluted with DCM and extracted with aq. 1N HCl. The layers were separated, the org. layer was washed with sat. aq. NaHCO₃ and sat. aq. NaCl, dried over MgSO₄, filtrated off and evaporated in vacuo. The crude was purified by CC (Büchi Sepacore, 2 g cartridge, solvent A: DCM, solvent B: MeOH, gradient in % B: 1 to 2, flow rate: 5 ml/min) to afford 120 mg of a beige foam. LC-MS (A): t_{R}=0.66 min; [M+H]⁺: 435.99.

### 2-(2,4-dichlorophenoxy)-1-(1-(3,4-dimethoxyphenyl)-7-(trifluoromethyl)-3,4-dihydroimidazo[1,2-a:5,4-c′]dipyridin-2(1H)-yl)ethan-1-one

A solution of 1-(1-(3,4-dimethoxyphenyl)-7-(trifluoromethyl)-3,4-dihydroimidazo[1,2-a:5,4-c′]dipyridin-2(1H)-yl)-2-hydroxyethan-1-one (120 mg), 2,4-dichlorophenol (0.066 ml), DIAD (0.064 ml) and Ph₃P (94 mg) in THF (3 ml) was stirred at rt overnight. The mixture was diluted with H₂O and extracted with EA. The layers were separated, the org. layer was washed with sat. aq. NaCl, dried over MgSO₄, filtrated off and evaporated in vacuo. The crude was purified by CC (Büchi Sepacore, 5 g cartridge, solvent A: heptane, solvent B: EA, gradient in % B: 10 to 40, flow rate: 10 ml/min) to afford 122 mg of a white solid. LC-MS (A): t_{R}=0.91 min; [M+H]⁺: 580.06.

### Example 3.1.26: 2-(2-chloro-4-(morpholinomethyl)phenoxy)-1-(1-(3,4-dimethoxyphe-nyl)-7-(trifluoromethyl)-3,4-dihydroimidazo[1,2-a:5,4-c′]dipyridin-2(1H)-yl)ethan-1-one

### 3-chloro-4-(2-(1-(3,4-dimethoxyphenyl)-7-(trifluoromethyl)-3,4-dihydroimidazo[1,2-a:5,4-c′]dipyridin-2(1H)-yl)-2-oxoethoxy)benzaldehyde

This compound has been prepared from 1-(3,4-dimethoxyphenyl)-7-(trifluoromethyl)-1,2,3,4-tetrahydroimidazo[1,2-a:5,4-c′]dipyridine and 2-(2-chloro-4-formylphenoxy)acetic acid according to the method B. LC-MS (A): t_{R}=0.72 min; [M+H]⁺: 573.97.

### 2-(2-chloro-4-(morpholinomethyl)phenoxy)-1-(1-(3,4-dimethoxyphenyl)-7-(trifluoromethyl)-3,4-dihydroimidazo[1,2-a:5,4-c]dipyridin-2(1H)-yl)ethan-1-one

This compound has been prepared from 3-chloro-4-(2-(1-(3,4-dimethoxyphenyl)-7-(trifluoromethyl)-3,4-dihydroimidazo[1,2-a:5,4-c′]dipyridin-2(1H)-yl)-2-oxoethoxy)benzaldehyde according to the procedure described for acid 6 (2. Step). LC-MS (A): t_{R}=0.58 min; [M+H]⁺: 645.16.

### Example 3.3.5: 1-(1-(2-fluoro-4-methoxyphenyl)-7-(trifluoromethyl)-3,4-dihydroimidazo[1,2-a:5,4-c′]dipyridin-2(1H)-yl)-2-((5-methyl-1,2,3,4-tetrahydroisoquinolin-6-yl)oxy)ethan-1-one

This compound has been prepared example 3.3.4 according to the procedure described for acid 1 (2. Step). LC-MS (A): t_{R}=0.70 min; [M+H]⁺: 569.09.

### Example 3.3.6: 2-((2-acetyl-5-methyl-1,2,3,4-tetrahydroisoquinolin-6-yl)oxy)-1-(1-(2-fluoro-4-methoxyphenyl)-7-(trifluoromethyl)-3,4-dihydroimidazo[1,2-a:5,4-c′]dipyridin-2(1H)-yl)ethan-1-one

To a solution of 1-(1-(2-fluoro-4-methoxyphenyl)-7-(trifluoromethyl)-3,4-dihydroimidazo[1,2-a:5,4-c′]dipyridin-2(1H)-yl)-2-((5-methyl-1,2,3,4-tetrahydroisoquinolin-6-yl)oxy)ethan-1-one (example 3.3.5) (19 mg) in DCM (2 ml) cooled down to 0° C. were added acetic acid anhydride (0.005 ml) and DIPEA (0.009 ml). The reaction mixture was stirred at rt for 15 min. The solution was diluted with DCM and sat. aq. NaHCO₃. The layers were separated using Phase Separator and the org. layer was evaporated in vacuo. The crude compound was purified by preparative LC-MS (I) to afford 9.6 mg of a white foam. LC-MS (A): t_{R}=0.87 min; [M+H]⁺: 611.22.

### Example 3.3.7: 2-((2,5-dimethyl-1,2,3,4-tetrahydroisoquinolin-6-yl)oxy)-1-(1-(2-fluoro-4-methoxyphenyl)-7-(trifluoromethyl)-3,4-dihydroimidazo[1,2-a:5,4-c′]dipyridin-2(1H)-yl)ethan-1-one

To a solution 1-(1-(2-fluoro-4-methoxyphenyl)-7-(trifluoromethyl)-3,4-dihydroimidazo[1,2-a:5,4-c′]dipyridin-2(1H)-yl)-2-((5-methyl-1,2,3,4-tetrahydroisoquinolin-6-yl)oxy)ethan-1-one (example 3.3.5) (19 mg) in DCM (2 ml) were added formaldehyde 36.5% in water (0.001 ml), acetic acid (0.003 ml) and sodium triacetoxyborhydride (12 mg). The reaction mixture was stirred at rt for 3 h. The mixture was diluted with sat. aq. NaHCO₃ and evaporated in vacuo. The crude compound was purified by preparative LC-MS (I) to afford 10 mg of a white solid. LC-MS (A): t_{R}=0.71 min; [M+H]⁺: 583.16.

### Example 3.18.4: 1-(1-(2-fluoro-4,5-dimethoxyphenyl)-7-(trifluoromethyl)-3,4-dihydroimidazo[1,2-a:5,4-c′]dipyridin-2(1H)-yl)-2-((1,2,3,4-tetrahydroisoquinolin-7-yl)oxy)ethan-1-one

This compound has been prepared example 3.18.3 according to the procedure described for acid 1 (2. Step). LC-MS (A): t_{R}=0.66 min; [M+H]⁺: 585.13.

### Example 3.18.5: 1-(1-(2-fluoro-4,5-dimethoxyphenyl)-7-(trifluoromethyl)-3,4-dihydroimidazo[1,2-a:5,4-c′]dipyridin-2(1H)-yl)-2-((2-methyl-1,2,3,4-tetrahydroisoquinolin-7-yl)oxy)ethan-1-one

This compound has been prepared from 1-(1-(2-fluoro-4,5-dimethoxyphenyl)-7-(trifluoromethyl)-3,4-dihydroimidazo[1,2-a:5,4-c′]dipyridin-2(1H)-yl)-2-((1,2,3,4-tetrahydroisoquinolin-7-yl)oxy)ethan-1-one (example 3.18.4) according to the procedure described or example 3.3.7. LC-MS (A): t_{R}=0.66 min; [M+H]⁺: 599.16.

### Example 3.18.6: 2-((2-acetyl-1,2,3,4-tetrahydroisoquinolin-7-yl)oxy)-1-(1-(2-fluoro-4,5-dimethoxyphenyl)-7-(trifluoromethyl)-3,4-dihydroimidazo[1,2-a:5,4-c′]dipyridin-2(1H)-yl)ethan-1-one

This compound has been prepared from 1-(1-(2-fluoro-4,5-dimethoxyphenyl)-7-(trifluoromethyl)-3,4-dihydroimidazo[1,2-a:5,4-c′]dipyridin-2(1H)-yl)-2-((1,2,3,4-tetrahydroisoquinolin-7-yl)oxy)ethan-1-one (example 3.18.4) according to the procedure described for example 3 3.6. LC-MS (A): t_{R}=0.80 min; [M+H]⁺: 627.19.

### Example 3.18.7: 1-(1-(2-fluoro-4,5-dimethoxyphenyl)-7-(trifluoromethyl)-3,4-dihydroimidazo[1,2-a:5,4-c′]dipyridin-2(1H)-yl)-2-((2-(2-hydroxyacetyl)-1,2,3,4-tetrahydroisoquinolin-7-yl)oxy)ethan-1-one

### 2-(7-(2-(1-(2-fluoro-4,5-dimethoxyphenyl)-7-(trifluoromethyl)-3,4-dihydroimidazo[1,2-a:5,4-c′]dipyridin-2(1H)-yl)-2-oxoethoxy)-3,4-dihydroisoquinolin-2(1H)-yl)-2-oxoethyl acetate

This compound has been prepared from example 3.18.4 and acetoxyacetic acid according to the method B. LC-MS (A): t_{R}=0.80 min; [M+H]⁺: 685.14.

### 1-(1-(2-fluoro-4,5-dimethoxyphenyl)-7-(trifluoromethyl)-3,4-dihydroimidazo[1,2-a:5,4-c′]dipyridin-2(1H)-yl)-2-((2-(2-hydroxyacetyl)-1,2,3,4-tetrahydroisoquinolin-7-yl)oxy)ethan-1-one

To a solution of 2-(7-(2-(1-(2-fluoro-4,5-dimethoxyphenyl)-7-(trifluoromethyl)-3,4-dihydroimidazo[1,2-a:5,4-c′]dipyridin-2(1H)-yl)-2-oxoethoxy)-3,4-dihydroisoquinolin-2(1H)-yl)-2-oxoethyl acetate (39 mg) in MeOH (2 ml) was added potassium carbonate (8 mg). The reaction mixture was stirred at rt for 1 h15 min. The suspension was filtrated off and the filtrate was evaporated in vacuo. The crude compound was purified by preparative LC-MS (I) to afford 19 mg of a white foam. LC-MS (A): t_{R}=0.75 min; [M+H]⁺: 642.99.

### Example 3.18.9: 2-(4-(3-aminooxetan-3-yl)-2-chlorophenoxy)-1-(1-(2-fluoro-4,5-dimethoxyphenyl)-7-(trifluoromethyl)-3,4-dihydroimidazo[1,2-a:5,4-c′]dipyridin-2(1H)-yl)ethan-1-one

To a solution of N-(3-(3-chloro-4-(2-(1-(2-fluoro-4,5-dimethoxyphenyl)-7-(trifluoromethyl)-3,4-dihydroimidazo[1,2-a:5,4-c′]dipyridin-2(1H)-yl)-2-oxoethoxy)phenyl)oxetan-3-yl)-2-methylpropane-2-sulfinamide (3.18.8) (in MeOH (63 mg) in MeOH (Iml) was adde at 0° C. HCl (4N in THF) (0.032 ml) and the mixture was stirred for 24 h. The crude was triturated with Et₂O and dried to afford 61 mg of beige solid. LC-MS (A): t_{R}=0.67 min; [M+H]⁺: 635.14.

### Example 3.18.10: 2-(2-chloro-4-(3-morpholinooxetan-3-yl)phenoxy)-1-(1-(2-fluoro-4,5-dimethoxyphenyl)-7-(trifluoromethyl)-3,4-dihydroimidazo[1,2-a:5,4-c′]dipyridin-2(1H)-yl)ethan-1-one

To a solution of 2-(4-(3-aminooxetan-3-yl)-2-chlorophenoxy)-1-(1-(2-fluoro-4,5-dimethoxyphenyl)-7-(trifluoromethyl)-3,4-dihydroimidazo[1,2-a:5,4-c′]dipyridin-2(1H)-yl)ethan-1-one (example 3.18.9) (60 mg) in DMF (Iml) were added DIPEA (0.046 ml) and 2-bromoethyl ether (0.014 ml). The reaction mixture was stirred at 100° C. for 40 h. The solution was diluted with EA and water. The layers were separated and the aq. layer was extracted twice with EA. The combined org. layer were dried over MgSO₄, filtrated off and evaporated in vacuo. The crude compound was purified by preparative LC-MS (I) to afford 13 mg of a white solid. LC-MS (A): t_{R}=0.73 min; [M+H]⁺: 705.18.

### Example 16.3.1: 5-(8-(2-((2-Chloro-6-morpholinopyridin-3-yl)oxy)acetyl)-6,7,8,9-tetrahydropyrido[4′,3′:4,5]imidazo[1,2-b]pyridazin-9-yl)-N,N-dimethylthiophene-3-carboxamide

### Methyl 5-(6,7,8,9-tetrahydropyrido[4′,3′:4,5]imidazo[1,2-b]pyridazin-9-yl)thiophene-3-carboxylate

This compound has been prepared from 2-(imidazo[1,2-b]pyridazin-2-yl)ethan-1-amine and aldehyde 16 according to the method A. LC-MS (A): t_{R}=0.49 min; [M+H]⁺: 315.37.

### Tert-butyl 9-(4-(methoxycarbonyl)thiophen-2-yl)-6,9-dihydropyrido[4′,3′:4,5]imidazo[1,2-b]pyridazine-8(7H)-carboxylate

This compound has been prepared from methyl 5-(6,7,8,9-tetrahydropyrido[4′,3′:4,5]imidazo[1,2-b]pyridazin-9-yl)thiophene-3-carboxylate according to the procedure described for example 2.6.1 (2.step). LC-MS (A): t_{R}=0.83 min; [M+H]⁺: 415.41.

### 5-(8-(Tert-butoxycarbonyl)-6,7,8,9-tetrahydropyrido[4′,3′:4,5]imidazo[1,2-b]pyridazin-9-yl)thiophene-3-carboxylic acid

This compound has been prepared from methyl 5-(6,7,8,9-tetrahydropyrido[4′,3′:4,5]imidazo[1,2-b]pyridazin-9-yl)thiophene-3-carboxylate according to the procedure described for example 2.6.1 (3.step). LC-MS (A): t_{R}=0.71 min; [M+H]⁺: 401.16.

### Tert-butyl 9-(4-(dimethylcarbamoyl)thiophen-2-yl)-6,9-dihydropyrido[4′,3′:4,5]imidazo[1,2-b]pyridazine-8(7H)-carboxylate

This compound has been prepared from 5-(8-(tert-butoxycarbonyl)-6,7,8,9-tetrahydropyrido[4′,3′:4,5]imidazo[1,2-b]pyridazin-9-yl)thiophene-3-carboxylic acid and dimethylamine according to the method C. LC-MS (A): t_{R}=0.73 min; [M+H]⁺: 427.93.

### N,N-Dimethyl-5-(6,7,8,9-tetrahydropyrido[4′,3′:4,5]imidazo[1,2-b]pyridazin-9-yl)thiophene-3-carboxamide

This amine has been prepared tert-butyl 9-(4-(dimethylcarbamoyl)thiophen-2-yl)-6,9-dihydropyrido[4′,3′:4,5]imidazo[1,2-b]pyridazine-8(7H)-carboxylate according to the procedure described for acid 1 (2.step). LC-MS (A): t_{R}=0.45 min; [M+H]⁺: 327.98.

### 5-(8-(2-((2-Chloro-6-morpholinopyridin-3-yl)oxy)acetyl)-6,7,8,9-tetrahydropyrido[4′,3′:4,5]imidazo[1,2-b]pyridazin-9-yl)-N,N-dimethylthiophene-3-carboxamide

This compound has been prepared from N,N-dimethyl-5-(6,7,8,9-tetrahydropyrido[4′,3′:4,5]imidazo[1,2-b]pyridazin-9-yl)thiophene-3-carboxamide and acid 4 according to the method C. LC-MS (A): t_{R}=0.73 min; [M+H]⁺: 581.99.

TABLE 2

| Example N^{o} | IC₅₀ [nM] |
|---|---|
| 1.1.11 | 248 |
| 1.1.12 | 141 |
| 1.4.10 | 87 |
| 1.22.1 | 80 |
| 1.22.2 | 38 |
| 2.1.3 | 402 |
| 2.6.1 | 225 |
| 2.8.1 | 219 |
| 2.8.2 | 154 |
| 2.9.1 | 30 |
| 2.10.1 | 34 |
| 2.10.2 | 19 |
| 2.11.1 | 32 |
| 2.12.1 | 22 |
| 2.13.1 | 34 |
| 2.13.2 | 23 |
| 2.14.1 | 88 |
| 3.1.2 | 30 |
| 3.1.26 | 17 |
| 3.3.5 | 217 |
| 3.3.6 | 11 |
| 3.3.7 | 117 |
| 3.18.4 | 72 |
| 3.18.5 | 13 |
| 3.18.6 | 6 |
| 3.18.7 | 4 |
| 3.18.9 | 10 |
| 3.18.10 | 25 |
| 16.3.1 | 43 |

### Special Substitution

### Example 3.1.3: 2-(2-chloro-4-(trifluoromethyl)phenoxy)-1-(1-(3,4-dimethoxyphenyl)-7-(trifluoromethyl)-3,4-dihydroimidazo[1,2-a:5,4-c′]dipyridin-2(1H)-yl)ethan-1-one

### 2-bromo-1-(1-(3,4-dimethoxyphenyl)-7-(trifluoromethyl)-3,4-dihydroimidazo[1,2-a:5,4-c′]dipyridin-2(1H)-yl)ethan-1-one

To a solution of 1-(3,4-dimethoxyphenyl)-7-(trifluoromethyl)-1,2,3,4-tetrahydroimidazo[1,2-a:5,4-c′]dipyridine (1.65 g) in DCM (18 ml) were added at 0° C. DIPEA (1.5 ml) and bromoacetylbromid (0.42 ml) in DCM (2 ml). The reaction mixture was stirred at rt overnight. The solution was diluted with DCM and sat. aq. NH₄Cl. The layers were separated, the aq. layer was extracted twice with DCM and the combined org. layers were washed with sat. aq. NaCl, dried over MgSO₄, filtrated off and evaporated in vacuo. The crude was purified by CC (Büchi Sepacore, 50 g cartridge, solvent A: heptane, solvent B: EA, gradient in % B: 10 to 50, flow rate: 30 ml/min) to afford 1.06 g of a yellow solid. LC-MS (A): t_{R}=0.76 min; [M+H]⁺: 499.94.

### 2-(2-chloro-4-(trifluoromethyl)phenoxy)-1-(1-(3,4-dimethoxyphenyl)-7-(trifluoromethyl)-3,4-dihydroimidazo[1,2-a:5,4-c′]dipyridin-2(1H)-yl)ethan-1-one

To a solution of 2-bromo-1-(1-(3,4-dimethoxyphenyl)-7-(trifluoromethyl)-3,4-dihydroimidazo[1,2-a:5,4-c′]dipyridin-2(1H)-yl)ethan-1-one (49.8 mg) in DMF (1 ml) were added 2-chloro-4-(trifluoromethyl)phenol (19.7 mg) and potassium carbonate (69 mg). The reaction mixture was stirred at 55° C. overnight. The suspension was filtrated off, the solid part was washed with DCM/MeOH 1:1 and the solvents were evaporated in genevac. The crude compound was purified by preparative LC-MS (I) to afford 37 mg of the expected product. LC-MS (D): t_{R}=1.35 min; [M+H]⁺: 613.80.

Following examples were synthesized starting from the appropriate phenol derivative and 2-bromo-1-(1-(3,4-dimethoxyphenyl)-7-(trifluoromethyl)-3,4-dihydroimidazo[1,2-a:5,4-c′]dipyridin-2(1H)-yl)ethan-1-one following the method described for example 3.1.3 (2. Step). LC-MS data are listed in table 3 below. The LC-MS conditions used were LC-MS (A).

TABLE 3

| | | | | IC₅₀ |
|---|---|---|---|---|
| Example | Name | t_{R} | [M + H]⁺ | [nM] |
| 3.1.3 | 2-(2-chloro-4-(trifluoromethyl)phenoxy)-1-(1-(3,4-dimethoxyphenyl)- | 1.35 | 613.80 | 36 |
| | 7-(trifluoromethyl)-3,4-dihydroimidazo[1,2-a:5,4-c′]dipyridin-2(1H)- | | | |
| | yl)ethan-1-one | | | |
| 3.1.4 | 2-(2-chloro-5-(trifluoromethyl)phenoxy)-1-(1-(3,4-dimethoxyphenyl)- | 1.33 | 613.84 | 202 |
| | 7-(trifluoromethyl)-3,4-dihydroimidazo[1,2-a:5,4-c′]dipyridin-2(1H)- | | | |
| | yl)ethan-1-one | | | |
| 3.1.5 | 1-(1-(3,4-dimethoxyphenyl)-7-(trifluoromethyl)-3,4- | 1.34 | 526.32 | 153 |
| | dihydroimidazo[1,2-a:5,4-c′]dipyridin-2(1H)-yl)-2-(o-tolyloxy)ethan-1- | | | |
| | one | | | |
| 3.1.6 | 1-(1-(3,4-dimethoxyphenyl)-7-(trifluoromethyl)-3,4- | 1.33 | 526.30 | 71 |
| | dihydroimidazo[1,2-a:5,4-c′]dipyridin-2(1H)-yl)-2-(p-tolyloxy)ethan-1- | | | |
| | one | | | |
| 3.1.7 | 1-(1-(3,4-dimethoxyphenyl)-7-(trifluoromethyl)-3,4- | 1.39 | 580.25 | 29 |
| | dihydroimidazo[1,2-a:5,4-c′]dipyridin-2(1H)-yl)-2-(2- | | | |
| | (trifluoromethyl)phenoxy)ethan-1-one | | | |
| 3.1.8 | 1-(1-(3,4-dimethoxyphenyl)-7-(trifluoromethyl)-3,4- | 1.40 | 580.28 | 132 |
| | dihydroimidazo[1,2-a:5,4-c′]dipyridin-2(1H)-yl)-2-(4- | | | |
| | (trifluoromethyl)phenoxy)ethan-1-one | | | |
| 3.1.9 | 1-(1-(3,4-dimethoxyphenyl)-7-(trifluoromethyl)-3,4- | 1.40 | 580.27 | 422 |
| | dihydroimidazo[1,2-a:5,4-c′]dipyridin-2(1H)-yl)-2-(3- | | | |
| | (trifluoromethyl)phenoxy)ethan-1-one | | | |
| 3.1.10 | 1-(1-(3,4-dimethoxyphenyl)-7-(trifluoromethyl)-3,4- | 1.27 | 530.30 | 251 |
| | dihydroimidazo[1,2-a:5,4-c′]dipyridin-2(1H)-yl)-2-(3- | | | |
| | (trifluoromethyl)phenoxy)ethan-1-one | | | |
| 3.1.11 | 1-(1-(3,4-dimethoxyphenyl)-7-(trifluoromethyl)-3,4- | 1.43 | 596.35 | 199 |
| | dihydroimidazo[1,2-a:5,4-c′]dipyridin-2(1H)-yl)-2-(4- | | | |
| | (trifluoromethoxy)phenoxy)ethan-1-one | | | |
| 3.1.12 | 2-(2-chloro-5-methylphenoxy)-1-(1-(3,4-dimethoxyphenyl)-7- | 1.40 | 560.28 | 64 |
| | (trifluoromethyl)-3,4-dihydroimidazo[1,2-a:5,4-c′]dipyridin-2(1H)- | | | |
| | yl)ethan-1-one | | | |
| 3.1.13 | 2-(2-chloro-3-(trifluoromethyl)phenoxy)-1-(1-(3,4-dimethoxyphenyl)- | 1.45 | 614.04 | 19 |
| | 7-(trifluoromethyl)-3,4-dihydroimidazo[1,2-a:5,4-c′]dipyridin-2(1H)- | | | |
| | yl)ethan-1-one | | | |
| 3.1.14 | 2-(2-chloro-5-fluorophenoxy)-1-(1-(3,4-dimethoxyphenyl)-7- | 1.36 | 564.00 | 103 |
| | (trifluoromethyl)-3,4-dihydroimidazo[1,2-a:5,4-c′]dipyridin-2(1H)- | | | |
| | yl)ethan-1-one | | | |
| 3.1.15 | 2-((2-chloropyridin-3-yl)oxy)-1-(1-(3,4-dimethoxyphenyl)-7- | 1.14 | 547.20 | 67 |
| | (trifluoromethyl)-3,4-dihydroimidazo[1,2-a:5,4-c′]dipyridin-2(1H)- | | | |
| | yl)ethan-1-one | | | |
| 3.1.16 | 1-(1-(3,4-dimethoxyphenyl)-7-(trifluoromethyl)-3,4- | 1.06 | 527.28 | 266 |
| | dihydroimidazo[1,2-a:5,4-c′]dipyridin-2(1H)-yl)-2-((2-methylpyridin-3- | | | |
| | yl)oxy)ethan-1-one | | | |
| 3.1.17 | 2-((2-bromopyridin-3-yl)oxy)-1-(1-(3,4-dimethoxyphenyl)-7- | 1.15 | 591.23 | 20 |
| | (trifluoromethyl)-3,4-dihydroimidazo[1,2-a:5,4-c′]dipyridin-2(1H)- | | | |
| | yl)ethan-1-one | | | |
| 3.1.18 | 2-((4-chloropyridin-3-yl)oxy)-1-(1-(3,4-dimethoxyphenyl)-7- | 1.13 | 547.23 | 235 |
| | (trifluoromethyl)-3,4-dihydroimidazo[1,2-a:5,4-c′]dipyridin-2(1H)- | | | |
| | yl)ethan-1-one | | | |
| 3.1.19 | 1-(1-(3,4-dimethoxyphenyl)-7-(trifluoromethyl)-3,4- | 1.12 | 563.32 | 117 |
| | dihydroimidazo[1,2-a:5,4-c′]dipyridin-2(1H)-yl)-2-(quinolin-6- | | | |
| | yloxy)ethan-1-one | | | |
| 3.1.20 | 1-(1-(3,4-dimethoxyphenyl)-7-(trifluoromethyl)-3,4- | 1.12 | 563.31 | 19 |
| | dihydroimidazo[1,2-a:5,4-c′]dipyridin-2(1H)-yl)-2-(isoquinolin-7- | | | |
| | yloxy)ethan-1-one | | | |
| 3.1.21 | 2-(benzo[d][1,3]dioxol-5-yloxy)-1-(1-(3,4-dimethoxyphenyl)-7- | 1.22 | 556.29 | 116 |
| | (trifluoromethyl)-3,4-dihydroimidazo[1,2-a:5,4-c′]dipyridin-2(1H)- | | | |
| | yl)ethan-1-one | | | |
| 3.1.22 | 1-(1-(3,4-dimethoxyphenyl)-7-(trifluoromethyl)-3,4- | 1.18 | 563.31 | 165 |
| | dihydroimidazo[1,2-a:5,4-c′]dipyridin-2(1H)-yl)-2-(quinolin-3- | | | |
| | yloxy)ethan-1-one | | | |
| 3.1.23 | 1-(1-(3,4-dimethoxyphenyl)-7-(trifluoromethyl)-3,4- | 0.96 | 567.31 | 382 |
| | dihydroimidazo[1,2-a:5,4-c′]dipyridin-2(1H)-yl)-2-((3-methyl-3H- | | | |
| | imidazo[4,5-b]pyridin-6-yl)oxy)ethan-1-one | | | |
| 3.1.24 | 1-(1-(3,4-dimethoxyphenyl)-7-(trifluoromethyl)-3,4- | 1.25 | 592.38 | 157 |
| | dihydroimidazo[1,2-a:5,4-c′]dipyridin-2(1H)-yl)-2-((1-methyl-3-phenyl- | | | |
| | 1H-pyrazol-5-yl)oxy)ethan-1-one | | | |
| 3.1.25 | 2-((4-chloro-1-methyl-5-(trifluoromethyl)-1H-pyrazol-3-yl)oxy)-1-(1- | 1.40 | 618.33 | 78 |
| | (3,4-dimethoxyphenyl)-7-(trifluoromethyl)-3,4-dihydroimidazo[1,2- | | | |
| | a:5,4-c′]dipyridin-2(1H)-yl)ethan-1-one | | | |
| 3.1.27 | 2-((2-chloro-6-(morpholinomethyl)pyridin-3-yl)oxy)-1-(1-(3,4- | 1.00 | 646.22 | 84 |
| | dimethoxyphenyl)-7-(trifluoromethyl)-3,4-dihydroimidazo[1,2-a:5,4- | | | |
| | c′]dipyridin-2(1H)-yl)ethan-1-one | | | |
| 3.1.28 | 1-(1-(3,4-dimethoxyphenyl)-7-(trifluoromethyl)-3,4- | 0.90 | 540.06 | 118 |
| | dihydroimidazo[1,2-a:5,4-c′]dipyridin-2(1H)-yl)-2-(2- | | | |
| | ethylphenoxy)ethan-1-one | | | |
| 3.1.29 | 2-(4-chloro-2-ethylphenoxy)-1-(1-(3,4-dimethoxyphenyl)-7- | 0.94 | 574.02 | 64 |
| | (trifluoromethyl)-3,4-dihydroimidazo[1,2-a:5,4-c′]dipyridin-2(1H)- | | | |
| | yl)ethan-1-one | | | |
| 3.1.30 | 1-(1-(3,4-dimethoxyphenyl)-7-(trifluoromethyl)-3,4- | 0.65 | 541.13 | 36 |
| | dihydroimidazo[1,2-a:5,4-c′]dipyridin-2(1H)-yl)-2-((2-ethylpyridin-3- | | | |
| | yl)oxy)ethan-1-one | | | |
| 3.1.35 | 2-((2-cyclopropylpyridin-3-yl)oxy)-1-(1-(3,4-dimethoxyphenyl)-7- | 0.68 | 553.09 | 336 |
| | (trifluoromethyl)-3,4-dihydroimidazo[1,2-a:5,4-c′]dipyridin-2(1H)- | | | |
| | yl)ethan-1-one | | | |

Following compounds were prepared from example 3.1.31 by Buchwald procedure described for acid (2. Step) with N-methylpiperazine, morpholine and aziridine, respectively. The LC-MS conditions used were LC-MS (A).

TABLE 4

| | | | | IC₅₀ |
|---|---|---|---|---|
| Example | Name | t_{R} | [M + H]⁺ | [nM] |
| 3.1.32 | 1-(1-(3,4-dimethoxyphenyl)-7-(trifluoromethyl)-3,4- | 0.70 | 638.30 | 21 |
| | dihydroimidazo[1,2-a:5,4-c′]dipyridin-2(1H)-yl)-2-(2-ethyl-4-(4- | | | |
| | methylpiperazin-1-yl)phenoxy)ethan-1-one | | | |
| 3.1.33 | 1-(1-(3,4-dimethoxyphenyl)-7-(trifluoromethyl)-3,4- | 0.76 | 625.09 | 12 |
| | dihydroimidazo[1,2-a:5,4-c′]dipyridin-2(1H)-yl)-2-(2-ethyl-4- | | | |
| | morpholinophenoxy)ethan-1-one | | | |
| 3.1.34 | 2-(4-(aziridin-1-yl)-2-ethylphenoxy)-1-(1-(3,4-dimethoxyphenyl)-7- | 0.86 | 581.09 | 36 |
| | (trifluoromethyl)-3,4-dihydroimidazo[1,2-a:5,4-c′]dipyridin-2(1H)- | | | |
| | yl)ethan-1-one | | | |

II. Biological Assays

Inhibitory activities on tryptophan hydroxylase 1 have been measured for each example compound using the following procedure:

Biochemical In Vitro Assay Using Fluorescence Readout

To generate the enzyme, full length human TPH1 is cloned into the plasmid pET20b(+) (Novagen) and expressed in E. coli. The bacterial cells are ruptured by sonication on ice and the lysate is cleared by centrifugation. The resulting protein in the pellet is re-extracted and TPH1 is purified from the obtained lysate by affinity chromatography using a pterin cosubstrate analog immobilized to the resin of the column. The protein is further purified by size exclusion chromatography to remove protein aggregates. The activity of TPH1 is determined by using a fluorescence assay. The enzyme activity assay is carried out at 15° C. with atmosphere oxygen for the duration of 60 minutes in a volume of 64 μl. The reaction is carried out in a 0.1 M Tris-HCl buffer, adjusted to pH 7.6, containing 1 mM DTT, 0.2 mg/mL catalase, 100 μM (±)-6-methyl-5,6,7,8-tetrahydropterine dihydrochloride, 40 μM L-tryptophan, and 40-80 nM of TPH1. The reaction is started by bringing together L-tryptophan with all the other reaction substituents and stopped by quenching with perchloric acid (HClO4). The amount of 5-hydroxy-L-tryptophan produced during the enzymatic reaction is determined by fluorescence readout. Fluorescence, as determined at 540 nm when excited at 300 nm wavelength, increases proportionally to the 5-hydroxy-L-tryptophan formed. Compounds are prepared as 10 mM stock solution in DMSO, then diluted in 384-well plates using DMSO followed by a transfer of the dilutions into the assay plate. Fluorescence is measured for each well and the fluorescence at 540 nm wavelength is compared to the fluorescence of the vehicle in place of compound. Inhibitory activities of example compounds with respect to the TPH1 protein are determined by calculating the IC₅₀ value (the concentration of compound needed to inhibit 50% of the enzyme activity). The calculated IC₅₀ values may fluctuate depending on the daily biochemical assay performance. Fluctuations of this kind are known to those skilled in the art. In the case where IC₅₀ values have been determined several times for the same compound, the mean is given. IC₅₀ values of exemplified compounds are displayed in the tables 1 to 4 above.

1. A compound of Formula (I) 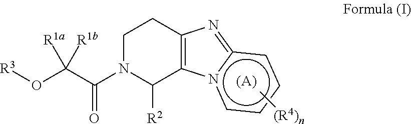
wherein
ring (A) represents a fused 6-membered aromatic ring containing the bridgehead nitrogen atom and optionally one additional ring nitrogen atom;
(R⁴)ₙ represents one or two optional substituents independently selected from (C₁₋₄)alkyl, (C₃₋₆)cycloalkyl, (C₁₋₃)trifluoroalkyl, halogen, or phenyl;
R^{1a} and R^{1b} independently represent hydrogen, methyl, ethyl; or R^{1a} and R^{1b} together with the carbon atom to which they are attached to form a cyclopropyl ring;
R² represents aryl, or heteroaryl, wherein said aryl or heteroaryl independently is unsubstituted, or mono-, di-, or tri-substituted, wherein the substituents are independently selected from:
(C₁₋₄)alkyl;
(C₁₋₄)alkoxy;
(C₃₋₆)cycloalkyl, optionally containing one or two ring oxygen atoms;
(C₁₋₃)fluoroalkyl;
(C₁₋₃)fluoroalkoxy;
halogen;
cyano;
hydroxy;
—O(CH₂)₂—NR²¹R²², wherein
R²¹ and R²² independently represent hydrogen or (C₁₋₃)alkyl; or
R²¹ and R²² together with the nitrogen atom to which they are attached to form a 4- to 7-membered saturated ring, wherein said ring optionally contains one ring oxygen atom, and wherein said ring is optionally substituted with one or two fluorine substituents;
—(CH₂)ₚ—NR²³R²⁴, wherein p represents the integer 0 or 1; and
R²³ and R²⁴ independently represent hydrogen or (C₁₋₃)alkyl; or
R²³ and R²⁴ together with the nitrogen atom to which they are attached to form a 4- to 7-membered saturated ring, wherein said ring optionally contains one ring oxygen atom, and wherein said ring is optionally substituted with one or two fluorine substituents;
carboxy;
—CO—NR²⁵R²⁶, wherein R²⁵ and R²⁶ independently represent hydrogen or (C₁₋₄)alkyl;
—OCH₂—CO—(C₁₋₄)alkoxy;
—CO—(C₁₋₄)alkoxy;
hydroxy-(C₁₋₄)alkyl;
(C₁₋₃)alkoxy-(C₁₋₄)alkyl;
(C₂₋₄)alkoxy substituted with one or two hydroxy;
(C₁₋₃)alkoxy-(C₂₋₄)alkoxy;
benzyloxy, wherein the phenyl group is optionally mono-substituted with methoxy; or
phenyl, optionally mono-substituted with halogen;
or two of said substituents together form a bivalent group selected from —O—CH₂—O—, or —O—CH₂—CH₂—O—;
R³ represents aryl, or 5- to 10-membered heteroaryl, wherein said aryl or heteroaryl independently is unsubstituted, or mono-, di-, or tri-substituted, wherein the substituents are independently selected from:
—NR³¹—SO₂—Y—R³², wherein
R³¹ represents hydrogen or (C₁₋₃)alkyl; Y represents a direct bond; and R³² represents (C₁₋₄)alkyl, or (C₃₋₆)cycloalkyl; or
R³¹ represents hydrogen or (C₁₋₃)alkyl; Y represents —NR^{Y}— wherein R^{v} represents hydrogen or (C₁₋₃)alkyl; and R³² represents (C₁₋₄)alkyl; or
R³¹ and R³² together with the nitrogen and the —SO₂—Y-group to which they are attached to form a 5-, 6-, or 7-membered ring, wherein Y represents a direct bond or —NR^{Y-}wherein R^{v} represents (C₁₋₃)alkyl;
—CO—NR³³R³⁴, wherein R³³ and R³⁴ independently represent hydrogen, (C₁₋₄)alkyl, or (C₃₋₆)cycloalkyl;
—SO₂—R³⁵ wherein R³⁵ represents (C₁₋₅)alkyl;
(C₁₋₄)alkyl;
(C₁₋₄)alkoxy;
(C₁₋₃)fluoroalkyl;
(C₁₋₃)fluoroalkoxy;
(C₃₋₆)cycloalkyl, optionally containing one oxygen ring atom, and optionally mono-substituted with amino, —NH—(SO)—(C₁₋₄)alkyl, or morpholin-4-yl;
halogen;
cyano;
nitro;
hydroxy-(C₁₋₄)alkyl;
—CO—(C₁₋₄)alkoxy;
5-membered heteroaryl;
phenyl;
—(CH₂)ₘ—NR³⁶R³⁷; wherein m represents the integer 0 or 1; and
R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
R³⁶ and R³⁷ together with the nitrogen to which they are attached to form a saturated 3- to 7-membered monocyclic ring; wherein said ring optionally contains an oxygen ring atom or a group —NR¹¹— wherein R¹¹ represents (C₁₋₄)alkyl; and wherein said ring independently is optionally substituted with:
one or two fluorine substituents; or
one oxo substituent attached to a ring carbon atom in alpha position to a ring nitrogen atom;
or two of said substituents together form a bivalent group selected from —O—CH₂—O—; —O—CH₂—CH₂—O—; or —CH₂—CH₂—NR³⁸—CH₂—, wherein R³⁸ represents hydrogen, (C₁₋₄)alkyl, —CO—(C₁₋₄)alkoxy, or —CO—(C₁₋₄)alkyl wherein the (C₁₋₄)alkyl is optionally mono-substituted with hydroxy; and the remaining of said substituents, if present, is (C₁₋₄)alkyl;
wherein in the particular case wherein R³ represents heteroaryl which is pyridinyl, such pyridinyl may additionally be present in form of the respective N-oxide;
or a pharmaceutically acceptable salt thereof.

2. A compound according to claim 1, wherein the absolute configuration of the carbon atom carrying the substituent R² is as depicted in Formula (I_{E}): 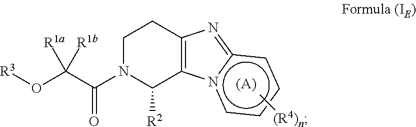
or a pharmaceutically acceptable salt thereof.

3. A compound according to claim 1, wherein R^{1a} and R^{1b} both represent hydrogen;
or a pharmaceutically acceptable salt thereof.

4. A compound according to claim 1, wherein the fragment 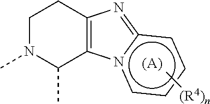
represents a fragment selected from:
A) 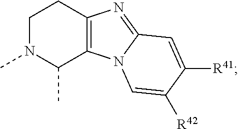
wherein R⁴¹ and R⁴² independently represent (C₁₋₄)alkyl, (C₃₋₆)cycloalkyl, (C₁₋₃)trifluoroalkyl, or halogen;
or
B) 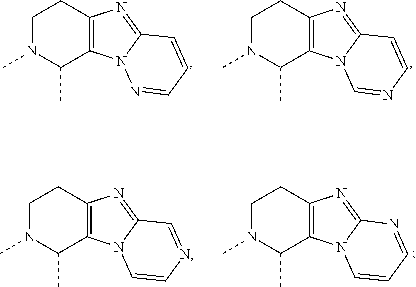
or a pharmaceutically acceptable salt thereof.

5. A compound according to claim 1, wherein the fragment 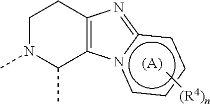
represents a fragment 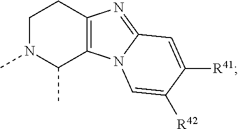
wherein R⁴¹ and R⁴² independently represent (C₁₋₄)alkyl, (C₁₋₃)trifluoroalkyl, or halogen;
or a pharmaceutically acceptable salt thereof.

6. A compound according to claim 1, wherein
R² represents phenyl, wherein said phenyl is mono-, di-, or tri-substituted, wherein the substituents are independently selected from:
(C₁₋₄)alkyl;
(C₁₋₄)alkoxy;
(C₃₋₆)cycloalkyl, optionally containing one or two ring oxygen atoms;
(C₁₋₃)fluoroalkyl;
(C₁₋₃)fluoroalkoxy;
halogen;
cyano;
hydroxy;
—O(CH₂)₂—NR²¹R²², wherein R²¹ and R²² independently represent hydrogen or (C₁₋₃)alkyl;
—CO—NR²⁵R²⁶, wherein R²⁵ and R²⁶ independently represent hydrogen or (C₁₋₄)alkyl;
—CO—(C₁₋₄)alkoxy;
hydroxy-(C₁₋₄)alkyl;
(C₁₋₃)alkoxy-(C₁₋₄)alkyl;
(C₂₋₄)alkoxy substituted with one or two hydroxy;
(C₁₋₃)alkoxy-(C₂₋₄)alkoxy;
benzyloxy, wherein the phenyl group is optionally mono-substituted with methoxy;
or two of said substituents together form a bivalent group selected from —O—CH₂—O—, or —O—CH₂—CH₂—O—;
or R² represents 5-membered heteroaryl, wherein said heteroaryl is mono-, or di-substituted, wherein the substituents are independently selected from:
(C₁₋₄)alkyl;
—(CH₂)ₚ—NR²³R²⁴, wherein p represents the integer 0 or 1; and
R²³ and R²⁴ independently represent hydrogen or (C₁₋₃)alkyl; or
R²³ and R²⁴ together with the nitrogen atom to which they are attached to form a 4- to 7-membered saturated ring, wherein said ring optionally contains one ring oxygen atom;
—CO—NR²⁵R²⁶, wherein R²⁵ and R²⁶ independently represent hydrogen or (C₁₋₃)alkyl;
phenyl, optionally mono-substituted with halogen;
or R² represents 6-membered heteroaryl, wherein said heteroaryl is unsubstituted, mono-, or di-substituted, wherein the substituents are independently selected from:
(C₁₋₄)alkyl;
(C₁₋₄)alkoxy;
(C₃₋₆)cycloalkyl, optionally containing one or two ring oxygen atoms;
(C₁₋₃)fluoroalkoxy;
halogen;
or R² represents unsubstituted 8- to 10-membered heteroaryl;
or a pharmaceutically acceptable salt thereof.

7. A compound according to claim 1, wherein
R² represents phenyl, wherein said phenyl is mono-, di-, or tri-substituted, wherein the substituents are independently selected from:
(C₁₋₄)alkyl;
(C₁₋₄)alkoxy;
(C₃₋₆)cycloalkyl;
halogen;
(C₂₋₄)alkoxy substituted with one or two hydroxy;
or R² represents 5-membered heteroaryl, wherein said 5-membered heteroaryl is mono-, or di-substituted, wherein the substituents are independently selected from:
(C₁₋₄)alkyl; or
phenyl, optionally mono-substituted with halogen;
or R² represents 6-membered heteroaryl, wherein said 6-membered heteroaryl is unsubstituted, mono-, or di-substituted, wherein the substituents are independently selected from:
(C₁₋₄)alkyl;
(C₁₋₄)alkoxy;
(C₃₋₆)cycloalkyl;
halogen;
or a pharmaceutically acceptable salt thereof.

8. A compound according to claim 1, wherein R³ represents a fragment 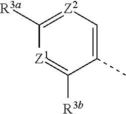
wherein
Z¹ and Z² independently represent CH or N;
R^{3a} represents:
—NR³¹—SO₂—Y—R³², wherein
R³¹ represents hydrogen or (C₁₋₃)alkyl; Y represents a direct bond; and R³² represents (C₁₋₄)alkyl, or (C₃₋₆)cycloalkyl; or
R³¹ represents hydrogen; Y represents —NR^{Y1}— wherein R^{Y1} represents hydrogen or (C₁₋₃)alkyl; and R³² represents (C₁₋₄)alkyl; or
R³¹ and R³² together with the nitrogen and the —SO₂—Y-group to which they are attached to form 1,1-dioxidoisothiazolidin-2-yl group;
—CO—NR³³R³⁴, wherein R³³ and R³⁴ independently represent hydrogen, (C₁₋₄)alkyl, or (C₃₋₆)cycloalkyl;
—SO₂—R³⁵ wherein R³⁵ represents (C-5)alkyl;
—(CH₂)ₘ—NR³⁶R³⁷; wherein m represents the integer 0 or 1; and
R³⁶ and R³⁷ independently represent hydrogen, (C₁₋₄)alkyl, (C₂₋₃)fluoroalkyl, hydroxy-(C₂₋₄)alkyl, or (C₁₋₄)alkoxy-(C₂₋₄)alkyl; or
R³⁶ and R³⁷ together with the nitrogen to which they are attached to form a saturated 3- to 7-membered monocyclic ring; wherein said ring optionally contains an oxygen ring atom or a group —NR¹¹— wherein R¹¹ represents (C₁₋₄)alkyl; and
R^{3b} represents (C₁₋₄)alkyl; halogen; or (C₃₋₆)cycloalkyl;
or a pharmaceutically acceptable salt thereof.

9. A compound according to claim 8, wherein Z¹ represents N and Z² represents CH;
or a pharmaceutically acceptable salt thereof.

10. A compound according to claim 8, wherein R^{3a} represents:
—NR³¹—SO₂—Y—R³², wherein
R³¹ represents hydrogen or (C₁₋₃)alkyl; Y represents a direct bond; and R³² represents (C₁₋₄)alkyl, or (C₃₋₆)cycloalkyl; or
R³¹ and R³² together with the nitrogen and the —SO₂—Y-group to which they are attached to form a 1,1-dioxidoisothiazolidin-2-yl group;
—CO—NR³³R³⁴, wherein R³³ and R³⁴ independently represent hydrogen, (C₁₋₄)alkyl, or (C₃₋₆)cycloalkyl;
—SO₂—R³⁵ wherein R³⁵ represents (C₁₋₅)alkyl;
and
R^{3b} represents (C₁₋₄)alkyl; halogen; or (C₃₋₆)cycloalkyl;
or a pharmaceutically acceptable salt thereof.

11. A compound according to claim 1; selected from the group consisting of:
ethyl 5-(2-(1-(3,4-dimethoxyphenyl)-3,4-dihydroimidazo [1,2-a:5,4-c′]dipyridin-2(1H)-yl)-2-oxoethoxy)-4-ethyl-2-methylbenzoate;
2-(2,4-dichlorophenoxy)-1-(1-(3,4-dimethoxyphenyl)-3,4-dihydroimidazo [1,2-a:5,4-c′]dipyridin-2(1H)-yl)ethan-1-one;
1-(1-(3,4-dimethoxyphenyl)-3,4-dihydroimidazo [1,2-a:5,4-c′]dipyridin-2(1H)-yl)-2-(naphthalen-2-yloxy)ethan-1-one;
1-(1-(3,4-dimethoxyphenyl)-3,4-dihydroimidazo[1,2-a:5,4-c′]dipyridin-2(1H)-yl)-2-((1-methylnaphthalen-2-yl)oxy)ethan-1-one;
1-(1-(3,4-dimethoxyphenyl)-3,4-dihydroimidazo[1,2-a:5,4-c′]dipyridin-2(1H)-yl)-2-((1-ethylnaphthalen-2-yl)oxy)ethan-1-one;
2-((1-bromonaphthalen-2-yl)oxy)-1-(1-(3,4-dimethoxyphenyl)-3,4-dihydroimidazo[1,2-a:5,4-c′]dipyridin-2(1H)-yl)ethan-1-one;
2-((1-chloronaphthalen-2-yl)oxy)-1-(1-(3,4-dimethoxyphenyl)-3,4-dihydroimidazo[1,2-a:5,4-c′]dipyridin-2(1H)-yl)ethan-1-one;
1-(1-(6-chloro-2-fluoropyridin-3-yl)-3,4-dihydroimidazo[,2-a:5,4-c′]dipyridin-2(1H)-yl)-2-((2-chloro-6-morpholinopyridin-3-yl)oxy)ethan-1-one;
2-((2-chloro-6-morpholinopyridin-3-yl)oxy)-1-(1-(5-cyclopropyl-3-fluoropyridin-2-yl)-3,4-dihydroimidazo[1,2-a:5,4-c′]dipyridin-2(1H)-yl)ethan-1-one;
2-(2-chloro-4-morpholinophenoxy)-1-(1-(5-cyclopropyl-3-fluoropyridin-2-yl)-3,4-dihydroimidazo[1,2-a:5,4-c′]dipyridin-2(1H)-yl)ethan-1-one;
2-((2-chloro-6-morpholinopyridin-3-yl)oxy)-1-(1-(6-cyclopropyl-2-fluoropyridin-3-yl)-3,4-dihydroimidazo[1,2-a:5,4-c′]dipyridin-2(1H)-yl)ethan-1-one;
1-(1-(5-chloro-3-fluoropyridin-2-yl)-3,4-dihydroimidazo[,2-a:5,4-c′]dipyridin-2(1H)-yl)-2-((2-chloro-6-morpholinopyridin-3-yl)oxy)ethan-1-one;
methyl-4-(2-(2-((2-chloro-6-(methylsulfonamido)pyridin-3-yl)oxy)acetyl)-1,2,3,4-tetrahydroimidazo[1,2-a:5,4-c′]dipyridin-1-yl)-3-fluorobenzoate;
methyl-4-(2-(2-((2-chloro-6-(cyclopropylcarbamoyl)pyridin-3-yl)oxy)acetyl)-1,2,3,4-tetrahydroimidazo[1,2-a:5,4-c′]dipyridin-1-yl)-3-fluorobenzoate;
6-chloro-N-cyclopropyl-5-(2-(1-(2-fluoro-4-(methoxymethyl)phenyl)-3,4-dihydroimidazo[1,2-a:5,4-c′]dipyridin-2(1H)-yl)-2-oxoethoxy)picolinamide;
N-(6-chloro-5-(2-(1-(2-fluoro-4-(methoxymethyl)phenyl)-3,4-dihydroimidazo [1,2-a:5,4-c′]dipyridin-2(1H)-yl)-2-oxoethoxy)pyridin-2-yl)methanesulfonamide;
6-chloro-N-cyclopropyl-5-(2-(1-(2-fluoro-4-(trifluoromethoxy)phenyl)-3,4-dihydroimidazo[1,2-a:5,4-c′]dipyridin-2(1H)-yl)-2-oxoethoxy)picolinamide;
N-(6-chloro-5-(2-(1-(4-cyano-2-fluorophenyl)-3,4-dihydroimidazo[1,2-a:5,4-c′]dipyridin-2(1H)-yl)-2-oxoethoxy)pyridin-2-yl)methanesulfonamide;
6-chloro-5-(2-(1-(4-cyano-2-fluorophenyl)-3,4-dihydroimidazo[1,2-a:5,4-c′]dipyridin-2(1H)-yl)-2-oxoethoxy)-N-cyclopropylpicolinamide;
6-chloro-N-cyclopropyl-5-(2-(1-(2-fluoro-4-(2-methoxyethoxy)phenyl)-3,4-dihydroimidazo[1,2-a:5,4-c′]dipyridin-2(1H)-yl)-2-oxoethoxy)picolinamide;
N-(6-chloro-5-(2-(1-(2-fluoro-4-(2-methoxyethoxy)phenyl)-3,4-dihydroimidazo [1,2-a:5,4-c′]dipyridin-2(1H)-yl)-2-oxoethoxy)pyridin-2-yl)methanesulfonamide;
6-chloro-N-cyclopropyl-5-(2-(1-(2-fluoro-4-(2-hydroxyethoxy)phenyl)-3,4-dihydroimidazo[1,2-a:5,4-c′]dipyridin-2(1H)-yl)-2-oxoethoxy)picolinamide;
N-(6-Chloro-5-(2-(1-(2-fluoro-4-(2-hydroxyethoxy)phenyl)-3,4-dihydroimidazo [1,2-a:5,4-c′]dipyridin-2(1H)-yl)-2-oxoethoxy)pyridin-2-yl)methanesulfonamide;
2-((2-chloro-6-(morpholinomethyl)pyridin-3-yl)oxy)-1-(1-(2-fluoro-4-methoxyphenyl)-3,4-dihydroimidazo[1,2-a:5,4-c′]dipyridin-2(1H)-yl)ethan-1-one;
N-(6-chloro-5-(2-(1-(2-fluoro-4-methoxyphenyl)-3,4-dihydroimidazo [1,2-a:5,4-c′]dipyridin-2(1H)-yl)-2-oxoethoxy)pyridin-2-yl)methanesulfonamide;
6-chloro-N-cyclopropyl-5-(2-(1-(2-fluoro-4-methoxyphenyl)-3,4-dihydroimidazo[1,2-a:5,4-c′]dipyridin-2(1H)-yl)-2-oxoethoxy)picolinamide;
1-(1-(4-chloro-2-fluorophenyl)-3,4-dihydroimidazo [1,2-a:5,4-c′]dipyridin-2(1H)-yl)-2-(4-chloro-2-methylphenoxy)ethan-1-one;
1-(1-(4-chloro-2-fluorophenyl)-3,4-dihydroimidazo [1,2-a:5,4-c′]dipyridin-2(1H)-yl)-2-((2-chloro-6-((2-hydroxyethyl)(methyl)amino)pyridin-3-yl)oxy)ethan-1-one;
1-(1-(4-chloro-2-fluorophenyl)-3,4-dihydroimidazo [1,2-a:5,4-c′]dipyridin-2(1H)-yl)-2-((2-chloro-6-(methyl(2,2,2-trifluoroethyl)amino)pyridin-3-yl)oxy)ethan-1-one;
1-(1-(4-chloro-2-fluorophenyl)-3,4-dihydroimidazo [1,2-a:5,4-c′]dipyridin-2(1H)-yl)-2-((2-chloro-6-(dimethylamino)pyridin-3-yl)oxy)ethan-1-one;
5-(2-(1-(4-chloro-2-fluorophenyl)-3,4-dihydroimidazo[1,2-a:5,4-c′]dipyridin-2(1H)-yl)-2-oxoethoxy)-N,6-dicyclopropylpicolinamide;
1-(1-(4-chloro-2-fluorophenyl)-3,4-dihydroimidazo [1,2-a:5,4-c′]dipyridin-2(1H)-yl)-2-((2-ethylpyridin-3-yl)oxy)ethan-1-one;
1-(1-(4-chloro-2-fluorophenyl)-3,4-dihydroimidazo [1,2-a:5,4-c′]dipyridin-2(1H)-yl)-2-((2-chloro-6-(morpholinomethyl)pyridin-3-yl)oxy)ethan-1-one;
1-(1-(4-chloro-2-fluorophenyl)-3,4-dihydroimidazo [1,2-a:5,4-c′]dipyridin-2(1H)-yl)-2-((2-chloro-6-morpholinopyridin-3-yl)oxy)ethan-1-one;
1-(1-(4-chloro-2-fluorophenyl)-3,4-dihydroimidazo [1,2-a:5,4-c′]dipyridin-2(1H)-yl)-2-((2-ethyl-6-methylpyridin-3-yl)oxy)ethan-1-one;
N-(6-chloro-5-(2-(1-(4-chloro-2-fluorophenyl)-3,4-dihydroimidazo [,2-a:5,4-c′]dipyridin-2(1H)-yl)-2-oxoethoxy)pyridin-2-yl)methanesulfonamide;
1-(1-(4-chloro-2-fluorophenyl)-3,4-dihydroimidazo [1,2-a:5,4-c′]dipyridin-2(1H)-yl)-2-((2-chloro-6-((2-methoxyethyl)(methyl)amino)pyridin-3-yl)oxy)ethan-1-one;
2-(2-chloro-4-morpholinophenoxy)-1-(1-(4-cyclopropyl-2-fluorophenyl)-3,4-dihydroimidazo[1,2-a:5,4-c′]dipyridin-2(1H)-yl)ethan-1-one;
2-((2-chloro-6-morpholinopyridin-3-yl)oxy)-1-(1-(4-cyclopropyl-2-fluorophenyl)-3,4-dihydroimidazo[1,2-a:5,4-c′]dipyridin-2(1H)-yl)ethan-1-one;
1-(1-(4-cyclopropyl-2-fluorophenyl)-3,4-dihydroimidazo [1,2-a:5,4-c′]dipyridin-2(1H)-yl)-2-((6-(dimethylamino)-2-methylpyridin-3-yl)oxy)ethan-1-one;
2-((2-chloropyridin-3-yl)oxy)-1-(1-(4-cyclopropyl-2-fluorophenyl)-3,4-dihydroimidazo[1,2-a:5,4-c′]dipyridin-2(1H)-yl)ethan-1-one;
1-(1-(4-cyclopropyl-2-fluorophenyl)-3,4-dihydroimidazo [1,2-a:5,4-c′]dipyridin-2(1H)-yl)-2-((2-ethyl-6-methylpyridin-3-yl)oxy)ethan-1-one;
1-(1-(4-cyclopropyl-2-fluorophenyl)-3,4-dihydroimidazo [1,2-a:5,4-c′]dipyridin-2(1H)-yl)-2-((2-(trifluoromethyl)pyridin-3-yl)oxy)ethan-1-one;
2-((2-chloro-6-morpholinopyridin-3-yl)oxy)-1-(1-(3-phenyl-1,2,4-oxadiazol-5-yl)-3,4-dihydroimidazo[1,2-a:5,4-c′]dipyridin-2(1H)-yl)ethan-1-one;
N-(6-chloro-5-(2-(1-(4-chloro-2-fluorophenyl)-8-fluoro-3,4-dihydroimidazo[1,2-a:5,4-c′]dipyridin-2(1H)-yl)-2-oxoethoxy)pyridin-2-yl)methanesulfonamide;
N-(5-(2-(1-(4-chloro-2-fluorophenyl)-8-fluoro-3,4-dihydroimidazo[1,2-a:5,4-c′]dipyridin-2(1H)-yl)-2-oxoethoxy)-6-ethylpyridin-2-yl)methanesulfonamide;
N-(5-(2-(1-(2,4-dimethylthiazol-5-yl)-8-fluoro-3,4-dihydroimidazo [,2-a:5,4-c′]dipyridin-2(1H)-yl)-2-oxoethoxy)-6-ethylpyridin-2-yl)methanesulfonamide;
1-(7-chloro-1-(3,4-dimethoxyphenyl)-8-methyl-3,4-dihydroimidazo[1,2-a:5,4-c′]dipyridin-2(1H)-yl)-2-(naphthalen-2-yloxy)ethan-1-one;
N-(6-chloro-5-(2-(1-(4-chloro-2-fluorophenyl)-7-fluoro-3,4-dihydroimidazo[1,2-a:5,4-c′]dipyridin-2(1H)-yl)-2-oxoethoxy)pyridin-2-yl)methanesulfonamide;
2-(2-chloro-4-morpholinophenoxy)-1-(9-(4-cyclopropyl-2-fluorophenyl)-6,9-dihydropyrido[4′,3′:4,5]imidazo [1,2-b]pyridazin-8(7H)-yl)ethan-1-one;
2-((2-chloro-6-morpholinopyridin-3-yl)oxy)-1-(9-(4-cyclopropyl-2-fluorophenyl)-6,9-dihydropyrido[4′,3′:4,5]imidazo [1,2-b]pyridazin-8(7H)-yl)ethan-1-one;
2-((2-chloro-6-morpholinopyridin-3-yl)oxy)-1-(9-(5-cyclopropyl-3-fluoropyridin-2-yl)-6,9-dihydropyrido [4′,3′:4,5]imidazo [1,2-b]pyridazin-8(7H)-yl)ethan-1-one;
5-(8-(2-((2-Chloro-6-morpholinopyridin-3-yl)oxy)acetyl)-6,7,8,9-tetrahydropyrido[4′,3′:4,5]imidazo[1,2-b]pyridazin-9-yl)-N,N-dimethylthiophene-3-carboxamide;
N-(5-(2-(9-(2,4-dimethylthiazol-5-yl)-6,9-dihydropyrido [4′,3′:4,5]imidazo[1,2-b]pyridazin-8(7H)-yl)-2-oxoethoxy)-6-ethylpyridin-2-yl)methanesulfonamide;
2-((2-chloro-6-morpholinopyridin-3-yl)oxy)-1-(1-(4-cyclopropyl-2-fluorophenyl)-3,4-dihydropyrido[4′,3′:4,5]imidazo[1,2-a]pyrazin-2(1H)-yl)ethan-1-one;
2-((2-chloro-6-morpholinopyridin-3-yl)oxy)-1-(6-(4-cyclopropyl-2-fluorophenyl)-8,9-dihydropyrido[4′,3′:4,5]imidazo[1,2-a]pyrimidin-7(6H)-yl)ethan-1-one;
1-(7-chloro-1-(3,4-dimethoxyphenyl)-3,4-dihydroimidazo[1,2-a:5,4-c′]dipyridin-2(1H)-yl)-2-(naphthalen-2-yloxy)ethan-1-one hydrochloride;
1-(7-chloro-1-(3,4-dimethoxyphenyl)-3,4-dihydroimidazo[1,2-a:5,4-c′]dipyridin-2(1H)-yl)-2-(2-chloro-4-(morpholinomethyl)phenoxy)ethan-1-one;
1-(7-chloro-1-(3,4-dimethoxyphenyl)-3,4-dihydroimidazo[1,2-a:5,4-c′]dipyridin-2(1H)-yl)-2-(2,4-dichlorophenoxy)ethan-1-one;
1-(7-chloro-1-(3,4-dimethoxyphenyl)-3,4-dihydroimidazo[1,2-a:5,4-c′]dipyridin-2(1H)-yl)-2-(2-chloro-4-morpholinophenoxy)ethan-1-one;
1-(7-chloro-1-(3-((R)-2,3-dihydroxypropoxy)-4-methoxyphenyl)-3,4-dihydroimidazo [1,2-a:5,4-c′]dipyridin-2(1H)-yl)-2-(naphthalen-2-yloxy)ethan-1-one;
1-(7-chloro-1-(3-((R)-2,3-dihydroxypropoxy)-4-methoxyphenyl)-3,4-dihydroimidazo [1,2-a:5,4-c′]dipyridin-2(1H)-yl)-2-(2-chloro-4-(morpholinomethyl)phenoxy)ethan-1-one;
1-(7-chloro-1-(4-methoxy-3-(3-methoxypropoxy)phenyl)-3,4-dihydroimidazo [1,2-a:5,4-c′]dipyridin-2(1H)-yl)-2-(naphthalen-2-yloxy)ethan-1-one;
1-(7-chloro-1-(3-(3-hydroxypropoxy)-4-methoxyphenyl)-3,4-dihydroimidazo[1,2-a:5,4-c′]dipyridin-2(1H)-yl)-2-(naphthalen-2-yloxy)ethan-1-one;
1-(7-chloro-1-(3-(2-hydroxyethoxy)-4-methoxyphenyl)-3,4-dihydroimidazo [1,2-a:5,4-c′]dipyridin-2(1H)-yl)-2-(naphthalen-2-yloxy)ethan-1-one;
1-(7-chloro-1-(3-(2-hydroxyethoxy)-4-methoxyphenyl)-3,4-dihydroimidazo [1,2-a:5,4-c′]dipyridin-2(1H)-yl)-2-(2-chloro-4-(morpholinomethyl)phenoxy)ethan-1-one;
Methyl 2-(5-(7-chloro-2-(2-(2-chloro-4-(morpholinomethyl) phenoxy)acetyl)-1,2,3,4-tetrahydroimidazo[1,2-a:5,4-c′]dipyridin-1-yl)-2-methoxyphenoxy)acetate;
N-(6-chloro-5-(2-(7-chloro-1-(2-fluoro-4-methylphenyl)-3,4-dihydroimidazo[1,2-a:5,4-c′]dipyridin-2(1H)-yl)-2-oxoethoxy)pyridin-2-yl)methanesulfonamide;
1-(7-chloro-1-(2-fluoro-4-methoxyphenyl)-3,4-dihydroimidazo [1,2-a:5,4-c′]dipyridin-2(1H)-yl)-2-(2-chloro-4-(morpholinomethyl)phenoxy)ethan-1-one;
1-(7-chloro-1-(2-fluoro-4-methoxyphenyl)-3,4-dihydroimidazo [1,2-a:5,4-c′]dipyridin-2(1H)-yl)-2-((2-chloro-6-(morpholinomethyl)pyridin-3-yl)oxy)ethan-1-one;
1-((1R)-7-chloro-1-(2-fluoro-4-methoxyphenyl)-3,10a-dihydroimidazo[1,2-a:5,4-c′]dipyridin-2(1H)-yl)-2-(2-chloro-4-morpholinophenoxy)ethan-1-one;
N-(6-chloro-5-(2-(7-chloro-1-(4-chloro-2-fluorophenyl)-3,4-dihydroimidazo[1,2-a:5,4-c′]dipyridin-2(1H)-yl)-2-oxoethoxy)pyridin-2-yl)methanesulfonamide;
1-((1R)-7-chloro-1-(6-methoxypyridin-3-yl)-3,10a-dihydroimidazo[1,2-a:5,4-c′]dipyridin-2(1H)-yl)-2-(2-chloro-4-morpholinophenoxy)ethan-1-one;
1-(7-chloro-1-(4-methoxy-3-((4-methoxybenzyl)oxy)phenyl)-3,4-dihydroimidazo[1,2-a:5,4-c′]dipyridin-2(1H)-yl)-2-(naphthalen-2-yloxy)ethan-1-one;
1-(7-chloro-1-(3-((S)-2,3-dihydroxypropoxy)-4-methoxyphenyl)-3,4-dihydroimidazo [1,2-a:5,4-c′]dipyridin-2(1H)-yl)-2-(naphthalen-2-yloxy)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-5-methylphenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(2-chloro-3-(trifluoromethyl)phenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-((2-chloropyridin-3-yl)oxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-((2-bromopyridin-3-yl)oxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(2,4-dichlorophenoxy)-1-(1-(4,5-dimethoxyphenyl)-7-(trifluoromethyl)-3,4-dihydroimidazo[1,2-a:5,4-c′]dipyridin-2(1H)-yl)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(isoquinolin-7-yloxy)ethan-1-one;
2-((4-chloro-1-methyl-5-(trifluoromethyl)-1H-pyrazol-3-yl)oxy)-1-(1-(3,4-dimethoxyphenyl)-7-(trifluoromethyl)-3,4-dihydroimidazo [1,2-a:5,4-c′]dipyridin-2(1H)-yl)ethan-1-one;
2-(2-chloro-4-(morpholinomethyl)phenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-((2-chloro-6-(morpholinomethyl)pyridin-3-yl)oxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(4-chloro-2-ethylphenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
2-(2-chloro-4-(trifluoromethyl)phenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
1-(1-(3,4-dimethoxyphenyl)-7-(trifluoromethyl)-3,4-dihydroimidazo [1,2-a:5,4-c′]dipyridin-2(1H)-yl)-2-((2-ethylpyridin-3-yl)oxy)ethan-1-one;
2-(4-bromo-2-ethylphenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(2-ethyl-4-(4-methylpiperazin-1-yl)phenoxy)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(2-ethyl-4-morpholinophenoxy)ethan-1-one;
2-(4-(aziridin-1-yl)-2-ethylphenoxy)-1-(1-(3,4-dimethoxyphenyl)-7-(trifluoromethyl)-3,4-dihydroimidazo[1,2-a:5,4-c′]dipyridin-2(1H)-yl)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(p-tolyloxy)ethan-1-one;
1-(1-(3,4-dimethoxyphenyl)-7-(trifluoromethyl)-3,4-dihydroimidazo[1,2-a:5,4-c′]dipyridin-2(1H)-yl)-2-(2-(trifluoromethyl)phenoxy)ethan-1-one;
1-(1-(3,4-dimethylphenyl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(3,4-dimethylphenyl)-7-(trifluoromethyl)-3,4-dihydroimidazo[1,2-a:5,4-c′]dipyridin-2(1H)-yl)ethan-1-one;
1-(1-(3-(difluoromethoxy)-4-methoxyphenyl)-7-(trifluoromethyl)-3,4-dihydroimidazo [1,2-a:5,4-c′]dipyridin-2(1H)-yl)-2-(naphthalen-2-yloxy)ethan-1-one;
1-(1-(4-methoxy-3-(trifluoromethoxy)phenyl)-7-(trifluoromethyl)-3,4-dihydroimidazo [1,2-a:5,4-c′]dipyridin-2(1H)-yl)-2-(naphthalen-2-yloxy)ethan-1-one;
1-(1-(4-(2-(dimethylamino)ethoxy)-3-methoxyphenyl)-7-(trifluoromethyl)-3,4-dihydroimidazo[1,2-a:5,4-c′]dipyridin-2(1H)-yl)-2-(naphthalen-2-yloxy)ethan-1-one;
1-(1-(2-fluoro-4,5-dimethoxyphenyl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(3-morpholinooxetan-3-yl)phenoxy)-1-(1-(2-fluoro-4,5-dimethoxyphenyl)-7-(trifluoromethyl)-3,4-dihydroimidazo [1,2-a:5,4-c′]dipyridin-2(1H)-yl)ethan-1-one;
2-(2-chloro-4-(morpholinomethyl)phenoxy)-1-(1-(2-fluoro-4,5-dimethoxyphenyl)-7-(trifluoromethyl)-3,4-dihydroimidazo [1,2-a:5,4-c′]dipyridin-2(1H)-yl)ethan-1-one;
tert-butyl 7-(2-(1-(2-fluoro-4,5-dimethoxyphenyl)-7-(trifluoromethyl)-3,4-dihydroimidazo [1,2-a:5,4-c′]dipyridin-2(1H)-yl)-2-oxoethoxy)-3,4-dihydroisoquinoline-2(1H)-carboxylate;
1-(1-(2-fluoro-4,5-dimethoxyphenyl)-7-(trifluoromethyl)-3,4-dihydroimidazo[1,2-a:5,4-c′]dipyridin-2(1H)-yl)-2-((1,2,3,4-tetrahydroisoquinolin-7-yl)oxy)ethan-1-one;
1-(1-(2-fluoro-4,5-dimethoxyphenyl)-7-(trifluoromethyl)-3,4-dihydroimidazo[1,2-a:5,4-c′]dipyridin-2(1H)-yl)-2-((2-methyl-1,2,3,4-tetrahydroisoquinolin-7-yl)oxy)ethan-1-one;
2-((2-acetyl-1,2,3,4-tetrahydroisoquinolin-7-yl)oxy)-1-(1-(2-fluoro-4,5-dimethoxyphenyl)-7-(trifluoromethyl)-3,4-dihydroimidazo [1,2-a:5,4-c′]dipyridin-2(1H)-yl)ethan-1-one;
1-(1-(2-fluoro-4,5-dimethoxyphenyl)-7-(trifluoromethyl)-3,4-dihydroimidazo[1,2-a:5,4-c′]dipyridin-2(1H)-yl)-2-((2-(2-hydroxyacetyl)-1,2,3,4-tetrahydroisoquinolin-7-yl)oxy)ethan-1-one;
N-(3-(3-chloro-4-(2-(1-(2-fluoro-4,5-dimethoxyphenyl)-7-(trifluoromethyl)-3,4-dihydroimidazo[1,2-a:5,4-c′]dipyridin-2(1H)-yl)-2-oxoethoxy)phenyl)oxetan-3-yl)-2-methylpropane-2-sulfinamide;
2-(4-(3-aminooxetan-3-yl)-2-chlorophenoxy)-1-(1-(2-fluoro-4,5-dimethoxyphenyl)-7-(trifluoromethyl)-3,4-dihydroimidazo [1,2-a:5,4-c′]dipyridin-2(1H)-yl)ethan-1-one;
1-(1-(5,6-dimethoxypyridin-3-yl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(2-fluoropyridin-3-yl)-7-(trifluoromethyl)-3,4-dihydroimidazo[1,2-a:5,4-c′]dipyridin-2(1H)-yl)ethan-1-one;
1-(1-(2-fluoropyridin-3-yl)-7-(trifluoromethyl)-3,4-dihydroimidazo [1,2-a:5,4-c′]dipyridin-2(1H)-yl)-2-(naphthalen-2-yloxy)ethan-1-one;
1-(1-(5,6-dimethoxypyridin-2-yl)-7-(trifluoromethyl)-3,4-dihydroimidazo [1,2-a:5,4-c′]dipyridin-2(1H)-yl)-2-(naphthalen-2-yloxy)ethan-1-one;
1-(1-(3-fluoropyridin-2-yl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-morpholinophenoxy)-1-(1-(3-fluoropyridin-2-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(2-fluoro-4-methylphenyl)-7-(trifluoromethyl)-3,4-dihydroimidazo[1,2-a:5,4-c′]dipyridin-2(1H)-yl)ethan-1-one;
2-((2-chloropyridin-3-yl)oxy)-1-(1-(2-fluoro-4-methylphenyl)-7-(trifluoromethyl)-3,4-dihydroimidazo[1,2-a:5,4-c′]dipyridin-2(1H)-yl)ethan-1-one;
2-((4-chloro-1-methyl-5-(trifluoromethyl)-1H-pyrazol-3-yl)oxy)-1-(1-(2-fluoro-4-methylphenyl)-7-(trifluoromethyl)-3,4-dihydroimidazo[1,2-a:5,4-c′]dipyridin-2(1H)-yl)ethan-1-one;
2-((2-ethyl-6-methylpyridin-3-yl)oxy)-1-(1-(2-fluoro-4-methylphenyl)-7-(trifluoromethyl)-3,4-dihydroimidazo[1,2-a:5,4-c′]dipyridin-2(1H)-yl)ethan-1-one;
N-(6-chloro-5-(2-(1-(2-fluoro-4-methylphenyl)-7-(trifluoromethyl)-3,4-dihydroimidazo[1,2-a:5,4-c′]dipyridin-2(1H)-yl)-2-oxoethoxy)pyridin-2-yl)methanesulfonamide;
2-(naphthalen-2-yloxy)-1-(1-(p-tolyl)-7-(trifluoromethyl)-3,4-dihydroimidazo [1,2-a:5,4-c′]dipyridin-2(1H)-yl)ethan-1-one;
2-(2-chloro-4-(morpholinomethyl)phenoxy)-1-(1-(p-tolyl)-7-(trifluoromethyl)-3,4-dihydroimidazo[1,2-a:5,4-c′]dipyridin-2(1H)-yl)ethan-1-one;
1-(1-(4-methoxyphenyl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(4-ethoxyphenyl)-7-(trifluoromethyl)-3,4-dihydroimidazo[1,2-a:5,4-c′]dipyridin-2(1H)-yl)ethan-1-one;
2-(2,3-dimethyl-4-(morpholinomethyl)phenoxy)-1-(1-(4-ethoxyphenyl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(4-(difluoromethoxy)phenyl)-7-(trifluoromethyl)-3,4-dihydroimidazo [1,2-a:5,4-c′]dipyridin-2(1H)-yl)ethan-1-one;
2-(2-chloro-4-(morpholinomethyl)phenoxy)-1-(7-(trifluoromethyl)-1-(4-(trifluoromethyl)phenyl)-3,4-dihydroimidazo[1,2-a:5,4-c′]dipyridin-2(1H)-yl)ethan-1-one;
1-(1-(2-fluoro-4-methoxyphenyl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(2-fluoro-4-methoxyphenyl)-7-(trifluoromethyl)-3,4-dihydroimidazo [1,2-a:5,4-c′]dipyridin-2(1H)-yl)ethan-1-one;
2-(2,3-dimethyl-4-(morpholinomethyl)phenoxy)-1-(1-(2-fluoro-4-methoxyphenyl)-7-(trifluoromethyl)-3,4-dihydroimidazo [1,2-a:5,4-c′]dipyridin-2(1H)-yl)ethan-1-one;
2-((2-acetyl-5-methyl-1,2,3,4-tetrahydroisoquinolin-6-yl)oxy)-1-(1-(2-fluoro-4-methoxyphenyl)-7-(trifluoromethyl)-3,4-dihydroimidazo[1,2-a:5,4-c′]dipyridin-2(1H)-yl)ethan-1-one;
1-(1-(4-chlorophenyl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(4-chlorophenyl)-7-(trifluoromethyl)-3,4-dihydroimidazo[1,2-a:5,4-c′]dipyridin-2(1H)-yl)ethan-1-one;
1-(1-(4-(aminomethyl)phenyl)-7-(trifluoromethyl)-3,4-dihydroimidazo [1,2-a:5,4-c′]dipyridin-2(1H)-yl)-2-(naphthalen-2-yloxy)ethan-1-one;
1-(1-(2-fluorophenyl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(2-fluorophenyl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(6-chloropyridin-3-yl)-7-(trifluoromethyl)-3,4-dihydroimidazo[1,2-a:5,4-c′]dipyridin-2(1H)-yl)ethan-1-one;
1-(1-(6-chloropyridin-3-yl)-7-(trifluoromethyl)-3,4-dihydroimidazo [1,2-a:5,4-c′]dipyridin-2(1H)-yl)-2-(naphthalen-2-yloxy)ethan-1-one;
1-(1-(6-methylpyridin-3-yl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(6-methylpyridin-3-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(6-methoxy-4-methylpyridin-3-yl)-7-(trifluoromethyl)-3,4-dihydroimidazo [1,2-a:5,4-c′]dipyridin-2(1H)-yl)ethan-1-one;
1-(1-(4-chloro-2-fluorophenyl)-7-(trifluoromethyl)-3,4-dihydroimidazo [1,2-a:5,4-c′]dipyridin-2(1H)-yl)-2-((2-chloropyridin-3-yl)oxy)ethan-1-one;
1-(1-(4-chloro-2-fluorophenyl)-7-(trifluoromethyl)-3,4-dihydroimidazo [1,2-a:5,4-c′]dipyridin-2(1H)-yl)-2-(2-chloro-4-(morpholinomethyl)phenoxy)ethan-1-one;
2-((4-chloro-1-methyl-5-(trifluoromethyl)-1H-pyrazol-3-yl)oxy)-1-(1-(4-chloro-2-fluorophenyl)-7-(trifluoromethyl)-3,4-dihydroimidazo[1,2-a:5,4-c′]dipyridin-2(1H)-yl)ethan-1-one;
1-(1-(4-chloro-2-fluorophenyl)-7-(trifluoromethyl)-3,4-dihydroimidazo [1,2-a:5,4-c′]dipyridin-2(1H)-yl)-2-((2-ethyl-6-methylpyridin-3-yl)oxy)ethan-1-one;
((1R)-1-(4-chloro-2-fluorophenyl)-7-(trifluoromethyl)-3,10a-dihydroimidazo[1,2-a:5,4-c′]dipyridin-2(1H)-yl)-2-(2-chloro-4-morpholinophenoxy)ethan-1-one;
1-(1-(4-chloro-2-fluorophenyl)-7-(trifluoromethyl)-3,4-dihydroimidazo [1,2-a:5,4-c′]dipyridin-2(1H)-yl)-2-((2-chloro-6-morpholinopyridin-3-yl)oxy)ethan-1-one;
N-(6-chloro-5-(2-(1-(4-chloro-2-fluorophenyl)-7-(trifluoromethyl)-3,4-dihydroimidazo[1,2-a:5,4-c′]dipyridin-2(1H)-yl)-2-oxoethoxy)pyridin-2-yl)methanesulfonamide;
2-(2-chloro-4-(morpholinomethyl)phenoxy)-1-(1-(1-phenyl-1H-pyrazol-4-yl)-7-(trifluoromethyl)-3,4-dihydroimidazo[1,2-a:5,4-c′]dipyridin-2(1H)-yl)ethan-1-one;
2-(2,3-dimethyl-4-(morpholinomethyl)phenoxy)-1-(1-(1-phenyl-1H-pyrazol-4-yl)-7-(trifluoromethyl)-3,4-dihydroimidazo[1,2-a:5,4-c′]dipyridin-2(1H)-yl)ethan-1-one;
1-(1-(benzo[d]thiazol-2-yl)-7-(trifluoromethyl)-3,4-dihydroimidazo [1,2-a:5,4-c′]dipyridin-2(1H)-yl)-2-(naphthalen-2-yloxy)ethan-1-one;
1-(1-(benzo[d]thiazol-2-yl)-7-(trifluoromethyl)-3,4-dihydroimidazo[1,2-a:5,4-c′]dipyridin-2(1H)-yl)-2-(2-chloro-4-(morpholinomethyl)phenoxy)ethan-1-one;
2-(2-chloro-4-(morpholinomethyl)phenoxy)-1-(1-(5-phenylisoxazol-3-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(5-(4-fluorophenyl)isoxazol-3-yl)-7-(trifluoromethyl)-3,4-dihydroimidazo [1,2-a:5,4-c′]dipyridin-2(1H)-yl)ethan-1-one;
2-(naphthalen-2-yloxy)-1-(1-(thieno [2,3-b]pyridin-2-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(thieno [2,3-b]pyridin-2-yl)-7-(trifluoromethyl)-3,4-dihydroimidazo[1,2-a:5,4-c′]dipyridin-2(1H)-yl)ethan-1-one;
1-(1-(4-cyclopropyl-2-fluorophenyl)-7-(trifluoromethyl)-3,4-dihydroimidazo [1,2-a:5,4-c′]dipyridin-2(1H)-yl)-2-((2-ethyl-6-methylpyridin-3-yl)oxy)ethan-1-one;
2-(2-chloro-4-morpholinophenoxy)-1-(1-(4-cyclopropyl-2-fluorophenyl)-7-(trifluoromethyl)-3,4-dihydroimidazo[1,2-a:5,4-c′]dipyridin-2(1H)-yl)ethan-1-one;
1-(1-(4-cyclopropyl-2-fluorophenyl)-7-(trifluoromethyl)-3,4-dihydroimidazo [1,2-a:5,4-c′]dipyridin-2(1H)-yl)-2-((2-ethyl-6-methyl-1-(l1-oxidanyl)-1l4-pyridin-3-yl)oxy)ethan-1-one;
1-(1-(5-methylpyridin-2-yl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(5-methylpyridin-2-yl)-7-(trifluoromethyl)-3,4-dihydroimidazo[1,2-a:5,4-c′]dipyridin-2(1H)-yl)ethan-1-one;
1-(1-(3,5-dimethylisoxazol-4-yl)-7-(trifluoromethyl)-3,4-dihydroimidazo [1,2-a:5,4-c′]dipyridin-2(1H)-yl)-2-(naphthalen-2-yloxy)ethan-1-one;
1-(1-(5-methoxypyridin-2-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(5-methoxypyridin-2-yl)-7-(trifluoromethyl)-3,4-dihydroimidazo[1,2-a:5,4-c′]dipyridin-2(1H)-yl)ethan-1-one;
1-(1-(2-methoxypyrimidin-5-yl)-7-(trifluoromethyl)-3,4-dihydroimidazo[1,2-a:5,4-c′]dipyridin-2(1H)-yl)-2-(naphthalen-2-yloxy)ethan-1-one;
2-(2-chloro-4-(morpholinomethyl)phenoxy)-1-(1-(2-methylpyridin-4-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(5-chloropyridin-2-yl)-7-(trifluoromethyl)-3,4-dihydroimidazo[1,2-a:5,4-c′]dipyridin-2(1H)-yl)ethan-1-one;
2-((2-chloro-6-(oxazol-2-yl)pyridin-3-yl)oxy)-1-(1-(2,4-dimethylthiazol-5-yl)-7-(trifluoromethyl)-3,4-dihydroimidazo[1,2-a:5,4-c′]dipyridin-2(1H)-yl)ethan-1-one;
6-chloro-N-cyclopropyl-5-(2-(1-(2,4-dimethylthiazol-5-yl)-7-(trifluoromethyl)-3,4-dihydroimidazo[1,2-a:5,4-c′]dipyridin-2(1H)-yl)-2-oxoethoxy)-N-methylpicolinamide;
1-(1-(6-methoxypyridin-3-yl)-7-(trifluoromethyl)-3,4-dihydroimidazo [1,2-a:5,4-c′]dipyridin-2(1H)-yl)-2-(naphthalen-2-yloxy)ethan-1-one;
2-(2-chloro-4-(morpholinomethyl)phenoxy)-1-(1-(6-methoxypyridin-3-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(6-ethoxypyridin-3-yl)-7-(trifluoromethyl)-3,4-dihydroimidazo[1,2-a:5,4-c′]dipyridin-2(1H)-yl)ethan-1-one;
2-(2-chloro-4-(morpholinomethyl)phenoxy)-1-(1-(6-(2,2,2-trifluoroethoxy)pyridin-3-yl)-7-(trifluoromethyl)-3,4-dihydroimidazo [1,2-a:5,4-c′]dipyridin-2(1H)-yl)ethan-1-one;
1-(1-(3-hydroxy-4-methoxyphenyl)-7-(trifluoromethyl)-3,4-dihydroimidazo [1,2-a:5,4-c′]dipyridin-2(1H)-yl)-2-(naphthalen-2-yloxy)ethan-1-one;
2-(2,4-dichlorophenoxy)-1-(1-(3,4-dimethoxyphenyl)-7-methyl-3,4-dihydroimidazo[1,2-a:5,4-c′]dipyridin-2(1H)-yl)ethan-1-one;
1-(1-(3,4-dimethoxyphenyl)-7-methyl-3,4-dihydroimidazo[1,2-a:5,4-c′]dipyridin-2(1H)-yl)-2-(naphthalen-2-yloxy)ethan-1-one;
1-(7-(tert-butyl)-1-(3,4-dimethoxyphenyl)-3,4-dihydroimidazo[1,2-a:5,4-c′]dipyridin-2(1H)-yl)-2-(2,4-dichlorophenoxy)ethan-1-one;
2-(2,4-dichlorophenoxy)-1-(1-(3,4-dimethoxyphenyl)-7-ethyl-8-methyl-3,4-dihydroimidazo[1,2-a:5,4-c′]dipyridin-2(1H)-yl)ethan-1-one;
1-(1-(3,4-dimethoxyphenyl)-7-ethyl-8-methyl-3,4-dihydroimidazo [1,2-a:5,4-c′]dipyridin-2(1H)-yl)-2-(naphthalen-2-yloxy)ethan-1-one;
1-(1-(3,4-dimethoxyphenyl)-7-ethyl-8-methyl-3,4-dihydroimidazo [1,2-a:5,4-c′]dipyridin-2(1H)-yl)-2-((1-ethylnaphthalen-2-yl)oxy)ethan-1-one;
1-(1-(3,4-dimethoxyphenyl)-7-ethyl-8-methyl-3,4-dihydroimidazo [1,2-a:5,4-c′]dipyridin-2(1H)-yl)-2-((1-methylnaphthalen-2-yl)oxy)ethan-1-one hydrochloride;
2-(2,4-dichlorophenoxy)-1-(1-(3,4-dimethoxyphenyl)-8-methyl-3,4-dihydroimidazo[1,2-a:5,4-c′]dipyridin-2(1H)-yl)ethan-1-one;
1-(1-(3,4-dimethoxyphenyl)-8-methyl-3,4-dihydroimidazo[1,2-a:5,4-c′]dipyridin-2(1H)-yl)-2-(naphthalen-2-yloxy)ethan-1-one; and
1-(1-(3,4-dimethoxyphenyl)-8-phenyl-3,4-dihydroimidazo[1,2-a:5,4-c′]dipyridin-2(1H)-yl)-2-(naphthalen-2-yloxy)ethan-1-one;
or a pharmaceutically acceptable salt thereof.

12. A pharmaceutical composition comprising, as active principle, one or more compounds according to claim 1, or a pharmaceutically acceptable salt thereof, and at least one therapeutically inert excipient.

13. (canceled)

14. A method for the prevention or treatment of diseases or disorders selected from lung disease including interstitial lung disease, chronic obstructive pulmonary disease, pulmonary embolism, pulmonary hypertension including pulmonary arterial hypertension, radiation pneumonitis, asthma, and adult respiratory distress syndrome; osteoporosis; gastrointestinal disorders including inflammatory bowel disease, postinfectious irritable bowel syndrome, coeliac disease, idiopathic constipation, and irritable bowel syndrome; ulcerative colitis; carcinoid syndrome; myxomatous valve disease; thrombosis; sleep disorders; pain; type 1 and type 2 diabetes; immune disorders; liver disease; acute and chronic hypertension; cancer including breast cancer, prostate cancer, and neuroendocrine tumors with elevated serotonin secretion; subarachnoid hemorrhage; abdominal migraine; CREST syndrome; Gilbert's syndrome; nausea; serotonin syndrome; functional anorectal disorders; functional bloating; and inflammatory diseases including multiple sclerosis and systemic sclerosis, the method comprising administering a compound according to claim 1, or a pharmaceutically acceptable salt thereof, to a subject in need thereof.

15. (canceled)

16. A method to treat a disease or disorder characterized by an altered rate of the tryptophan-serotonin metabolism; comprising administering to a subject in need thereof, the compound of claim 1 in free or pharmaceutically acceptable salt form.
